(19) United States
(12) Patent Application Publication
(10) Pub. No.: US 2009/0156577 A1
(43) Pub. Date: Jun. 18, 2009

(54) 7-AMINO ALKYLIDENYL-HETEROCYCLIC QUINOLONES AND NAPHTHYRIDONES

(76) Inventors: Benjamin Davis, Birmingham, AL (US); Eugene B. Grant, III, Flemington, NJ (US); Shu-Chen Lin, Doylestown, PA (US); Mark J. Macielag, Branchburg, NJ (US); Michele Ann Weidner-Wells, Hillsborough, NJ (US); Xiaoqing Xu, Plainsboro, NJ (US); Xiaodong Xu, Doylestown, PA (US); Bin Zhu, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/268,529

(22) Filed: Nov. 11, 2008

Related U.S. Application Data
(63) Continuation-in-part of application No. 11/084,987, filed on Mar. 21, 2005.
Application No. 11/084,987 is a continuation-in-part of application No. 10/937,238, filed on Sep. 9, 2004, now Pat. No. 7,179,805.

(51) Int. Cl. A61K 31/5365; C07D 471/04; A61K 31/4375; C07D 401/04; A61K 31/4709; C07D 498/16

(52) U.S. Cl. 514/210.21; 546/123; 514/300; 546/178; 514/312; 544/101; 514/230.2

(57) ABSTRACT

The present invention relates to compounds having a structure according to Formula (I)

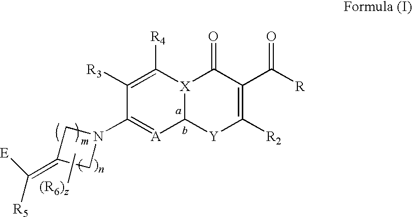

wherein n, m, z, R, R₂, R₃, R₄, R₅, R₆, A, E, X, Y, a and b are as defined above; or an optical isomer, diastereomer or enantiomer thereof; a pharmaceutically acceptable salt, hydrate, or prodrug thereof.

### CROSS-REFERENCE TO RELATED APPLICATION

This application is a continuation-in-part of U.S. patent application Ser. No. 11/084,987, filed Mar. 21, 2005, which is a continuation-in-part of U.S. patent application Ser. No. 10/937,238, filed Sep. 9, 2004 (now U.S. Pat. No. 7,179,805 granted Feb. 20, 2007) and claims benefit of U.S. Provisional Patent Application Ser. No. 60/504,924, filed Sep. 22, 2003, which are each incorporated herein by reference in their entirety and for all purposes.

### FIELD OF INVENTION

The subject invention relates to novel antimicrobial compounds, their compositions and their uses.

### BACKGROUND

The chemical and medical literature describes compounds that are said to be antimicrobial, i.e., capable of destroying or suppressing the growth or reproduction of microorganisms, such as bacteria. For example, such antibacterial agents are described in Antibiotics, Chemotherapeutics, and Antibacterial Agents for Disease Control (M. Greyson, editor, 1982), E. Gale et al., The Molecular Basis of Antibiotic Action 2d edition (1981), Recent Research Developments in Antimicrobial Agents & Chemotherapy (S. G. Pandalai, Editor, 2001), Quinolone Antimicrobial Agents (John S Wolfson, David C Hooper, Editors, 1989), and F. O'Grady, H. P. Lambert, R. G. Finch, D. Greenwood, Martin Dedicoat, “Antibiotic and Chemotherapy, 7th edn.” (1997).

The mechanisms of action of these antibacterial agents vary. However, they are generally believed to function in one or more ways: by inhibiting cell wall synthesis or repair; by altering cell wall permeability; by inhibiting protein synthesis; or by inhibiting the synthesis of nucleic acids. For example, beta-lactam antibacterial agents act through inhibiting essential penicillin binding proteins (PBPs) in bacteria, which are responsible for cell wall synthesis. As another example, quinolones act, at least in part by inhibiting synthesis of DNA, thus preventing the cell from replicating.

The pharmacological characteristics of antimicrobial agents, and their suitability for any given clinical use, vary. For example, the classes of antimicrobial agents (and members within a class) may vary in 1) their relative efficacy against different types of microorganisms, 2) their susceptibility to development of microbial resistance and 3) their pharmacological characteristics such as their bioavailability and biodistribution. Accordingly, selection of an appropriate antimicrobial agent in a given clinical situation requires analysis of many factors, including the type of organism involved, the desired method of administration, the location of the infection to be treated and other considerations.

However, many such attempts to produce improved antimicrobial agents yield equivocal results. Indeed, few antimicrobial agents are produced that are truly clinically acceptable in terms of their spectrum of antimicrobial activity, avoidance of microbial resistance, and pharmacology. Thus, there is a continuing need for broad-spectrum antimicrobial agents, which are effective against resistant microbes.

Some 1,4-dihydroquinolone, naphthyridine or related heterocyclic moieties are known in the art to have antimicrobial activity and are described in the following references: R. Albrecht Prog. Drug Research, Vol. 21, p. 9 (1977); J. Wolfson et al., “The Fluoroquinolones: Structures, Mechanisms of Action and Resistance, and Spectra of Activity In Vitro”, Antimicrob. Agents and Chemother., Vol. 28, p. 581 (1985); G. Klopman et al. Antimicrob. Agents and Chemother., Vol. 31, p. 1831 (1987); M. P. Wentland et al., Ann. Rep. Med. Chem., Vol. 20, p. 145 (1986); J. B. Cornett et al., Ann. Rep. Med. Chem., Vol. 21, p. 139 (1986); P. B. Fernandes et al. Ann. Rep. Med. Chem., Vol. 22, p. 117 (1987); A. Koga, et al. “Structure-Activity Relationships of Antibacterial 6,7- and 7,8-Disubstituted 1-alkyl-1,4-dihydro-4-oxoquinoline-3-carboxylic Acids” J. Med. Chem. Vol. 23, pp. 1358-1363 (1980); J. M. Domagala et al., J. Med. Chem. Vol. 31, p. 991 (1988); T. Rosen et al., J. Med. Chem. Vol. 31, p. 1598 (1988); B. Ledoussal et al., “Non 6-Fluoro Substituted Quinolone Antibacterials: Structure and Activity”, J. Med. Chem. Vol. 35, p. 198-200 (1992); U.S. Pat. No. 6,329,391; A. M Emmerson et al., “The quinolones: Decades of development and use”, J. Antimicrob. Chemother., Vol 51, pp 13-20 (2003); J. Ruiz, “Mechanisms of resistance to quinolones: target alterations, decreased accumulation and DNA gyrase protection” J. Antimicrob. Chemother. Vol. 51, pp 1109-1117 (2003); Y. Kuramoto et al., “A Novel Antibacterial 8-Chloroquinolone with a Distorted Orientation of the N1-(5-Amino-2,4-difluorophenyl) Group” J. Med. Chem. Vol. 46, pp 1905-1917 (2003); Japanese Patent Publication 06263754; European Patent Publication 487030; International Patent Publication WO0248138; International Patent Publication WO9914214; U.S. Patent Publication 2002/0049192; International Patent Publication WO02085886; European Patent Publication 572259; International Patent Publication WO0136408; U.S. Pat. No. 5,677,456; European Patent Publication 362759; U.S. Pat. No. 5,688,791; U.S. Pat. No. 4,894,458; European Patent Publication 677522; U.S. Pat. No. 4,822,801; U.S. Pat. No. 5,256,662; U.S. Pat. No. 5,017,581; European Patent Publication 304087; International Patent Publication WO0136408; International Patent Publication WO02085886; Japanese Patent Publication 01090184; International Patent Publication WO9209579; International Patent Publication WO0185728; European Patent Publication 343524; Japanese Patent Publication 10130241; European Patent Publication 413-455; International Patent Publication WO0209758; International Patent Publication WO0350107; International Patent Publication WO9415933; International Patent Publication WO9222550; Japanese Patent Publication 07300472; International Patent Publication WO0314108; International Patent Publication WO0071541; International Patent Publication WO0031062; and U.S. Pat. No. 5,869,670.

WO03050107 describes a series of dihydroquinolone, naphthyridine and related heterocyclic antibacterial agents. Of particular interest is the disclosure of compounds of the formula,

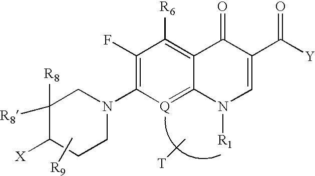

wherein R₈ and R_{8′} are hydrogen, alkyl, substituted alkyl, alkylamino, or arylalkyl, R₉ is hydrogen, alkyl, alkylamino, dialkylamino, aryl, arylalkyl, or trihaloalkyl, and X is hydroxy, alkoxy, acyloxy, amino or substituted amino. European Patent Publication 362759 discloses 1,4-dihydroquinolone and naphthyridine antibacterial agents of the formula,

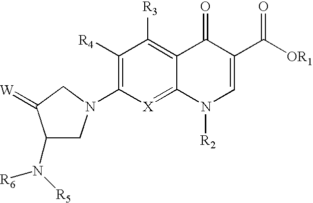

wherein W is C1-3 alkylidene and R₅ and R₆ are hydrogen or alkyl.

International Patent Publication WO 99/14214 and U.S. Pat. No. 6,329,391 disclose quinolone antibacterial agents with C₇-piperidinyl, C₇-azetidinyl, or C₇-pyrrolidinyl substituents of the formula,

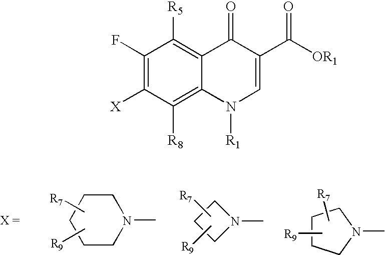

Of particular interest are those compounds wherein R₇ is amino, aminoalkyl, or substituted aminoalkyl and R₉ is selected from hydrogen, C₁-C₄ alkyl, C₂-C₆ alkenyl, C₂-C₆ alkynyl, or a C₃-C₆ fused or spirocyclic alkyl ring. For compounds with a substituted piperidine at the 7-position of the quinolonecarboxylic acid, among the preferred substituents are 3-amino-4-methyl, 3-amino-4,4-dimethyl, 3-amino-4-spirocyclopropyl, 3-amino-6-cyclopropyl, 3-aminomethyl, 4-aminomethyl and 3-methylamino. For compounds with a substituted pyrrolidine at the 7-position of the quinolonecarboxylic acid nucleus, preferred substituents include 3-(1-aminoethyl), 3-aminomethyl, 4-(1-aminoethyl)-2,2-dimethyl, and 2-aminomethyl. For compounds with an azetidine substituent at the 7-position of the quinolonecarboxylic acid, the compounds having the substituents, 3-amino, 3-aminomethyl and 3-(1-amino-1-methyl)ethyl, are included among the preferred examples.

European Patent Publication 241206A2 discloses compounds of the formula,

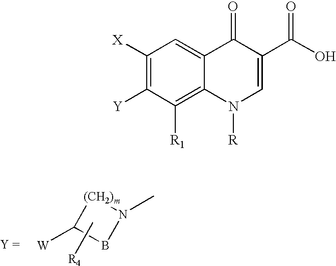

wherein B is —CH₂—, —(CH₂)₂—, or —(CH₂)₃—, R₄ is hydrogen, C₁-C₃ alkyl, hydroxy, or C₁-C₃ alkoxy, W is hydroxy, C₁-C₃ alkoxy, or a group of the formula R₅R₆N—(CH₂)ₙ— in which n is 0 or 1 and R₅ and R₆ are the same or different and each represents a hydrogen atom, a C₁-C₃ alkyl group or an arylalkyl group, and m is 1 or 2. each symbol is as defined in the specification of the above mention publication. For the piperidine substituent at the 7-position of the quinolonecarboxylic acid, the compounds having substituents of 4-amino-3-methyl, 4-methylamino-3-methyl, 4-hydroxy-3-methyl are included in the preferred examples therein.

European Patent Publication 0394553B1 discloses anti-viral compounds of the formula,

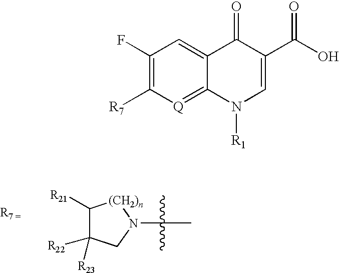

wherein R₂₁, R₂₂ and R₂₃ are each independently is a hydrogen atom, a halogen atom, amino, C₁-C₆ alkyl, C₁-C₈ alkoxy, or amino C₁-C₈ alkyl and two of them may be combined with each other to form a spiro ring, and n is 1 or 2.

European Patent Publication 0572259A1 discloses anti-viral compounds of the formula,

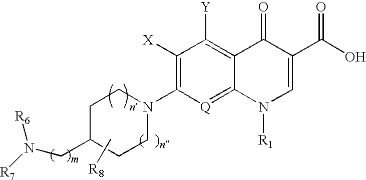

wherein R₆ and R₇ may be the same or different and each represents a hydrogen atom or a lower alkyl group, m is 0 or 1, n′ is 1 or 2, n″ is 1, 2, 3 or 4, and R8 is a hydrogen atom, a lower alkyl group, a hydroxy group or a lower alkoxy group.

International Patent Publication WO9324479 discloses compounds of the formula,

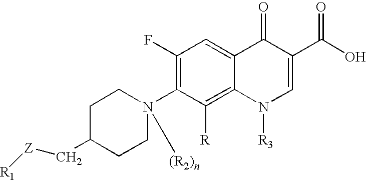

wherein Z is an amino radical, R₁ is hydrogen, an (optionally hydroxylated lower alkyl) radical, an acyl radical derived from a carboxylic acid, an alkyl carbonic acid or an arylsulfonic acid or an arylamino carbonyl radical, R₂ is an oxygen atom, and n is 0 or 1.

Examples of bacterial infections resistant to antibiotic therapy have been reported in the past; they are now a significant threat to public health in the developed world. The development of microbial resistance (perhaps as a result of the intense use of antibacterial agents over extended periods of time) is of increasing concern in medical science. “Resistance” can be defined as existence of organisms, within a population of a given microbial species, that are less susceptible to the action of a given antimicrobial agent. This resistance is of particular concern in environments such as hospitals and nursing homes, where relatively high rates of infection and intense use of antibacterial agents are common. See, e.g., W. Sanders, Jr. et al., “Inducible Beta-lactamases: Clinical and Epidemiologic Implications for the Use of Newer Cephalosporins”, Review of Infectious Diseases, p. 830 (1988).

Pathogenic bacteria are known to acquire resistance via several distinct mechanisms including inactivation of the antibiotic by bacterial enzymes (e.g., β-lactamases hydrolyzing penicillin and cephalosporins); removal of the antibiotic using efflux pumps; modification of the target of the antibiotic via mutation and genetic recombination (e.g., penicillin-resistance in Neiserria gonorrhoeae); and acquisition of a readily transferable gene from an external source to create a resistant target (e.g., methicillin-resistance in Staphylococcus aureus). There are certain Gram-positive pathogens, such as vancomycin-resistant Enterococcus faecium, which are resistant to virtually all commercially available antibiotics.

Hence existing antibacterial agents have limited capacity in overcoming the threat of resistance. Thus it would be advantageous to provide new antibacterial agents that can be used against resistant microbes.

### SUMMARY OF INVENTION

Applicants have found a novel series of quinolones and related compounds that are effective against resistant microbes, and provide significant activity advantages over the art. In particular, the invention relates to compounds having a structure according to Formula (I)
Formula (I)
   wherein:
n is an integer from 1 to 3;
m is an integer from 1 to 3;
z is an integer from 0 to 3;
R is selected from hydrogen, hydroxy, and alkoxy;
R₂ is hydrogen;
R₃ and R₄ are independently selected from hydrogen, halogen, amino, hydroxy, alkoxy, alkylthio, alkyl, alkenyl and alkynyl;
R₅ is selected from hydrogen, halogen, alkyl, aryl, alkoxy, and alkylthio;
R₆ is independently selected from alkyl, hydroxy, alkoxy, alkylthio, alkenyl, alkynyl, aryl, alkoxyimino, and halogen; or R₅ and R₆ join to form a 4- to 7-membered carbocyclic ring wherein each carbon atom of the ring can be optionally substituted with R₁₂, wherein R₁₂ is selected from the group consisting of halogen, amino, hydroxy, alkoxy, alkylthio, alkyl, alkenyl, alkynyl, oxo, alkoxyimino and hydroxyimino;
E is selected from the group consisting of:

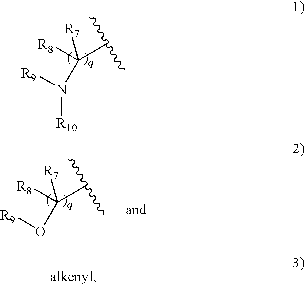

wherein,
q is an integer from 1 to 3;
In the case of 1), R₇ and R₈ are each independently selected from hydrogen or alkyl, or R₇ and R₈ join to form a 3- to 6-membered carbocyclic ring, or either of R₇ or R₈ can be joined independently to either of R₉ or R₁₀ to form a heterocyclic ring containing the nitrogen atom to which R₉ or R₁₀ are bonded, wherein
R₉ and R₁₀ are each independently selected from hydrogen, alkyl, aryl, arylalkyl, acyl, alkoxycarbonyl, or sulfonyl, or alternatively R₉ and R₁₀ join to form a heterocyclic ring containing the nitrogen atom to which they are bonded;
In the case of 2), R₇ and R₈ are each independently selected from hydrogen or alkyl, or R₇ and R₈ join to form a 3- to 6-membered carbocyclic ring, and R₉ is selected from hydrogen, alkyl, acyl, alkoxycarbonyl, or sulfonyl;
A is selected from N and C(R₁₁), wherein R₁₁ is selected from hydrogen, alkyl, halogen, hydroxy, alkoxy, alkylthio or cyano;
X is selected from C and N, where if X is C, then a is a double bond and b is a single bond, and if X is N, then a is a single bond and b is a double bond; and
Y is selected from N(R₁) and C(R₁), with the proviso that when Y is N(R₁), X is C and when Y is C(R₁), X is N; wherein
R₁ is selected from C3 to C6 cycloalkyl, C4 to C6 heterocycloalkyl, alkyl, alkene, a 6-membered aryl and a 6-membered heteroaryl; provided that
if A is C(R₁₁), X is C and Y is N(R₁), then R₁₁ and R₁ can join to form a 6-membered heterocyclic ring optionally substituted by one or more alkyl groups for a corresponding hydrogen atom; or
if A is C(R₁₁), X is C and Y is N(R₁), then R₂ and R₁ can join to form a monocyclic or bicyclic heterocyclic ring optionally substituted by one or more alkyl groups for a corresponding hydrogen atom; or
if A is C(R₁₁), X is C and Y is N(R₁), then R₂ and R can join to form a 5-membered heterocyclic ring optionally substituted by one or more alkyl groups for a corresponding hydrogen atom;
or an optical isomer, diastereomer or enantiomer thereof or a pharmaceutically acceptable salt, hydrate, or prodrug thereof.

In addition, methods of using compounds of the invention as starting materials are also contemplated in this invention.

It has been found that the compounds of this invention, and compositions containing these compounds, are effective antimicrobial agents against a broad range of pathogenic microorganisms with advantages of activity against resistant microbes.

Accordingly, the present invention is also directed to a method of treating a subject having a condition caused by or contributed to by bacterial infection, which comprises administering to said mammal a therapeutically effective amount of the compound of Formula 1.

The present invention is further directed to a method of preventing a subject from suffering from a condition caused by or contributed to by bacterial infection, which comprises administering to the subject a prophylactically effective dose of the pharmaceutical composition of a compound of Formula 1.

### DETAILED DESCRIPTION

The subject invention provides compounds of Formula (I)

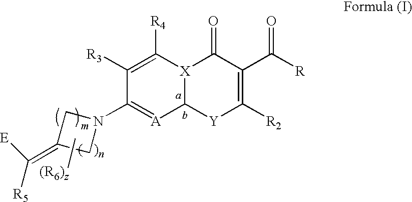

wherein a, b, n, m, z, R, R₂, R₃, R₄, R₅, R₆, A, E, X and Y are as defined in the Summary of the Invention section above.

The invention further relates to compounds having a structure according to Formula (Ia):

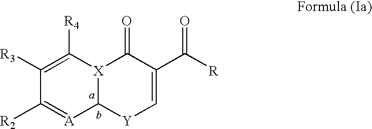

wherein:
R is selected from hydrogen, hydroxy or alkoxy;
R₂ is selected from the group consisting of:

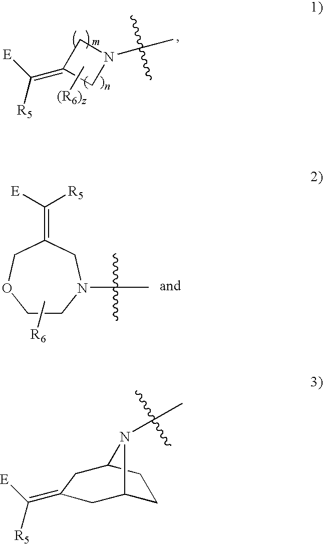

n is an integer selected from 1, 2 or 3;
m is an integer selected from 1, 2 or 3;
z is an integer selected from 0, 1, 2 or 3;
R₃ and R₄ are independently selected from hydrogen, halogen, amino, hydroxy, alkoxy, alkylthio, alkyl, alkenyl or alkynyl;
R₅ is selected from hydrogen, halogen, alkyl, aryl, alkoxy, cyano, CO₂R₁₃ or alkylthio;
R₆ is independently selected from alkyl, hydroxy, alkoxy, alkylthio, alkenyl, alkynyl, aryl, alkoxyimino or halogen, wherein alkyl is optionally mono- or disubstituted on an R₂ carbon atom;
alternatively, R₅ and R₆ join to form a 4- to 7-membered carbocyclic ring wherein each carbon atom of the ring can be optionally substituted with R₁₂;
R₁₂ is selected from the group consisting of halogen, amino, hydroxy, alkoxy, alkylthio, alkyl, alkenyl, alkynyl, oxo, alkoxyimino and hydroxyimino;
R₁₃ is hydrogen, alkyl, aryl, or a protecting group;
E is selected from the group consisting of:

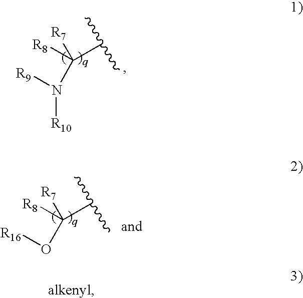

wherein,
q is an integer selected from 1, 2 or 3;
R₇ and R₈ are each independently selected from hydrogen or alkyl;
alternatively, R₇ and R₈ join to form a 3- to 6-membered carbocyclic ring;
alternatively, either of R₇ or R₈ can be joined independently to either of R₉ or R₁₀ to form a heterocyclic ring containing the nitrogen atom to which R₉ or R₁₀ are bonded;
R₉ and R₁₀ are each independently selected from hydrogen, alkyl, acyl, aminoacetyl, arylalkyl, alkoxycarbonyl or sulfonyl;
alternatively, R₉ and R₁₀ can be joined to form a heterocyclic ring containing the nitrogen atom to which they are bonded, wherein the heterocyclic ring is optionally substituted on one or two carbon atoms with oxo;
R₁₆ is selected from hydrogen, alkyl, acyl, alkoxycarbonyl or sulfonyl;
A is selected from N and C(R₁₁);
R₁₁ is selected from hydrogen, alkyl, haloalkyl, halogen, hydroxy, alkoxy, haloalkoxy, alkylthio or cyano;
X is selected from C and N, wherein if X is C, then a is a double bond and b is a single bond, and if X is N, then a is a single bond and b is a double bond;
Y is selected from N(R₁) and C(R₁), with the proviso that when Y is N(R₁), then X is C and when Y is C(R₁), then X is N;
R₁ is selected from an optionally substituted C3 to C6 cycloalkyl, C4 to C6 heterocycloalkyl, alkyl, alkene, 6-membered aryl or 6-membered heteroaryl;
wherein C3 to C6 cycloalkyl, aryl and heteroaryl are each optionally substituted with one, two or three substituents each selected from halogen, alkyl or amino;
provided that if A is C(R₁₁), X is C and Y is N(R₁), then R₁₁ and R₁ can join to form a 6-membered heterocyclic ring substituted with R₁₇;
R₁₇ is selected from hydrogen or alkyl;
or an optical isomer, diastereomer or enantiomer thereof; a pharmaceutically acceptable salt, hydrate, or prodrug thereof.

The invention further relates to compounds having a structure according to Formula (Ia) wherein:
R is hydroxy;
R₂ is selected from the group consisting of:

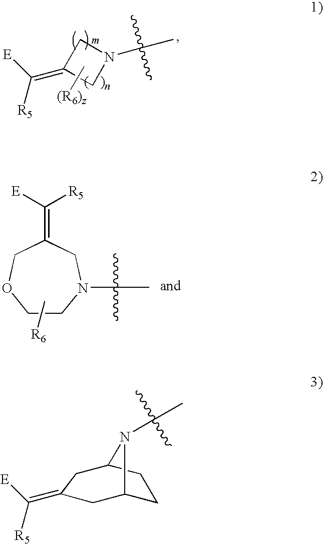

n is an integer selected from 1, 2 or 3;
m is an integer selected from 1 or 2;
z is an integer selected from 0, 1 or 2;
R₃ and R₄ are independently selected from hydrogen, halogen, amino or alkyl;
R₅ is selected from hydrogen, halogen, alkyl or cyano;
R₆ is independently selected from alkyl;
E is selected from the group consisting of:

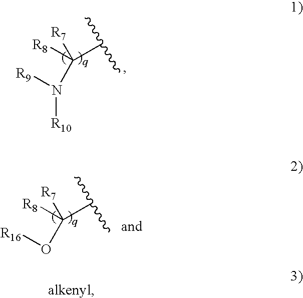

wherein,
q is 1;
R₇ and R₈ are each independently selected from hydrogen or alkyl;
R₉ and R₁₀ are each independently selected from hydrogen or alkyl;
alternatively, R₉ and R₁₀ can be joined to form a heterocyclic ring containing the nitrogen atom to which they are bonded, wherein the heterocyclic ring is optionally substituted on two carbon atoms with oxo;
R₁₆ is hydrogen;
A is selected from N and C(R₁₁);
R₁₁ is selected from hydrogen, alkyl, halogen, alkoxy or haloalkoxy;
X is C, wherein a is a double bond and b is a single bond;
Y is N(R₁);
R₁ is selected from an optionally substituted C₃ to C₆ cycloalkyl, 6-membered aryl or 6-membered heteroaryl;
wherein C₃ to C₆ cycloalkyl, aryl and heteroaryl are each optionally substituted with one, two or three substituents each selected from halogen or amino;
provided that if A is C(R₁₁), X is C and Y is N(R₁), then R₁₁ and R₁ can join to form a 6-membered heterocyclic ring substituted with R₁₇;
R₁₇ is alkyl;
or an optical isomer, diastereomer or enantiomer thereof; a pharmaceutically acceptable salt, hydrate, or prodrug thereof.

An example of the invention includes compounds of Formula (Ia) selected from the group consisting of:

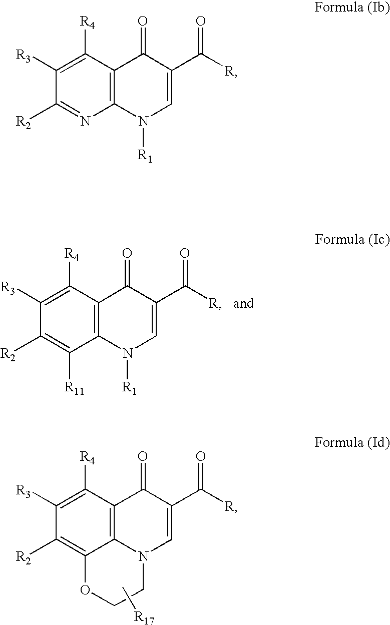

wherein all variables are as previously defined.

An example of the invention includes compounds of Formula (Ia), wherein R₂ is selected from the group consisting of:

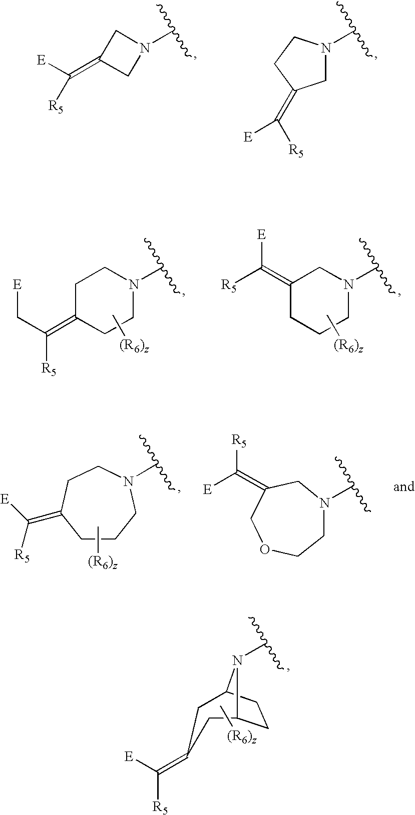

wherein all variables are as previously defined.

Relative to the above description, certain definitions apply as follows.

Unless otherwise noted, under standard nomenclature used throughout this disclosure the terminal portion of the designated side chain is described first, followed by the adjacent functionality toward the point of attachment.

Unless specified otherwise, the terms “alkyl”, “alkenyl”, and “alkynyl,” whether used alone or as part of a substituent group, include straight and branched chains having 1 to 8 carbon atoms, or any number within this range. The term “alkyl” refers to straight or branched chain hydrocarbons. “Alkenyl” refers to a straight or branched chain hydrocarbon with at least one carbon-carbon double bond. “Alkynyl” refers to a straight or branched chain hydrocarbon with at least one carbon-carbon triple bound. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. “Alkoxy” radicals are oxygen ethers formed from the previously described straight or branched chain alkyl groups. “Cycloalkyl” groups contain 3 to 8 ring carbons and preferably 5 to 7 ring carbons.

The alkyl, alkenyl, alkynyl, cycloalkyl group and alkoxy groups may be independently substituted with one or more members of the group including, but not limited to, hydroxyimino, halogen, alkyl, alkenyl, alkynyl, cycloalkyl, alkoxy, oxo, alkoxyimino aryl, heteroaryl, heterocyclo, CN, nitro, —OCOR₁₃, —OR₁₃, —SR₁₃, —SOR₁₃, —SO₂R₁₃, —COOR₁₃, —NR₁₃R₁₄, —CONR₁₃R₁₄, —OCONR₁₃R₁₄, —NHCOR₁₃, —NHCOOR₁₃, and —NHCONR₁₃R₁₄, wherein R₁₃ and R₁₄ are independently selected from hydrogen, alkyl, alkenyl, alkynyl, cycloalkyl, aryl, heteroaryl, heterocyclo, arylalkyl, heteroarylalkyl, and heterocycloalkyl, or alternatively R₁₄ and R₁₅ may join to form a heterocyclic ring containing the nitrogen atom to which they are attached.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

The term “halo” or “halogen” means fluoro, chloro, bromo or iodo. (Mono-, di-, tri-, and per-)halo-alkyl is an alkyl radical substituted by independent replacement of the hydrogen atoms thereon with halogen.

The term “Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 3 of the hydrogen atoms thereon with aryl, heteroaryl, halogen, OH, CN, mercapto, nitro, amino, C₁-C₈-alkyl, C₂-C₈-alkenyl, C₁-C₈-alkoxyl, C₁-C₈-alkylthio, C₁-C₈-alkyl-amino, di (C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl. “Bz” denotes benzoyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms.

Exemplary heteroaryl groups include, for example, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, and thienothienyl. The heteroaryl group may be substituted by independent replacement of 1 to 3 of the hydrogen atoms thereon with aryl, heteroaryl, halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, or carboxamide.

Heteroaryl may be substituted with a mono-oxo to give for example a 4-oxo-1H-quinoline.

The terms “heterocycle,” “heterocyclic,” and “heterocyclo” refer to an optionally substituted, fully saturated, partially saturated, or non-aromatic cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom. The heterocyclic group may be substituted by independent replacement of 1 to 3 of the hydrogen atoms thereon with aryl, heteroaryl, halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, or carboxamide.

Exemplary monocyclic heterocyclic groups include pyrrolidinyl; oxetanyl; pyrazolinyl; imidazolinyl; imidazolidinyl; oxazolinyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydroth iopyranyl; tetrahydroth iopyranyl sulfone; morpholinyl; thiomorpholinyl; thiomorpholinyl sulfoxide; thiomorpholinyl sulfone; 1,3-dioxolane; dioxanyl; thietanyl; thiiranyl; 2-oxazepinyl; azepinyl; and the like.

Exemplary bicyclic heterocyclic groups include quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; benzothiopyranyl; dihydrobenzothiopyranyl; dihydrobenzothiopyranyl sulfone; benzopyranyl; dihydrobenzopyranyl; indolinyl; chromonyl; coumarinyl; isochromanyl; isoindolinyl; piperonyl; tetrahydroquinolinyl; and the like.

The term “carbocyclic” refers to a saturated or unsaturated, non-aromatic, monocyclic, hydrocarbon ring of 3 to 7 carbon atoms.

Substituted aryl, substituted heteroaryl, and substituted heterocycle may also be substituted with a second substituted aryl, a second substituted heteroaryl, or a second substituted heterocycle to give, for example, a 4-pyrazol-1-yl-phenyl or 4-pyridin-2-yl-phenyl.

Designated numbers of carbon atoms (e.g., C₁-C₈ or C₁₋₈) shall refer independently to the number of carbon atoms in an alkyl or cycloalkyl moiety or to the alkyl portion of a larger substituent in which alkyl appears as its prefix root.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2nd edition, John Wiley & Sons, New York (1991), which is incorporated herein by reference. Illustrative hydroxyl protecting groups include but are not limited to tetrahydropyranyl; benzyl; methylthiomethyl; ethylthiomethyl; pivaloyl; phenylsulfonyl; triphenylmethyl; trisubstituted silyl such as trimethylsilyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl; acyl and aroyl such as acetyl, benzoyl, pivaloylbenzoyl, 4-methoxybenzoyl, 4-nitrobenzoyl and arylacyl.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

The phrase “a pharmaceutically acceptable salt” denotes one or more salts of the free base or free acid which possess the desired pharmacological activity of the free base or free acid as appropriate and which are neither biologically nor otherwise undesirable. These salts may be derived from inorganic or organic acids. Examples of inorganic acid salt forms are hydrochloric acid (or hydrochloride salt), nitric acid, hydrobromic acid, sulfuric acid, or phosphoric acid. Examples of organic acid salt forms are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, benzenesulfonic acid, salicylic acid and the like. Suitable salts are furthermore those of inorganic or organic bases, such as KOH, NaOH, Ca(OH)₂, Al(OH)₃, piperidine, morpholine, ethylamine, triethylamine and the like.

Included within the scope of the invention are the hydrated forms of the compounds that contain various amounts of water, for instance, the hydrate, hemihydrate, and sesquihydrate forms. The present invention also includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “subject” includes, without limitation, any animal or artificially modified animal. As a particular embodiment, the subject is a human. The term “drug-resistant” or “drug-resistance” refers to the characteristics of a microbe to survive in the presence of a currently available antimicrobial agent such as an antibiotic at its routine, effective concentration.

The present invention provides compounds of Formula (I) selected from the group consisting of:

| Cpd | Name |
|---|---|
| 1 | 7-[4-(2-Amino-1-fluoro-ethylidene)-piperidin-1-yl]-1-cyclopropyl-6-fluoro-4- |
| | oxo-1,4-dihydro-[1,8]naphthyridine-3-carboxylic acid, |
| 2 | 7-[4-(2-Amino-1-methyl-ethylidene)-piperidin-1-yl]-1-cyclopropyl-6-fluoro- |
| | 4-oxo-1,4-dihydro-[1,8]naphthyridine-3-carboxylic acid, |
| 3 | 7-[4-(2-Amino-1-chloro-ethylidene)-piperidin-1-yl]-1-cyclopropyl-6-fluoro-4- |
| | oxo-1,4-dihydro-[1,8]naphthyridine-3-carboxylic acid, |
| 4 | 7-[4-(2-Amino-1-fluoro-ethylidene)-piperidin-1-yl]-1-(2,4-difluoro-phenyl)-6- |
| | fluoro-4-oxo-1,4-dihydro-[1,8]naphthyridine-3-carboxylic acid, |
| 5 | 7-[4-(2-Amino-1-fluoro-ethylidene)-piperidin-1-yl]-1-cyclopropyl-6-fluoro-4- |
| | oxo-1,4-dihydro-quinoline-3-carboxylic acid, |
| 6 | 7-[4-(2-Amino-1-methyl-ethylidene)-piperidin-1-yl]-1-cyclopropyl-6-fluoro- |
| | 8-methoxy-4-oxo-1,4-dihydro-quinoline-3-carboxylic acid, |
| 7 | 7-[4-(2-Amino-1-chloro-ethylidene)-piperidin-1-yl]-1-cyclopropyl-6-fluoro-8- |
| | methoxy-4-oxo-1,4-dihydro-quinoline-3-carboxylic acid, |
| 8 | 9-[4-(2-Amino-1-fluoro-ethylidene)-piperidin-1-yl]-8-fluoro-3-methyl-6-oxo- |
| | 2,3-dihydro-6H-1-oxa-3a-aza-phenalene-5-carboxylic acid, |
| 9 | 9-[4-(2-Amino-1-methyl-ethylidene)-piperidin-1-yl]-8-fluoro-3-methyl-6- |
| | oxo-2,3-dihydro-6H-1-oxa-3a-aza-phenalene-5-carboxylic acid, |
| 10 | 7-[4-(2-Amino-1-fluoro-ethylidene)-piperidin-1-yl]-1-cyclopropyl-8- |
| | methoxy-4-oxo-1,4-dihydro-quinoline-3-carboxylic acid, |
| 11 | (Z)-7-[4-(2-Amino-1-fluoro-ethylidene)-3-methyl-piperidin-1-yl]-1- |
| | cyclopropyl-6-fluoro-8-methoxy-4-oxo-1,4-dihydro-quinoline-3-carboxylic |
| | acid, |
| 12 | (E)-7-[4-(2-Amino-1-fluoro-ethylidene)-piperidin-1-yl]-1-cyclopropyl-7-6- |
| | fluoro-8-methoxy-4-oxo-1,4-dihydro-quinoline-3-carboxylic acid, |
| 14 | (E)-7-[3-(2-Amino-1-chloro-ethylidene)-pyrrolidin-1-yl]-1-cyclopropyl-6- |
| | fluoro-8-methoxy-4-oxo-1,4-dihydro-quinoline-3-carboxylic acid, |
| 15 | 7-[4-(2-Amino-1-chloro-propylidene)-piperidin-1-yl]-1-cyclopropyl-6-fluoro- |
| | 8-methoxy-4-oxo-1,4-dihydro-quinoline-3-carboxylic acid, |
| 59 | 7-[4-(2-Amino-ethylidene)-piperidin-1-yl]-1-cyclopropyl-6-fluoro-4-oxo-1,4- |
| | dihydro-[1,8]naphthyridine-3-carboxylic acid, |
| 60 | 7-[4-(2-Amino-ethylidene)-3-hydroxy-piperidin-1-yl]-1-cyclopropyl-6-fluoro- |
| | 4-oxo-1,4-dihydro-[1,8]naphthyridine-3-carboxylic acid, |
| 61 | 7-[4-(2-Amino-ethylidene)-3-methoxyimino-piperidin-1-yl]-1-cyclopropyl-6- |
| | fluoro-4-oxo-1,4-dihydro-[1,8]naphthyridine-3-carboxylic acid, |
| 62 | 7-[4-(2-Amino-1-methyl-ethylidene)-3-hydroxy-piperidin-1-yl]-1- |
| | cyclopropyl-6-fluoro-4-oxo-1,4-dihydro-[1,8]naphthyridine-3-carboxylic |
| | acid, |
| 64 | 7-[4-(2-Amino-1-methyl-ethylidene)-piperidin-1-yl]-1-cyclopropyl-6-fluoro- |
| | 4-oxo-1,4-dihydro-quinoline-3-carboxylic acid, |
| 65 | 7-[4-(2-Amino-1-chloro-ethylidene)-piperidin-1-yl]-1-1-cyclopropyl-6-fluoro- |
| | 4-oxo-1,4-dihydro-quinoline-3-carboxylic acid, |
| 66 | 9-[4-(2-Amino-1-methyl-ethylidene)-3-hydroxy-piperidin-1-yl]-1-8-fluoro-3- |
| | methyl-6-oxo-2,3-dihydro-6H-1-oxa-3a-aza-phenalene-5-carboxylic acid, |
| 68 | 7-[4-(2-Amino-1-fluoro-ethylidene)-piperidin-1-yl]-1-cyclopropyl-8- |
| | difluoromethoxy-4-oxo-1,4-dihydro-quinoline-3-carboxylic acid, |
| 69 | 7-[4-(1-Aminomethyl-propylidene)-piperidin-1-yl]-1-cyclopropyl-6-fluoro-4- |
| | oxo-1,4-dihydro-[1,8]naphthyridine-3-carboxylic acid, |
| 70 | 7-[4-(2-Amino-1-methyl-ethylidene)-piperidin-1-yl]-1-cyclopropyl-6-fluoro- |
| | 8-methoxy-4-oxo-1,4-dihydro-quinoline-3-carboxylic acid, |
| 71 | 7-[4-(1-Aminomethyl-propylidene)-piperidin-1-yl]-1-cyclopropyl-6-fluoro-8- |
| | methoxy-4-oxo-1,4-dihydro-quinoline-3-carboxylic acid, |
| 72 | 7-[4-(1-Chloro-2-methylamino-ethylidene)-piperidin-1-yl]-1-cyclopropyl-6- |
| | fluoro-8-methoxy-4-oxo-1,4-dihydroquinoline-3-carboxylic acid, |
| 73 | 7-[4-(2-Amino-1-chloro-ethylidene)-piperidin-1-yl]-1-cyclopropyl-8- |
| | difluoromethoxy-6-fluoro-4-oxo-1,4-dihydro-quinoline-3-carboxylic acid, |
| 74 | 1-Cyclopropyl-8-difluoromethoxy-6-fluoro-7-[4-(1-methyl-2-methylamino- |
| | ethylidene)-piperidin-1-yl]-4-oxo-1,4-dihydro-quinoline-3-carboxylic acid, |
| 75 | 7-[4-(1-Chloro-2-ethylamino-ethylidene)-piperidin-1-yl]-1-cyclopropyl-6- |
| | fluoro-8-methoxy-4-oxo-1,4-dihydro-quinoline-3-carboxylic acid, |
| 76 | 7-[4-(2-Amino-1-chloro-ethylidene)-azetidin-1-yl}-1-cyclopropyl-6-fluoro-4- |
| | oxo-1,4-dihydro-quinoline-3-carboxylic acid, |
| 77 | 9-[3-(2-Amino-1-chloro-ethylidene)-azetidin-1-yl}-8-fluoro-(3S)-3-methyl-6- |
| | oxo-2,3-dihydro-6H-1-oxa-3a-aza-phenalene-5-carboxylic acid, |
| 78 | 7-[4-(2-Amino-1-fluoro-ethylidene)-3,3-dimethyl-piperidin-1-yl]-1- |
| | cyclopropyl-6-fluoro-8-methoxy-4-oxo-1,4-dihydro-quinoline-3-carboxylic |
| | acid, |
| 79 | 1-Cyclopropyl-6-fluoro-7-[4-(1-methyl-2-methylamino-ethylidene)- |
| | piperidin-1-yl]-4-oxo-1,4-dihydro-[1,8]naphthyridine-3-carboxylic acid, |
| 80 | 7-[3-(2-Amino-1-fluoro-ethylidene)-azetidin-1-yl]-1-cyclopropyl-6-fluoro-4- |
| | oxo-1,4-dihydro-[1,8]naphthyridine-3-carboxylic acid, |
| 81 | 7-[3-(2-Amino-1-chloro-ethylidene)-azetidin-1-yl}-1-cyclopropyl-6-fluoro-4- |
| | oxo-1,4-dihydro-[1,8]naphthyridine-3-carboxylic acid, |
| 160 | 7-[3-(2-Amino-1-fluoro-ethylidene)-azetidin-1-yl]-1-cyclopropyl-8- |
| | difluoromethoxy-6-fluoro-4-oxo-1,4-dihydro-quinoline-3-carboxylic acid, |
| 161 | 7-{4-[2-(Benzyl-methyl-amino)-1-chloro-ethylidene]-piperidin-1-yl}-1- |
| | cyclopropyl-6-fluoro-8-methoxy-4-oxo-1,4-dihydro-quinoline-3-carboxylic |
| | acid, |
| 163 | 1-Cyclopropyl-6-fluoro-7-[4-(2-hydroxy-ethylidene)-piperidin-1-yl]-4-oxo- |
| | 1,4-dihydro-[1,8]naphthyridine-3-carboxylic acid, |
| 164 | 1-Cyclopropyl-6-fluoro-7-[4-(2-hydroxy-ethylidene)-piperidin-1-yl]-4-oxo- |
| | 1,4-dihydro-quinoline-3-carboxylic acid, |
| 166 | 7-[4-(1-Chloro-2-isopropylamino-ethylidene)-piperidin-1-yl]-1-cyclopropyl- |
| | 6-fluoro-4-oxo-1,4-dihydro-[1,8]naphthyridine-3-carboxylic acid, |
| 172 | 1-Cyclopropyl-6-fluoro-7-[4-(1-fluoro-2-hydroxy-ethylidene)-piperidin-1-yl]- |
| | 4-oxo-1,4-dihydro-[1,8]naphthyridine-3-carboxylic acid, |
| 173 | 7-[4-(1-Aminomethyl-but-3-enylidene)-piperidin-1-yl]-1-cyclopropyl-6- |
| | fluoro-4-oxo-1,4-dihydro-[1,8]naphthyridine-3-carboxylic acid, |
| 175 | 7-[4-(2-Amino-1-fluoro-ethylidene)-3,3-dimethyl-piperidin-1-yl]-1- |
| | cyclopropyl-6-fluoro-4-oxo-1,4-dihydro-[1,8]naphthyridine-3-carboxylic |
| | acid, |
| 176 | 7-[4-(2-Amino-1-bromo-ethylidene)-3,3-dimethyl-piperidin-1-yl]-1- |
| | cyclopropyl-6-fluoro-4-oxo-1,4-dihydro-[1,8]naphthyridine-3-carboxylic |
| | acid, |
| 177 | 7-{4-[1-Chloro-2-(2-dimethylamino-ethylamino)-ethylidene]-piperidin-1-yl}- |
| | 1-cyclopropyl-6-fluoro-4-oxo-1,4-dihydro-[1,8]naphthyridine-3-carboxylic |
| | acid, |
| 178 | 7-[4-(1-Chloro-allylidene)-piperidin-1-yl]-1-cyclopropyl-6-fluoro-4-oxo-1,4- |
| | dihydro-[1,8]naphthyridine-3-carboxylic acid, |
| 179 | 1-Cyclopropyl-7-[4-(2-ethoxycarbonyloxy-1-fluoro-ethylidene)-piperidin-1- |
| | yl]-6-fluoro-4-oxo-1,4-dihydro-[1,8]naphthyridine-3-carboxylic acid, |
| 180 | 7-[4-(1-Chloro-2-dimethylamino-ethylidene)-piperidin-1-yl]-1-cyclopropyl- |
| | 6-fluoro-4-oxo-1,4-dihydro-[1,8]naphthyridine-3-carboxylic acid, |
| 181 | 7-[4-(1-Chloro-2-piperidin-1-yl-ethylidene)-piperidin-1-yl]-1-cyclopropyl-6- |
| | fluoro-4-oxo-1,4-dihydro-[1,8]naphthyridine-3-carboxylic acid, |
| 182 | 1-Cyclopropyl-7-[4-(2-dimethylamino-1-fluoro-ethylidene)-piperidin-1-yl]-6- |
| | fluoro-4-oxo-1,4-dihydro-[1,8]naphthyridine-3-carboxylic acid, |
| 183 | 7-[3-(2-Amino-1-fluoro-ethylidene)-azetidin-1-yl]-1-(2,4-difluoro-phenyl)-6- |
| | fluoro-4-oxo-1,4-dihydro-[1,8]naphthyridine-3-carboxylic acid, |
| 185 | 1-(2,4-Difluoro-phenyl)-6-fluoro-7-[4-(2-hydroxy-ethylidene)-piperidin-1-yl]- |
| | 4-oxo-1,4-dihydro-[1,8]naphthyridine-3-carboxylic acid, |
| 189 | 7-[3-(2-Amino-1-chloro-ethylidene)-azetidin-1-yl]-1-cyclopropyl-6-fluoro-8- |
| | methoxy-4-oxo-1,4-dihydro-quinoline-3-carboxylic acid, |
| 191 | 7-[4-(1-Chloro-2-dimethylamino-ethylidene)-piperidin-1-yl]-1-cyclopropyl- |
| | 6-fluoro-8-methoxy-4-oxo-1,4-dihydro-quinoline-3-carboxylic acid, |
| 193 | 7-[4-(1-Chloro-2-pyrrolidin-1-yl-ethylidene)-piperidin-1-yl]-1-cyclopropyl-6- |
| | fluoro-8-methoxy-4-oxo-1,4-dihydro-quinoline-3-carboxylic acid, |
| 194 | 7-[4-(1-Chloro-2-piperidin-1-yl-ethylidene)-piperidin-1-yl]-1-cyclopropyl-6- |
| | fluoro-8-methoxy-4-oxo-1,4-dihydro-quinoline-3-carboxylic acid, |
| 195 | 7-[4-(1-Chloro-2-morpholin-4-yl-ethylidene)-piperidin-1-yl]-1-cyclopropyl-6- |
| | fluoro-8-methoxy-4-oxo-1,4-dihydro-quinoline-3-carboxylic acid, |
| 196 | 7-[4-(1-Chloro-2-isopropylamino-ethylidene)-piperidin-1-yl]-1-cyclopropyl- |
| | 6-fluoro-8-methoxy-4-oxo-1,4-dihydro-quinoline-3-carboxylic acid, |
| 199 | 1-Cyclopropyl-7-[4-(2-dimethylamino-1-fluoro-ethylidene)-piperidin-1-yl]-6- |
| | fluoro-8-methoxy-4-oxo-1,4-dihydro-quinoline-3-carboxylic acid, |
| 201 | 7-[4-(1-Aminomethyl-3-hydroxy-propylidene)-piperidin-1-yl]-1-cyclopropyl- |
| | 6-fluoro-8-methoxy-4-oxo-1,4-dihydro-quinoline-3-carboxylic acid, |
| 202 | 7-[4-(1-Aminomethyl-3-methyl-but-3-enylidene)-piperidin-1-yl]-1- |
| | cyclopropyl-6-fluoro-8-methoxy-4-oxo-1,4-dihydro-quinoline-3-carboxylic |
| | acid, |
| 203 | 7-[4-(1-Aminomethyl-but-3-enylidene)-piperidin-1-yl]-1-cyclopropyl-6- |
| | fluoro-8-methoxy-4-oxo-1,4-dihydro-quinoline-3-carboxylic acid, |
| 204 | 7-[4-(2-Amino-1-bromo-ethylidene)-piperidin-1-yl]-1-cyclopropyl-6-fluoro- |
| | 8-methoxy-4-oxo-1,4-dihydro-quinoline-3-carboxylic acid, |
| 205 | 7-[3-(2-Amino-1-chloro-ethylidene)-azetidin-1-yl]-1-cyclopropyl-8- |
| | difluoromethoxy-6-fluoro-4-oxo-1,4-dihydro-quinoline-3-carboxylic acid, |
| 206 | 7-{4-[2-(Benzyl-methyl-amino)-1-chloro-ethylidene]-piperidin-1-yl}-1- |
| | cyclopropyl-8-difluoromethoxy-6- |
| | fluoro-4-oxo-1,4-dihydro-quinoline-3-carboxylic acid |
| 207 | 7-{4-[2-(Benzyl-methyl-amino)-1-fluoro-ethylidene]-piperidin-1-yl}-1- |
| | cyclopropyl-8-difluoromethoxy-6-fluoro-4-oxo-1,4-dihydro-quinoline-3- |
| | carboxylic acid, |
| 208 | 7-[4-(2-Amino-1-fluoro-ethylidene)-piperidin-1-yl]-1-cyclopropyl-6-fluoro-4- |
| | oxo-1,4-dihydro-quinoline-3-carboxylic acid, |
| 209 | 7-[3-(2-Amino-1-chloro-ethylidene)-azetidin-1-yl]-1-cyclopropyl-8- |
| | methoxy-4-oxo-1,4-dihydro-quinoline-3-carboxylic acid, |
| 210 | 9-[4-(2-Amino-ethylidene)-3-hydroxy-piperidin-1-yl]-8-fluoro-3-methyl-6- |
| | oxo-2,3-dihydro-6H-1-oxa-3a-aza-phenalene-5-carboxylic acid, |
| 211 | 8-Fluoro-9-[4-(2-hydroxy-1-methyl-ethylidene)-piperidin-1-yl]-3-methyl-6- |
| | oxo-2,3-dihydro-6H-1-oxa-3a-aza-phenalene-5-carboxylic acid, |
| 212 | 7-[4-(2-Amino-ethylidene)-piperidin-1-yl]-1-cyclopropyl-6-fluoro-8- |
| | methoxy-4-oxo-1,4-dihydro-quinoline-3-carboxylic acid, |
| 213 | 1-Cyclopropyl-6-fluoro-7-[4-(1-fluoro-2-methylamino-ethylidene)-piperidin- |
| | 1-yl]-8-methoxy-4-oxo-1,4-dihydro-quinoline-3-carboxylic acid, |
| 214 | 1-Cyclopropyl-7-[4-(2-ethylamino-1-fluoro-ethylidene)-piperidin-1-yl]-6- |
| | fluoro-8-methoxy-4-oxo-1,4-dihydro-quinoline-3-carboxylic acid, |
| 215 | 7-[4-(1-Chloro-2-methylamino-ethylidene)-piperidin-1-yl]-1-cyclopropyl-6- |
| | fluoro-4-oxo-1,4-dihydro-[1,8]naphthyridine-3-carboxylic acid, |
| 216 | 7-[4-(1-Chloro-2-methylamino-ethylidene)-piperidin-1-yl]-1-cyclopropyl-6- |
| | fluoro-4-oxo-1,4-dihydro-[1,8]naphthyridine-3-carboxylic acid, |
| 217 | 1-Cyclopropyl-6-fluoro-7-[4-(1-methyl-2-methylamino-ethylidene)- |
| | piperidin-1-yl]-4-oxo-1,4-dihydro-[1,8]naphthyridine-3-carboxylic acid, |
| 218 | 1-Cyclopropyl-6-fluoro-7-[4-(1-methyl-2-methylamino-ethylidene)- |
| | piperidin-1-yl]-4-oxo-1,4-dihydro-quinoline-3-carboxylic acid, |
| 219 | 1-Cyclopropyl-6-fluoro-7-[4-(1-methyl-2-methylamino-ethylidene)- |
| | piperidin-1-yl]-4-oxo-1,4-dihydro-quinoline-3-carboxylic acid, |
| 220 | 1-Cyclopropyl-8-difluoromethoxy-6-fluoro-7-[4-(1-methyl-2-methylamino- |
| | ethylidene)-piperidin-1-yl]-4-oxo-1,4-dihydro-quinoline-3-carboxylic acid, |
| 221 | 1-Cyclopropyl-6-fluoro-8-methoxy-7-[4-(2-methylamino-ethylidene)- |
| | piperidin-1-yl]-4-oxo-1,4-dihydro-quinoline-3-carboxylic acid, |
| 222 | 7-[4-(2-Acetylamino-ethylidene)-piperidin-1-yl]-1-cyclopropyl-6-fluoro-4- |
| | oxo-1,4-dihydro-[1,8]naphthyridine-3-carboxylic acid, |
| 247 | 1-Cyclopropyl-6-fluoro-7-[4-(1-fluoro-2-isopropylamino-ethylidene)- |
| | piperidin-1-yl]-4-oxo-1,4-dihydro-[1,8]naphthyridine-3-carboxylic acid, |
| 248 | 7-[4-(1-Chloro-2-isopropylamino-ethylidene)-piperidin-1-yl]-1-cyclopropyl- |
| | 6-fluoro-4-oxo-1,4-dihydro-quinoline-3-carboxylic acid, |
| 249 | 1-Cyclopropyl-6-fluoro-7-[4-(1-fluoro-2-isopropylamino-ethylidene)- |
| | piperidin-1-yl]-4-oxo-1,4-dihydro-quinoline-3-carboxylic acid, |
| 250 | 7-[4-(1-Chloro-2-isopropylamino-ethylidene)-piperidin-1-yl]-1-cyclopropyl- |
| | 8-difluoromethoxy-6-fluoro-4-oxo-1,4-dihydro-quinoline-3-carboxylic acid, |
| 251 | 1-Cyclopropyl-8-difluoromethoxy-6-fluoro-7-[4-(1-fluoro-2-isopropylamino- |
| | ethylidene)-piperidin-1-yl]-4-oxo-1,4-dihydro-quinoline-3-carboxylic acid, |
| 252 | 1-Cyclopropyl-6-fluoro-7-[4-(1-fluoro-2-isopropylamino-ethylidene)- |
| | piperidin-1-yl]-8-methoxy-4-oxo-1,4-dihydro-quinoline-3-carboxylic acid, |
| 253 | 7-[3-(2-Amino-1-fluoro-ethylidene)-piperidin-1-yl]-1-cyclopropyl-6-fluoro-4- |
| | oxo-1,4-dihydro-[1,8]naphthyridine-3-carboxylic acid, |
| 254 | (E)-7-[3-(2-Amino-1-chloro-ethylidene)-piperidin-1-yl]-1-cyclopropyl-6- |
| | fluoro-4-oxo-1,4-dihydro-[1,8]naphthyridine-3-carboxylic acid, |
| 255 | (Z)-7-[3-(2-Amino-1-chloro-ethylidene)-piperidin-1-yl]-1-cyclopropyl-6- |
| | fluoro-4-oxo-1,4-dihydro-[1,8]naphthyridine-3-carboxylic acid, |
| 256 | 7-[3-(2-Amino-1-fluoro-ethylidene)-piperidin-1-yl]-1-cyclopropyl-6-fluoro-4- |
| | oxo-1,4-dihydro-quinoline-3-carboxylic acid, |
| 257 | (E)-7-[3-(2-Amino-1-chloro-ethylidene)-piperidin-1-yl]-1-cyclopropyl-6- |
| | fluoro-4-oxo-1,4-dihydro-quinoline-3-carboxylic acid, |
| 258 | (Z)-7-[3-(2-Amino-1-chloro-ethylidene)-piperidin-1-yl]-1-cyclopropyl-6- |
| | fluoro-4-oxo-1,4-dihydro-quinoline-3-carboxylic acid, |
| 259 | (E)-7-[3-(2-Amino-1-fluoro-ethylidene)-piperidin-1-yl]-1-cyclopropyl-8- |
| | difluoromethoxy-6-fluoro-4-oxo-1,4-dihydro-quinoline-3-carboxylic acid, |
| 260 | (E)-7-[3-(2-Amino-1-chloro-ethylidene)-piperidin-1-yl]-1-cyclopropyl-8- |
| | difluoromethoxy-6-fluoro-4-oxo-1,4-dihydro-quinoline-3-carboxylic acid, |
| 261 | 7-[3-(2-Amino-1-chloro-ethylidene)-piperidin-1-yl]-1-cyclopropyl-6-fluoro-8- |
| | methoxy-4-oxo-1,4-dihydro-quinoline-3-carboxylic acid, |
| 262 | 7-[4-(2-Amino-1-chloro-ethylidene)-piperidin-1-yl]-6-fluoro-(1R,2S)-1-(2- |
| | fluoro-cyclopropyl)-8-methoxy-4-oxo-1,4-dihydro-quinoline-3-carboxylic |
| | acid, |
| 263 | 7-[4-(2-Amino-1-chloro-ethylidene)-piperidin-1-yl]-1-cyclopropyl-6-fluoro-5- |
| | methyl-4-oxo-1,4-dihydro-quinoline-3-carboxylic acid, |
| 267 | 7-[4-(1-Chloro-2-methylamino-ethylidene)-piperidin-1-yl]-1-cyclopropyl-6- |
| | fluoro-8-hydroxy-4-oxo-1,4-dihydro-quinoline-3-carboxylic acid, |
| 268 | 7-[3-(2-Amino-1-chloro-ethylidene)-azetidin-1-yl]-1-(6-amino-3,5-difluoro- |
| | pyridin-2-yl)-8-chloro-6-fluoro-4-oxo-1,4-dihydro-quinoline-3-carboxylic |
| | acid, |
| 269 | 1-(6-Amino-3,5-difluoro-pyridin-2-yl)-7-[3-(2-amino-1-fluoro-ethylidene)- |
| | azetidin-1-yl]-8-chloro-6-fluoro-4-oxo-1,4-dihydro-quinoline-3-carboxylic |
| | acid, |
| 270 | 7-[4-(2-Amino-1-chloro-ethylidene)-piperidin-1-yl]-1-(6-amino-3,5-difluoro- |
| | pyridin-2-yl)-8-chloro-6-fluoro-4-oxo-1,4-dihydro-quinoline-3-carboxylic |
| | acid, |
| 271 | 7-[4-(2-Amino-1-chloro-ethylidene)-piperidin-1-yl]-1-(2,4-difluoro-phenyl)- |
| | 6-fluoro-4-oxo-1,4-dihydro-[1,8]naphthyridine-3-carboxylic acid, |
| 272 | 7-[3-(2-amino-1-fluoro-ethylidene)-azetidin-1-yl]-1-cyclopropyl-6-fluoro-4- |
| | oxo-1,4-dihydro-quinoline-3-carboxylic acid, |
| 273 | 7-[3-(2-amino-1-fluoro-ethylidene)-azetidin-1-yl]-1-cyclopropyl-6-fluoro-8- |
| | methoxy-4-oxo-1,4-dihydro-quinoline-3-carboxylic acid, |
| 274 | 7-[3-(2-amino-1-fluoro-ethylidene)-azetidin-1-yl]-1-cyclopropyl-8-methoxy- |
| | 4-oxo-1,4-dihydro-quinoline-3-carboxylic acid, |
| 275 | 7-[4-(2-Amino-1-chloro-ethylidene)-piperidin-1-yl]-1-cyclopropyl-8- |
| | methoxy-4-oxo-1,4-dihydro-quinoline-3-carboxylic acid, |
| 276 | 9-[3-(2-Amino-1-fluoro-ethylidene)-azetidin-1-yl]-8-fluoro-(3S)-3-methyl-6- |
| | oxo-2,3-dihydro-6H-1-oxa-3a-aza-phenalene-5-carboxylic acid, |
| 277 | 9-[4-(2-Amino-1-chloro-ethylidene)-piperidin-1-yl]-8-fluoro-(3S)-3-methyl- |
| | 6-oxo-2,3-dihydro-6H-1-oxa-3a-aza-phenalene-5-carboxylic acid, |
| 283 | 7-[4-(2-Amino-1-cyano-ethylidene)-piperidin-1-yl]-1-cyclopropyl-6-fluoro-4- |
| | oxo-1,4-dihydro-[1,8]naphthyridine-3-carboxylic acid, |
| 285 | 7-[4-(2-Amino-1-cyano-ethylidene)-piperidin-1-yl]-1-cyclopropyl-6-fluoro-4- |
| | oxo-1,4-dihydro-quinoline-3-carboxylic acid, |
| 286 | 7-[4-(2-Amino-1-carboxy-ethylidene)-piperidin-1-yl]-1-cyclopropyl-6-fluoro- |
| | 4-oxo-1,4-dihydro-[1,8]naphthyridine-3-carboxylic acid, |
| 289 | 7-{4-[2-(2-Amino-ethylamino)-1-chloro-ethylidene]-piperidin-1-yl}-1- |
| | cyclopropyl-6-fluoro-4-oxo-1,4-dihydro-[1,8]naphthyridine-3-carboxylic |
| | acid, |
| 291 | 7-{4-[1-Chloro-2-(4-formyl-piperazin-1-yl)-ethylidene]-piperidin-1-yl}-1- |
| | cyclopropyl-6-fluoro-4-oxo-1,4-dihydro-[1,8]naphthyridine-3-carboxylic |
| | acid, |
| 292 | 7-[4-(1-chloro-2-piperazin-1-yl-ethylidene)-piperidin-1-yl]-1-cyclopropyl-6- |
| | fluoro-4-oxo-1,4-dihydro-[1,8]naphthyridine-3-carboxylic acid, |
| 294 | 1-Cyclopropyl-6-fluoro-7-[4-(1-fluoro-2-piperazin-1-yl-ethylidene)- |
| | piperidin-1-yl]-4-oxo-1,4-dihydro-[1,8]naphthyridine-3-carboxylic acid, |
| 295 | 9-[4-(2-Dimethylamino-1-fluoro-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 297 | (Z)-7-[3-(1-Amino-2-propylidene)-piperidin-1-yl]-1-cyclopropyl-6-fluoro-4- |
| | oxo-1,4-dihydro-[1,8]naphthyridine-3-carboxylic acid, |
| 298 | 7-[4-(1-Chloro-2-dimethylamino-ethylidene)-piperidin-1-yl]-1-cyclopropyl- |
| | 6-fluoro-4-oxo-1,4-dihydro-quinoline-3-carboxylic acid, |
| 299 | 1-Cyclopropyl-7-[4-(2-dimethylamino-1-fluoro-ethylidene)-piperidin-1-yl]-6- |
| | fluoro-4-oxo-1,4-dihydro-quinoline-3-carboxylic acid, |
| 300 | 7-[4-(1-Chloro-2-piperidin-1-yl-ethylidene)-piperidin-1-yl]-1-cyclopropyl-6- |
| | fluoro-4-oxo-1,4-dihydro-quinoline-3-carboxylic acid, |
| 301 | (Z)-7-[3-(2-Amino-1-methyl-ethylidene)-piperidin-1-yl]-1-cyclopropyl-6- |
| | fluoro-4-oxo-1,4-dihydro-quinoline-3-carboxylic acid, |
| 302 | (Z)-7-[3-(2-Amino-1-methyl-ethylidene)-piperidin-1-yl]-1-cyclopropyl-6- |
| | fluoro-8-methoxy-4-oxo-1,4-dihydro-quinoline-3-carboxylic acid, |
| 303 | (E)-7-[3-(2-Amino-1-fluoro-ethylidene)-piperidin-1-yl]-1-cyclopropyl-6- |
| | fluoro-8-methoxy-4-oxo-1,4-dihydro-quinoline-3-carboxylic acid, |
| 356 | 7-[3-(1-Chloro-2-hydroxy-ethylidene)-azetidin-1-yl]-1-cyclopropyl-6-fluoro- |
| | 8-methoxy-4-oxo-1,4-dihydro-quinoline-3-carboxylic acid, |
| 357 | 7-[3-(1-Chloro-2-hydroxy-ethylidene)-azetidin-1-yl]-1-cyclopropyl-6-fluoro- |
| | 4-oxo-1,4-dihydro-[1,8]naphthyridine-3-carboxylic acid, |
| 358 | (E)-1-(6-Amino-3,5-difluoro-pyridin-2-yl)-7-[3-(2-amino-1-fluoro- |
| | ethylidene)-piperidin-1-yl]-8-chloro-6-fluoro-4-oxo-1,4-dihydro-quinoline-3- |
| | carboxylic acid trifluoroacetate, |
| 359 | (Z)-1-(6-Amino-3,5-difluoro-pyridin-2-yl)-8-chloro-6-fluoro-7-[3-(1-fluoro-2- |
| | hydroxy-ethylidene)-piperidin-1-yl]-4-oxo-1,4-dihydroquinoline-3- |
| | carboxylic acid trifluoroacetate, |
| 360 | 1-(6-Amino-3,5-difluoro-pyridin-2-yl)-8-chloro-7-[3-(1-chloro-2- |
| | methylamino-ethylidene)-azetidin-1-yl]-6-fluoro-4-oxo-1,4-dihydro- |
| | quinoline-3-carboxylic acid, |
| 361 | 1-(6-Amino-3,5-difluoro-pyridin-2-yl)-8-chloro-7-[3-(1-chloro-2-hydroxy- |
| | ethylidene)-azetidin-1-yl]-6-fluoro-4-oxo-1,4-dihydro-quinoline-3-carboxylic |
| | acid, |
| 362 | (S)-9-[3-(1-Chloro-2-hydroxy-ethylidene)-azetidin-1-yl]-8-fluoro-3-methyl- |
| | 6-oxo-2,3-dihydro-6H-1-oxa-3a-aza-phenalene-5-carboxylic acid, |
| 363 | 7-[3-(1-Chloro-2-hydroxy-ethylidene)-azetidin-1-yl]-6-fluoro-(1R,2S)-1-(2- |
| | fluoro-cyclopropyl)-8-methoxy-4-oxo-1,4-dihydro-quinoline-3-carboxylic |
| | acid, |
| 364 | 7-[3-(1-Chloro-2-hydroxy-ethylidene)-azetidin-1-yl]-1-cyclopropyl-6-fluoro- |
| | 5-methyl-4-oxo-1,4-dihydro-quinoline-3-carboxylic acid, |
| 365 | 9-[3-(2-Amino-ethylidene)-azetidin-1-yl]-8-fluoro-(3S)-3-methyl-6-oxo-2,3- |
| | dihydro-6H-1-oxa-3a-aza-phenalene-5-carboxylic acid, |
| 366 | 9-[3-(2-Amino-1-methyl-ethylidene)-azetidin-1-yl]-8-fluoro-(3S)-3-methyl- |
| | 6-oxo-2,3-dihydro-6H-1-oxa-3a-aza-phenalene-5-carboxylic acid, |
| 367 | 7-[3-(2-Amino-1-methyl-ethylidene)-azetidin-1-yl]-1-cyclopropyl-6-fluoro-5- |
| | methyl-4-oxo-1,4-dihydro-quinoline-3-carboxylic acid, |
| 368 | 7-[3-(2-Amino-1-methyl-ethylidene)-azetidin-1-yl]-6-fluoro-(1R,2S)-1-(2- |
| | fluoro-cyclopropyl)-8-methoxy-4-oxo-1,4-dihydro-quinoline-3-carboxylic |
| | acid, |
| 369 | 5-Amino-7-[4-(2-amino-1-chloro-ethylidene)-piperidin-1-yl]-1-cyclopropyl- |
| | 6,8-difluoro-4-oxo-1,4-dihydro-quinoline-3-carboxylic acid, |
| 370 | 7-[4-(2-Amino-1-chloro-ethylidene)-piperidin-1-yl]-1-cyclopropyl-6-fluoro-8- |
| | fluoromethoxy-4-oxo-1,4-dihydro-quinoline-3-carboxylic acid, |
| 371 | 7-[4-(2-Amino-1-chloro-ethylidene)-piperidin-1-yl]-1-cyclopropyl-6-fluoro-8- |
| | methyl-4-oxo-1,4-dihydro-quinoline-3-carboxylic acid, |
| 372 | (Z)-9-[4-(2-Amino-1-chloro-ethylidene)-azepan-1-yl]-8-fluoro-(3S)-3- |
| | methyl-6-oxo-2,3-dihydro-6H-1-oxa-3a-aza-phenalene-5-carboxylic acid, |
| 373 | 9-[(E)-4-(2-Amino-1-chloro-ethylidene)-azepan-1-yl]-8-fluoro-(3S)-3- |
| | methyl-6-oxo-2,3-dihydro-6H-1-oxa-3a-aza-phenalene-5-carboxylic acid, |
| 374 | (Z)-7-[3-(2-Amino-1-fluoro-ethylidene)-piperidin-1-yl]-1-cyclopropyl-6- |
| | fluoro-4-oxo-1,4-dihydro-[1,8]naphthyridine-3-carboxylic acid, |
| 375 | (Z)-7-[3-(2-Amino-ethylidene)-piperidin-1-yl]-1-cyclopropyl-6-fluoro-4-oxo- |
| | 1,4-dihydro-[1,8]naphthyridine-3-carboxylic acid, |
| 376 | (E)-7-[3-(2-Amino-ethylidene)-piperidin-1-yl]-1-cyclopropyl-6-fluoro-4-oxo- |
| | 1,4-dihydro-[1,8]naphthyridine-3-carboxylic acid, |
| 377 | (E)-7-[3-(2-Amino-1-methyl-ethylidene)-piperidin-1-yl]-1-cyclopropyl-6- |
| | fluoro-4-oxo-1,4-dihydro-[1,8]naphthyridine-3-carboxylic acid, |
| 378 | (Z)-7-[4-(2-Amino-1-chloro-ethylidene)-azepan-1-yl]-1-cyclopropyl-6- |
| | fluoro-4-oxo-1,4-dihydro-[1,8]naphthyridine-3-carboxylic acid, |
| 379 | (E)-7-[4-(2-Amino-1-chloro-ethylidene)-azepan-1-yl]-1-cyclopropyl-6- |
| | fluoro-4-oxo-1,4-dihydro-[1,8]naphthyridine-3-carboxylic acid, |
| 380 | (Z)-7-[3-(2-Amino-1-methyl-ethylidene)-piperidin-1-yl]-1-(2,4-difluoro- |
| | phenyl)-6-fluoro-4-oxo-1,4-dihydro-[1,8]naphthyridine-3-carboxylic acid, |
| 381 | (E)-7-[3-(2-Amino-1-methyl-ethylidene)-piperidin-1-yl]-1-(2,4-difluoro- |
| | phenyl)-6-fluoro-4-oxo-1,4-dihydro-[1,8]naphthyridine-3-carboxylic acid, |
| 382 | (Z)-7-[3-(2-Amino-1-fluoro-ethylidene)-piperidin-1-yl]-1-cyclopropyl-6- |
| | fluoro-4-oxo-1,4-dihydro-quinoline-3-carboxylic acid, |
| 383 | (Z)-7-[3-(2-Amino-ethylidene)-piperidin-1-yl]-1-cyclopropyl-6-fluoro-4-oxo- |
| | 1,4-dihydro-quinoline-3-carboxylic acid, |
| 384 | (E)-7-[3-(2-Amino-ethylidene)-piperidin-1-yl]-1-cyclopropyl-6-fluoro-4-oxo- |
| | 1,4-dihydro-quinoline-3-carboxylic acid, |
| 385 | (E)-7-[3-(2-Amino-1-methyl-ethylidene)-piperidin-1-yl]-1-cyclopropyl-6- |
| | fluoro-4-oxo-1,4-dihydro-quinoline-3-carboxylic acid, |
| 386 | (Z)-7-[4-(2-Amino-1-chloro-ethylidene)-azepan-1-yl]-1-cyclopropyl-6- |
| | fluoro-4-oxo-1,4-dihydro-quinoline-3-carboxylic acid, |
| 387 | (E)-7-[4-(2-Amino-1-chloro-ethylidene)-azepan-1-yl]-1-cyclopropyl-6- |
| | fluoro-4-oxo-1,4-dihydro-quinoline-3-carboxylic acid, |
| 388 | (Z)-7-[3-(2-Amino-1-fluoro-ethylidene)-piperidin-1-yl]-1-cyclopropyl-6- |
| | fluoro-8-methoxy-4-oxo-1,4-dihydro-quinoline-3-carboxylic acid, |
| 389 | (Z)-7-[3-(2-Amino-ethylidene)-piperidin-1-yl]-1-cyclopropyl--6-fluoro-8- |
| | methoxy-4-oxo-1,4-dihydro-quinoline-3-carboxylic acid, |
| 390 | (E)-7-[3-(2-Amino-ethylidene)-piperidin-1-yl]-1-cyclopropyl-6-fluoro-8- |
| | methoxy-4-oxo-1,4-dihydro-quinoline-3-carboxylic acid, |
| 391 | 7-[3-(2-Amino-1-methyl-ethylidene)-azetidin-1-yl]-1-cyclopropyl-6-fluoro-8- |
| | methoxy-4-oxo-1,4-dihydro-quinoline-3-carboxylic acid, |
| 392 | (E)-7-[3-(2-Amino-1-methyl-ethylidene)-piperidin-1-yl]-1-cyclopropyl-6- |
| | fluoro-8-methoxy-4-oxo-1,4-dihydro-quinoline-3-carboxylic acid, |
| 393 | (Z)-7-[4-(2-Amino-1-chloro-ethylidene)-azepan-1-yl]-1-cyclopropyl-6- |
| | fluoro-8-methoxy-4-oxo-1,4-dihydro-quinoline-3-carboxylic acid, |
| 394 | (E)-7-[4-(2-Amino-1-chloro-ethylidene)-azepan-1-yl]-1-cyclopropyl-6- |
| | fluoro-8-methoxy-4-oxo-1,4-dihydro-quinoline-3-carboxylic acid, |
| 395 | (Z)-7-[3-(2-Amino-1-fluoro-ethylidene)-piperidin-1-yl]-1-cyclopropyl-8- |
| | difluoromethoxy-6-fluoro-4-oxo-1,4-dihydro-quinoline-3-carboxylic acid, |
| 396 | (Z)-7-[3-(2-Amino-ethylidene)-piperidin-1-yl]-1-cyclopropyl-8- |
| | difluoromethoxy-6-fluoro-4-oxo-1,4-dihydro-quinoline-3-carboxylic acid, |
| 397 | (E)-7-[3-(2-Amino-ethylidene)-piperidin-1-yl]-1-cyclopropyl-8- |
| | difluoromethoxy-6-fluoro-4-oxo-1,4-dihydro-quinoline-3-carboxylic acid, |
| 398 | (E)-9-[3-(2-Amino-1-fluoro-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 399 | (Z)-9-[3-(2-Amino-1-fluoro-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 400 | (E)-9-[3-(2-Amino-1-chloro-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 401 | (E)-9-[3-(2-Amino-ethylidene)-piperidin-1-yl]-8-fluoro-(3S)-3-methyl-6-oxo- |
| | 2,3-dihydro-6H-1-oxa-3a-aza-phenalene-5-carboxylic acid, |
| 402 | (Z)-9-[3-(2-Amino-ethylidene)-piperidin-1-yl]-8-fluoro-(3S)-3-methyl-6-oxo- |
| | 2,3-dihydro-6H-1-oxa-3a-aza-phenalene-5-carboxylic acid, |
| 403 | 9-[3-(1-Chloro-2-hydroxy-ethylidene)-azetidin-1-yl]-8-fluoro-(3S)-3-methyl- |
| | 6-oxo-2,3-dihydro-6H-1-oxa-3a-aza-phenalene-5-carboxylic acid, |
| 404 | (Z)-9-[3-(2-Amino-1-methyl-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 405 | (E)-7-[3-(2-Amino-1-fluoro-ethylidene)-piperidin-1-yl]-6-fluoro-(1R,2S)-1- |
| | (2-fluoro-cyclopropyl)-8-methoxy-4-oxo-1,4-dihydro-quinoline-3-carboxylic |
| | acid, |
| 406 | (Z)-7-[3-(2-Amino-1-fluoro-ethylidene)-piperidin-1-yl]-6-fluoro-(1R,2S)-1- |
| | (2-fluoro-cyclopropyl)-8-methoxy-4-oxo-1,4-dihydro-quinoline-3-carboxylic |
| | acid, |
| 407 | (E)-7-[3-(2-Amino-1-chloro-ethylidene)-piperidin-1-yl]-6-fluoro-(1R,2S)-1- |
| | (2-fluoro-cyclopropyl)-8-methoxy-4-oxo-1,4-dihydro-quinoline-3-carboxylic |
| | acid, |
| 408 | (Z)-7-[3-(2-Amino-ethylidene)-piperidin-1-yl]-6-fluoro-(1R,2S)-1-(2-fluoro- |
| | cyclopropyl)-8-methoxy-4-oxo-1,4-dihydro-quinoline-3-carboxylic acid, |
| 409 | (E)-7-[3-(2-Amino-ethylidene)-piperidin-1-yl]-6-fluoro-(1R,2S)-1-(2-fluoro- |
| | cyclopropyl)-8-methoxy-4-oxo-1,4-dihydro-quinoline-3-carboxylic acid, |
| 410 | (Z)-7-[3-(2-Amino-1-methyl-ethylidene)-piperidin-1-yl]-6-fluoro-(1R,2S)-1- |
| | (2-fluoro-cyclopropyl)-8-methoxy-4-oxo-1,4-dihydro-quinoline-3-carboxylic acid, |
| 411 | (Z)-1-(6-Amino-3,5-difluoro-pyridin-2-yl)-7-[3-(2-amino-1-fluoro- |
| | ethylidene)-piperidin-1-yl]-8-chloro-6-fluoro-4-oxo-1,4-dihydro-quinoline-3- |
| | carboxylic acid, |
| 412 | (E)-1-(6-Amino-3,5-difluoro-pyridin-2-yl)-7-[3-(2-amino-1-chloro- |
| | ethylidene)-piperidin-1-yl]8-chloro-6-fluoro-4-oxo-1,4-dihydro-quinoline-3- |
| | carboxylic acid, |
| 413 | (Z)-1-(6-Amino-3,5-difluoro-pyridin-2-yl)-7-[3-(2-amino-ethylidene)- |
| | piperidin-1-yl]8-chloro-6-fluoro-4-oxo-1,4-dihydro-quinoline-3-carboxylic |
| | acid, |
| 414 | (E)-1-(6-Amino-3,5-difluoro-pyridin-2-yl)-7-[3-(2-amino-ethylidene)- |
| | piperidin-1-yl]8-chloro-6-fluoro-4-oxo-1,4-dihydro-quinoline-3-carboxylic |
| | acid, |
| 415 | (E)-1-(6-Amino-3,5-difluoro-pyridin-2-yl)-8-chloro-6-fluoro-7-[3-(1-fluoro-2- |
| | hydroxy-ethylidene)-piperidin-1-yl]-4-oxo-1,4-dihydro-quinoline-3- |
| | carboxylic acid, |
| 416 | (Z)-1-(6-Amino-3,5-difluoro-pyridin-2-yl)-8-chloro-7-[3-(1-chloro-2-hydroxy- |
| | ethylidene)-piperidin-1-yl]-6-fluoro-4-oxo-1,4-dihydro-quinoline-3- |
| | carboxylic acid, |
| 417 | 1-(6-Amino-3,5-difluoro-pyridin-2-yl)-8-chloro-6-fluoro-7-[4-(1-fluoro-2- |
| | hydroxy-ethylidene)-piperidin-1-yl]-4-oxo-1,4-dihydro-quinoline-3- |
| | carboxylic acid, |
| 418 | 1-(6-Amino-3,5-difluoro-pyridin-2-yl)-8-chloro-6-fluoro-7-[3-(1-fluoro-2- |
| | methylamino-ethylidene)-azetidin-1-yl]-4-oxo-1,4-dihydro-quinoline-3- |
| | carboxylic acid, |
| 419 | 1-(6-Amino-3,5-difluoro-pyridin-2-yl)-8-chloro-6-fluoro-7-[3-(1-fluoro-2- |
| | hydroxy-ethylidene)-azetidin-1-yl]-4-oxo-1,4-dihydro-quinoline-3- |
| | carboxylic acid, |
| 420 | 1-(6-Amino-3,5-difluoro-pyridin-2-yl)-7-[3-(2-amino-ethylidene)-azetidin-1- |
| | yl]-8-chloro-6-fluoro-4-oxo-1,4-dihydro-quinoline-3-carboxylic acid, |
| 421 | 1-(6-Amino-3,5-difluoro-pyridin-2-yl)-7-[3-(2-amino-1-methyl-ethylidene)- |
| | azetidin-1-yl]-8-chloro-6-fluoro-4-oxo-1,4-dihydro-quinoline-3-carboxylic |
| | acid, |
| 422 | (Z)-1-(6-Amino-3,5-difluoro-pyridin-2-yl)-7-[3-(2-amino-1-methyl- |
| | ethylidene)-piperidin-1-yl]-8-chloro-6-fluoro-4-oxo-1,4-dihydro-quinoline-3- |
| | carboxylic acid, |
| 423 | (E)-1-(6-Amino-3,5-difluoro-pyridin-2-yl)-7-[3-(2-amino-1-methyl- |
| | ethylidene)-piperidin-1-yl]-8-chloro-6-fluoro-4-oxo-1,4-dihydro-quinoline-3- |
| | carboxylic acid, |
| 424 | (E)-7-[3-(2-Amino-1-fluoro-ethylidene)-piperidin-1-yl]-1-cyclopropyl-6- |
| | fluoro-5-methyl-4-oxo-1,4-dihydro-quinoline-3-carboxylic acid, |
| 425 | (Z)-7-[3-(2-Amino-1-fluoro-ethylidene)-piperidin-1-yl]-1-cyclopropyl-6- |
| | fluoro-5-methyl-4-oxo-1,4-dihydro-quinoline-3-carboxylic acid, |
| 426 | (E)-7-[3-(2-Amino-1-chloro-ethylidene)-piperidin-1-yl]-1-cyclopropyl-6- |
| | fluoro-5-methyl-4-oxo-1,4-dihydro-quinoline-3-carboxylic acid, |
| 427 | (Z)-7-[3-(2-Amino-ethylidene)-piperidin-1-yl]-1-cyclopropyl-6-fluoro-5- |
| | methyl-4-oxo-1,4-dihydro-quinoline-3-carboxylic acid, |
| 428 | (E)-7-[3-(1-Chloro-2-methylamino-ethylidene)-piperidin-1-yl]-1- |
| | cyclopropyl-6-fluoro-4-oxo-1,4-dihydroquinoline-3-carboxylic acid, |
| 430 | 1-Cyclopropyl-6-fluoro-7-[3-(1-fluoro-2-methylamino-ethylidene)-azetidin- |
| | 1-yl]-4-oxo-1,4-dihydro-quinoline-3-carboxylic acid, |
| 431 | 7-[3-(1-Chloro-2-methylamino-ethylidene)-azetidin-1-yl]-1-cyclopropyl-6- |
| | fluoro-4-oxo-1,4-dihydro-quinoline-3-carboxylic acid, |
| 433 | 7-[4-(2-Amino-1-cyano-ethylidene)-piperidin-1-yl]-1-cyclopropyl-6-fluoro-8- |
| | methoxy-4-oxo-1,4-dihydro-quinoline-3-carboxylic acid, |
| 435 | 9-[4-(2-Amino-1-cyano-ethylidene)-piperidin-1-yl]-8-fluoro-(3S)-3-methyl- |
| | 6-oxo-2,3-dihydro-6H-1-oxa-3a-aza-phenalene-5-carboxylic acid, |
| 437 | 7-[4-(2-Amino-1-cyano-ethylidene)-piperidin-1-yl]-1-cyclopropyl-8- |
| | difluoromethoxy-6-fluoro-4-oxo-1,4-dihydro-quinoline-3-carboxylic acid, |
| 439 | 7-[4-(2-Amino-1-cyano-ethylidene)-piperidin-1-yl]-6-fluoro-(1R,2S)-1-(2- |
| | fluoro-cyclopropyl)-8-methoxy-4-oxo-1,4-dihydro-quinoline-3-carboxylic |
| | acid, |
| 441 | (E)-7-[3-(2-Amino-1-cyano-ethylidene)-piperidin-1-yl]-1-cyclopropyl-6- |
| | fluoro-4-oxo-1,4-dihydro-[1,8]naphthyridine-3-carboxylic acid, |
| 443 | (Z)-7-[3-(2-Amino-1-cyano-ethylidene)-piperidin-1-yl]-1-cyclopropyl-6- |
| | fluoro-4-oxo-1,4-dihydro-[1,8]naphthyridine-3-carboxylic acid, |
| 445 | 7-{4-[2-(2-Amino-acetylamino)-1-chloro-ethylidene]-piperidin-1-yl}-1- |
| | cyclopropyl-6-fluoro-8-methoxy-4-oxo-1,4-dihydro-quinoline-3-carboxylic |
| | acid, |
| 446 | (Z)-7-[3-(2-Amino-1-chloro-ethylidene)-piperidin-1-yl]-1-cyclopropyl-6- |
| | fluoro-8-methoxy-4-oxo-1,4-dihydro-quinoline-3-carboxylic acid, |
| 447 | (Z)-7-[3-(2-Amino-1-chloro-ethylidene)-piperidin-1-yl]-1-cyclopropyl-8- |
| | difluoromethoxy-6-fluoro-4-oxo-1,4-dihydro-quinoline-3-carboxylic acid, |
| 448 | (Z)-9-[3-(2-Amino-1-chloro-ethylidene)-piperidin-1-yl]-8-fluoro-(3S)-3- |
| | methyl-6-oxo-2,3-dihydro-6H-1-oxa-3a-aza-phenalene-5-carboxylic acid, |
| 449 | (Z)-7-[3-(2-Amino-1-chloro-ethylidene)-piperidin-1-yl]-6-fluoro-(1R,2S)-1- |
| | (2-fluoro-cyclopropyl)-8-methoxy-4-oxo-1,4-dihydro-quinoline-3-carboxylic |
| | acid, |
| 450 | (Z)-7-[3-(2-Amino-1-chloro-ethylidene)-piperidin-1-yl]-1-(6-amino-3,5- |
| | difluoro-pyridin-2-yl)-8-chloro-6-fluoro-4-oxo-1,4-dihydro-quinoline-3- |
| | carboxylic acid, |
| 451 | (E)-7-[3-(2-Amino-ethylidene)-piperidin-1-yl]-1-cyclopropyl-6-fluoro-5- |
| | methyl-4-oxo-1,4-dihydro-quinoline-3-carboxylic acid, |
| 452 | (Z)-7-[3-(2-Amino-1-chloro-ethylidene)-piperidin-1-yl]-1-cyclopropyl-6- |
| | fluoro-5-methyl-4-oxo-1,4-dihydro-quinoline-3-carboxylic acid, |
| 453 | (E)-1-Cyclopropyl-6-fluoro-7-[3-(1-fluoro-2-methylamino-ethylidene)- |
| | piperidin-1-yl]-8-methoxy-4-oxo-1,4-dihydro-quinoline-3-carboxylic acid, |
| 455 | 7-[4-(2-Amino-1-chloro-ethylidene)-piperidin-1-yl]-1-cyclopropyl-6-fluoro-8- |
| | methoxy-5-methyl-4-oxo-1,4-dihydro-quinoline-3-carboxylic acid, |
| 456 | 5-Amino-7-[(E)-3-(2-amino-1-fluoro-ethylidene)-piperidin-1-yl]-1- |
| | cyclopropyl-6,8-difluoro-4-oxo-1,4-dihydro-quinoline-3-carboxylic acid, |
| 457 | 7-[(E)-3-(2-Amino-1-fluoro-ethylidene)-piperidin-1-yl]-1-cyclopropyl-6- |
| | fluoro-8-fluoromethoxy-4-oxo-1,4-dihydro-quinoline-3-carboxylic acid, |
| 458 | 7-[(E)-3-(2-Amino-1-fluoro-ethylidene)-piperidin-1-yl]-1-cyclopropyl-6- |
| | fluoro-8-methoxy-5-methyl-4-oxo-1,4-dihydro-quinoline-3-carboxylic acid, |
| 459 | 7-[(E)-3-(2-Amino-1-fluoro-ethylidene)-piperidin-1-yl]-1-cyclopropyl-6- |
| | fluoro-8-methyl-4-oxo-1,4-dihydro-quinoline-3-carboxylic acid, |
| 467 | 7-{4-[3-Amino-1-fluoro-propylidene]-piperidin-1-yl}-1-cyclopropyl-6-fluoro- |
| | 8-methoxy-4-oxo-1,4-dihydro-quinoline-3-carboxylic acid, |
| 491 | (3S)-9-[(Z)-4-(2-Amino-1-fluoro-ethylidene)-azepan-1-yl]-8-fluoro-3- |
| | methyl-6-oxo-2,3-dihydro-6H-1-oxa-3a-aza-phenalene-5-carboxylic acid, |
| 492 | (3S)-9-[(E)-4-(2-Amino-1-fluoro-ethylidene)-azepan-1-yl]-8-fluoro-3- |
| | methyl-6-oxo-2,3-dihydro-6H-1-oxa-3a-aza-phenalene-5-carboxylic acid, |
| 493 | 9-[(Z)-6-(2-Amino-1-fluoro-ethylidene)-[1,4]oxazepan-4-yl]-8-fluoro-(3S)-3- |
| | methyl-6-oxo-2,3-dihydro-6H-1-oxa-3a-aza-phenalene-5-carboxylic acid, |
| 494 | 9-[(E)-6-(2-Amino-1-fluoro-ethylidene)-[1,4]oxazepan-4-yl]-8-fluoro-(3S)-3- |
| | methyl-6-oxo-2,3-dihydro-6H-1-oxa-3a-aza-phenalene-5-carboxylic acid, |
| 495 | (Z)-7-[4-(2-Amino-1-fluoro-ethylidene)-azepan-1-yl]-1-cyclopropyl-6-fluoro- |
| | 4-oxo-1,4-dihydro-[1,8]naphthyridine-3-carboxylic acid, |
| 496 | (E)-7-[4-(2-Amino-1-fluoro-ethylidene)-azepan-1-yl]-1-cyclopropyl-6- |
| | fluoro-4-oxo-1,4-dihydro-[1,8]naphthyridine-3-carboxylic acid, |
| 497 | (Z)-7-[6-(2-Amino-1-fluoro-ethylidene)-[1,4]oxazepan-4-yl]-1-cyclopropyl- |
| | 6-fluoro-4-oxo-1,4-dihydro-[1,8]naphthyridine-3-carboxylic acid, |
| 498 | (E)-7-[6-(2-Amino-1-fluoro-ethylidene)-[1,4]oxazepan-4-yl]-1-cyclopropyl- |
| | 6-fluoro-4-oxo-1,4-dihydro-[1,8]naphthyridine-3-carboxylic acid, |
| 499 | (Z)-7-[6-(2-Amino-1-fluoro-ethylidene)-(2S)-2-methyl-[1,4]oxazepan-4-yl]- |
| | 1-cyclopropyl-6-fluoro-4-oxo-1,4-dihydro-[1,8]naphthyridine-3-carboxylic |
| | acid, |
| 500 | (Z)-7-[6-(2-Amino-1-fluoro-ethylidene)-(2R)-2-methyl-[1,4]oxazepan-4-yl]- |
| | 1-cyclopropyl-6-fluoro-4-oxo-1,4-dihydro-[1,8]naphthyridine-3-carboxylic |
| | acid, |
| 501 | (Z)-7-[4-(2-Amino-1-fluoro-ethylidene)-azepan-1-yl]-1-cyclopropyl-6-fluoro- |
| | 4-oxo-1,4-dihydro-quinoline-3-carboxylic acid, |
| 502 | (E)-7-[4-(2-Amino-1-fluoro-ethylidene)-azepan-1-yl]-1-cyclopropyl-6- |
| | fluoro-4-oxo-1,4-dihydro-quinoline-3-carboxylic acid, |
| 503 | (Z)-7-[6-(2-Amino-1-fluoro-ethylidene)-[1,4]oxazepan-4-yl]-1-cyclopropyl- |
| | 6-fluoro-4-oxo-1,4-dihydro-quinoline-3-carboxylic acid, |
| 504 | (E)-7-[6-(2-Amino-1-fluoro-ethylidene)-[1,4]oxazepan-4-yl]-1-cyclopropyl- |
| | 6-fluoro-4-oxo-1,4-dihydro-quinoline-3-carboxylic acid, |
| 505 | (Z)-7-[4-(2-Amino-1-fluoro-ethylidene)-azepan-1-yl]-1-cyclopropyl-6-fluoro- |
| | 8-methoxy-4-oxo-1,4-dihydro-quinoline-3-carboxylic acid, |
| 506 | (E)-7-[4-(2-Amino-1-fluoro-ethylidene)-azepan-1-yl]-1-cyclopropyl-6- |
| | fluoro-8-methoxy-4-oxo-1,4-dihydro-quinoline-3-carboxylic acid, |
| 507 | (Z)-7-[6-(2-Amino-1-fluoro-ethylidene)-[1,4]oxazepan-4-yl]-1-cyclopropyl- |
| | 6-fluoro-8-methoxy-4-oxo-1,4-dihydro-quinoline-3-carboxylic acid, and |
| 508 | (E)-7-[6-(2-Amino-1-fluoro-ethylidene)-[1,4]oxazepan-4-yl]-1-cyclopropyl- |
| | 6-fluoro-8-methoxy-4-oxo-1,4-dihydro-quinoline-3-carboxylic acid. |

Table 1 contains a non-limiting list of preferred compounds of Formula (I).

TABLE 1

| Structure | R5 Substituent | Compound Number |
|---|---|---|
| 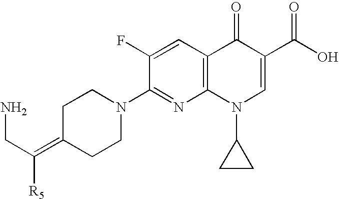 | F CH₃ Cl CH₃CH₂ | 1 2 3 69 |
| 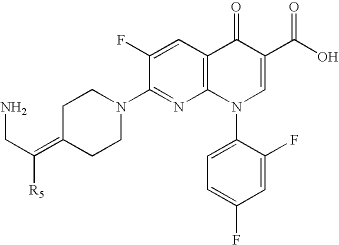 | F | 4 |
| 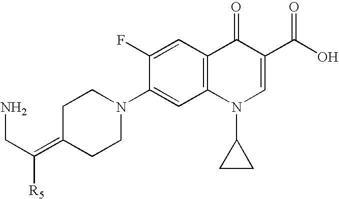 | F Cl | 5 65 |
| 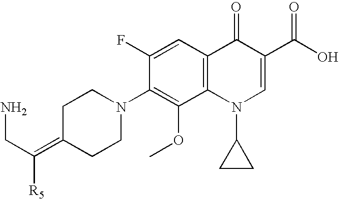 | F Cl CH₃ CH₃CH₂ CN | 6 7 70 71 433 |
| 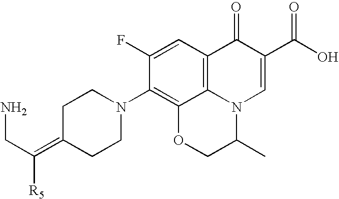 | F CH₃ | 8 9 |
| 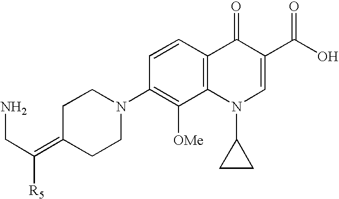 | F | 10 |
| 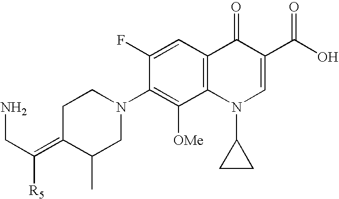 | F | 11 |
| 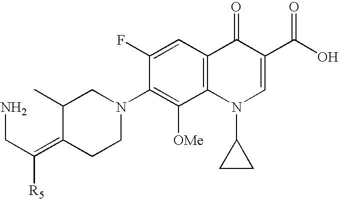 | F | 12 |
| 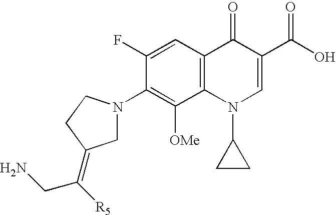 | Cl | 13 |
| 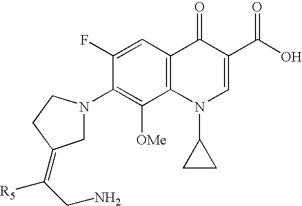 | Cl | 14 |
| 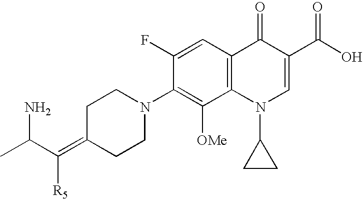 | Cl | 15 |
| 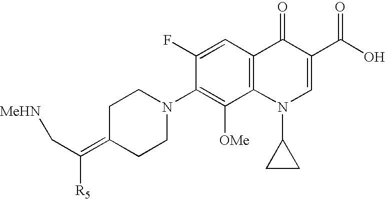 | Cl | 72 |
| 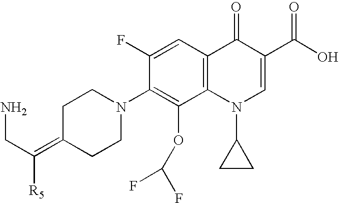 | Cl CN | 73 437 |
| 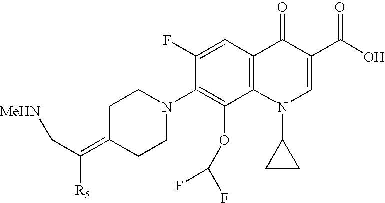 | Cl | 74 |
| 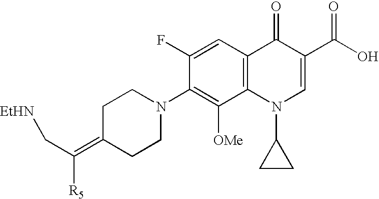 | Cl | 75 |
| 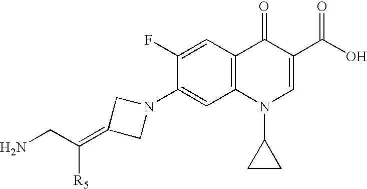 | Cl | 76 |
| 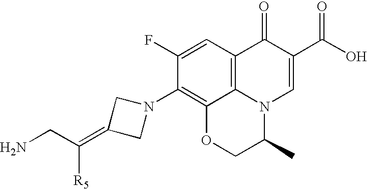 | Cl | 77 |
| 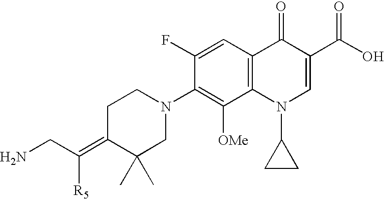 | F | 78 |
| 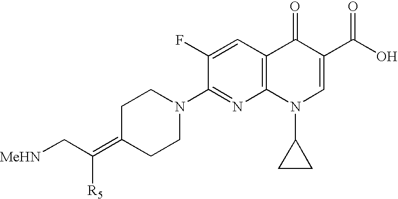 | F | 79 |
| 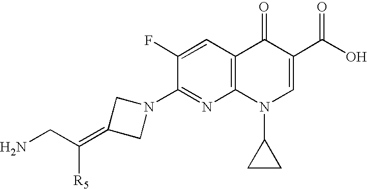 | F Cl | 80 81 |
| 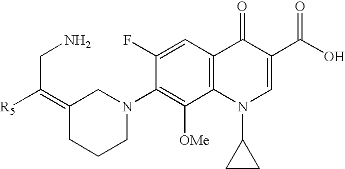 | F Cl CH₃ H | 303 261 302 389 |
| 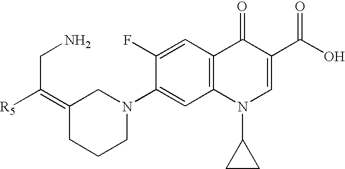 | Cl | 257 |
| 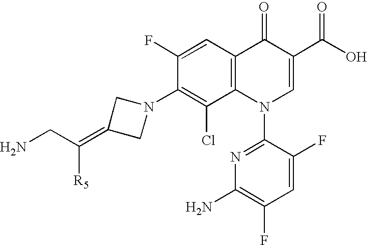 | F Cl H | 269 268 420 |
| 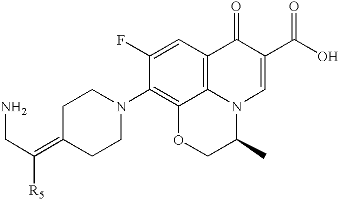 | Cl | 277 |
| 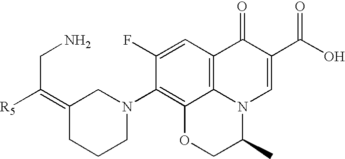 | F Cl | 398 400 |
| 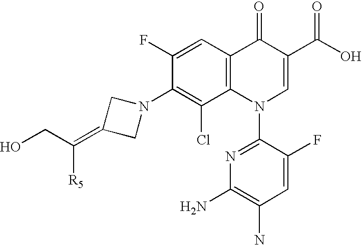 | Cl F | 361 419 |
| 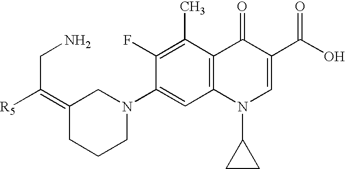 | Cl F | 426 424 |
| 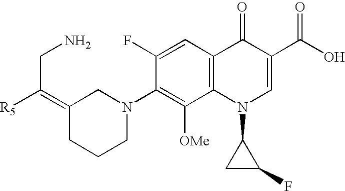 | Cl F CH₃ | 407 405 410 |
| 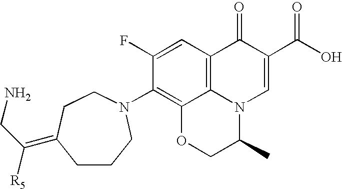 | Cl | 373 |
| 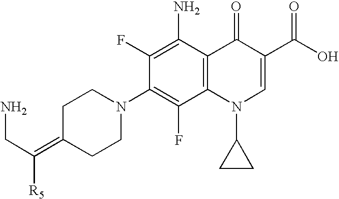 | Cl | 369 |
| 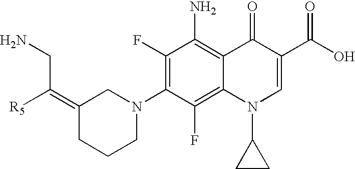 | F | 456 |
| 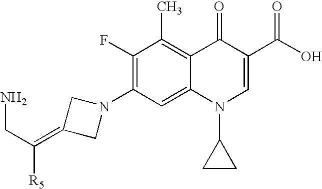 | CH₃ | 367 |
| 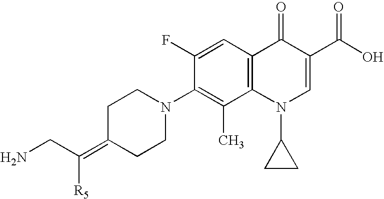 | Cl F | 371 459 |
| 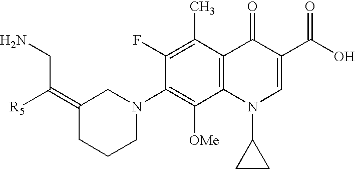 | F | 458 |

### General Reaction Scheme for Compound Preparation

In making the compounds of the invention, the order of synthetic steps may be varied to increase the yield of desired product. In addition, the skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

The starting materials used in preparing the compounds of the invention are known, made by published synthetic methods or available from commercial vendors.

It is recognized that the skilled artisan in the art of organic chemistry can readily carry out standard manipulations of the organic compounds without further direction; that is, it is well within the scope and practice of the skilled artisan to carry out such manipulations. These include, but are not limited to, reductions of carbonyl compounds to their corresponding alcohols, oxidations, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2), Feiser & Feiser, Reagents for Organic Synthesis (16 volumes), L. Paquette, Encyclopedia of Reagents for Organic Synthesis (8 volumes), Frost & Fleming, Comprehensive Organic Synthesis (9 volumes) and the like.

The skilled artisan will readily appreciate that certain reactions are best carried out when certain functional groups are masked or protected in the molecule, thus avoiding any undesirable side reactions and/or increasing the yield of the reaction. Often the skilled artisan utilizes protecting groups to obtain such increased yields or to avoid such undesired reactions. Examples of these manipulations can be found, for example, in T. Greene, Protecting Groups in Organic Synthesis.

General procedures for preparing heterocyclic nuclei useful in making the compounds of the invention are described in the following references, all incorporated by reference herein (including articles listed within the references): U.S. Pat. No. 6,329,391, European Patent Publication 342849, International Patent Publication WO9711068, European Patent Publication 195316, European Patent Publication 1031569, U.S. Pat. No. 6,025,370, European Patent Publication 153828, European Patent Publication 191451, European Patent Publication 153163, European Patent Publication 230053, European Patent Publication 976749, International Patent Publication WO0118005, International Patent Publication WO9407873, U.S. Pat. No. 4,777,253, European Patent Publication 421668, International Patent Publication WO0248138, European Patent Publication 230295, International Patent Publication WO9914214, U.S. Patent Publication 20020049223, International Patent Publication WO9921849, International Patent Publication WO9729102, International Patent Publication WO0334980, International Patent Publication WO0209758, International Patent Publication WO9619472, German Patent Publication DE 3142854, International Patent Publication WO0334980, International Patent Publication WO0328665, European Patent Publication 47005, International Patent Publication WO0311450, and European Patent Publication 688772.

The compounds of the subject invention may be prepared in several ways. Versatile methodologies for preparation of the compounds of the invention are shown in Scheme I below, where L is a leaving group such as fluoro or chloro:

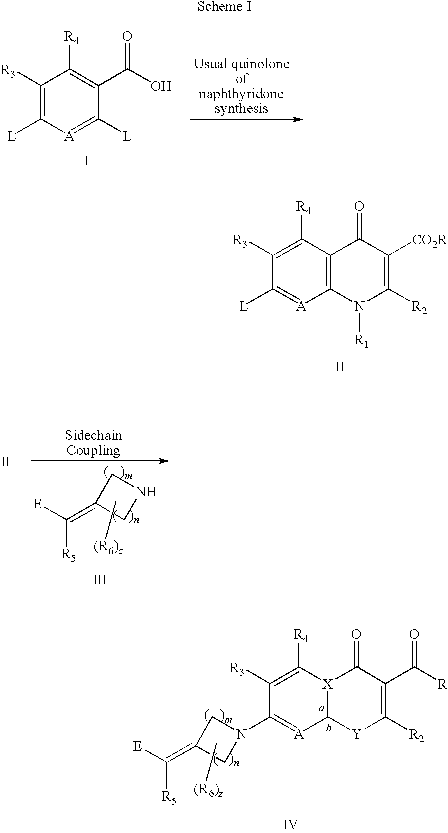

Scheme Ia shows a preparation of the compounds of the invention of formula (Ia) in the case wherein X is C, Y is N(R₁) and R₂ is:

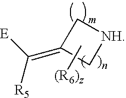

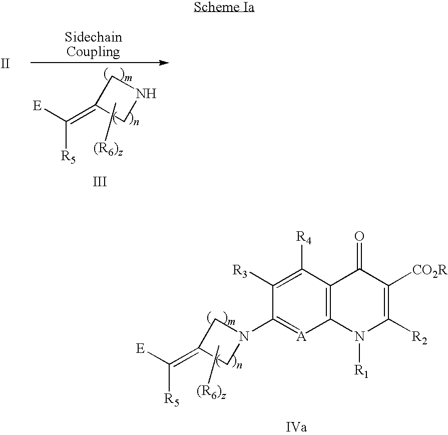

Scheme II shows a preparation of the compounds of the invention in the case where E is:

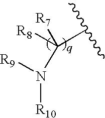

and at least one of R₉ and R₁₀ is hydrogen, wherein it may be necessary to protect the terminal nitrogen to effect selective conversion to the desired product.

In such case, standard amine protecting groups known to those skilled in the art, such as t-butoxycarbonyl (Boc), benzyloxycarbonyl (Cbz), benzyl (Bn), 9-fluorenylmethoxycarbonyl (Fmoc), allyloxycarbonyl (Alloc), 2-trimethylsilylethoxycarbonyl (Teoc), N-formyl, N-acetyl, N-benzoyl, or phthalimide, may be used to mask the terminal amine, as in compound V.

Following side chain coupling, the protecting group may be removed under standard conditions known to those skilled in the art to obtain the desired product VII. VII may be further elaborated, for example by alkylation, to other compounds of the invention VII.

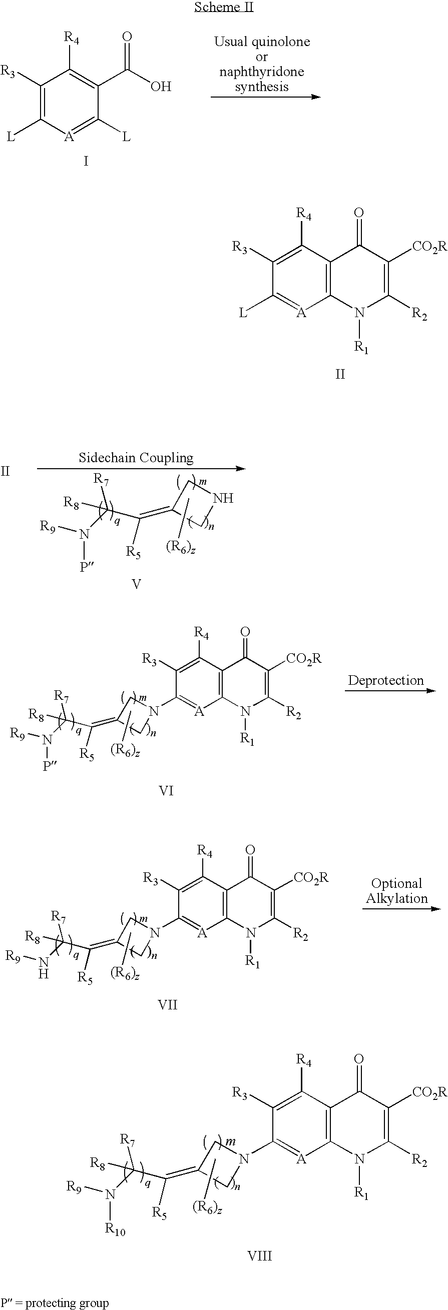

Methodologies for providing the compounds of the invention where X is N and Y is C(R₁) are shown in Scheme III below:

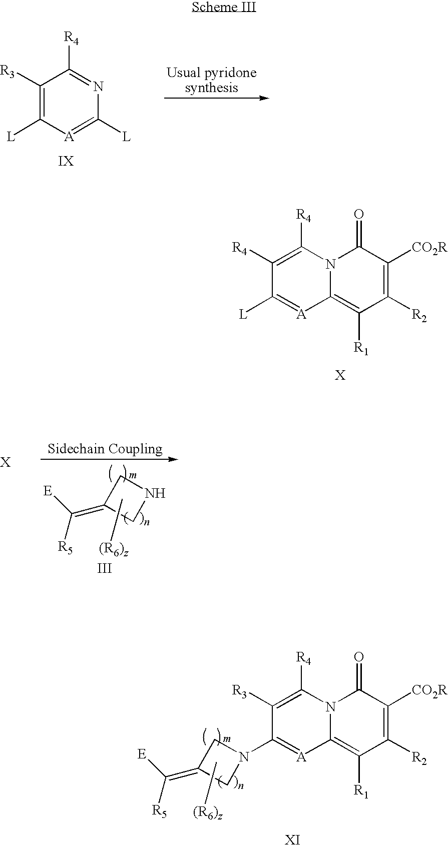

Scheme IV shows a preparation of the compounds of the invention in the case where E is

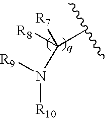

and at least one of R₉ and R₁₀ is hydrogen, wherein it may be necessary to protect the terminal nitrogen to effect selective conversion to the desired product.

In such case, standard amine protecting groups known to those skilled in the art, such as t-butoxycarbonyl (Boc), benzyloxycarbonyl (Cbz), benzyl (Bn), 9-fluorenylmethoxycarbonyl (Fmoc), allyloxycarbonyl (Alloc), 2-trimethylsilylethoxycarbonyl (Teoc), N-formyl, N-acetyl, N-benzoyl, or phthalimide, may be used to mask the terminal amine, as in compound V. Following side chain coupling, the protecting group may be removed under standard conditions known to those skilled in the art to obtain the desired product XIII. XIII may be further elaborated, for example by alkylation, to other compounds of the invention XIV.

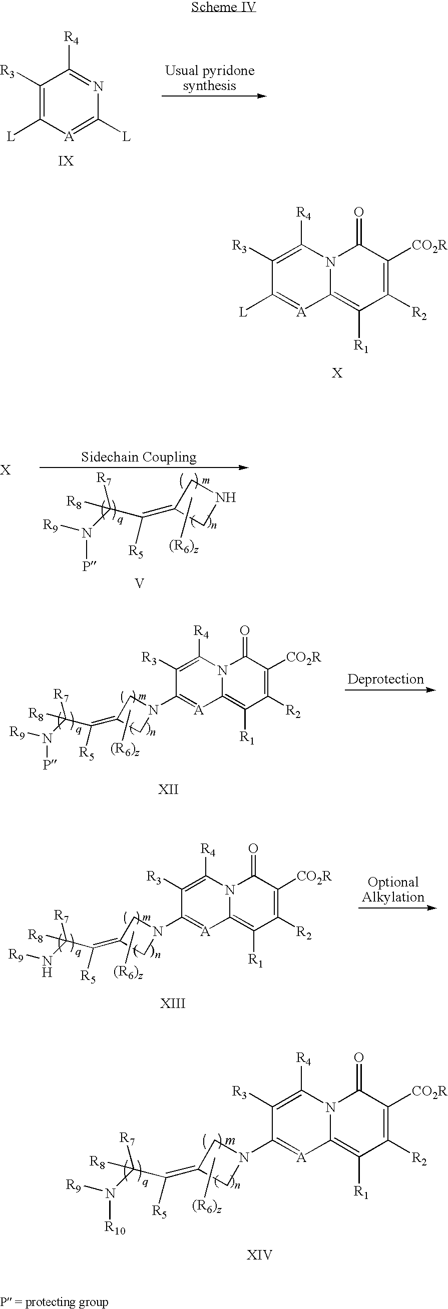

In the case where E is:

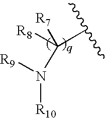

and both R₉ and R₁₀ are hydrogen, selective alkylation of the side chain amine of LXI may also be accomplished by protection of the amine with a standard protecting group, such as Boc, utilizing reagents and conditions apparent to those skilled in the art to give LXII (Scheme XXXVI). The protected amine (LXII) is then treated with an excess (>2 equivalents) of a base, such as, but not limited to, sodium hydride, in an appropriate inert solvent, such as dimethylformamide or tetrahydrofuran, followed by the appropriate alkylating agent R₉X to yield the boc-protected secondary amine as the corresponding ester LXIII. Typically, the reaction is run at temperatures ranging from −20° C. to 60° C. for from 1 to 48 hours depending on the reactivity of the alkylating agent. Typical alkylating agents including alkyl iodides (such as methyl iodide), alkyl bromides and alkyl sulfonates. The ester LXIII may be hydrolyzed under basic conditions to afford the corresponding carboxylic acid LXIV. Ester hydrolysis may be conducted by methods familiar to those skilled in the art, in particular, by employing a base such as an alkali metal hydroxide (for example, sodium hydroxide) or an alkali metal carbonate in a suitable solvent, such as water, methanol, ethanol, or aqueous alcohol mixtures, at a temperature ranging from 20° C. to 100° C. for from 1 to 48 hours. Removal of the amine-protecting group under conditions apparent to one skilled in the art affords the secondary amine LXV. In the case where the protecting group is Boc, for example, reagents such as with trifluoroacetic acid, optionally with methylene chloride as co-solvent, or hydrochloric acid in dioxane, may be used for deprotection.

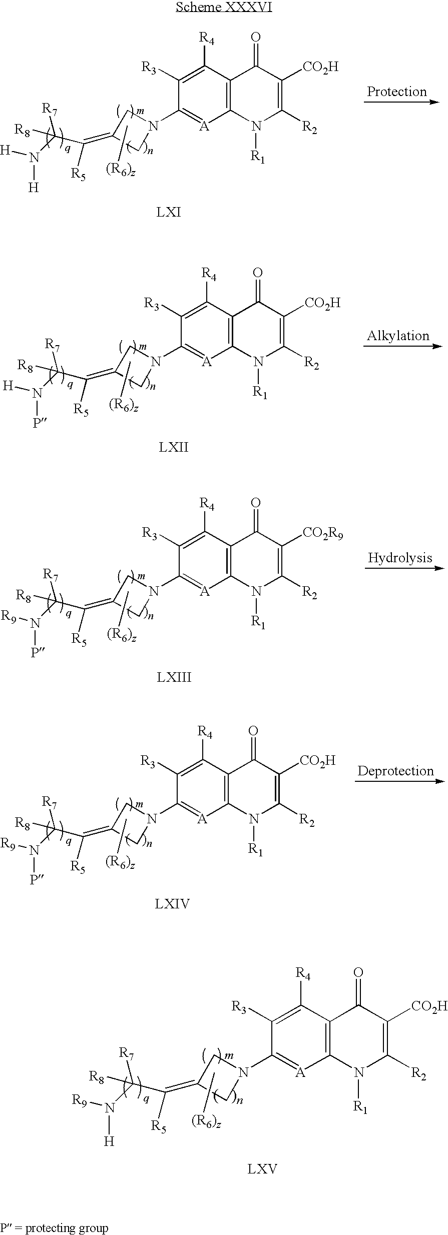

In the case where E is:

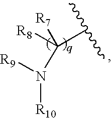

wherein, R₇, R₈ and R₉ are hydrogen, and R₁₀ is either hydrogen or alkyl, compounds of the instant invention may be converted to other compounds of the instant invention wherein R₉ is acyl, alkoxycarbonyl, or sulfonyl (LXXIV) (Scheme LIII).

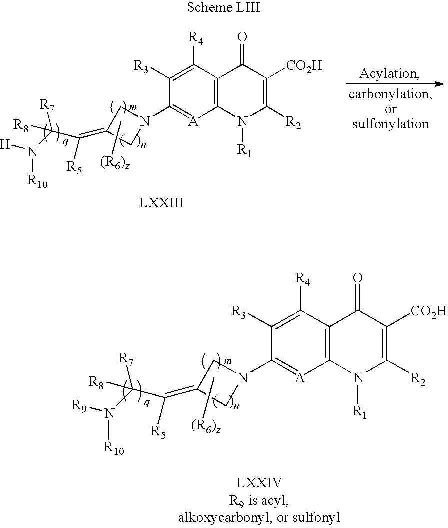

Reaction of amine LXXIII with an acylating agent optionally in the presence of an amine base, such as triethylamine or diisopropylethylamine, in an inert solvent such as dichloromethane, tetrahydrofuran or toluene at temperatures ranging from −20° C. to 60° C. for from 1-48 hours provides compounds of formula LXXIV, wherein E is:

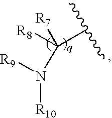

wherein, R₇, and R₈ are hydrogen, R₉ is acyl and R₁₀ is hydrogen or alkyl. Acylating agents include acid halides, acid anhydrides, and acids in the presence of an activating agent such as dicyclohexylcarbodiimide, EDCI, BOP—Cl, BOP, PyBOP, and the like.

Reaction of amine LXXIII with a carbonylating agent in the presence of an amine base, such as such as triethylamine or diisopropylethylamine, in an inert solvent such as dichloromethane, tetrahydrofuran or toluene at temperatures ranging from −20° C. to 60° C. for from 1-48 hours provides compounds of formula LXXIV, wherein E is:

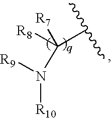

wherein, R₇ and R₈ are hydrogen, R₉ is alkoxycarbonyl and R₁₀ is hydrogen or alkyl. Carbonylating agents include chloroformates, fluoroformates, azidoformates, and pyrocarbonates.

Reaction of amine LXXIII with a sulfonyl chloride or sulfonic anhydride in the presence of an amine base, such as triethylamine or diisopropylethylamine, in an inert solvent such as dichloromethane, tetrahydrofuran or toluene at temperatures ranging from −20° C. to 60° C. for from 1-48 hours provides compounds of formula LXXIV, wherein E is:

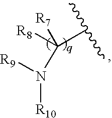

wherein, R₇, and R₈ are hydrogen, R₉ is sulfonyl and R₁₀ is hydrogen or alkyl.

Occasionally, side chain amines are insufficiently reactive to add efficiently to the heterocyclic nuclei (II or X) under the conditions illustrated in Schemes I-IV, particularly when A is C(R₁₁), wherein R₁₁ is alkoxy. The nucleus can be activated towards nucleophilic attack by the addition of a Lewis acid such as, but not limited to, boron trifluoride, triacetoxyborate, and lithium chloride. The preferred method of activation is described in U.S. Pat. No. 5,157,117. The quinolone nucleus is treated with triacetoxyborate, prepared in situ, in solvent such as, but not limited to, acetic acid or propionic acid and is heated for 1 to 24 h at a temperature between 60° C. and 120° C. The diacyl quinolinylborate (XV) is isolated by filtration after removal of the solvent. Scheme V illustrates this preferred method of activation.

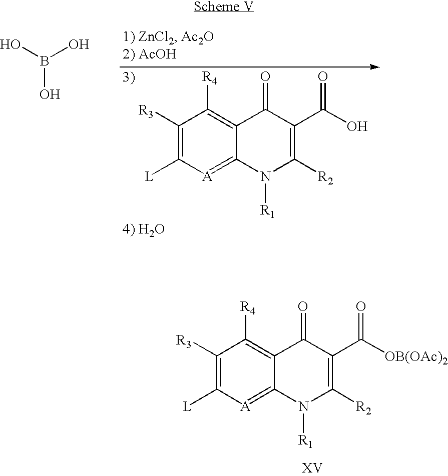

Another preferred method for activating the heterocyclic nucleus toward nucleophilic attack is illustrated in Scheme XXXVII. In this method, a quinolone carboxylic acid or ester derivative (i.e., compound II wherein R is hydrogen or lower alkyl and L is a leaving group) is treated with boron trifluoride etherate, preferably in a suitable solvent, such as THF, for from 1 hour to 48 hours at temperatures ranging from 0° C. to 60° C. After cooling, the product LXVI may be precipitated from the reaction mixture by the addition of a suitable solvent, such as diethyl ether, and the chelate isolated by filtration of the resulting solid.

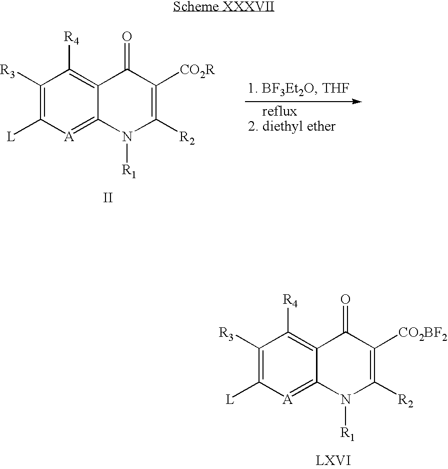

### Precursor Preparation

### Side Chain Amine III

Scheme VI illustrates the synthesis of the side chain amine III wherein E is

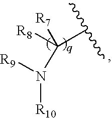

wherein, R₇ and R₈ are hydrogen, and q is 1

The trisubstituted or tetrasubstituted alkylidenes XX can be prepared by a Peterson, Wittig or Wadsworth-Horner-Emmons olefination of an appropriately substituted ketone (XVI) in a solvent such as, but not limited to, tetrahydrofuran, dimethylsulfoxide, or methylene chloride for 1 to 24 h at a temperature between −78° C. to 120° C. in the presence of a base such as, but not limited to n-butyl lithium, sodium hydride or potassium carbonate. The resulting ester (XVII) can be reduced with a reducing agent such as, but not limited to, diisobutylaluminum hydride, lithium triethylborohydride or sodium borohydride in a solvent such as, but not limited to, toluene, methylene chloride, ethanol or tetrahydrofuran for 1 to 24 h at a temperature between 0° C. and 120° C. to afford the corresponding alcohol XVIII, where q=1. Converting the alcohol XVIII to leaving group XIX, such as, but not limited to, chloride, bromide, mesylate or tosylate under standard conditions and displacing the leaving group with an appropriately substituted amine in a solvent such as, but not limited to, dimethylformamide, dimethylsulfoxide, or tetrahydrofuran for 1 to 24 h at a temperature between 0° C. and 120° C. converts the alcohol XVIII to an amine XX. Removal of the protecting group, P, under standard conditions known to those skilled in the art affords amine III, wherein E is:

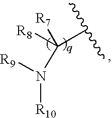

wherein, R₇ and R₈ are hydrogen, and q is 1. Alternatively, direct replacement of the alcohol XVIII can be accomplished via a Mitsunobu reaction with phthalimide and dialkyl azodicarboxylate to afford XXI. Deprotection of the phthalimide (XXI) with hydrazine in a solvent such as methanol or ethanol affords the amine (XX), wherein R₉ and R₁₀ are hydrogen. Alternative methods of deprotection include treatment with methylamine in methanol or with 6N hydrochloric acid. The protecting group, P, may be removed from XXI under standard conditions known to those skilled in the art to provide the amine V, wherein R₇ and R₈ are hydrogen and R₉ and P″ together with the nitrogen to which they are attached form a phthalimide group.

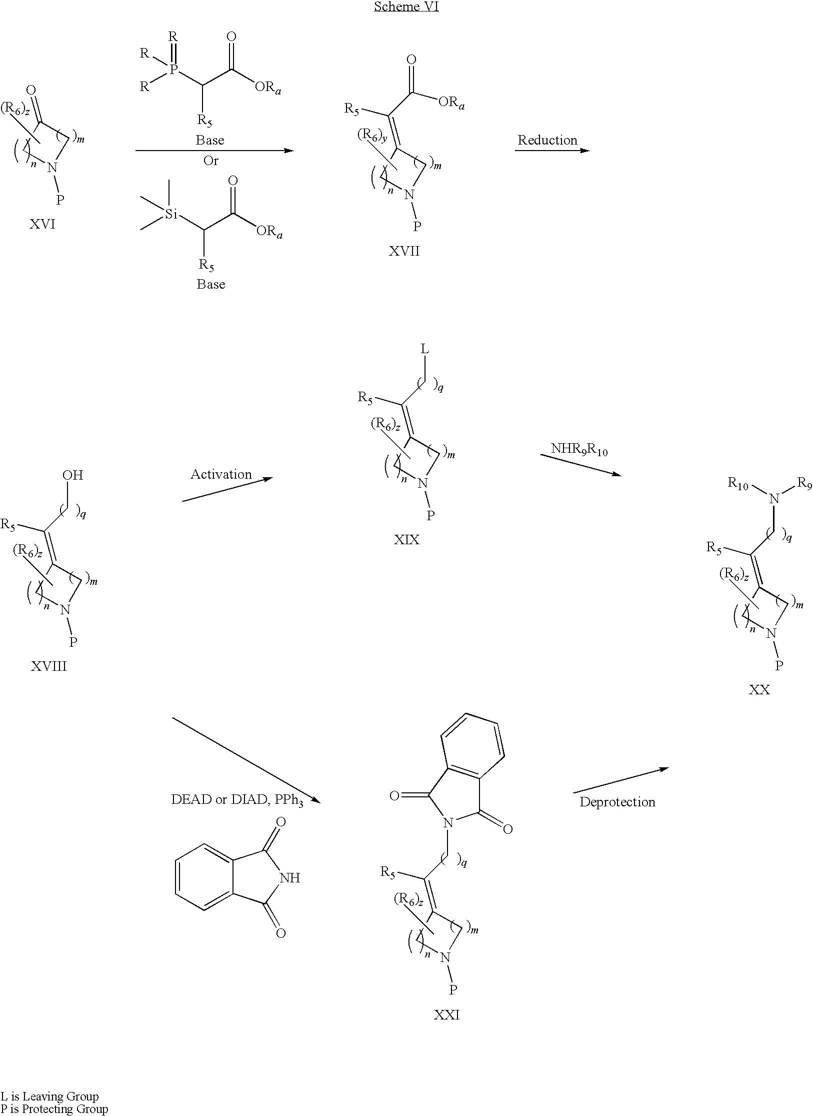

It will be apparent to one skilled in the art that the conversion of ketone XVI to olefin XVII may lead to geometrical isomers (Scheme VI), specifically in the case where XVI is asymmetric (i.e., the value of m is not equal to n). In such a case, the geometrical isomers may be separated by a number of methods known to those skilled in the art, including selective recrystallization, flash chromatography, high-performance liquid chromatography, and the like. It should also be apparent that separation may be achieved at various stages in the synthetic process, including at intermediates XVII, XVIII, XIX, or XXI, or alternatively at the final product stage XX.

Scheme XXXVIII illustrates the synthesis of the side chain amine LXX,

wherein E is:

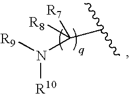

wherein, R₅ is cyano, R₇ and R₈ are hydrogen, and q is 1. The tetrasubstituted alkylidenes LXVII can be prepared by a Wadsworth-Horner-Emmons olefination of an appropriately substituted ketone (XVI) in a solvent such as, but not limited to, tetrahydrofuran, dimethylsulfoxide, or methylene chloride for from 1 to 24 h at a temperature between −78° C. to 120° C. in the presence of a base such as, but not limited to n-butyl lithium, sodium hydride or potassium carbonate. The cyano-substituted alkenyl bromides can undergo bromine-magnesium exchange with i-PrMgBr in an inert solvent, such as THF, at temperatures ranging from −78° C. to −20° C. The resulting organomagnesium species, as a solution in a suitable solvent such as THF, may be treated with an electrophile such as formaldehyde, optionally stabilized with methylaluminum bis(2,6-diphenylphenoxide), in a suitable solvent, such as methylene chloride, for from 1 to 24 hours at temperatures ranging from −20° C. to 37° C. to give the alcohol LXVIII. Direct replacement of the alcohol LXVIII can be accomplished via a Mitsunobu reaction with phthalimide and a dialkyl azodicarboxylate to afford LXIX. Deprotection of the phthalimide (LXIX) with hydrazine in a solvent such as methanol or ethanol affords the amine (LXX), wherein R₉ and R₁₀ are hydrogen. Alternative methods of deprotection include treatment with methylamine in methanol or heating with 6N hydrochloric acid. The protecting group, P, may be removed from LXIX under standard conditions known to those skilled in the art to provide the amine V, wherein R₅ is cyano, R₇ and R₈ are hydrogen and R₉ and P″ together with the nitrogen to which they are attached form a phthalimide group.

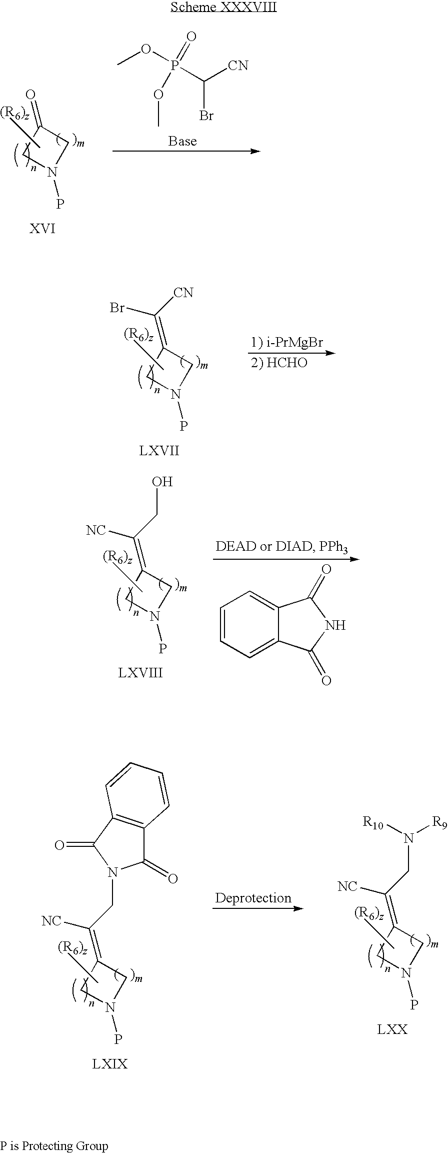

The cyano group of compound LXIX may also be converted to alternate functionalities, such as carboxy or alkoxycarbonyl, to afford amines LXXI or LXXII (Scheme XXXIX). For example, basic hydrolysis of nitrile LXIX with an alkali metal hydroxide, such as potassium hydroxide, in a suitable solvent, such as water, methanol, ethanol, or aqueous alcohol mixtures, at a temperature ranging from 20° C. to 100° C. for from 1 to 48 hours, followed by acid hydrolysis of the phthalamido group, with, for example, 6N hydrochloric acid at a temperature ranging from 60° C. to 100° C. for from 1 to 48 hours affords the corresponding amino acid derivative LXXI where R₉ and R₁₀ are hydrogen. Alternatively, acid hydrolysis of nitrile LXIX with a mineral acid in the presence of an alcohol at a temperature ranging from 20° C. to 200° C. for from 30 minutes to 48 hours, optionally under microwave irradiation, provides the corresponding amino ester derivative LXXII where R₉ and R₁₀ are hydrogen. Suitable mineral acids include, but are not limited to, sulfuric acid. Suitable alcohols include, but are not limited to, ethanol. Although Scheme XXXIX illustrates the conversion of nitrile LXIX to amino acid derivative LXXI and amino ester derivative LXXII with the ring nitrogen attached to a protecting group, the ring nitrogen may also be bound to the quinolone or naphthyridine nucleus as in compound VIII while performing the above transformations.

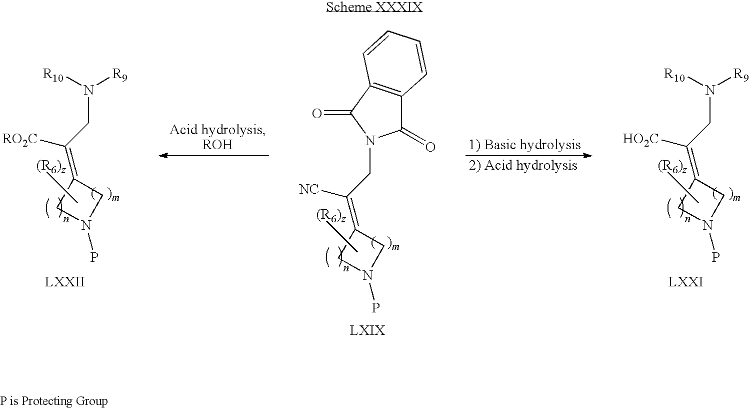

Scheme XXII illustrates the conversion of alcohols of formula XVIII to compounds of formula III, wherein E is alkenyl (LVIII). In addition, the Scheme outlines the synthesis of compounds of formula III, wherein E is:

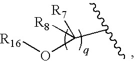

R₇ and R₈ are hydrogen and R₁₆ is acyl, alkoxycarbonyl, or sulfonyl (LX). Oxidation of alcohol XVIII with any of a number of suitable oxidizing agents, such as Dess-Martin periodinane, the Corey-Kim reagent, or the Swern reagent, affords the corresponding aldehyde (LVI). The aldehyde may be subjected to a base promoted olefination reaction, such as, but not limited to, the Wittig reaction to give LVII, wherein R_{c} is hydrogen or alkyl. Removal of the protecting group, P, from LVII under standard conditions known to those skilled in the art affords amine III, wherein E is alkenyl (LVIII). Scheme XX also illustrates the conversion of alcohols of formula XVIII to compounds of formula III, wherein E is:

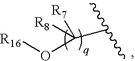

wherein, R₇ and R₈ are hydrogen, and R₁₆ is acyl, alkoxycarbonyl, or sulfonyl (LX). Reaction of alcohol XVIII with an acylating agent in the presence of an amine base, such as pyridine, in an inert solvent such as dichloromethane, tetrahydrofuran or toluene at temperatures ranging from −20° C. to 60° C. for from 1-48 hours provides compounds of formula III, wherein E is:

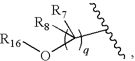

wherein, R₇ and R₈ are hydrogen, and R₁₆ is acyl (LIX). Acylating agents include acid halides, acid anhydrides, and acids in the presence of an activating agent such as dicyclohexylcarbodiimide, EDCl, BOP—Cl, BOP, PyBOP, and the like. Alcohols of formula XVIII may be converted into compounds of formula III, wherein E is:

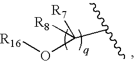

wherein, R₇ and R₈ are hydrogen and R₁₆ is alkoxycarbonyl (LIX) by reaction with a carbonylating agent in the presence of an amine base, such as pyridine, in an inert solvent such as dichloromethane, tetrahydrofuran or toluene at temperatures ranging from −20° C. to 60° C. for from 1-48 hours. Carbonylating agents include chloroformates, fluoroformates, azidoformates, and pyrocarbonates. Alcohols of formula XVIII may be converted into compounds of formula III, wherein E is

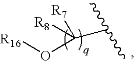

R₇ and R₈ are hydrogen and R₁₆ is sulfonyl (LIX) by reaction with a sulfonyl chloride or sulfonic anhydride in the presence of an amine base, such as pyridine, in an inert solvent such as dichloromethane, tetrahydrofuran or toluene at temperatures ranging from −20° C. to 60° C. for from 1-48 hours. Removal of the protecting group, P, from LIX under standard conditions known to those skilled in the art affords amine III, wherein E is:

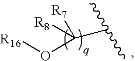

wherein, R₇ and R₈ are hydrogen, and R₁₆ is acyl, alkoxycarbonyl, or sulfonyl (LX).

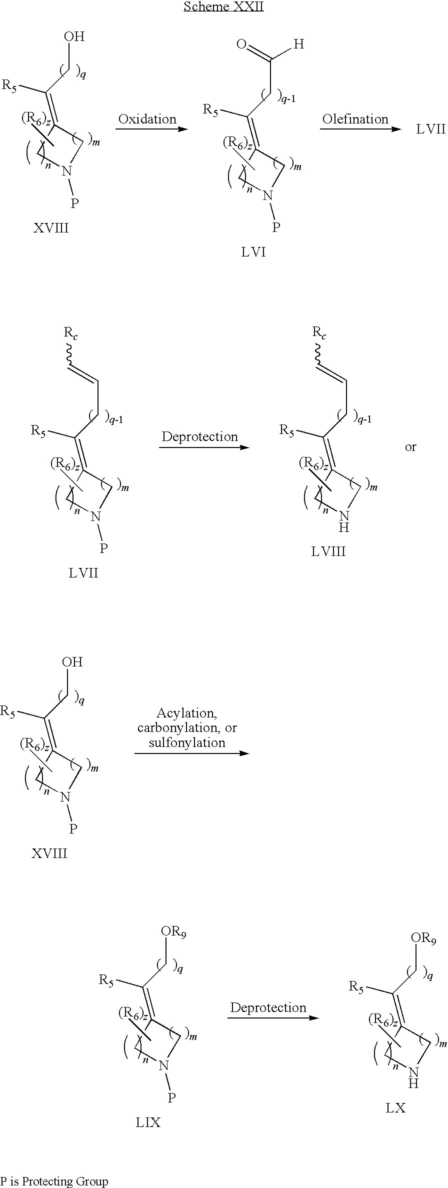

Scheme LXXIV illustrates the conversion of alkenes of formula LXXV to amines of formula LXXVIII. Alkene LXXV may be converted to alcohols of formula LXXVI by hydroboration with an appropriate reagent such as diborane, borane-ammonia complex, borane-N,N-diisopropylethylamine complex, borane-methyl sulfide complex, borane-pyridine complex, 9-borabicyclo[3.3.1]nonane, dicyclohexylborane, thexylborane, or disiamylborane in a suitable solvent such as tetrahydrofuran, toluene, diethyl ether, dichloromethane and the like, followed by oxidation of the organoborane intermediate with alkaline hydrogen peroxide, molecular oxygen, or amine oxides. A preferred oxidant is sodium hydroxide in 30% hydrogen peroxide. Typically, the hydroboration reaction is conducted for from 1 to 48 hours at a temperature ranging from −78° C. to 100° C. depending on the reactivity of the olefin and the hydroborating reagent. The subsequent oxidation may be conducted at temperatures ranging from 0° C. to 100° C. for from 30 minutes to 24 hours. Direct replacement of the alcohol LXXVI can be accomplished via a Mitsunobu reaction with phthalimide and a dialkyl azodicarboxylate to afford LXXVII. Deprotection of the phthalimide (LXXVII) with hydrazine in a solvent such as methanol or ethanol affords the amine (LXXVIII), wherein R₉ and R₁₀ are hydrogen. Alternative methods of deprotection include treatment with methylamine in methanol or heating with 6N hydrochloric acid. The protecting group, P, may be removed from LXXVII under standard conditions known to those skilled in the art to provide the amine V, wherein R₇ and R₈ are hydrogen and R₉ and P″ together with the nitrogen to which they are attached form a phthalimide group.

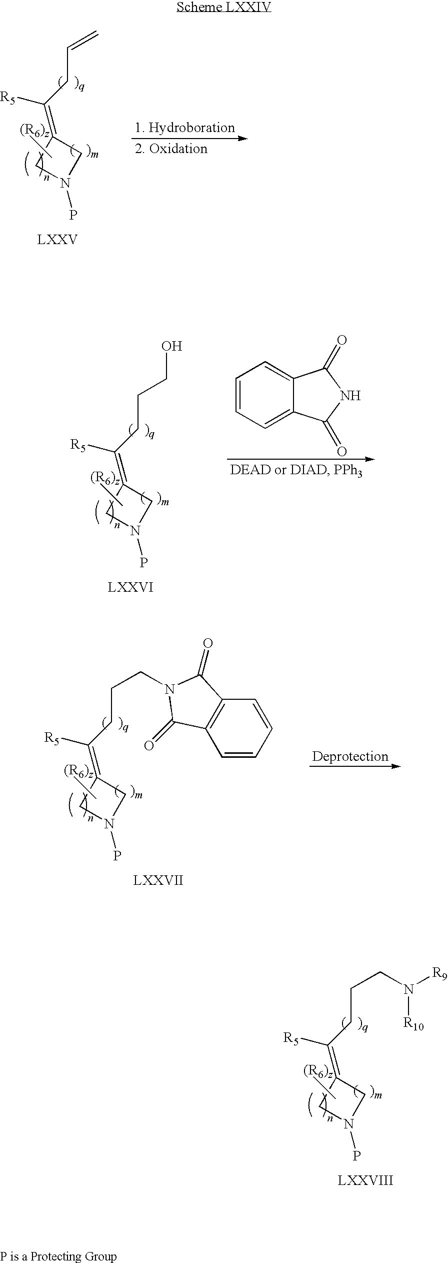

Scheme LXXV illustrates the preparation of oxazepanones of formula LXXXI, which are useful intermediates in the preparation of certain compounds of the instant invention. Suitably protected β-aminoalcohols of formula LXXIX may be converted to oxazepanes of formula LXXX by reaction with a bis-electrophile such as 2-methylene-1,3-dichloropropane, 2-methylene-1,3-dibromopropane, or 2-methylene-1,3-propanediol-bis(4-methylbenzenesulfonate) in the presence of a suitable base, such as sodium hydride, potassium hexamethyldisilazide, LDA, or lithium tetramethylpiperidide in an appropriate solvent, such as DMF, N-methylpyrrolidinone, or THF. Typically, the reaction is conducted for from 2 to 48 hours at a temperature ranging from −78° C. to 60° C. Suitable protecting groups for the β-aminoalcohol include carbamates, such as t-butyloxycarbonyl (Boc), benzyloxycarbonyl (Cbz), or allyloxycarbonyl (alloc). Conversion of alkene LXXX to the corresponding ketone LXXXI may be effected by oxidants such as sodium periodate/osmium tetroxide in mixed aqueous media, such as dioxane/water or t-butanol/water mixtures. The reaction may be conducted for from 2 to 96 hours at a temperature ranging from 0° C. to 100° C. Alternative methods of oxidation include, for example, ozonolysis in an inert solvent such as methylene chloride, ethyl acetate, chloroform, or THF, followed by reductive workup in the presence of dimethyl sulfide, triphenylphosphine, or sodium sulfite.

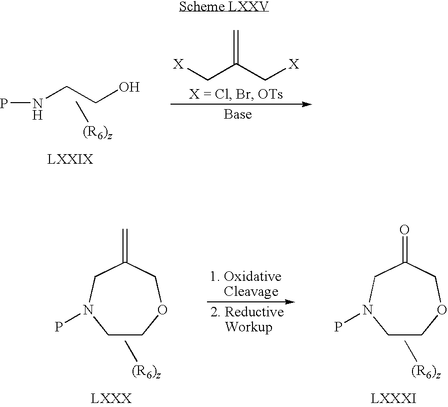

Scheme VII illustrates a direct conversion of ketone XVI to olefin XX using a base promoted olefination reaction such as, but not limited to, the Wittig, Wadsworth-Horner-Emmons, or Peterson olefination procedures. Alternatively, amine XX could be prepared by an olefin metathesis procedure from terminal olefin XXII using an appropriately substituted amine XXIII. Removal of the protecting group, P, from XX under standard conditions known to those skilled in the art affords amine III, wherein E is:

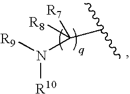

and R₇ and R₈ are hydrogen.

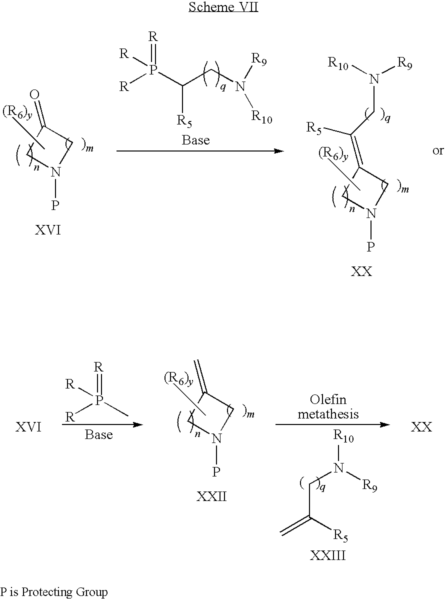

Scheme VIII illustrates the hydroxylation of XXIV with selenium dioxide to afford the allylic alcohol XXV. The transformation is performed in a solvent such as, but not limited to, methylene chloride, toluene or tetrahydrofuran at a temperature between 25° C. and 150° C., optionally in the presence of a co-oxidant such as tert-butyl hydroperoxide. Removal of the protecting group, P, from XXV under standard conditions known to those skilled in the art affords amine III, wherein E is:

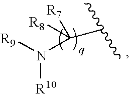

and one of R₆ is hydroxy.

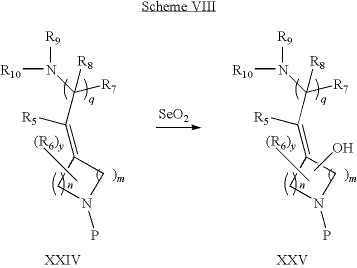

Scheme IX illustrates the preparation of α,β-unsaturated carbonyl compound XXVI, where R₇ is as defined previously, using a Peterson, Wittig or Wadsworth-Horner-Emmons olefination procedure of an appropriately substituted ketone (XVI) in a solvent such as, but not limited to, tetrahydrofuran, dimethylsulfoxide, or methylene chloride for from 1 to 24 h at a temperature between −78° C. to 120° C. in the presence of a base such as, but not limited to, n-butyl lithium, sodium hydride or potassium carbonate. The resulting carbonyl compound (XXVI) can be reduced with a reducing agent such as, but not limited to, diisobutylaluminum hydride, lithium triethylborohydride or sodium borohydride in a solvent such as, but not limited to, toluene, methylene chloride, or tetrahydrofuran for from 1 to 24 h at a temperature between 0° C. and 120° C. to afford the corresponding alcohol XXVII. Alternatively, the carbonyl compound may undergo nucleophilic addition with an appropriately substituted organometallic agent (R₈M, wherein M is a metal), such as an organolithium species or a Grignard reagent, to afford the corresponding alcohol XXVII, where R₈ is alkyl. Suitable solvents for the latter transformation include, diethyl ether, tetrahydrofuran, or toluene, at temperatures ranging from −78° C. to 20° C. for from 30 minutes to 48 hours. Where one of R₇ or R₈ are hydrogen, converting the alcohol functionality in XXVII to a leaving group, such as, but not limited to, bromide, mesylate or tosylate as in XXVIII under standard conditions and displacing the leaving group with an appropriately substituted amine in a solvent such as, but not limited to, dimethylformamide, dimethylsulfoxide, or tetrahydrofuran for from 1 to 24 h at a temperature between 0° C. and 120° C. converts the alcohol XXVII to an amine XXX. Removal of the protecting group, P, from XXX under standard conditions known to those skilled in the art affords amine III, wherein E is:

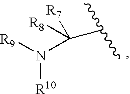

and one R₇ and R₈ is hydrogen.

Alternatively, where one of R₇ or R₈ is hydrogen, direct replacement of the alcohol XXVII can be accomplished via a Mitsunobu reaction with phthalimide and a dialkyl azodicarboxylate followed by deprotection of the phthalimide with hydrazine in a solvent such as methanol or ethanol to afford amine XXX. The protecting group, P, may be removed from XXIX under standard conditions known to those skilled in the art to provide the amine V, wherein R₈ is hydrogen and R₉ and P″ together with the nitrogen to which they are attached form a phthalimide group.

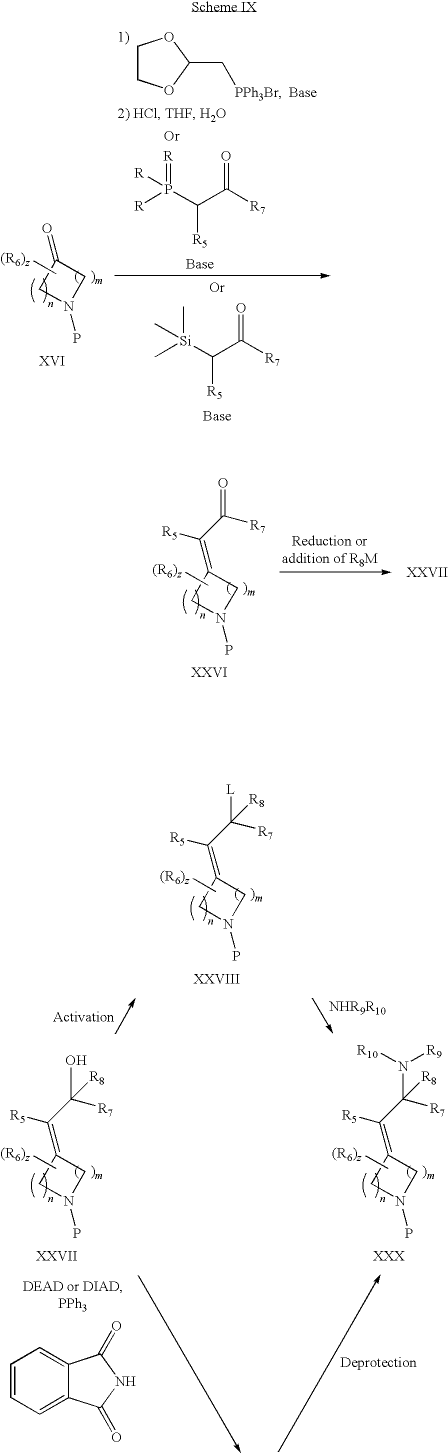

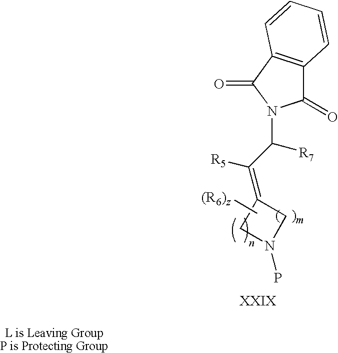

Scheme X depicts the preparation of XXXVI, wherein R₅ is halogen. Alkylidenes XXXI, wherein R₅ is hydrogen, can be halogenated with an appropriate halogenating agent such as, but not limited to, 1-bromo-2,5-pyrrolidinedione, 1,1,1-tris(acetoxy)-1,1-dihydro-2-benzodioxol-3(1H)-one and a tetraalkylammonium bromide, or thionyl chloride to provide XXXII. Alkylidene XXXII can be reduced with a reducing agent such as, but not limited to, diisobutylaluminum hydride, lithium triethylborohydride or sodium borohydride in a solvent such as, but not limited to, toluene, methylene chloride, or tetrahydrofuran for from 1 to 24 h at a temperature between 0° C. and 120° C. to afford the corresponding alcohol XXXIII.

Alternatively, the carbonyl compound may undergo nucleophilic addition with an appropriately substituted organometallic agent, such as an organolithium species or a Grignard reagent, to afford the corresponding alcohol XXXIII, where R₈ is alkyl. Suitable solvents for the latter transformation include, diethyl ether, tetrahydrofuran, or toluene, at temperatures ranging from −78° C. to 20° C. for from 30 minutes to 48 hours. Where one of R₇ or R₈ is hydrogen, converting the alcohol functionality in XXXIII to a leaving group, such as, but not limited to, bromide, mesylate or tosylate as in XXXIV under standard conditions and displacing the leaving group with an appropriately substituted amine in a solvent such as, but not limited to, dimethylformamide, dimethylsulfoxide, or tetrahydrofuran for from 1 to 24 h at a temperature between 0° C. and 120° C. converts XXXIV to an amine XXXVI. Removal of the protecting group, P, from XXXVI under standard conditions known to those skilled in the art affords amine III, wherein E is:

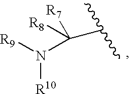

and one of R₇ and R₈ is hydrogen.

Alternatively, where one of R₇ or R₈ is hydrogen, direct replacement of the alcohol XXXIII can be accomplished via a Mitsunobu reaction with phthalimide and a dialkyl azodicarboxylate followed by deprotection of the phthalimide with hydrazine in a solvent such as methanol or ethanol to afford the amine XXXVI. The protecting group, P, may be removed from XXXV under standard conditions known to those skilled in the art to provide the amine V, wherein R₈ is hydrogen and R₉ and P″ together with the nitrogen to which they are attached form a phthalimide group.

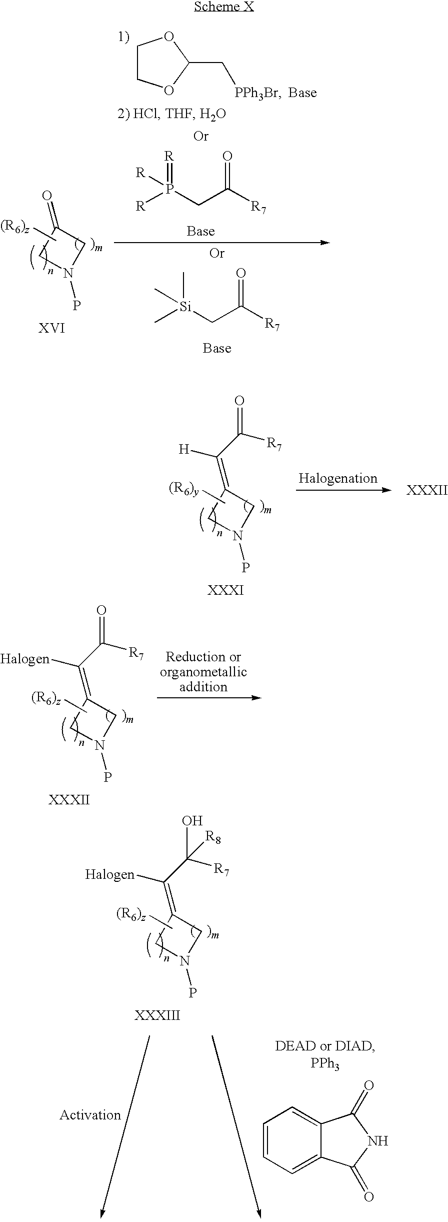

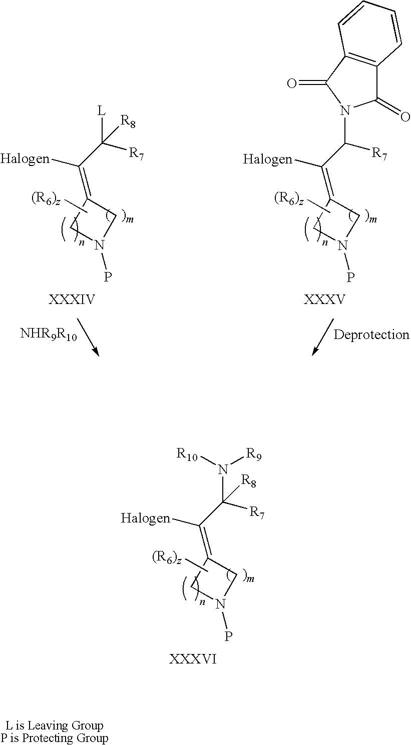

Scheme XI illustrates the synthesis of the side chain amine III wherein E is

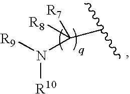

wherein, R₇ and R₈ are hydrogen and R₅ is a substituted or branched-chain alkyl. In Scheme XI, halogenated carbonyl compound XXXVII, wherein Rₐ is hydrogen or alkyl, may be prepared in a similar fashion as halogenated carbonyl compound XXXII. Carbonyl compound XXXVII, wherein Ra is hydrogen or alkyl, may be reduced with a reducing agent such as, but not limited to, diisobutylaluminum hydride, lithium triethylborohydride or sodium borohydride in a solvent such as, but not limited to, toluene, methylene chloride, or tetrahydrofuran for from 1 to 24 h at a temperature between 0° C. and 120° C. to afford the corresponding alcohol XXXVIII where Rₐ is hydrogen or alkyl, one of R_{b} is hydrogen, and the other R_{b} is hydroxyl.

Alternatively, the carbonyl compound XXXVII, wherein Rₐ is alkyl, may undergo nucleophilic addition with an appropriately substituted organometallic agent, such as an organolithium species or a Grignard reagent, to afford the corresponding alcohol XXXVIII where Rₐ is alkyl, one of R_{b} is alkyl, and the other R_{b} is hydroxyl. Finally, carbonyl compound XXXVII, wherein Rₐ is hydrogen or alkyl, or alcohol XXXVIII, wherein Rₐ is hydrogen or alkyl, one of R_{b} is hydrogen, and the other R_{b} is hydroxyl, may be fluorinated using a nucleophilic fluorinating reagent, such as but not limited to, (N-ethylethanaminato)trifluorosulfur (DAST) or bis(2-methoxyethyl)aminosulfur trifluoride (Deoxofluor), in a suitable solvent, such as methylene chloride, for from 1 to 24 h at a temperature between 0° C. and 60° C. to afford XXXVIII, where in the case of the carbonyl compound XXXVII as substrate, Rₐ is hydrogen or alkyl and R_{b} is fluorine, and where in the case of the alcohol XXXVIII as substrate, Rₐ is hydrogen or alkyl, one of R_{b} is hydrogen, and the other R_{b} is fluorine. Halogenated alkylidene XXXVIII may be carbonylated in the presence of a transition metal catalyst, such as but not limited to palladium acetate, dicarbonylbis(triphenylphosphine)nickel, or tetrakis (triphenylphosphine)palladium, under an atmosphere of carbon monoxide in the presence of a second additive such as methanol, optionally as solvent, or in a solvent such as, but not limited to, dimethylsulfoxide or tetrahydrofuran, for 1 to 24 h at a temperature between 0° C. and 120° C. to afford ester XXXIX. XXXIX may be reduced with a reducing agent such as, but not limited to, diisobutylaluminum hydride, lithium triethylborohydride or sodium borohydride in a solvent such as, but not limited to, toluene, methylene chloride, or tetrahydrofuran for 1 to 24 h at a temperature between 0° C. and 120° C. to afford the corresponding alcohol XL, where q=1. Converting the alcohol XL to leaving group XLI, such as, but not limited to, bromide, mesylate or tosylate, under standard conditions and displacing the leaving group with an appropriately substituted amine in a solvent such as, but not limited to, dimethylformamide, dimethylsulfoxide, or tetrahydrofuran for from 1 to 24 h at a temperature between 0° C. and 120° C. converts the alcohol XL to an amine XLIII. Removal of the protecting group, P, from XLIII under standard conditions known to those skilled in the art affords amine III, wherein E is:

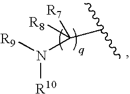

wherein, R₇ and R₈ are hydrogen and R₅ is CRₐRₐR_{b}. Alternatively, direct replacement of the alcohol XL may be accomplished via a Mitsunobu reaction with phthalimide and dialkyl azodicarboxylate to afford XLII. Deprotection of the phthalimide XLII with hydrazine in a solvent such as methanol or ethanol affords the amine XLIII. The protecting group, P, may be removed from XLII under standard conditions known to those skilled in the art to provide the amine V, wherein R₇ and R₈ are hydrogen, R₉ and P″ together with the nitrogen to which they are attached form a phthalimide group, and R₅ is CRₐRₐR_{b}.

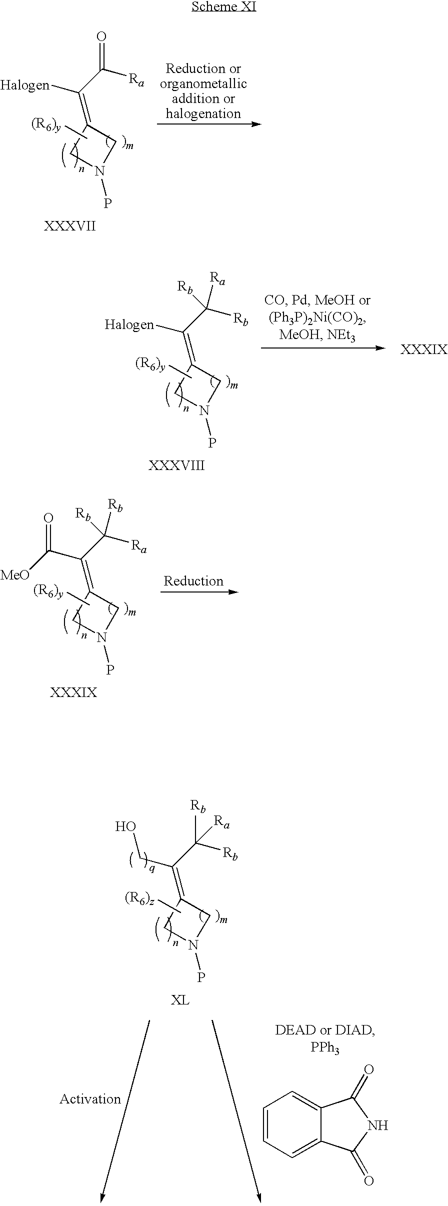

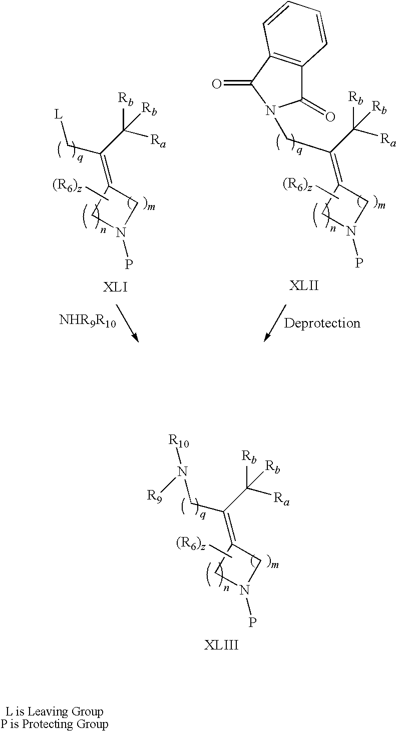

Scheme XII illustrates the synthesis of the side chain amine III wherein E is:

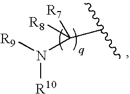

wherein, one of R₇ or R₈ is hydrogen and the other is alkyl, R₅ is substituted or branched-chain alkyl, and q is 1. Compound XXXVIII, prepared as described above, may be carbonylated in the presence of a transition metal catalyst, such as but not limited to palladium acetate, dicarbonylbis(triphenylphosphine)nickel, or tetrakis (triphenylphosphine)palladium, under an atmosphere of carbon monoxide in the presence of an organometallic reagent R₇M, wherein R₇ is defined previously and includes reagents such as tributyltinhydride or alkyl indium agents (Organic Letters 2003, 5(7), 1103-1106), in a solvent such as, but not limited to, methanol, dimethylsulfoxide, or tetrahydrofuran for 1 to 24 h at a temperature between 0° C. and 120° C. to afford XLIV, where R₇ is as previously defined. Carbonyl compound XLIV may be reduced with a reducing agent such as, but not limited to, diisobutylaluminum hydride, lithium triethylborohydride or sodium borohydride in a solvent such as, but not limited to, toluene, methylene chloride, or tetrahydrofuran for from 1 to 24 h at a temperature between 0° C. and 120° C. to afford the corresponding alcohol XLV.

Alternatively, the carbonyl compound may undergo nucleophilic addition with an appropriately substituted organometallic reagent, such as an organolithium species or a Grignard reagent, to afford the corresponding alcohol XLV, where R₈ is alkyl. Suitable solvents for the latter transformation include, diethyl ether, tetrahydrofuran, or toluene, at temperatures ranging from −78° C. to 20° C. for from 30 minutes to 48 hours. Where one of R₇ or R₈ are hydrogen, converting the alcohol functionality in XLV to a leaving group, such as, but not limited to, bromide, mesylate or tosylate as in XLVI under standard conditions and displacing the leaving group with an appropriately substituted amine in a solvent such as, but not limited to, dimethylformamide, dimethylsulfoxide, or tetrahydrofuran for from 1 to 24 h at a temperature between 0° C. and 120° C. converts the alcohol XLV to an amine XLVIII. Removal of the protecting group, P, from XLVIII under standard conditions known to those skilled in the art affords amine III, wherein E is:

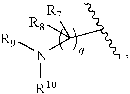

wherein, one of R₇ and R₈ is hydrogen and the other is alkyl, R₅ is substituted or branched-chain alkyl, and q is 1.

Alternatively, where one of R₇ or R₈ is hydrogen, direct replacement of the alcohol XLV can be accomplished via a Mitsunobu reaction with phthalimide and a dialkyl azodicarboxylate followed by deprotection of the phthalimide with hydrazine in a solvent such as methanol or ethanol to afford amine XLVIII. The protecting group, P, may be removed from XLVIII under standard conditions known to those skilled in the art to provide the amine V, wherein one of R₇ and R₈ is hydrogen and the other is alkyl, R₉ and P″ together with the nitrogen to which they are attached form a phthalimide group, R₅ is substituted or branched-chain alkyl, and q is 1.

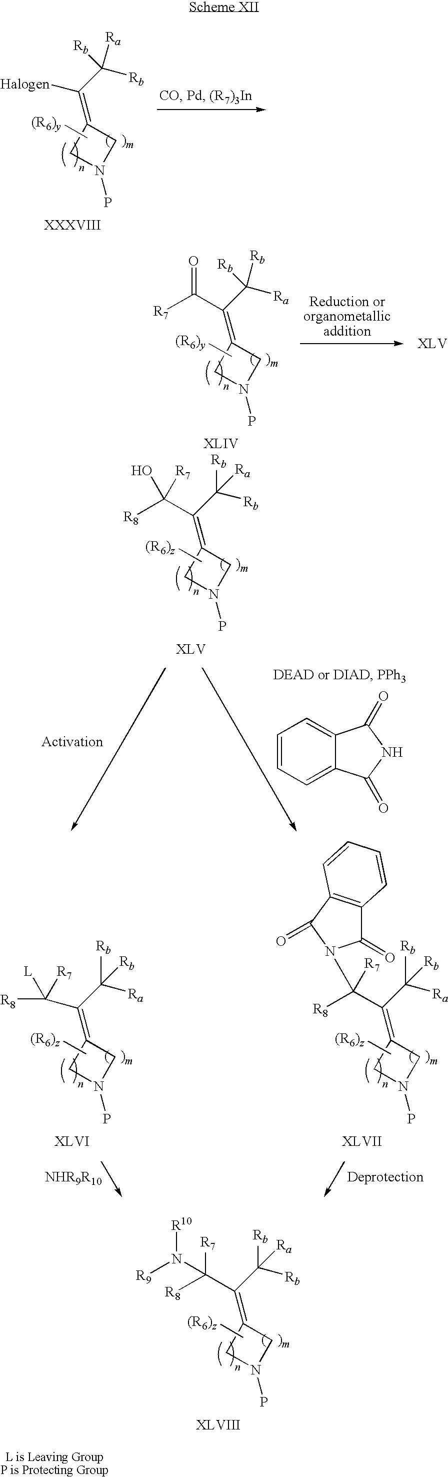

Scheme XIII illustrates the conversion of ketone XVIa to olefin LIII using a base promoted Stork-Jung vinylsilane Robinson annulation protocol (Tetrahedron Letters, 2001, 42, 9123). Condensation of ketone XVIa with allyl iodide XLIX, wherein R_{c} is an alkyl group and P′ is a hydroxy protecting group, (Tetrahedron Letters, 2001, 42, 9123) affords alkylated ketone L. Epoxidation of ketone L with epoxidizing agents such as, but not limited to, dimethyl dioxirane or m-chloroperbenzoic acid, affords oxirane LI. Protodesilylation of LI with agents such as, but not limited to, tetra-n-butylammonium fluoride or pyridinium poly(hydrogen fluoride) and aqueous acid, with concomitant epoxide ring opening affords ketone LII. Ring annulation of LII may be accomplished by treatment of LII with a base, such as but not limited to, sodium methoxide to afford LIII. α,β-Unsaturated ketone LIII may be reduced with a reducing agent such as, but not limited to, diisobutylaluminum hydride, lithium triethylborohydride or sodium borohydride in a solvent such as, but not limited to, toluene, methylene chloride, or tetrahydrofuran for from 1 to 24 h at a temperature between 0° C. and 120° C. to afford, following removal of the hydroxy protecting group, the corresponding alcohol LIV, wherein one of R₁₂ is hydrogen and the other R₁₂ is hydroxy.

Alternatively, LIII may undergo nucleophilic addition with an appropriately substituted organometallic reagent, such as an organolithium species or a Grignard reagent, to afford, following removal of the hydroxy protecting group, the corresponding alcohol LIV, where one of R₁₂ is alkyl and the other R₁₂ is hydroxy. Suitable solvents for the latter transformation include, diethyl ether, tetrahydrofuran, or toluene, at temperatures ranging from −78° C. to 20° C. for from 30 minutes to 48 hours. Finally, carbonyl compound LIII, may be fluorinated using a nucleophilic fluorinating reagent, such as but not limited to, (N-ethylethanaminato)trifluorosulfur (DAST) or bis(2-methoxyethyl)aminosulfur trifluoride (Deoxofluor), in a suitable solvent, such as methylene chloride, for from 1 to 24 h at a temperature between 0° C. and 60° C. to afford, following removal of the hydroxy protecting group, alcohol LIV, where R₁₂ is fluorine.

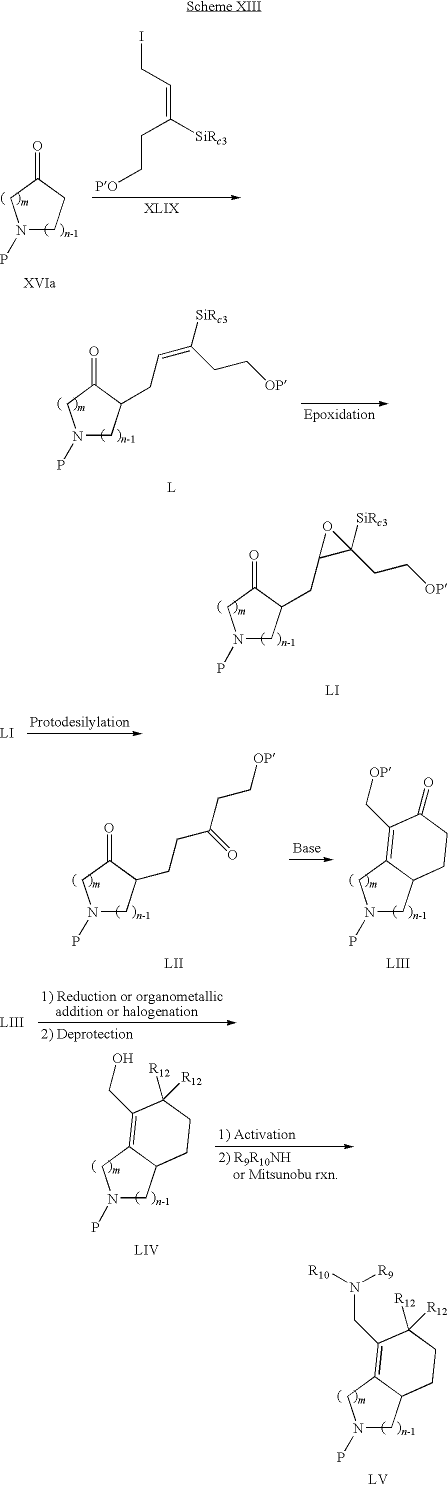

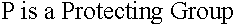

Alcohol LIV may be converted to leaving group, such as, but not limited to, bromide, mesylate or tosylate under standard conditions. Displacement of the leaving group with an appropriately substituted amine in a solvent such as, but not limited to, dimethylformamide, dimethylsulfoxide, or tetrahydrofuran for from 1 to 24 h at a temperature between 0° C. and 120° C. converts LIV to amine LV. Removal of the protecting group, P, from LV under standard conditions known to those skilled in the art affords the corresponding secondary amine III, wherein E is:

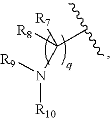

wherein, R₇ and R₈ are hydrogen, and R₅ and R₆ join to form a 6-membered carbocyclic ring, and q is 1.

Alternatively, direct replacement of the hydroxyl group of alcohol LIV can be accomplished via a Mitsunobu reaction with phthalimide and a dialkyl azodicarboxylate, followed by deprotection of the phthalimide with hydrazine in a solvent such as methanol or ethanol, to afford the amine LV, wherein R₉ and R₁₀ are hydrogen.

### Experimental Section

### Precursor Preparation

### Heterocyclic Nuclei

Most heterocyclic nuclei such as 1-cyclopropyl-1,4-dihydro-6,7-difluoro-8-methoxy-4-oxo-quinoline-3-carboxylic acid, 7-chloro-1-cyclopropyl-6-fluoro-4-oxo-1,4-dihydro-naphthpyridine-3-carboxylic acid, 9,10-difluoro-2,3-dihydro-3-methyl-7-oxo-7H-pyrido[1,2,3-de]-1,4-benzoxazine-6-carboxylic acid, (3S)-9,10-difluoro-2,3-dihydro-3-methyl-7-oxo-7H-pyrido[1,2,3-de]-1,4-benzoxazine-6-carboxylic acid, 1-cyclopropyl-1,4-dihydro-6,7-difluoro-4-oxo-quinoline-3-carboxylic acid, 7-chloro-1-(2,4-difluorophenyl)-6-fluoro-4-oxo-1,4-dihydro-naphthyridine-3-carboxylic acid, 1-cyclopropyl-1,4-dihydro-6,7-difluoro-5-methyl-4-oxo-quinoline carboxylic acid, 1-[(1R,2S)-2-fluorocyclopropy]I-1,4-dihydro-6,7-difluoro-5-methyl-4-oxo-quinoline carboxylic acid, 1-(6-amino-3,5-difluoro-2-pyridinyl)-8-chloro-6,7-difluoro-1,4-dihydro-4-oxo-quinoline-3-carboxylic acid, 1-cyclopropyl-1,4-dihydro-7-fluoro-8-methoxy-4-oxo-quinoline-3-carboxylic acid, 5-amino-1-cyclopropyl-6,7,8-trifluoro-4-oxo-1,4-dihydroquinoline-3-carboxylic acid, and 1-cyclopropyl-6,7-difluoro-8-methyl-4-oxo-1,4-dihydroquinoline-3-carboxylic acid were prepared according to literature methods (see above discussion about general procedures for preparing heterocyclic nuclei) or were purchased from commercial sources.

1-Cyclopropyl-6,7-difluoro-8-fluoromethoxy-4-oxo-1,4-dihydroquinoline-3-carboxylic acid and 1-cyclopropyl-6,7-fluoro-8-methoxy-5-methyl-4-oxo-1,4-dihydroquinoline-3-carboxylic acid were prepared as follows (Schemes LIV and LV).

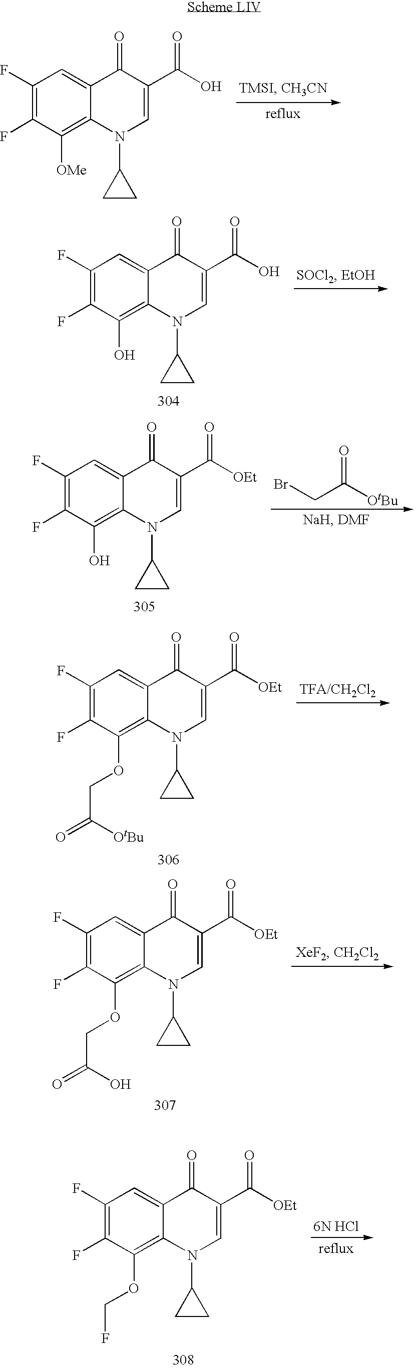

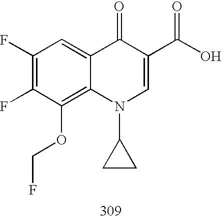

### 1-Cyclopropyl-6 ,7-fluoro-8-fluoromethoxy-4-oxo-1,4- dihydroquinoline-3-carboxylic acid difluoroborate ester (Cpd 309)

### Step 1

A solution of 1-cyclopropyl-6,7-difluoro-8-methoxy-4-oxo-1,4-dihydroquinoline-3-carboxylic acid (5.0 g, 16.9 mmol) in CH₃CN (100 mL) was treated with TMSI (7.0 mL, 51.5 mmol) under nitrogen and the reaction mixture was heated to reflux temperature for 23 hours. The resulting mixture was allowed to cool to room temperature and concentrated. The residue was washed with 10% aq. Na₂SO₃ (100 mL), H₂O (2×100 mL) and ethyl ether (50 mL), and allowed to dry overnight to afford 304 as a white solid. This compound was used without purification in the next step.

### Step 2

The above obtained 304 was suspended in EtOH (150 mL), cooled to 0° C., and treated with thionyl chloride (5.0 mL, 68.7 mmol). The reaction mixture was allowed to warm up to room temperature and stir for 48 hours before another portion of thionyl chloride (5.0 mL, 68.7 mmol) was added. The reaction was stirred for another 24 hours before it was concentrated in vacuo. The residue was purified by silica gel chromatography (4% MeOH in CH₂Cl₂) to afford 305 as a off-white solid (4.3 g, 82%). MS 310 (M+H).

### Step 3

To a solution of 305 (0.64 g, 2.1 mmol) in DMF (10 mL) at 0° C. was added NaH (60% in mineral oil, 0.1 g, 2.5 mmol). The reaction was stirred at 0° C. for 45 min before tert-butyl bromoacetate (0.46 mL, 3.1 mmol) was added. The reaction mixture was allowed to warm up to room temperature and stir for 18 hours. The mixture was diluted with aq. NH₄Cl (50 mL), and extracted with CH₂Cl₂ (2×100 mL). The organic layers were combined, washed with brine, dried over MgSO₄ and concentrated. Purification by silica gel chromatography (2% MeOH in CH₂Cl₂) gave 306 as a white solid (0.77 g, 88%). This compound was used without purification in the next step.

### Step 4

A solution of 306 (0.76 g, 1.8 mmol) in CH₂Cl₂ (6 mL) was treated with TFA (3 mL). The reaction mixture was stirred for 5 hours before being concentrated. The resulting 307 was dissolved in CH₂Cl₂ (10 mL), and to the solution was added XeF₂ (0.5 g, 3.0 mmol). The reaction mixture was stirred for 18 hours at room temperature, diluted with sat. aq. NaHCO₃, and extracted with CH₂Cl₂ (2×100 mL). The organic layers were combined, washed with brine, dried over MgSO₄, and concentrated. Purification by silica gel chromatography (2% MeOH in CH₂Cl₂) gave 308 as a brown solid (0.4 g, 65%). MS 342 (M+H).

### Step 5

A solution of 308 (0.3 g, 0.88 mmol) in 6N HCl was heated to reflux temperature for 1.5 h. The reaction mixture was allowed to cool to room temperature and concentrated. The residue was purified by silica gel chromatography (2% MeOH in CH₂Cl₂) to give 309 as a yellow solid (0.15 g, 55%). MS 314 (M+H).

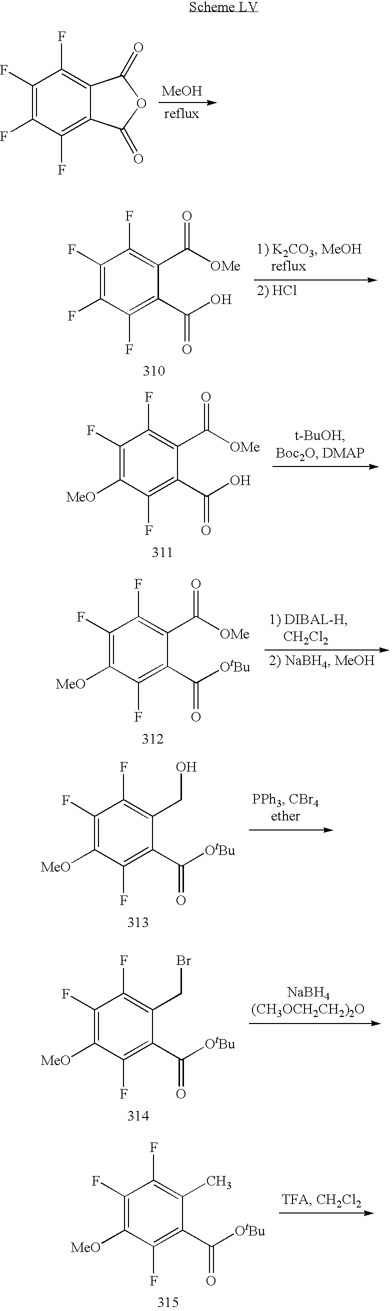

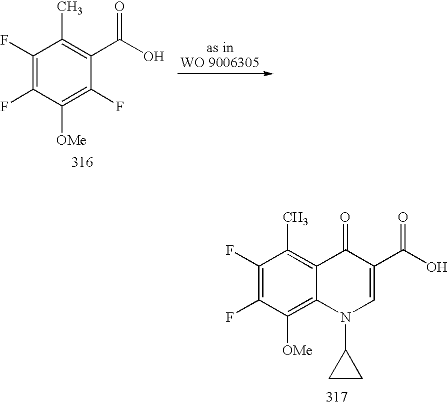

### 2,4,5-Trifluoro-3-methoxy-5-methyl-benzoic acid (316) 1-Cyclopropyl-6,7-difluoro-8-methoxy-5-methyl-4-oxo- 1,4-dihydroquinoline-3-carboxylic acid (317)

### Steps 1 and 2

A solution of tetrafluorophthalic anhydride (5.0 g, 22.7 mmol) in MeOH (50 mL) was heated to reflux temperature for 4 hours to generate half ester 310 in situ. The reaction was allowed to cool to room temperature and K₂CO₃ (4.7 g, 34.1 mmol) was added slowly. The mixture was heated again to reflux temperature for 6 hours before being cooled to room temperature and concentrated. The residue was diluted with 10% HCl aqueous solution (70 mL) and the mixture was extracted with EtOAc (3×100 mL). The organic layers were combined, dried over MgSO₄, and concentrated to give 311 as a off-white solid (6.0 g, 100%), which was used as such in the next step.

### Step 3

A solution of 311 (5.0 g, 18.9 mmol), di-tert-butyl-dicarbonate (8.3 g, 38.0 mmol) and 4-dimethylaminopyridine (0.46 g, 3.8 mmol) in tert-butanol (70 mL) was stirred at room temperature for 48 hours. The reaction mixture was concentrated and diluted with ethyl ether (200 mL). The resulting solution was washed with H₂O (50 mL), aq. NH₄Cl (50 mL) and brine, dried over MgSO₄, and concentrated. The residue was purified by silica gel chromatography (10% EtOAc in hexane) to give 312 as a colorless oil (4.0 g, 66%).

### Step 4

To a solution of 312 (4.0 g, 12.5 mmol) in CH₂Cl₂ (100 mL) at −78° C. was added diisobutyl aluminum hydride (1.0 M in hexane, 25 mL, 25 mmol). The reaction was maintained at −78° C. for 1 h before being quenched with 1N NaOH aqueous solution. The mixture was extracted with CH₂Cl₂ (2×100 mL). The organic layers were combined, washed with brine, dried over MgSO₄ and concentrated. The resulting residue was dissolved in MeOH (50 mL). The solution was cooled to 0° C. and NaBH₄ (0.48 g, 12.6 mmol) was added. The reaction was maintained at 0° C. for 30 min before being quenched with H₂O. The mixture was extracted with CH₂Cl₂ (2×100 mL). The organic layers were combined, washed with brine, dried over MgSO₄ and concentrated. Purification by silica gel chromatography (10% EtOAc in hexane) afforded 313 as a colorless oil (2.2 g, 60%).

### Step 5

A solution of 313 (2.2 g, 7.5 mmol) in ethyl ether (80 mL) was treated with PPh₃ (3.85 g, 14.7 mmol) and CBr₄ (5.0 g, 15.1 mmol). The reaction was stirred at room temperature for 48 hours. The mixture was filtered and the solution was concentrated. Purification by silica gel chromatography (5% EtOAc in hexane) yielded 314 as a yellow oil (2.05 g, 77%).

### Step 6

To a solution of 314 (2.6 g, 7.3 mmol) in 2-methoxyethyl ether (35 mL) at room temperature was added NaBH₄ (0.56 g, 14.7 mmol). The reaction was stirred for 2 hours at room temperature before being quenched with H₂O. The mixture was extracted with CH₂Cl₂ (2×100 mL). The organic layers were combined, washed with brine, dried over MgSO₄ and concentrated. Purification by silica gel chromatography (5% EtOAc in hexane) afforded 315 as a colorless oil (1.8 g, 90%).

### Step 7

A solution of 315 (1.8 g, 6.5 mmol) in CH₂Cl₂ (10 mL) was treated with TFA (5 mL). The reaction was stirred at room temperature for 3.5 h. The solution was concentrated to give 316 as a white solid (1.4 g, 98%).

### Step 8

317 was prepared from 316 following the literature procedure (WO 9006305).

### Precursor Preparation A

### Preparation of Diacyl Quinolinyl Borates

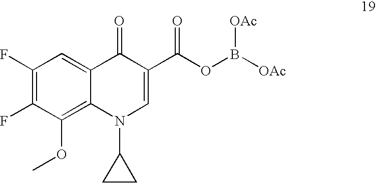

### Compound 19 (Formula XV: L=F, A=C-OMe, R₁=Cyclopropyl, R₂=H, R₃=F, R₄=H)

The diacyl quinolinyl borates were prepared by the procedure reported in U.S. Pat. No. 5,157,117. A mixture of boric acid (2.4 g, 38.7 mmol), acetic anhydride (13.8 mL, 146 mmol) and zinc chloride (52 mg, 0.38 mmol) was warmed to 110° C. for 1.5 h, treated with acetic acid (51 mL) and was allowed to stir an additional hour at 110° C. The resulting mixture was allowed to cool to 60° C., treated with 1-cyclopropyl-1,4-dihydro-6,7-difluoro-8-methoxy-4-oxo-quinoline-3-carboxylic acid (18) (7.3 g, 25.9 mmol) and acetic acid (26 mL). The resulting solution was warmed to 60° C. for 5 h, cooled to room temperature, and was concentrated in vacuo. The residue was treated with water (50 mL) and the solid was collected by filtration. The resulting solid was washed with water (3×50 mL), and dried to afford the title compound as a white solid, which was used as such in the next reaction.

The same procedure as above was used to convert each of the respective heterocyclic carboxylic acids listed in Table 2 to the corresponding diacylborate derivative (17, 21, 23, 83, and 319).

TABLE 2

| Compound | Quinolone | Compound | Diacyl Quinolinyl Borate |
|---|---|---|---|
| 16 | 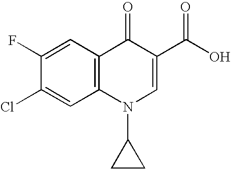 | 17 | 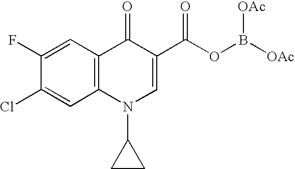 |
| 20 | 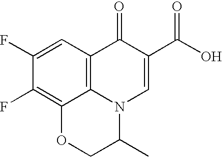 | 21 | 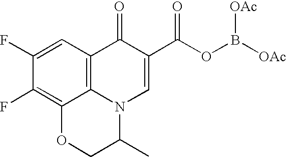 |
| 22 | 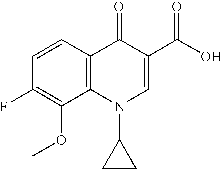 | 23 | 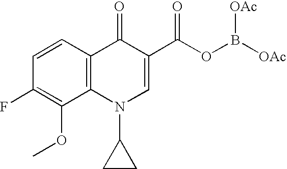 |
| 82 | 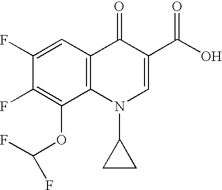 | 83 | 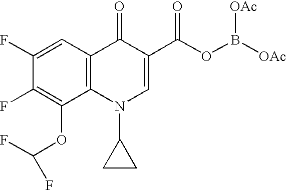 |
| 318 | 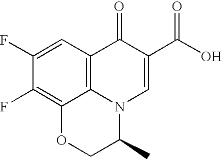 | 319 | 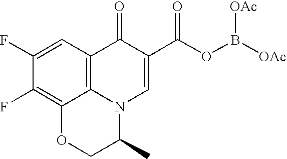 |

### Precursor Preparation C

### Preparation of Difluoro Quinolinyl Borates

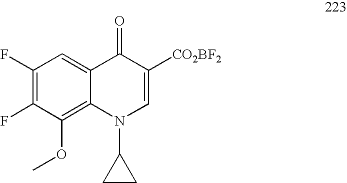

### 1-Cyclopropyl-1,4-dihydro-6,7-difluoro-8-methoxy-4- oxo-quinoline-3-carboxylic acid difluoroboryl ester (223)

1-Cyclopropyl-1,4-dihydro-6,7-difluoro-8-methoxy-4-oxo-quinoline-3-carboxylic acid (18) (10.08 g; 34.14 mmol) and boron trifluoride etherate (30 mL; 236 mmol) in anhydrous THF (150 mL) were heated at reflux temperature under a nitrogen atmosphere for 36 hours. After cooling, ether (250 mL) was added. The resulting white solid was collected by filtration, washed with ether and dried to give 1-cyclopropyl-1,4-dihydro-6,7-difluoro-8-methoxy-4-oxo-quinoline-3-carboxylic acid difluoroboryl ester (223) as a white solid (7.29 g; 63% yield). MS 344 (M+H).

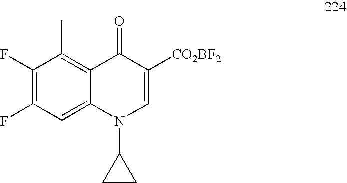

### 1-Cyclopropyl-1,4-dihydro-6,7-difluoro-5-methyl-4- oxo-quinoline carboxylic acid difluoroboryl ester (224)

This was prepared in a manner analogous to 1-cyclopropyl-1,4-dihydro-6,7-difluoro-8-methoxy-4-oxo-quinoline-3-carboxylic acid difluoroboryl ester (223) but starting with 1-cyclopropyl-1,4-dihydro-6,7-difluoro-5-methyl-4-oxo-quinoline carboxylic acid (prepared as described in Bioorganic and Medicinal Chemistry 1995, 3, 1699) to afford (224) as a white powder (58%). MS 328 (M+H).

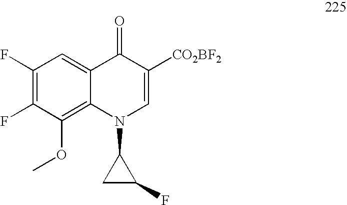

### (1R,2S)-1-[2-Fluorocyclopropyl]-1,4-dihydro-6,7- difluoro-8-methoxy-4-oxo-quinoline-3-carboxylic acid difluoroboryl ester (225)

This was prepared in a manner analogous to 1-cyclopropyl-1,4-dihydro-6,7-difluoro-8-methoxy-4-oxo-quinoline-3-carboxylic acid difluoroboryl ester (223) but starting with 1-[(1R,2S)-2-fluorocyclopropyl]I-1,4-dihydro-6,7-difluoro-5-methyl-4-oxo-quinoline carboxylic acid (prepared as described in WO 01/072738) to afford (225) as a grey solid (49%). MS 362 (M+H).

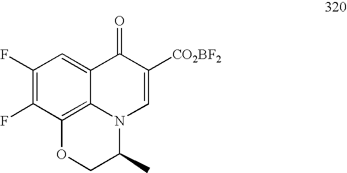

### S-(-)-9, 10-difluoro-2,3-dihydro-3-methyl-7-oxo-7H- pyrido-[1,2,3-de][1,4]-benzoxazine-6-carboxylic acid difluoroboryl ester (320)

This was prepared in an analogous manner as difluoroboryl ester 223, but starting with S-(−)-9,10-difluoro-2,3-dihydro-3-methyl-7-oxo-7H-pyrido-[1,2,3-de][1,4]-benzoxazine-6-carboxylic acid to afford 320 as an off-white powder (65%). MS 330 (M+H).

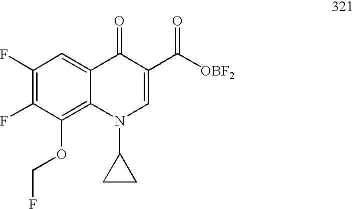

### 1-Cyclopropyl-1,4-dihydro-6,7-difluoro-8- fluoromethoxy-4-oxo-quinoline-3-carboxylic acid difluoroboryl ester (321)

To a solution of 309 (0.15 g, 0.48 mmol) in THF (5 mL) was added K₂CO₃ (73 mg, 0.53 mmol) followed by BF₃.Et₂O (0.066 mL, 0.52 mmol). The reaction was heated to reflux temperature for 6.5 hours. The reaction mixture was allowed to cool to room temperature, diluted with ethyl ether (50 mL), and filtered. The resulting solid was washed with CH₃CN (3×30 mL). The CH₃CN solutions were combined and concentrated to give the title compound 321 as a yellow solid (0.1 g, 58%). MS 362 (M+H).

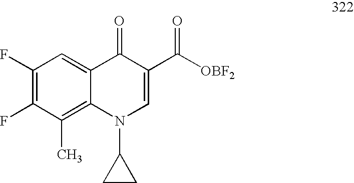

### 1-Cyclopropyl-1,4-dihydro-6,7-difluoro-8-methyl-4- oxo-quinoline-3-carboxylic acid difluoroboryl ester (322)

This was prepared in a manner analogous to 1-cyclopropyl-1,4-dihydro-6,7-difluoro-8-fluoromethoxy-4-oxo-quinoline-3-carboxylic acid difluoroboryl ester (321) but starting with 1-cyclopropyl-1,4-dihydro-6,7-difluoro-8-methyl-4-oxo-quinoline carboxylic acid (prepared as described in EP 237955). 322 was obtained as a white powder (93%). MS 328 (M+H).

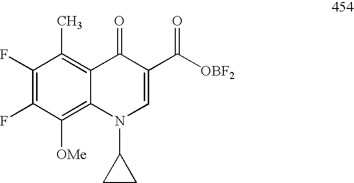

### 1-Cyclopropyl-1,4-dihydro-6,7-difluoro-8-methoxy-5- methyl-4-oxo-quinoline-3-carboxylic acid difluoroboryl ester (454)

This was prepared in a manner analogous to 1-cyclopropyl-1,4-dihydro-6,7-difluoro-8-fluoromethoxy-4-oxo-quinoline-3-carboxylic acid difluoroboryl ester (321) but starting with 1-cyclopropyl-1,4-dihydro-6,7-difluoro-8-methoxy-5-methyl-4-oxo-quinoline carboxylic acid (317). Compound 454 was obtained as a white powder (99%). MS 358 (M+H).

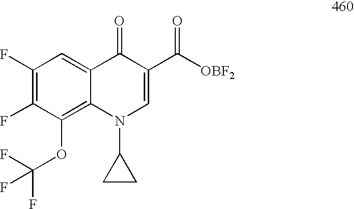

### 1-Cyclopropyl-1,4-dihydro-6,7-difluoro-8- trifluoromethoxy-4-oxo-quinoline-3-carboxylic acid difluoroboryl ester (460)

This was prepared in a manner analogous to 1-cyclopropyl-1,4-dihydro-6,7-difluoro-8-fluoromethoxy-4-oxo-quinoline-3-carboxylic acid difluoroboryl ester (321) but starting with 1-cyclopropyl-1,4-dihydro-6,7-difluoro-8-trifluoromethoxy-4-oxo-quinoline carboxylic acid (prepared as described in EP0352123). Compound 460 was obtained as a white powder (95%). MS 398 (M+H).

### Precursor Preparation B

### Side Chain III

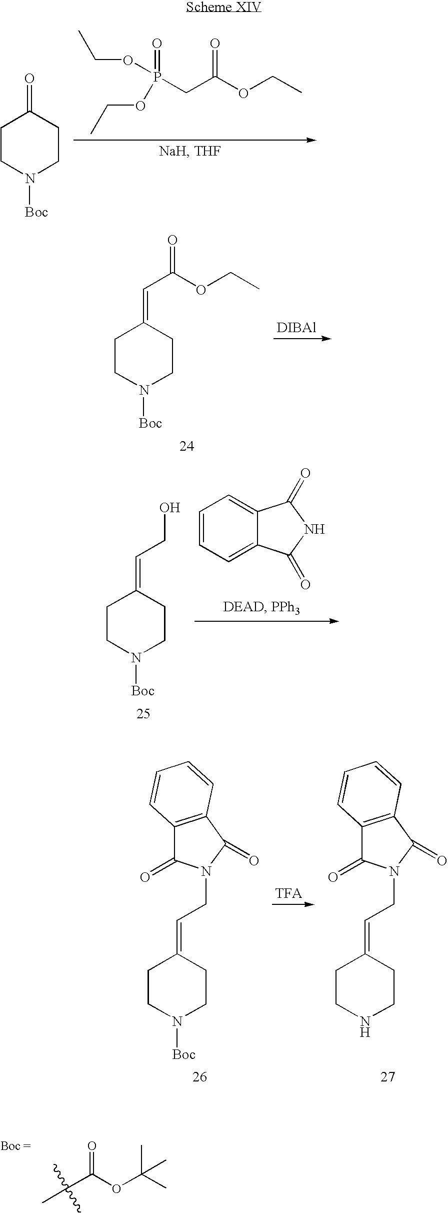

### 4-[2-(1,3-dihydro-1,3-dioxo-2H-isoindol-2- yl)ethylidene]-1-piperidine trifluoroacetate (27)

### Step 1

t-Butyl 4-(2-Ethoxy-2-oxoethylidene)piperidinyl-1-carboxylate (24) was prepared according the procedure described in Sato et al. Heterocycles, 2001, 54, 747.

### Step 2

t-Butyl 4-(2-Hydroxyethylidene)piperidinyl-1-carboxylate (25) was prepared according the procedure described in Sato et al. Heterocycles, 2001, 54, 747.

### Step 3

t-Butyl 4-[2-(1,3-Dihydro-1,3-dioxo-2H-isoindol-2-yl)ethylidene]-piperidinyl-1-carboxylate (26) was prepared by a procedure adapted from Synthesis 1995, 756. A solution of 25 (250 mg, 1.10 mmol), phthalimide (208 mg, 1.40 mmol), and triphenylphosphine (366 mg, 1.40 mmol) in dry THF (10 mL) was treated with diethyl azodicarboxylate (0.25 mL, 1.40 mmol) added via syringe in the dark under nitrogen. After 5 h, the reaction mixture was treated with water (10 mL), diluted with ethyl acetate (50 mL), washed with 10% aqueous sodium bicarbonate (2×25 mL), and dried (MgSO₄). Purification by flash chromatography (0-30% ethyl acetate/hexanes) afforded the title compound (389 mg, 78%) as a white foam. MS 357 (M+H).

### Step 4: 4-[2-(1,3-dihydro-1,3-dioxo-2H-isoindol-2-yl)ethylidene]-1-piperidine trifluoroacetate (27)

A solution of 26 (380 mg, 1.03 mmol) was dissolved in CH₂Cl₂ (50 mL) and was treated with trifluoroacetic acid (1 mL) at room temperature. After 1 h, the reaction mixture was concentrated in vacuo to afford the title Compound 27 (363 mg, 100%) as an oil. MS 257 (M+H).

1-(tert-Butoxycarbonyl)-4-piperidinone was reacted with each of the respective phosphonoacetates listed in Table 3, and the products subjected to analogous procedures as in the synthesis of 27, to prepare the corresponding alcohols (28-30, 84) and the derived amines (31-33, 85).

TABLE 3

| Phosphono | | | | | | |
|---|---|---|---|---|---|---|
| acetates | Cpd | Alcohols | (M + H) | Cpd | Amines | (M + H) |
| 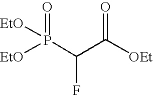 | 28 | 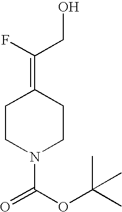 | 246 | 31 | 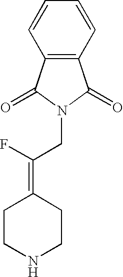 | 275 |
| 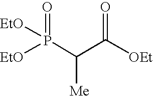 | 29 | 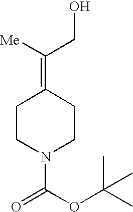 | 242 | 32 | 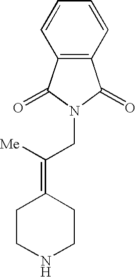 | 271 |
| 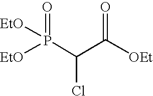 | 30 | 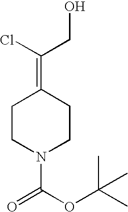 | 262 | 33 | 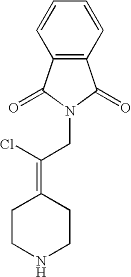 | 291 |
| 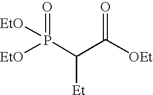 | 84 | 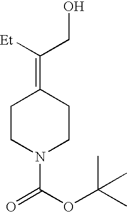 | 256 | 85 | 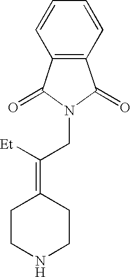 | 285 |

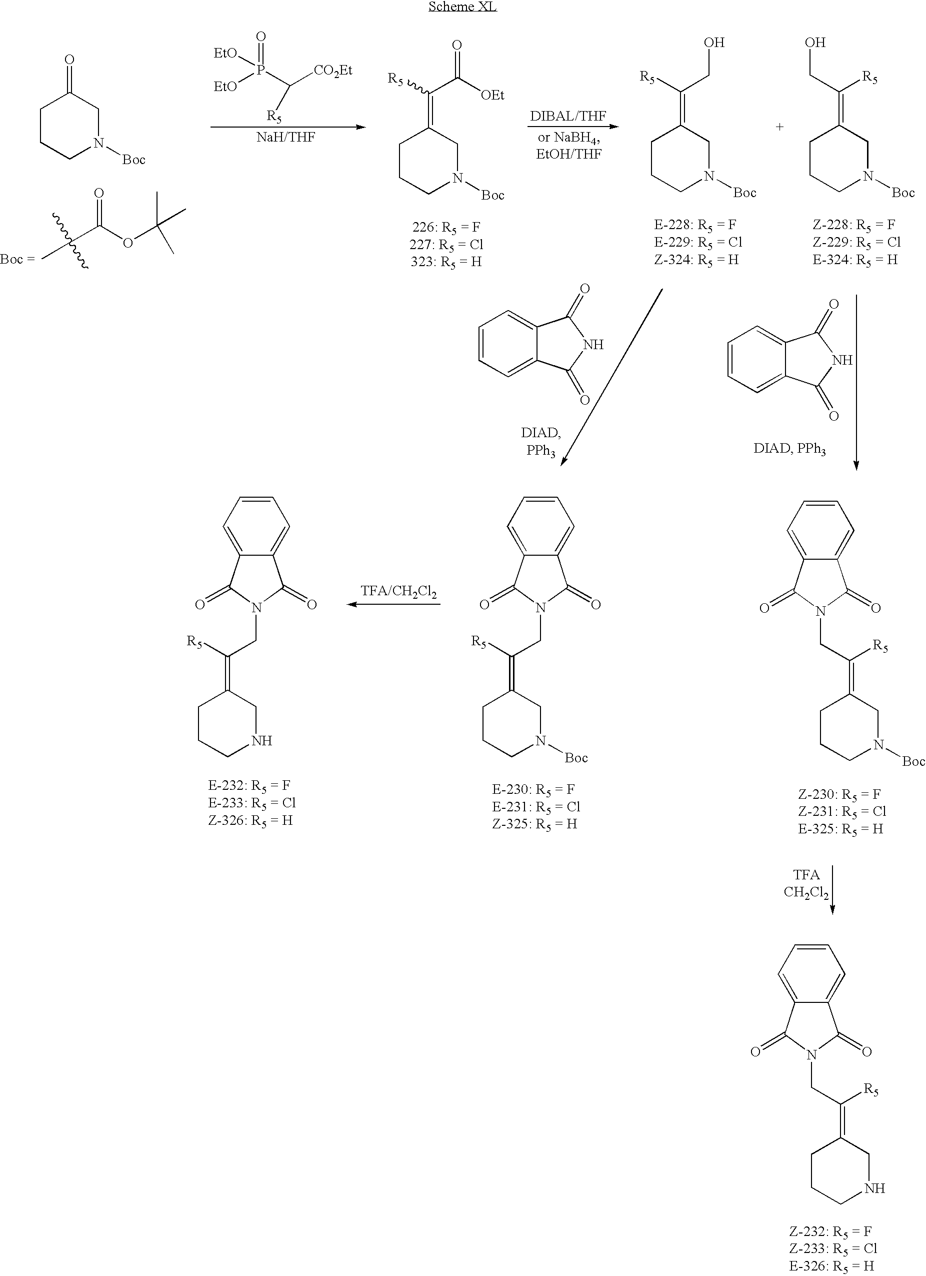

### Compounds E-232, Z-232, E-233, Z-233, Z-326, and E-326 of Scheme XL:

### (E/Z)-t-Butyl 3-(1-fluoro-2-ethoxy-2-oxoethylidene)- piperidinyl-1-carboxylate (226; R5=F)

This was prepared by the same procedure as in the synthesis of 24 except that 1-(tert-butoxycarbonyl)-3-piperidinone was used in place of 1-(tert-butoxycarbonyl)-4-piperidinone and triethyl 2-fluorophosphonoacetate was used in place of triethyl phosphonoacetate. MS 310 (M+Na).

### (E/Z)-t-Butyl 3-(1-chloro-2-ethoxy-2-oxoethylidene)-piperidinyl-1-carboxylate (227; R₅═Cl)

This was prepared by the same procedure as in the synthesis of 24 except that 1-(tert-butoxycarbonyl)-3-piperidinone was used in place of 1-(tert-butoxycarbonyl)-4-piperidinone and triethyl 2-chlorophosphonoacetate was used in place of triethyl phosphonoacetate. MS 326 (M+Na).

### (E/Z)-t-Butyl 3-(2-ethoxy-2-oxoethylidene)-piperidinyl-1-carboxylate (323; R₅═H)

This was prepared by the same procedure as in the synthesis of 24 except that 1-(tert-butoxycarbonyl)-3-piperidinone was used in place of 1-(tert-butoxycarbonyl)-4-piperidinone. MS 292 (M+Na).

### (E and Z)-t-Butyl 3-(1-chloro-2-hydroxyethylidene)-piperidinyl-1-carboxylate (E-229 and Z-229; R₅═Cl)

A 1.0 M solution of DIBAL-H in toluene (8.2 mL, 8.23 mmol) was added to a solution of 227 (1.0 g, 3.29 mmol) in tetrahydrofuran (10 mL) at −78° C. under nitrogen and the mixture was stirred at the same temperature for 5 h, then warmed to room temperature and stirred overnight. 0.5 M Rochelle's salt solution (40 mL) and EtOAc (80 mL) was added to the reaction at 0° C. The resulting mixture was stirred at rt for 3 h. After phase separation the organic layer was concentrated. The E/Z isomers were separated by flash chromatography (0-40% ethyl acetate/hexanes) to afford E-229 [200 mg, 23%, MS 284 (M+Na)] as a yellow oil and Z-229 [250 mg, 29%, MS 284 (M+Na)] as a white solid.

### (E and Z)-t-Butyl 3-(1-fluoro-2-hydroxyethylidene)-piperidinyl-1-carboxylate (E-228 and Z-228; R₅═F)

### Method 1:

This was prepared in a similar manner to the procedure described above except that 226 was used in place of 227. MS 268 (M+Na) and MS 268 (M+Na).

### Method 2:

To the mixture of regioisomeric esters 226 (E and Z; 21.0 g; 73.09 mmoles) in ethanol/THF (85 mL/300 mL) at 0° C., was added sodium borohydride (9.60 g; 253.7 mmoles) in several portions over a period of about 45 minutes. The reaction was allowed to come to room temperature and stir overnight. The reaction mixture was recooled to 0° C. and quenched by the careful addition of saturated ammonium chloride (125 mL). The reaction mixture was poured into water (350 mL) and extracted with ethyl acetate (3×250 mL). The combined organic layers were washed with water, brine, dried (Na₂SO₄), filtered and evaporated. The regioisomeric alcohols (E-228 and Z-228) were separated by column chromatography (15% ethyl acetate/hexanes). The less polar spot was the E alcohol and the lower spot the Z alcohol. Both were isolated as clear oils (79% combined yield).

### (Z)-t-Butyl 3-(2-hydroxyethylidene)-piperidinyl-1-carboxylate (Z-324; R₅═H)

This was prepared by the same procedure as in the synthesis of E-229 except that 323 was used in place of 227. MS 250 (M+Na).

### (E)-t-Butyl 3-(2-hydroxyethylidene)-piperidinyl-1-carboxylate (E-324; R₅═H)

This was prepared by the same procedure as in the synthesis of Z-229 except that 323 was used in place of 227. MS 250 (M+Na).

### (E)-t-Butyl 3-[1-fluoro-2-(1,3-dioxo-1,3-dihydro-2H-isoindol-2-yl)-ethylidene]-piperidinyl-1-carboxylate (E-230; R₅═F)

This was prepared by a similar procedure as in the synthesis of 26 except that E-228 was used in place of 25 and diisopropyl azodicarboxylate was used in place of diethyl azodicarboxylate. MS 397 (M+Na).

### (Z)-t-Butyl 3-[1-fluoro-2-(1,3-dioxo-1,3-dihydro-2H-isoindol-2-yl)-ethylidene]-piperidinyl-1-carboxylate (Z-230; R₅═F)

This was prepared by a similar procedure as in the synthesis of 26 except that Z-228 was used in place of 25 and diisopropyl azodicarboxylate was used in place of diethyl azodicarboxylate. MS 397 (M+Na).

### (E)-t-Butyl 3-[1-chloro-2-(1,3-dioxo-1,3-dihydro-2H-isoindol-2-yl)-ethylidene]-piperidinyl-1-carboxylate (E-231; R₅═Cl)

This was prepared by a similar procedure as in the synthesis of 26 except that E-229 was used in place of 25 and diisopropyl azodicarboxylate was used in place of diethyl azodicarboxylate. MS 413 (M+Na).

### (Z)-t-Butyl 3-[1-chloro-2-(1,3-dioxo-1,3-dihydro-2H-isoindol-2-yl)-ethylidene]-piperidinyl-1-carboxylate (Z-231; R₅═Cl)

This was prepared by a similar procedure as in the synthesis of 26 except that Z-229 was used in place of 25 and diisopropyl azodicarboxylate was used in place of diethyl azodicarboxylate. MS 413 (M+Na).

### (Z)-t-Butyl 3-[2-(1,3-dioxo-1,3-dihydro-2H-isoindol-2-yl)-ethylidene]-piperidinyl-1-carboxylate (Z-325; R₅═H)

This was prepared by a similar procedure as in the synthesis of 26 except that Z-324 was used in place of 25 and diisopropyl azodicarboxylate was used in place of diethyl azodicarboxylate. MS 379 (M+Na).

### (E)-t-Butyl 3-[2-(1,3-dioxo-1,3-dihydro-2H-isoindol-2-yl)-ethylidene]-piperidinyl-1-carboxylate (E-325; R₅═H)

This was prepared by a similar procedure as in the synthesis of 26 except that E-324 was used in place of 25 and diisopropyl azodicarboxylate was used in place of diethyl azodicarboxylate. MS 379 (M+Na).

### (E)-3-[1-fluoro-2-(1,3-dioxo-1,3-dihydro-2H-isoindol-2-yl)-ethylidene]-piperidine Trifluoroacetate (E-232; R₅═F)

This was prepared by the same procedure as in the synthesis of 135 except that E-230 was used in place of 133. MS 275 (M+H).

### (Z)-3-[1-fluoro-2-(1,3-dioxo-1,3-dihydro-2H-isoindol-2-yl)-ethylidene]-piperidine Trifluoroacetate (Z-232; R₅═F)

This was prepared by the same procedure as in the synthesis of 135 except that Z-230 was used in place of 133. MS 275 (M+H).

### (E)-3-[1-chloro-2-(1,3-dioxo-1,3-dihydro-2H-isoindol-2-yl)-ethylidene]-piperidine Trifluoroacetate (E-233; R₅═Cl)

This was prepared by the same procedure as in the synthesis of 135 except that E-231 was used in place of 133. MS 291 (M+H).

### (Z)-3-[1-chloro-2-(1,3-dioxo-1,3-dihydro-2H-isoindol-2-yl)-ethylidene]-piperidine Trifluoroacetate (Z-233; R₅═Cl)

This was prepared by the same procedure as in the synthesis of 135 except that Z-231 was used in place of 133. MS 291 (M+H).

### (Z)-3-[2-(1,3-dioxo-1,3-dihydro-2H-isoindol-2-yl)-ethylidene]-piperidine Trifluoroacetate (Z-326; R₅═H)

This was prepared by the same procedure as in the synthesis of 135 except that Z-325 was used in place of 133. MS 257 (M+H).

### (E)-3-[2-(1,3-dioxo-1,3-dihydro-2H-isoindol-2-yl)-ethylidene]-piperidine Trifluoroacetate (E-326; R₅═H)

This was prepared by the same procedure as in the synthesis of 135 except that E-325 was used in place of 133. MS 257 (M+H).

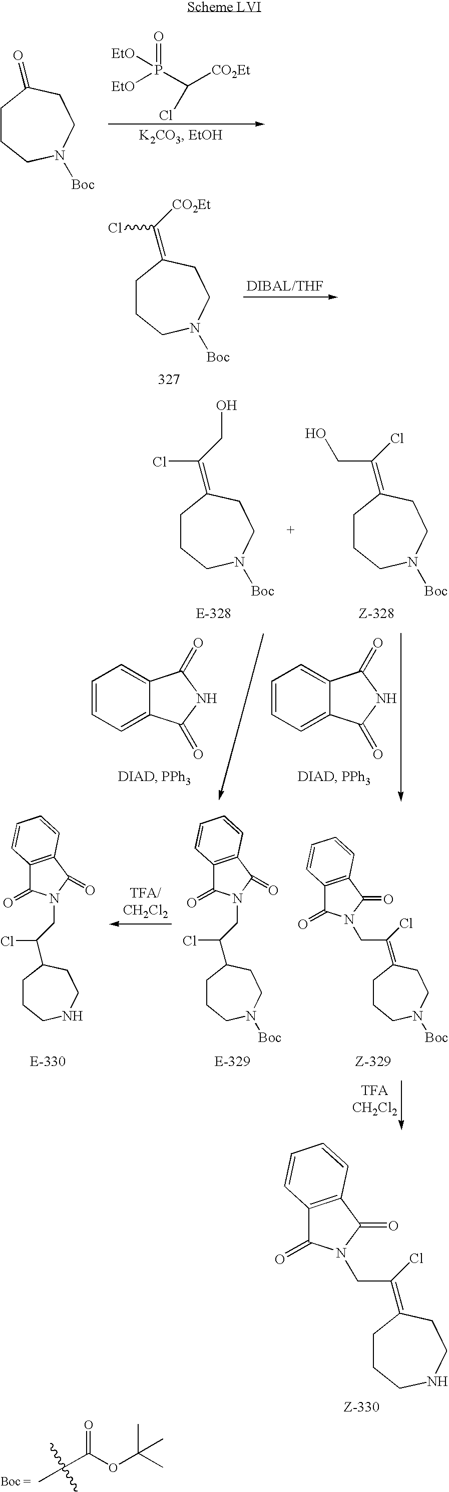

The following compounds were prepared using the procedure illustrated in Scheme LVI.

### (E/Z)-t-Butyl 4-(1-chloro-2-ethoxy-2-oxoethylidenyl)-hexahydroazepine-1-carboxylate (327)

This was prepared by the same procedure as in the synthesis of 24 except that 1-(tert-butoxycarbonyl)hexahydroazepin-4-one was used in place of 1-(tert-butoxycarbonyl)-4-piperidinone and triethyl 2-chlorophosphonoacetate was used in place of triethyl phosphonoacetate. MS 338 (M+H).

### (E and Z)-t-Butyl 4-(1-chloro-2-hydroxy-ethylidenyl)-hexahydroazepine-1-carboxylate (E-328 and Z-328)

This was prepared by the same procedure as in the synthesis of E-229 and Z-229 except that (E/Z)-t-butyl 4-(1-chloro-2-ethoxy-2-oxoethylidenyl)-hexahydroazepine-1-carboxylate (B327) was used in place of 227. MS 276 (M+H) for both isomers.

### (E)-t-Butyl 4-[1-chloro-2-(1,3-dioxo-1,3-dihydro-2H-isoindol-2-yl)-ethylidenyl]-hexahydroazepine-1-carboxylate (E-329)

This was prepared by a similar procedure as in the synthesis of 26 except that E-328 was used in place of 25. MS 405 (M+H).

### (Z)-t-Butyl 4-[1-chloro-2-(1,3-dioxo-1,3-dihydro-2H-isoindol-2-yl)-ethylidenyl]-hexahydroazepine-1-carboxylate (Z-329)

This was prepared by a similar procedure as in the synthesis of 26 except that Z-328 was used in place of 25. MS 405 (M+H).

### (E)-4-[1-chloro-2-(1,3-dioxo-1,3-dihydro-2H-isoindol-2-yl)-ethylidenyl]-hexahydroazepine Trifluoroacetate (E-330)

This was prepared by the same procedure as in the synthesis of 135 except that E-329 was used in place of 133. MS 305 (M+H).

### (Z)-4-[1-chloro-2-(1,3-dioxo-1,3-dihydro-2H-isoindol-2-yl)-ethylidenyl]-hexahydroazepine Trifluoroacetate (Z-330)

This was prepared by the same procedure as in the synthesis of 135 except that Z-329 was used in place of 133. MS 305 (M+H).

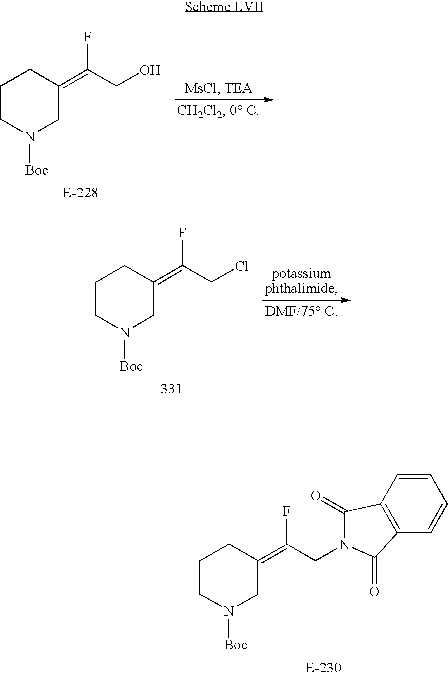

An alternative two-step procedure for the synthesis of E-230 from E-228 is depicted in Scheme LVII.

### Step 1

To alcohol E-228 (6.90 grams; 28.13 mmoles) in methylene chloride (200 mL) at 0° C., was added triethylamine (7.5 mL; 53.81 mmoles) and methanesulfonyl chloride (3.0 mL; 38.76 mmoles). After warming to room temperature and stirring overnight, the reaction was poured into water (150 mL) and the organic layer separated. The organic layer was washed with saturated sodium bicarbonate (100 mL), brine (100 mL), dried (Na₂SO₄), filtered and evaporated to give chloride 331 as an oil. MS 264, 266 (M+H).

### Step 2

Chloride 331 (28.13 mmoles) and potassium phthalimide (5.16 grams; 27.86 mmoles) in anhydrous DMF (75 mL) were heated at 75° C. overnight. The reaction was slowly poured into ice water with stirring. The resulting solid was collected, washed with water and dried to afford phthalimide E-230 as a cream-colored powder (74%). MS 397 (M+Na).

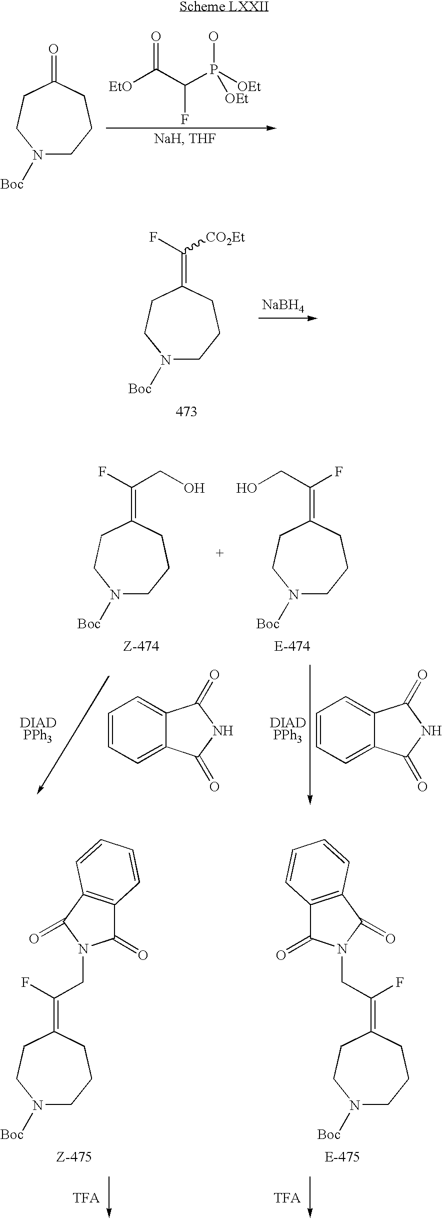

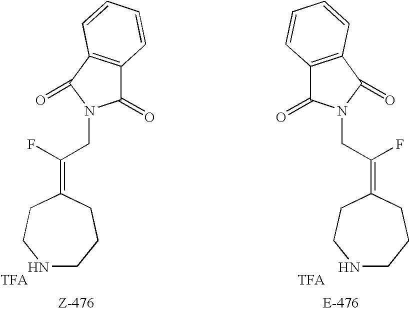

### 4-(Ethoxycarbonyl-fluoro-methylene)-azepane-1- carboxylic acid tert-butyl ester (473)

Ester 473 was prepared by an analogous procedure as for compound 122 except that N-Boc-azepan-4-one was used in place of ketone 121. MS 302 (M+H).

### (Z)-4-(1-Fluoro-2-hydroxy-ethylidene)-azepane-1-carboxylic Acid tert-butyl Ester (Z-474) and (E)-4-(1-Fluoro-2-hydroxy-ethylidene)-azepane-1-carboxylic Acid tert-butyl Ester (E-474)

Alcohols Z-474 and E-474 were prepared by an analogous procedure as for compounds E-228 and Z-228, method 2 except that ester 473 was used in place of ester 226. MS 260 (M+H).

### (Z)-4-[2-(1,3-Dioxo-1,3-dihydro-isoindol-2-yl)-1-fluoro-ethylidene]-azepane-1-carboxylic Acid tert-butyl Ester (Z-475)

Phthalimide Z-475 was prepared by an analogous procedure as for phthalimide 26 except that alcohol Z-474 was used in place of 25 and diisopropyl azodicarboxylate was used in place of diethyl azodicarboxylate. MS 389 (M+H).

### (E)-4-[2-(1,3-Dioxo-1,3-dihydro-isoindol-2-yl)-1-fluoro-ethylidene]-azepane-1-carboxylic Acid tert-butyl Ester (E-475)

Phthalimide Z-475 was prepared by an analogous procedure as for phthalimide 26 except that alcohol E-474 was used in place of 25 and diisopropyl azodicarboxylate was used in place of diethyl azodicarboxylate. MS 389 (M+H).

### (Z)-2-(2-Azepan-4-ylidene-2-fluoro-ethyl)-isoindole-1,3-dione (Z-476)

Amine Z-476 was prepared by an analogous procedure as for amine Z-355 except that phthalimide Z-475 was used in place of phthalimide Z-354. MS 289 (M+H).

### (E)-2-(2-Azepan-4-yl idene-2-fluoro-ethyl)-isoindole-1,3-dione (E-476)

Amine E-476 was prepared by an analogous procedure as for amine E-355 except that phthalimide E-475 was used in place of phthalimide E-354. MS 289 (M+H).

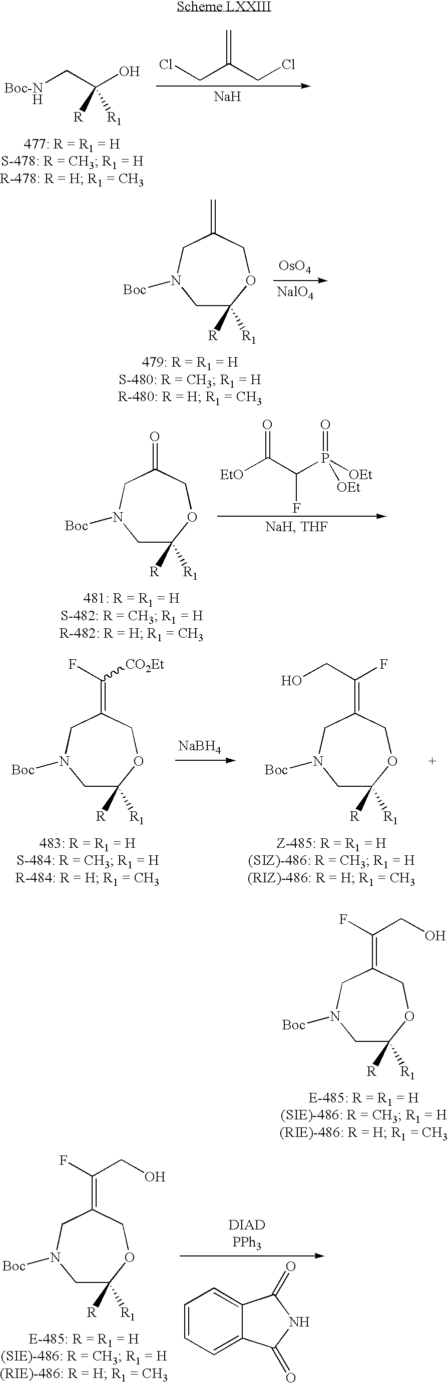

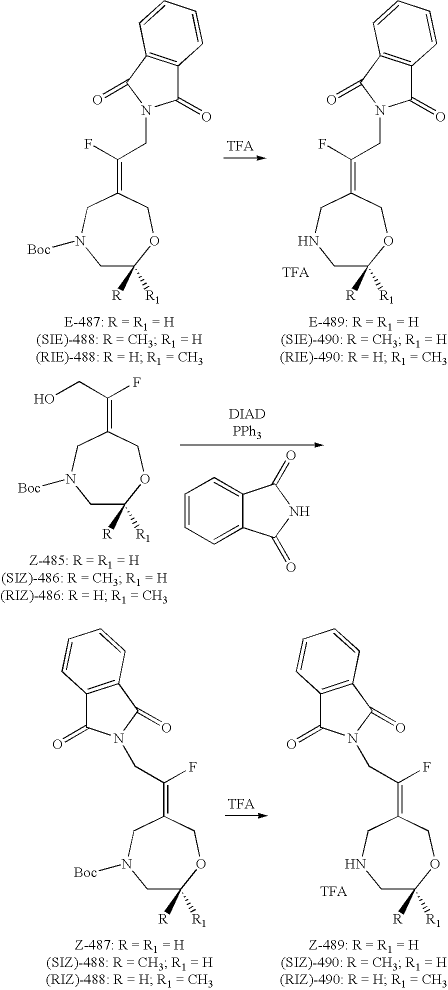

### 6-Methylene-[1,4]oxazepane-4-carboxylic acid tert- butyl ester (479)

Oxazepane 479 was prepared from aminoalcohol 477 by the procedure described in WO04074291. MS 214 (M+H).

### (2S)-2-Methyl-6-methylene-[1,4]oxazepane-4-carboxylic Acid tert-butyl Ester (S-480)

Oxazepane S-480 was prepared by an analogous procedure as for compound 479 except that aminoalcohol S-478 was used in place of 477. MS 228 (M+H).

### (2R)-2-Methyl-6-methylene-[1,4]oxazepane-4-carboxylic Acid tert-butyl Ester (R-480)

Oxazepane R-480 was prepared by an analogous procedure as for compound 479 except that aminoalcohol R-478 was used in place of 477. MS 228 (M+H).

### 6-Oxo-[1,4]oxazepane-4-carboxylic Acid tert-butyl Ester (481)

Oxazepanone 481 was prepared from 479 as described in WO04074291. MS 216 (M+H).

### (2S)-2-Methyl-6-oxo-[1,4]oxazepane-4-carboxylic Acid tert-butyl Ester (S-482)

Oxazepanone S-482 was prepared by an analogous procedure as for compound 481 except that oxazepane S-480 was used in place of 479. MS 230 (M+H).

### (2R)-2-Methyl-6-oxo-[1,4]oxazepane-4-carboxylic Acid tert-butyl Ester (R-482)

Oxazepanone R-482 was prepared by an analogous procedure as for compound 481 except that oxazepane R-480 was used in place of 479. MS 230 (M+H).

### (E/Z)-6-(Ethoxycarbonyl-fluoro-methylene)-[1,4]oxazepane-4-carboxylic Acid tert-butyl Ester (483)

Ester 483 was prepared by an analogous procedure as for compound 122 except that oxazepanone 481 was used in place of ketone 121. MS 304 (M+H).

### (2S)-(E/Z)-6-(Ethoxycarbonyl-fluoro-methylene)-2-methyl-[1,4]oxazepane-4-carboxylic Acid tert-butyl Ester (S-484)

Ester S-484 was prepared by an analogous procedure as for compound 483 except that oxazepanone S-482 was used in place of oxazepanone 481. MS 318 (M+H).

### (2R)-(E/Z)-6-(Ethoxycarbonyl-fluoro-methylene)-2-methyl-[1,4]oxazepane-4-carboxylic Acid tert-butyl Ester (R-484)

Ester R-484 was prepared by an analogous procedure as for compound 483 except that oxazepanone R-482 was used in place of oxazepanone 481. MS 318 (M+H).

### (Z)-6-(1-Fluoro-2-hydroxy-ethylidene)-[1,4]oxazepane-4-carboxylic Acid tert-butyl Ester (Z-485) and (E)-6-(1-Fluoro-2-hydroxy-ethylidene)-[1,4]oxazepane-4-carboxylic Acid tert-butyl Ester (E-485)

Alcohols Z-485 and E-485 were prepared by an analogous procedure as for compounds E-228 and Z-228, method 2 except that ester 483 was used in place of ester 226. MS 262 (M+H).

### (2S)-(Z)-6-(1-Fluoro-2-hydroxy-ethylidene)-2-methyl-[1,4]oxazepane-4-carboxylic Acid tert-butyl Ester (SIZ-486) and (2S)-(E)-6-(1-Fluoro-2-hydroxy-ethylidene)-2-methyl-[1,4]oxazepane-4-carboxylic Acid tert-butyl Ester (S/E-486)

Alcohols SIZ-486 and S/E-486 were prepared by an analogous procedure as for compounds Z-485 and E-485, method 2 except that ester S-484 was used in place of ester 483. MS 276 (M+H).

### (2R)-(Z)-6-(1-Fluoro-2-hydroxy-ethylidene)-2-methyl-[1,4]oxazepane-4-carboxylic Acid tert-butyl Ester (R/Z-486) and (2R)-(E)-6-(1-Fluoro-2-hydroxy-ethylidene)-2-methyl-[1,4]oxazepane-4-carboxylic Acid tert-butyl Ester (R/E-486)

Alcohols R/Z-486 and R/E-486 were prepared by an analogous procedure as for compounds Z-485 and E-485, method 2 except that ester R-484 was used in place of ester 483. MS 276 (M+H).

### (Z)-6-[2-(1,3-Dioxo-1,3-dihydro-isoindol-2-yl)-1-fluoro-ethylidene]-[1,4]oxazepane-4-carboxylic Acid tert-butyl Ester (Z-487)

Phthalimide Z-487 was prepared by an analogous procedure as for phthalimide 26 except that alcohol Z-485 was used in place of 25 and diisopropyl azodicarboxylate was used in place of diethyl azodicarboxylate. MS 391 (M+H).

### (E)-6-[2-(1,3-Dioxo-1,3-dihydro-isoindol-2-yl)-1-fluoro-ethylidene]-[1,4]oxazepane-4-carboxylic Acid tert-butyl Ester (E-487)

Phthalimide E-487 was prepared by an analogous procedure as for phthalimide 26 except that alcohol E-485 was used in place of 25 and diisopropyl azodicarboxylate was used in place of diethyl azodicarboxylate. MS 391 (M+H).

### (2S)-(Z)-6-[2-(1,3-Dioxo-1,3-dihydro-isoindol-2-yl)-1-fluoro-ethylidene]-2-methyl-[1,4]oxazepane-4-carboxylic Acid tert-butyl Ester (SIZ-488)

Phthalimide SIZ-488 was prepared by an analogous procedure as for phthalimide Z-487 except that alcohol SIZ-486 was used in place of Z-485. MS 405 (M+H).

### (2R)-(Z)-6-[2-(1,3-Dioxo-1,3-dihydro-isoindol-2-yl)-1-fluoro-ethylidene]-2-methyl-[1,4]oxazepane-4-carboxylic Acid tert-butyl Ester (R/Z-488)

Phthalimide R/Z-488 was prepared by an analogous procedure as for phthalimide Z-487 except that alcohol R/Z-486 was used in place of Z-485. MS 405 (M+H).

### (2S)-(E)-6-[2-(1,3-Dioxo-1,3-dihydro-isoindol-2-yl)-1-fluoro-ethylidene]-2-methyl-[1,4]oxazepane-4-carboxylic Acid tert-butyl Ester (S/E-488)

Phthalimide S/E-488 was prepared by an analogous procedure as for phthalimide E-487 except that alcohol S/E-486 was used in place of E-485. MS 405 (M+H).

### (2R)-(E)-6-[2-(1,3-Dioxo-1,3-dihydro-isoindol-2-yl)-1-fluoro-ethylidene]-2-methyl-[1,4]oxazepane-4-carboxylic Acid tert-butyl Ester (R/E-488)

Phthalimide R/E-488 was prepared by an analogous procedure as for phthalimide E-487 except that alcohol R/E-486 was used in place of E-485. MS 405 (M+H).

### (Z)-2-(2-Fluoro-2-[1,4]oxzepan-6-ylidene-ethyl)-isoindole-1,3-dione (Z-489)

Amine Z-489 was prepared by an analogous procedure as for amine Z-355 except that phthalimide Z-487 was used in place of phthalimide Z-354. MS 291 (M+H).

### (E)-2-(2-Fluoro-2-[1,4]oxazepan-6-ylidene-ethyl)-isoindole-1,3-dione (E-489)

Amine E-489 was prepared by an analogous procedure as for amine E-355 except that phthalimide E-487 was used in place of phthalimide E-354. MS 291 (M+H).

### (2S)-(Z)-2-[2-Fluoro-2-(2-methyl-[1,4]oxazepan-6-ylidene)-ethyl]-isoindole-1,3-dione (SIZ-490),

Amine SIZ-490 was prepared by an analogous procedure as for amine Z-489 except that phthalimide SIZ-488 was used in place of phthalimide Z-487. MS 305 (M+H).

### (2R)-(Z)-2-[2-Fluoro-2-(2-methyl-[1,4]oxazepan-6-ylidene)-ethyl]-isoindole-1,3-dione (R/Z-490)

Amine R/Z-490 was prepared by an analogous procedure as for amine Z-489 except that phthalimide R/Z-488 was used in place of phthalimide Z-487. MS 305 (M+H).

### (2S)-(E)-2-[2-Fluoro-2-(2-methyl-[1,4]oxazepan-6-ylidene)-ethyl]-isoindole-1,3-dione (S/E-490)

Amine S/E-490 was prepared by an analogous procedure as for amine E-489 except that phthalimide S/E-488 was used in place of phthalimide E-487. MS 305 (M+H).

### (2R)-(E)-2-[2-Fluoro-2-(2-methyl-[1,4]oxazepan-6-ylidene)-ethyl]-isoindole-1,3-dione (R/E-490)

Amine R/E-490 was prepared by an analogous procedure as for amine E-489 except that phthalimide R/E-488 was used in place of phthalimide E-487. MS 305 (M+H).

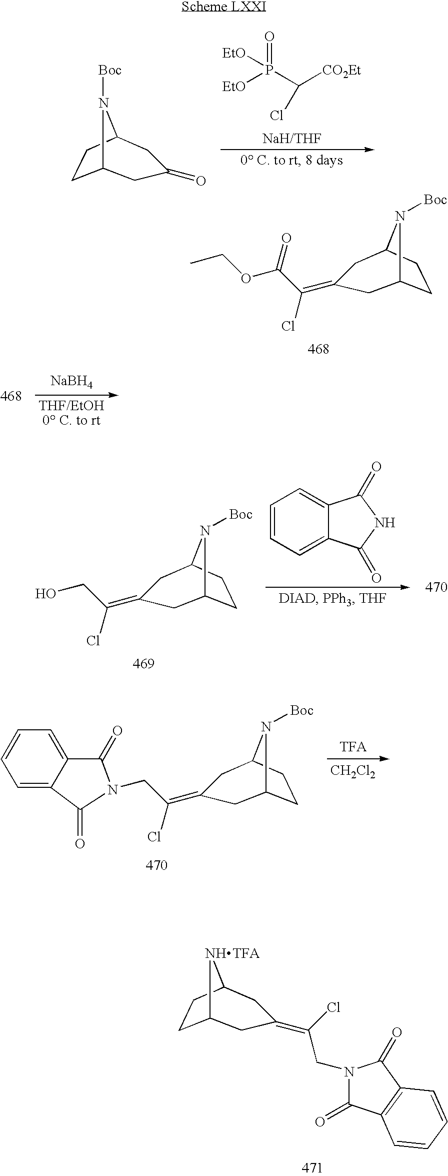

### 3-[2-(1,3-dioxo-1,3-dihydro-2H-isoindol-2-yl)-1-chloro- ethylidene]-8-azabicyclo[3.2.1]octane trifluoroacetate salt (471).

### Step 1

Ester 468 was prepared in a similar fashion as 24 except that N-Boc-nortropinone was used in place of 4-piperidinone and triethyl 2-chlorophosphonoacetate was used in place of triethyl phosphonoacetate. In the case of 468, the reaction mixture was stirred at room temperature for 8 days in order to drive the reaction to completion. MS 330, 332 (M+H).

### Step 2

Alcohol 469 was prepared in a similar manner as E-228 and Z-228, method 2 except that ester 468 was used in place of 226. MS 288, 290 (M+H).

### Step 3:

Phthalimide 470 was prepared in a similar manner as 26 except that alcohol 469 was used in place of 25 and diisopropyl azodicarboxylate was used in place of diethyl azodicarboxylate. MS 417, 419 (M+H).

### Step 4

Amine 471 was prepared in a similar manner as 27 except that phthalimide 470 was used in place of 26. MS 317, 319 (M+H).

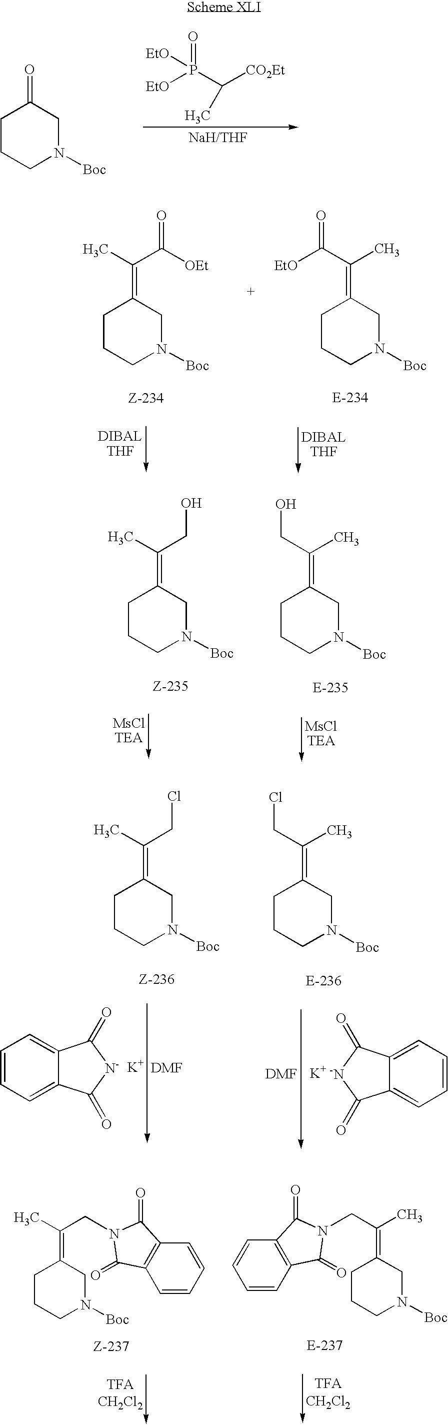

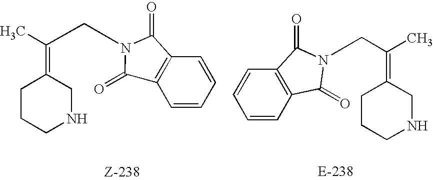

### (Compounds Z-238 and E-238 of Scheme XLI:

### (Z)-t-Butyl 3-(1-ethoxy-1-oxo-2-propylidene)- piperidinyl-1-carboxylate (Z-234) and (E)-t-Butyl 3-(1- ethoxy-1-oxo-2-propylidene)-piperidinyl-1 - carboxylate (E-234)

To sodium hydride (1.10 g, mmol) (60% in oil) in tetrahydrofuran (50 mL) at 0° C. was added triethyl-2-phosphonopropionate (7.00 mL, 32.0 mmols) and the resulting mixture was stirred for 20 min whereupon 1-(tert-butoxycarbonyl)-3-piperidinone carboxylate (5.00 g, mmol) was added and stirred a further 5 hours at room temperature. The mixture was concentrated, diluted with half saturated aq. NaHCO₃ (200 mL) and extracted with ethyl acetate (6×40 mL). The organic extracts were dried over Na₂SO₄, concentrated and chromatographed on silica gel using 10% ethyl acetate/hexanes as eluent. The geometric isomers were isolated as clear oils. The reaction provided 1.66 g (23%) of the higher R_{f} E-isomer and 3.71 g (50%) of the lower R_{f} Z-isomer. Both: MS 284 (M+H).

### (Z)-t-Butyl 3-(1-hydroxy-2-propylidene)-piperidinyl-1-carboxylate (Z-235)

To Z-234 from the above reaction (0.4982 g, 1.758 mmols) in tetrahydrofuran (6 mL) at −78° C. was added DIBAL (4.0 mL, 1.0 M in toluene). The mixture was stirred for 5 hours at −78° C. and then 30 min at room temperature. After concentrating and cooling to 0° C., 0.5M aq. Rochelle's Salt (50 mL) and ethyl acetate (20 mL) were added and the mixture stirred for 3 hours. The mixture was extracted with ethyl acetate (5×20 mL), dried over Na₂SO₄, concentrated and chromatographed on silica gel with 30% ethyl acetate/hexanes as eluent. The product was obtained as a clear oil (0.3163 g, 74% yield). MS 242 (M+H).

### (Z)-t-Butyl 3-(1-chloro-2-propylidene)-piperidinyl-1-carboxylate (Z-236)

To Z-235 from the above reaction (0.1607 g, 0.6658 mmol) in CH₂Cl₂ (3 mL) was added triethylamine (0.28 mL, 2.0 mmols) and then methanesulfonyl chloride (0.08 mL, 1.0 mmol), and the resulting mixture stirred for 2 hours. This mixture was treated with sat. aq. NH₄Cl, extracted with CH₂Cl₂ (4×7 mL), dried over Na₂SO₄, concentrated, and chromatographed on silica gel (0-100% ethyl acetate/hexanes gradient as eluent) to give 0.1118 g (65%) of a clear oil. MS 260 (M+H).

### (Z)-t-Butyl 3-[1-(1,3-dioxo-1,3-dihydro-2H-isoindol-2-yl)-2-propylidene]-piperidinyl-1-carboxylate (Z-237)

To Z-236 from the above reaction (0.0859 g, 0.331 mmol) in dimethylformamide (3 mL) was added potassium phthalimide (0.075 g, 4.0 mmols). The mixture was stirred for 3 days at 50° C. Water (10 mL) and brine (10 mL) were added and the mixture was extracted with CH₂Cl₂ (4×8 mL), dried over Na₂SO₄, concentrated, and chromatographed on silica gel (0-40% ethyl acetate/hexanes gradient as eluent) to give Z-237 as a white solid (0.0459 g, 38%). MS 371 (M+H).

### (Z)-3-[1-(1,3-dioxo-1,3-dihydro-2H-isoindol-2-yl)-2-propyl idene]-piperidinyl-1-carboxylate (Z-238)

This was prepared by the same procedure as in the synthesis of 135 except that Z-237 was used in place of 133. MS 271 (M+H).

### (E)-3-[1-(1,3-dioxo-1,3-dihydro-2H-isoindol-2-yl)-2-propyl idene]-piperidinyl-1-carboxylate (E-238)

This geometrical isomer was prepared from E-234 through an analogous series of reactions as for the conversion of Z-234 to Z-238. MS 271 (M+H).

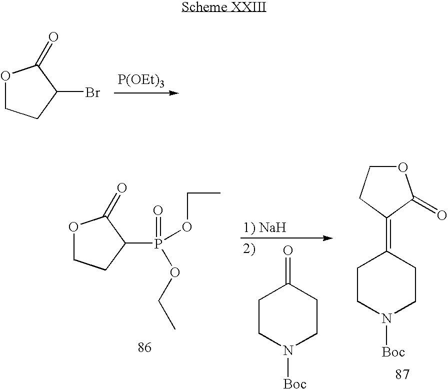

(2-Oxo-tetrahydro-furan-3-yl)-phosphonic acid diethyl ester (86; Scheme XXIII) was prepared according the procedure described in Murphy et al. Chemical Communications 1996, 6, 737-8.

4-(2-Oxo-dihydrofuran-3-ylidene)piperidine-1-carboxylic acid tert-butyl ester (87; Scheme XXIII) was prepared by an analogous procedure to that described in Sato et al. Heterocycles, 2001, 54, 747; MS=267 (M+H).

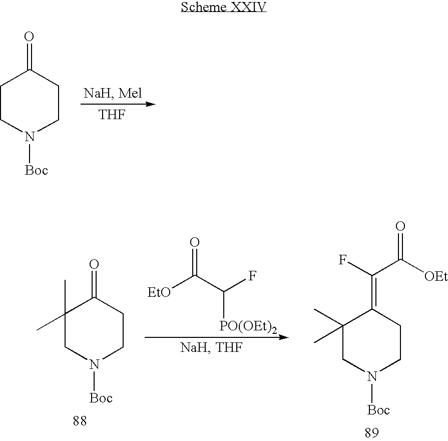

3,3-Dimethyl-4-oxo-piperidine-1-carboxylic acid tert-butyl ester (88; Scheme XXIV) was prepared according the procedure described in Vice et al. J. Org. Chem. 2001, 66, 2487-2492.

4-(2-Ethoxy-1-fluoro-2-oxoethylidene)-3,3-dimethylpiperidine-1-carboxylic acid tert-butyl ester (89; Scheme XXIV) was prepared by a procedure analogous to that described in Sato et al. Heterocycles, 2001, 54, 747.

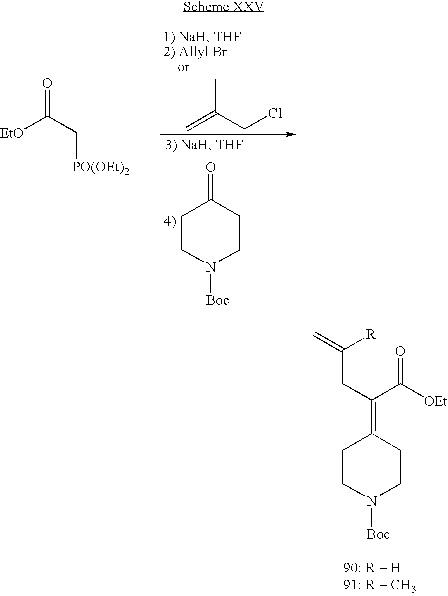

### 4-(1-Ethoxycarbonyl-but-3-enylidene)piperidine-1 - carboxylic acid tert-butyl ester (90; Scheme XXV)

A slurry of sodium hydride (1.50 g, 37.6 mmol) in THF (100 mL) at 0° C. under nitrogen was carefully treated with triethyl phosphonoacetate (8.12 mL, 37.6 mmol) via a syringe. After 30 min, the reaction mixture was treated with allyl bromide (3.3 mL, 37.6 mmol) and the resulting mixture was allowed to warm to 25° C. over 12 h. The resulting mixture was recooled to 0° C., treated with sodium hydride (1.50 g, 37.6 mmol), and the resulting slurry was allowed to stir for 30 min at 0° C. A solution of 1-(tert-butoxycarbonyl)-4-piperidinone (5.0 g, 25 mmol) in THF (50 mL) was added via a cannula over 10 min and the resulting solution was allowed to warm to 25° C. over 12 h. The reaction was quenched by the addition of 15% aqueous sodium bicarbonate (50 mL) and the resulting mixture was diluted with ethyl acetate (100 mL), washed with 15% aqueous sodium bicarbonate (2×100 mL), and concentrated in vacuo. Purification by chromatography (0-50% EtOAc/hexanes) afforded title compound (1.93 g, 25%) as a yellow oil: MS (M+H)=310.

4-(1-Ethoxycarbonyl-3-methyl-but-3-enylidene)piperidine-1-carboxylic acid tert-butyl ester (91; Scheme XXV) was prepared according to the procedure described for 90 except methylallyl chloride was used instead of allyl bromide.

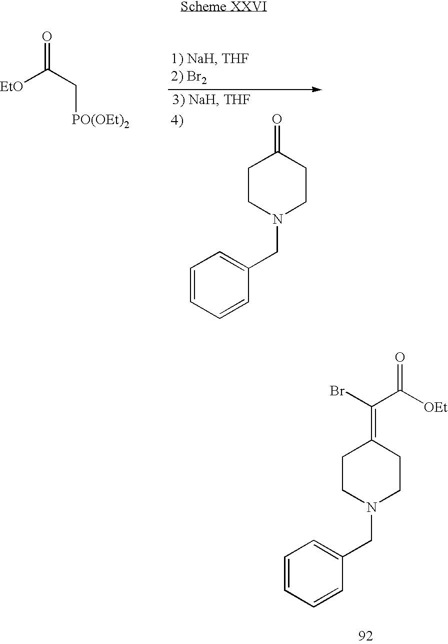

### (1-Benzyl-piperidin-4-ylidene)bromoacetic acid ethyl ester (92; Scheme XXVI)

A slurry of sodium hydride (1.50 g, 37.6 mmol) in THF (100 mL) at 0° C. under nitrogen was carefully treated with triethyl phosphonoacetate (8.12 mL, 37.6 mmol) via a syringe. After 30 min, the reaction mixture was treated with bromine (1.95 mL, 37.6 mmol) via a dropping funnel over 10 min and the resulting mixture was allowed to stir for 3 h. The reaction mixture was treated with sodium hydride (1.50 g, 37.6 mmol) and the resulting slurry was allowed to stir for 30 min at 0° C. A solution of 1-benzylpiperidin-4-one (5.0 g, 25 mmol) in THF (50 mL) was added via a cannula over 10 min and the resulting solution was allowed to warm to 25° C. over 12 h. The reaction was quenched by the addition of 15% aqueous sodium bicarbonate (50 mL) and the resulting mixture was diluted with ethyl acetate (100 mL), washed with 15% aqueous sodium bicarbonate (2×100 mL), and concentrated in vacuo. Purification by chromatography (0-50% EtOAc/hexanes) afforded the title compound (6.35 g, 74%) as a red-orange oil: MS (M+=H)=339.

The alcohols listed in Table 6 were prepared in a similar fashion as described for t-butyl 4-(2-hydroxyethylidene)piperidinyl-1-carboxylate (25), except the corresponding ethylidene carboxylate was used instead of t-butyl 4-(2-ethoxy-2-oxoethylidene)piperidinyl-1-carboxylate (24).

TABLE 6

| | | Com- | (M + |
|---|---|---|---|
| Ethylidine Carboxylate | Alcohols | pound | H) |
| 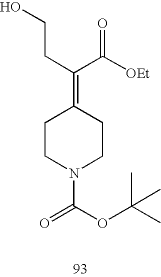 | 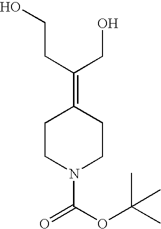 | 94 | 272 |
| 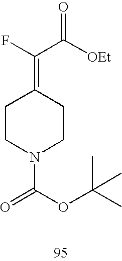 | 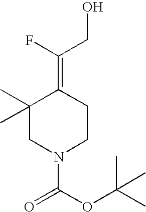 | 96 | 274 |
| 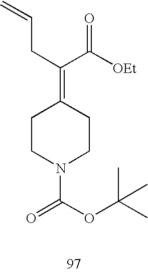 | 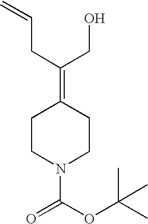 | 98 | 268 |
| 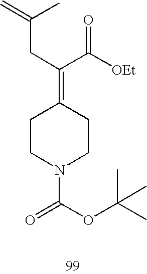 | 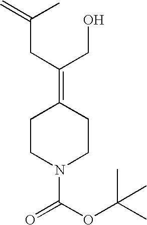 | 100 | 282 |
| 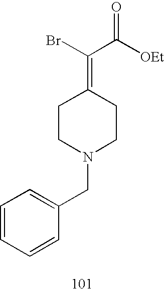 | 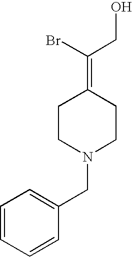 | 102 | 297 |

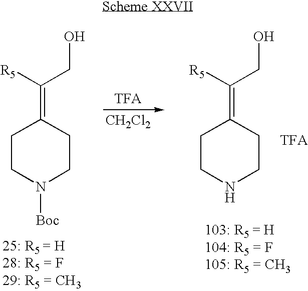

### 2-Piperidin-4-ylidene-ethanol trifluoroacetate (103; Scheme XXVII)

A solution of 25 (191 mg, 0.5 mmol) was dissolved in CH₂Cl₂ (10 mL) and was treated with trifluoroacetic acid (0.5 mL) at room temperature. After 1 h, the reaction mixture was concentrated in vacuo to afford the title compound (64 mg, 100%) as an oil. MS 129 (M+H).

2-Piperidin-4-ylidene-propan-1-ol trifluoroacetate (105; Scheme XXVII) was prepared according the procedure described for 103 except 29 was used. MS 142 (M+H).

2-Fluoro-2-piperidin-4-ylidene-ethanol trifluoroacetate (104; Scheme XXVII) was prepared according the procedure described for 103 except 28 was used. MS 146 (M+H).

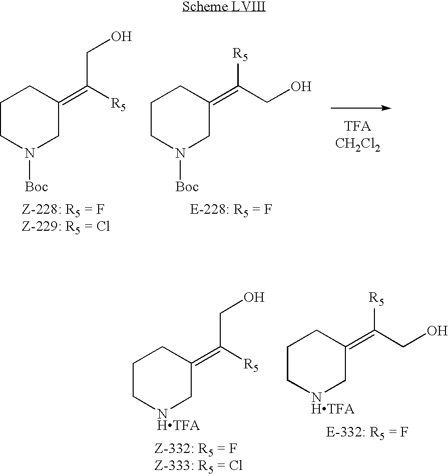

### (Z)-2-Fluoro-2-piperidin-3-ylidene-ethanol trifluoroacetate (Z-332; R5=F).

This was prepared by the same procedure as in the synthesis of 135 except that Z-228 was used in place of 133. MS 146 (M+H).

### (E)-2-Fluoro-2-piperidin-3-ylidene-ethanol Trifluoroacetate (E-332; R₅═F)

This was prepared by the same procedure as in the synthesis of 135 except that E-228 was used in place of 133. MS 146 (M+H).

### (Z)-2-Chloro-2-piperidin-3-ylidene-ethanol Trifluoroacetate (Z-333; R₅=Cl)

This was prepared by the same procedure as in the synthesis of 135 except that Z-229 was used in place of 133. MS 162 (M+H).

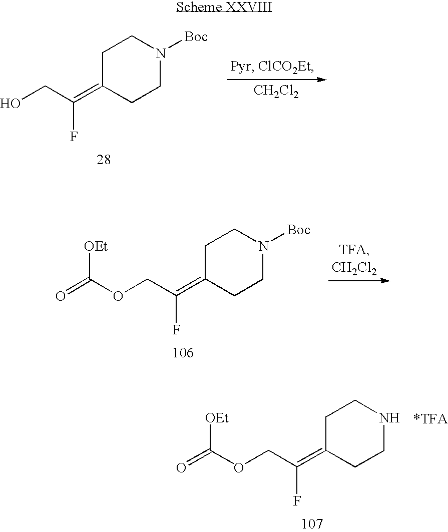

### t-Butyl 4-(2-ethoxycarbonyloxy-1- fluoroethylidene)piperidine-1-carboxylate (106; Scheme XXVIII).

To alcohol 28 (0.5064 g, 2.064 mmols) in CH₂Cl₂ (10 mL) at RT was added pyridine (0.23 mL, 2.8 mmols) and then ethyl chloroformate (0.22 mL, 2.2 mmols). After stirring overnight, sat. aq. NH₄Cl (10 mL) was added and the mixture extracted with CH₂Cl₂ (5×10 mL), dried over Na₂SO₄, concentrated and chromatographed on silica (20% EtOAc/hexane as eluent) to provide the title compound 106 (0.4546 g, 69%) as a clear oil. MS 318 (M+H).

### 4-(2-Ethoxycarbonyloxy-1-fluoro-ethylidene)-piperidine (107; Scheme XXVIII)

To compound 106 (0.1787 g, 0.5631 mmol) in CH₂Cl₂ (3 mL) was added TFA (0.56 mL, 7.3 mmols) and the mixture stirred for 3 hrs whereupon all volatile materials were removed in vacuo to provide the crude title compound, which was used without further purification. MS 218 (M+H).

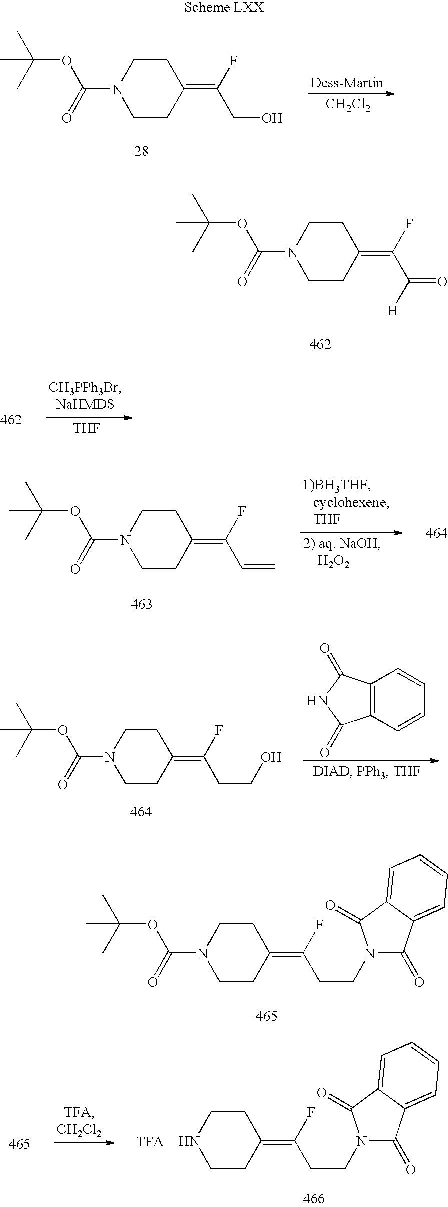

### 4-[1-Fluoro-3-(1 ,3-dihydro-1,3-dioxo-2H-isoindol-2- yl)propylidene]-piperidine trifluoroacetate salt (466).

### Step 1

To a solution of alcohol 28 (2.0 g, 8.2 mmol) in CH₂Cl₂ (100 mL) at room temperature was added Dess-Martin periodinane (5.0 g, 11.8 mmol). The reaction mixture was stirred at room temperature for 16 hours before it was diluted with hexane and filtered. The resulting solution was washed with H₂O and brine, dried over MgSO₄, and concentrated. Purification by silica gel chromatography (10% EtOAc in hexane) afforded aldehyde 462 as a colorless oil (1.7 g, 86%).

### Step 2

To a solution of CH₃PPh₃Br (5.0 g, 14 mmol) in THF (60 mL) at room temperature was added NaHMDS (1.0 M in THF, 12.6 mL, 12.6 mmol). The reaction mixture was kept at room temperature for 15 minutes. It was then cooled to 0° C., and a solution of aldehyde 462 (1.7 g, 7.0 mmol) in THF (10 mL) was added. The reaction was stirred at 0° C. for 30 minutes before it was quenched with aq. NaHCO₃. The mixture was extracted with hexane (2×100 mL). The combined organic solution was washed with H₂O and brine, dried over MgSO₄, and concentrated. Purification by silica gel chromatography (5% EtOAc in hexane) afforded diene 463 as a colorless oil (1.3 g, 77%).

### Step 3

To BH₃THF (1N in THF, 8.1 mL, 8.1 mmol) at 0° C. was added cyclohexene (1.64 mL, 16.2 mmol). The mixture was stirred at 0° C. for 1 hour before a solution of diene 463 (1.3 g, 5.4 mmol) in THF (5 mL) was added. The reaction was slowly warmed up to room temperature and stirred for 3 hours. To the reaction was then added aq. 1N NaOH (24 mL) followed by slow addition of aq. 30% H₂O₂ (3 mL). The resulting mixture was stirred at room temperature for 45 minutes before it was extracted with EtOAc (3×50 mL). The combined organic solution was washed with H₂O and brine, dried over MgSO₄, and concentrated. Purification by silica gel chromatography (25% EtOAc in hexane) afforded alcohol 464 as a colorless oil (0.96 g, 69%).

### Step 4

A solution of alcohol 464 (0.92 g, 3.55 mmol) with phthalimide (0.78 g, 5.3 mmol), PPh₃ (1.4 g, 5.3 mmol), and diisopropyl azodicarboxylate (1.03 mL, 5.3 mmol) in THF (35 mL) was stirred at room temperature for 20 hours. It was concentrated and diluted with EtOAc (100 mL). The resulting solution was washed with aq. 1N HCl, aq. NaHCO₃ and brine, dried over MgSO₄, and concentrated. Purification by silica gel chromatography (20% EtOAc in hexane) afforded phthalimide 465 as a white solid (1.2 g, 87%).

### Step 5

To a solution of phthalimide 465 (0.45 g, 1.16 mmol) in CH₂Cl₂ (8 mL) at room temperature was added CF₃CO₂H (2 mL). The solution was stirred at room temperature for 4 hours. It was concentrated to give amine salt 466 as a crude product, which was used in the next step of reaction without further purification.

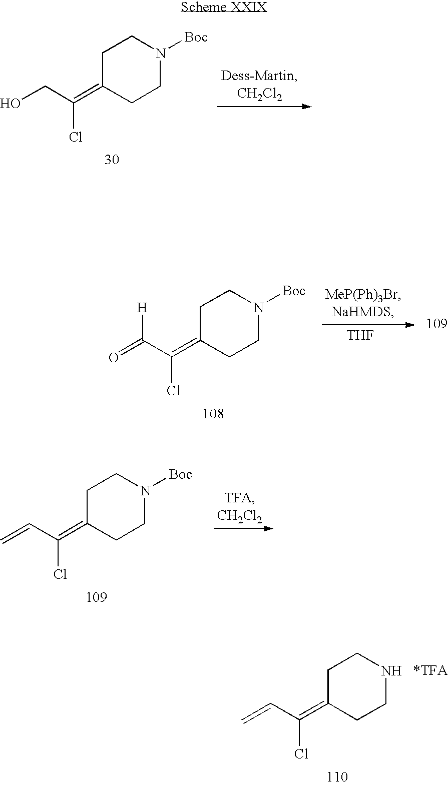

### t-Butyl 4-(1 -chloro-2-oxoethylidene)-piperidine-1- carboxylate (108; Scheme XXIX)

To alcohol 30 (6.01 g, 23.0 mmols) in CH₂Cl₂ at RT and open to the air was added the Dess-Martin reagent (21.17 g, 49.9 mmols) and the reaction mixture stirred overnight whereupon the mixture was washed with sat. aq. Na₂S₂O₃ (60 mL) and sat. aq. NaHCO₃ (3×30 mL). The organic layer was dried over Na₂SO₄, concentrated and chromatographed on silica (25% EtOAc/Hexane as eluent) to provide the title compound 108 (5.22 g, 88%) as a white crystalline solid. MS 260 (M+H).

### t-Butyl 4-(1-Chloro-2-propenylidene)piperidine-1-carboxylate (109; Scheme XXIX)

Methyltriphenylphosphonium bromide (5.51 g, 15.4 mmols) in THF (40 mL) at 0° C. was treated with sodium bis(trimethylsilyl)amide (15.4 mL, 1.0 M in THF) and stirred for 20 min whereupon compound 108 (2.05 g, 7.89 mmols) in THF (15 mL) was added via cannula and the mixture stirred for 3 hrs, warming to RT. The mixture was quenched by adding sat. aq. NH₄Cl (20 mL) and the aqueous layer was extracted with EtOAc (6×20 mL). The combined organic layers were dried over Na₂SO₄, concentrated and chromatographed on silica (gradient elution with 0-10% MeOH/CH₂Cl₂) to provide the title compound 109 (1.94 g, 96%) as a white crystalline solid. MS 258 (M+H).

### 4-(1-Chloro-2-propenylidene)piperidine TFA Salt (110; Scheme XXIX)

To compound 109 (0.1415 g, 0.5489 mmol) in CH₂Cl₂ (5 mL) was added TFA (0.55 mL, 7.1 mmols) and the mixture stirred for 3 hrs whereupon all volatile materials were removed in vacuo. The crude title compound so obtained was used without further purification. MS 158 (M+H).

The protected amines listed in Table 7 were prepared in a similar fashion as described for t-butyl 4-[2-(1,3-dihydro-1,3-dioxo-2H-isoindol-2-yl)ethylidene]-piperidinyl-1-carboxylate (26), except the corresponding alcohol was used instead of t-butyl 4-(2-hydroxyethylidene)piperidinyl-1-carboxylate (25).

TABLE 7

| Alcohol | Protected Amine | Compound | (M + H) |
|---|---|---|---|
| 94 | 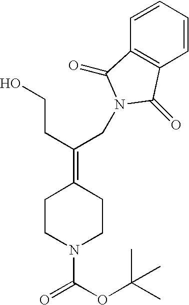 | 111 | 401 |
| 96 | 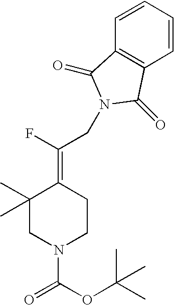 | 112 | 403 |
| 98 | 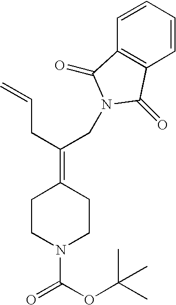 | 113 | 397 |
| 100 | 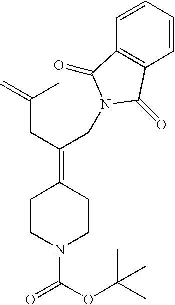 | 114 | 411 |
| 102 | 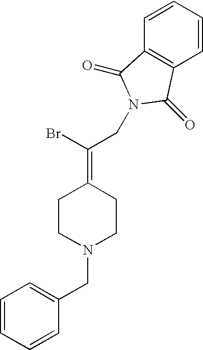 | 115 | 426 |

The amines listed in Table 8 were prepared in a similar fashion as described for 4-[2-(1,3-dihydro-1,3-dioxo-2H-isoindol-2-yl)ethylidene]-1-piperidine trifluoroacetate (27), except the corresponding protected amine was used instead of t-butyl 4-[2-(1,3-dihydro-1,3-dioxo-2H-isoindol-2-yl)ethylidene]-piperidinyl-1-carboxylate (26).

TABLE 8

| Protected | | | |
|---|---|---|---|
| Amine | Amine | Compound | (M + H) |
| 111 | 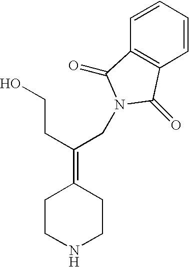 | 116 | 301 |
| 112 | 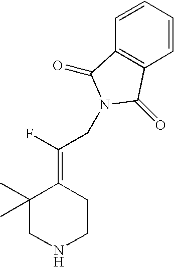 | 117 | 303 |
| 113 | 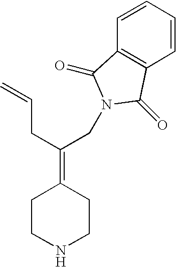 | 118 | 297 |
| 114 | 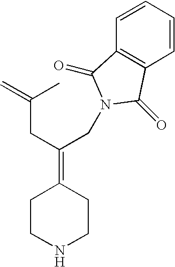 | 119 | 311 |

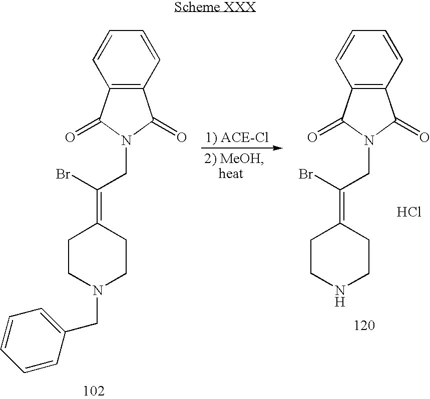

### 2-(2-Bromo-2-piperidin-4-ylidenylethyl)isoindole-1,3- dione hydrochloride (120: Scheme XXX)

A mixture of 102 (0.50 g, 1.17 mmol) and 1-chloroethyl chloroformate (0.7 mL, 6.2 mmol) in dichloroethane (10 mL) was warmed to reflux temperature for 2 h. The resulting solution was allowed to cool to room temperature and concentrated in vacuo. The residue was dissolved in methanol (50 mL) and warmed to reflux temperature for 2 h. The reaction mixture was allowed to cool to room temperature and concentrated in vacuo to afford a white solid. The residue was washed with diethyl ether (2×) and dried to afford title compound (432 mg, 100%) as an orange oil. MS 336 (M+H).

### Compounds Z-37 and E-37 of Scheme XV

### (E/Z)-Ethyl chloro(1-benzyl-3-pyrrolidinylidene)acetate (34)

Prepared by the same procedure as in the synthesis of 24 except that 1-benzyl-pyrrolidin-3-one was used in place of 1-(tert-butoxycarbonyl)-4-piperidinone and triethyl 2-chlorophosphonoacetate was used in place of triethyl phosphonoacetate. MS 280 (M+H).

### (E/Z)-2-(1-Benzyl-3-pyrrolidinylidene)-2-chloroethanol (35)

Prepared by the same procedure as in the synthesis of 25 except that 34 was used in place of 24. MS 283 (M+H).

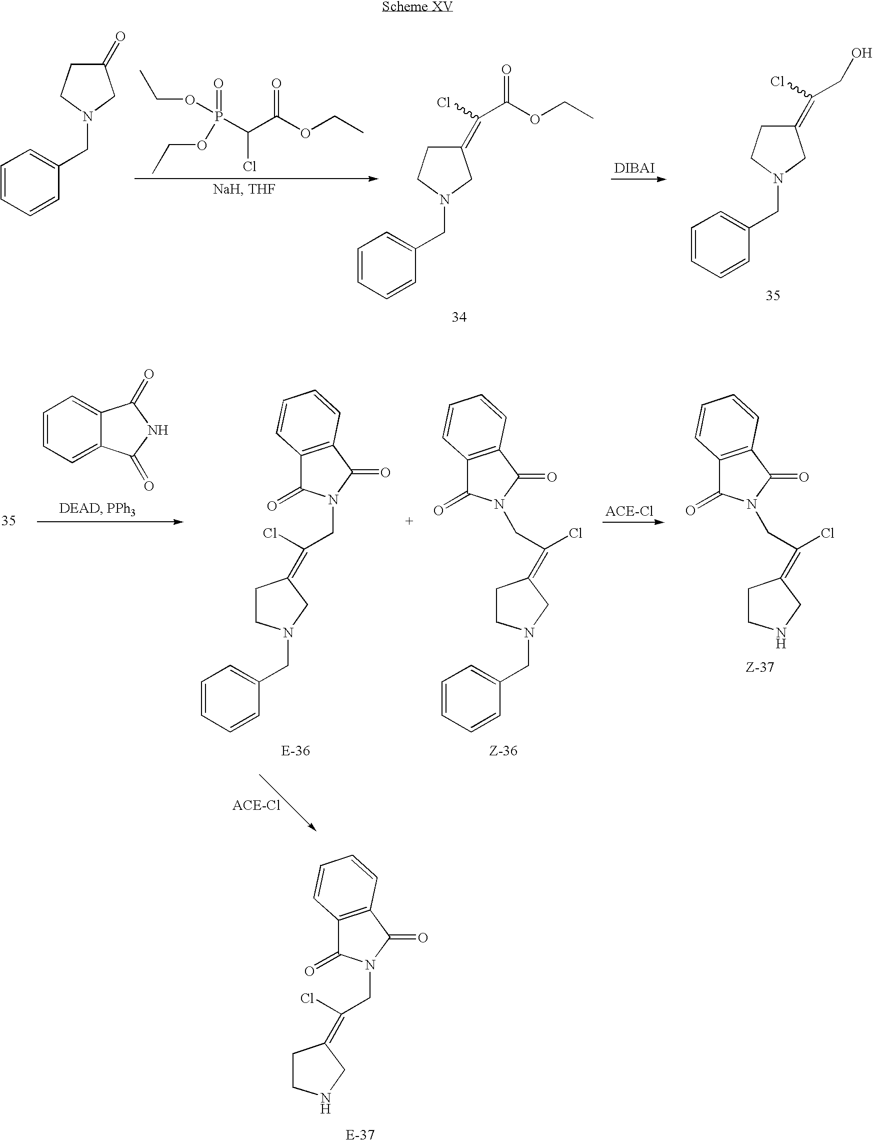

### E/Z)-2-[2-(1-Benzyl-3-pyrrolidinylidene)-2- chloroethyl]-1 H-isoindole-1,3(2H)-dione (E-36 and Z-36)

Prepared by the same procedure as in the synthesis of 26 except that 35 (1.58 g) was used in place of 25. The E/Z isomers were separated by MPLC (0-45% ethyl acetate/hexanes) to afford Z-36 (430 mg, MS 367 (M+H)) as a reddish oil and E-36 (420 mg, MS 367 (M+H)) as a reddish oil.

### (E)-2-[2-Chloro-2-(3-pyrrolidinylidene)ethyl]-1H-isoindole-1,3(2H)-dione Hydrochloride (E-37)

A mixture of E-36 (0.430 g, 1.45 mmol) and 1-chloroethyl chloroformate (0.7 mL, 6.2 mmol) in dichloroethane (10 mL) was warmed to reflux temperature for 2 h. The resulting solution was allowed to cool to room temperature, and concentrated in vacuo. The residue was dissolved in methanol (50 mL) and warmed to reflux temperature for 2 h. The reaction mixture was allowed to cool to room temperature and concentrated in vacuo to afford a white solid. The residue was washed with diethyl ether (2×) and dried to afford E-37 (200 mg, 50%) as a brown oil. MS 277 (M+H).

### (Z)-2-[2-Chloro-2-(3-pyrrolidinylidene)ethyl]-1H-isoindole-1,3(2H)-dione Hydrochloride (Z-37)

Prepared by the same procedure as in the synthesis of E-37 except that Z-36 was used in place of E-36. MS 277 (M+H).

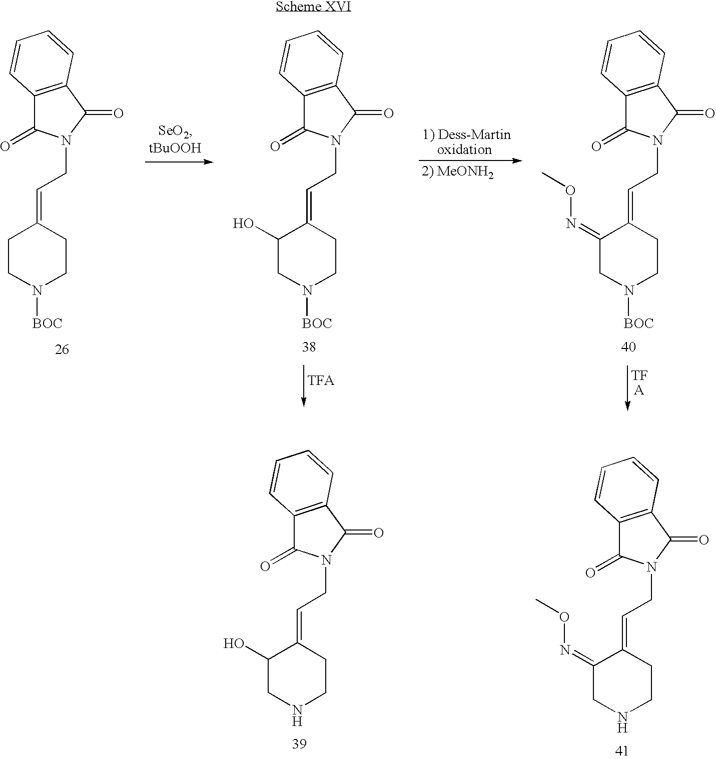

### Compounds 39 and 41 of Scheme XVI:

### t-Butyl (E)-4-[2-(1,3-dioxo-1,3-dihydro-2H-isoindol-2- yl)ethylidene]-3-hydroxy-piperidinyl-1-carboxylate (38)

A slurry of SeO₂ (0.5 g, 6.06 mmol) in CH₂Cl₂ (5 mL) at 0° C. was treated with tert-butyl hydroperoxide (2.5 mL, 9.09 mmol, 5-6 M, 10% in undecane) via a syringe. After 20 min, the reaction mixture was treated with a solution of ethylidene 26 (1.44 g, 4.04 mmol) in CH₂Cl₂ (15 mL) and the resulting mixture was allowed to stir for 12 h at room temperature. The reaction was carefully quenched by the addition of 15% aqueous sodium thiosulfate (15 mL), and the reaction mixture was diluted with CH₂Cl₂ (25 mL). The layers are separated, and the organic layer was washed with 15% aqueous sodium thiosulfate (15 mL), dried (MgSO₄), filtered and concentrated in vacuo. Purification by flash chromatography (silica gel, 0-75% ethyl acetate/hexanes) afforded the title compound 38 (0.51 g, 33%) as a white solid. MS 373 (M+H).

### (E)-4-[2-(1,3-dioxo-1,3-dihydro-2H-isoindol-2-yl)ethylidene]-3-hydroxypiperidine (39)

Prepared by the same procedure as in the synthesis of 27 except that 38 was used in place of 26. MS 273 (M+H).

### (E)-t-Butyl 4-[2-(1,3-dioxo-1,3-dihydro-2H-isoindol-2-yl)ethylidene]-3-methoxyimino-piperidinyl-1-carboxylate (40)

A solution of 38 (0.51 g, 1.37 mmol) in CH₂Cl₂ (15 mL) at 25° C. was treated with Dess-Martin periodinane (0.254 g, 0.60 mmol). After 1 h, the reaction mixture was diluted with CH₂Cl₂ (25 mL), washed with 10% aqueous NaHCO₃ (3×25 mL), dried (MgSO₄), filtered and concentrated in vacuo. The residue was used in the next step without further purification. A solution of the residue in pyridine (6 mL) in methanol (36 mL) at 25° C. was treated with methoxyamine hydrochloride (0.835 g, 6.0 mmol). After 2 min, the reaction mixture was warmed to reflux for 5 h, diluted with ethyl acetate (25 mL), washed with 10% aqueous NaHCO₃ (3×25 mL), dried (MgSO₄), filtered and concentrated in vacuo to afford 40 (230 mg, 42%) as an orange residue. The residue was used in the next step without further purification. MS 400 (M+H).

### (E)-4-[2-(1,3-dioxo-1,3-dihydro-2H-isoindol-2-yl)ethylidene]-3-methoxyimino-piperidine (41)

Prepared by the same procedure as in the synthesis of 27 except that 40 was used in place of 26. MS 300 (M+H).

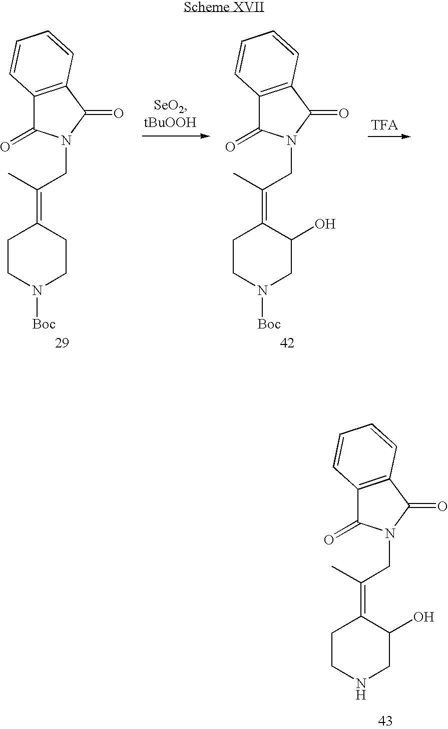

### Compound 43 of Scheme XVII:

### (Z)-t-Butyl 4-[2-(1,3-dioxo-1,3-dihydro-2H-isoindol-2- 5 yl)-1-methylethylidene]-3-hydroxy-piperidinyl-1 - carboxylate (42)

A slurry of SeO₂ (1.3 g, 11.4 mmol) in CH₂Cl₂ (15 mL) at 0° C. was treated with tert-butyl hydroperoxide (4 mL, 22 mmol, 5-6 M, 10% in undecane) via a syringe. After 20 min, the reaction mixture was treated with a solution of ethylidene 29 (3.4 g, 9.1 mmol) in CH₂Cl₂ (15 mL) and the resulting mixture was allowed to stir for 12 h at room temperature. The reaction was carefully quenched by the addition of 15% aqueous sodium thiosulfate (15 mL), and the reaction mixture was diluted with CH₂Cl₂ (25 mL). The layers were separated, and the organic layer was washed with 15% aqueous sodium thiosulfate (15 mL), dried (MgSO₄), filtered and concentrated in vacuo. Purification by flash chromatography (silica gel, 0-75% ethyl acetate/hexanes) afforded the title compound 42 (1.2 g, 34%) as a white solid. MS 387 (M+H).

### (Z)-4-[2-(1,3-dioxo-1,3-dihydro-2H-isoindol-2-yl)1-methyl-ethylidene]-3-hydroxy-piperidine (43)

Prepared by the same procedure as in the synthesis of 27 except that 42 was used in place of 26. MS 287 (M+H).

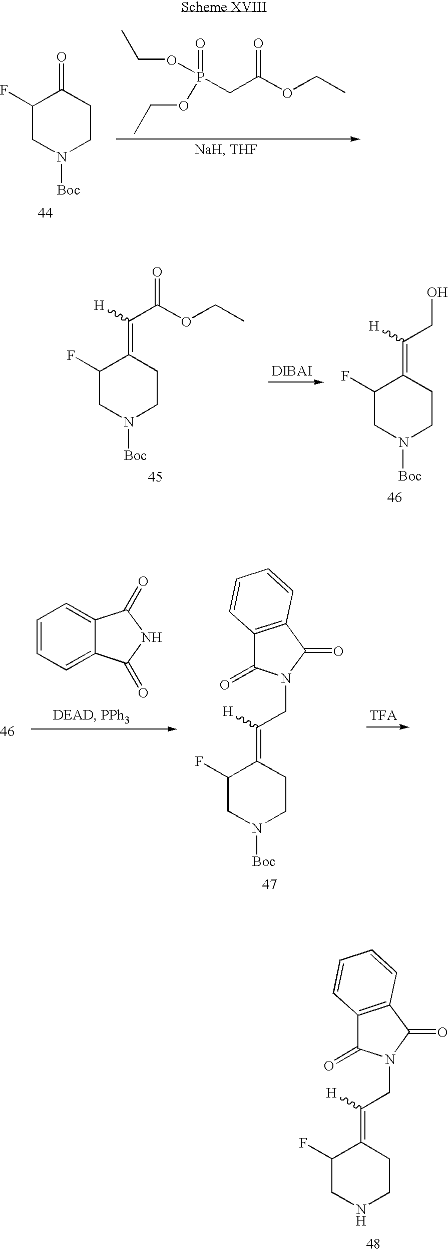

### Compound 48 of Scheme XVIII:

### t-Butyl-3-fluoro-4-oxopiperidinyl-1-carboxylate (44) was prepared according to U.S. Pat. No. 5,837,715.

### (E/Z)-t-Butyl 4-(2-ethoxy-2-oxoethylidene)-3-fluoropiperidinyl-1-carboxylate (45)

Prepared by the same procedure as in the synthesis of 24 except that 44 was used in place of 1-(tert-butoxycarbonyl)-4-piperidinone. MS 288 (M+H).

### (E/Z)-t-Butyl 4-(2-hydroxyethylidene)-3-fluoropiperidinyl-1-carboxylate (46)

Prepared by the same procedure as in the synthesis of 25 except that 45 was used in place of 24. MS 246 (M+H).

### (E/Z)-t-Butyl 4-[2-(1,3-dihydro-1,3-dioxo-2H-isoindol-2-yl)ethylidene]-3-fluoro-piperidinyl-1-carboxylate (47)

Prepared by the same procedure as in the synthesis of 26 except that 46 was used in place of 25. MS 375 (M+H).

### (E/Z)-4-[2-(1,3-dihydro-1,3-dioxo-2H-isoindol-2-yl)ethylidene]-3-fluoropiperidine Trifluoroacetate (48)

Prepared by the same procedure as in the synthesis of 27 except 47 was used in place of 26. MS 275 (M+H).

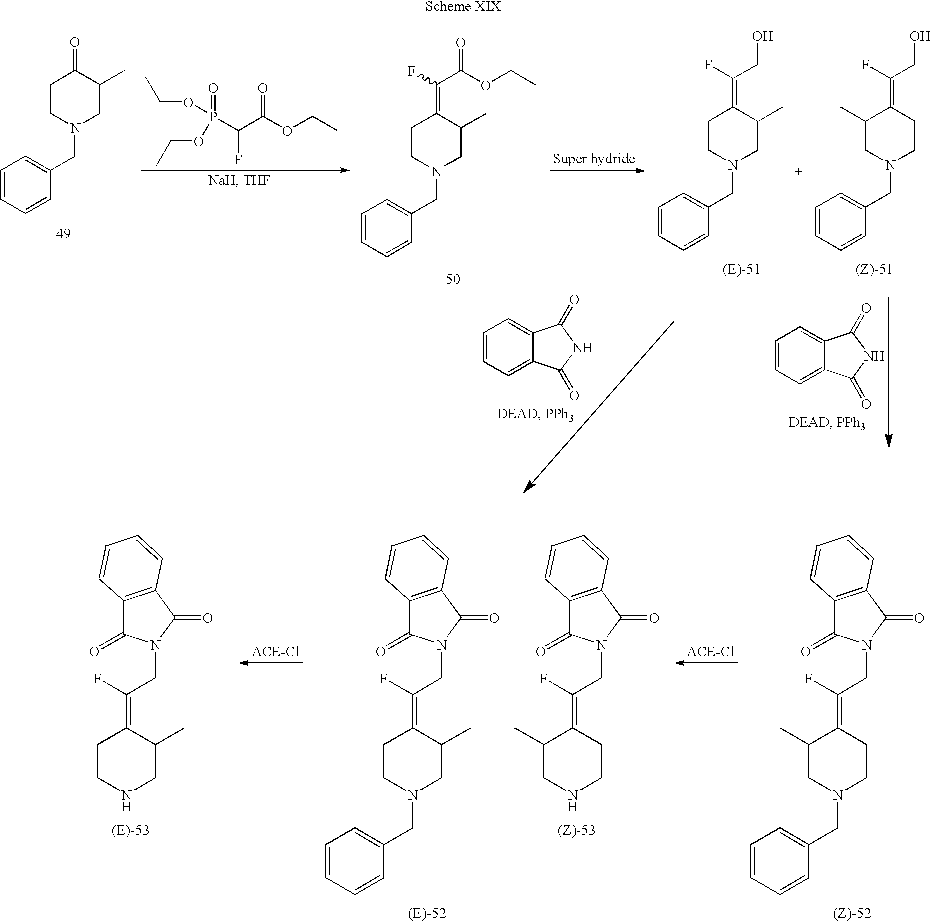

### Compounds Z-53 and E-53 of Scheme XIX:

### Ethyl 1-[3-methyl-i-(phenylmethyl)-4- piperidinylidenyl]-i-fluoroacetate (50)

Prepared by the same procedure as in the synthesis of 24 except that 49 was used in place of 1-(tert-butoxycarbonyl)-4-piperidinone and triethyl 2-fluorophosphonoacetate was used in place of triethyl phosphonoacetate. MS 292 (M+H).

### 2-[3-methyl-1-(phenylmethyl)-4-piperidinylidenyl]-2-fluoroethanol (E-51 and Z-51)

A solution of 50 (2.68 g, 9.19 mmol) in tetrahydrofuran (50 mL) at 0° C. was treated with a solution of Super-Hydride™ (23 mL, 23 mmol, 1.0 M in tetrahydrofuran, Aldrich) under nitrogen. After 1 h, the reaction mixture was carefully treated with methanol (10 mL), diluted with ethyl acetate (50 mL), washed with 10% aqueous NaHCO₃ (3×50 mL), dried (MgSO₄) and concentrated in vacuo. Purification by MPLC (silica gel, 0-50% ethyl acetate/hexanes) afforded the Z-isomer 51 (0.84 g, 37%) (MS 250 (M+H)) as a colorless oil and the E-isomer 51 (0.97 g, 42%) (MS 250 (M+H)) as a colorless oil.

### (Z)-2-[2-(3-methyl-1-(phenylmethyl)-4-piperidinylidenyl)-2-fluoroethyl]-1H-isoindole-1,3(2H)-dione (Z-52)

Prepared by the same procedure as in the synthesis of 26 except that Z-51 was used in place of 25. MS 379 (M+H).

### (E)-2-[2-(3-methyl-1-(phenylmethyl)-4-piperidinylidenyl)-2-fluoroethyl]-1H-isoindole-1,3(2H)-dione (E-52)

Prepared by the same procedure as in the synthesis of 26 except that E-51 was used in place of 25. MS 379 (M+H).

### (Z)-2-[2-fluoro-2-(3-methyl-4-piperidinylidenyl)-ethyl]-1H-isoindole-1,3(2H)-dione Hydrochloride (Z-53)

A mixture of Z-52 (0.550 g, 1.45 mmol) and 1-chloroethyl chloroformate (0.63 mL, 5.8 mmol) in dichloroethane (15 mL) was warmed to reflux temperature for 2 h. The resulting solution was allowed to cool to room temperature, and concentrated in vacuo. The residue was dissolved in methanol (50 mL) and warmed to reflux temperature for 2 h. The reaction mixture was allowed to cool to room temperature and concentrated in vacuo to afford a white solid. The residue was washed with diethyl ether (2×) and dried to afford Z-53 (260 mg, 55%) as a white solid. MS 289 (M+H).

### (E)-2-[2-fluoro-2-(3-methyl-4-piperidinylidenyl)-ethyl]-1H-isoindole-1,3(2H)-dione Hydrochloride (E-53)

Prepared by the same procedure as in the synthesis of Z-52 except that E-52 was used in place of Z-52. MS 289 (M+H).

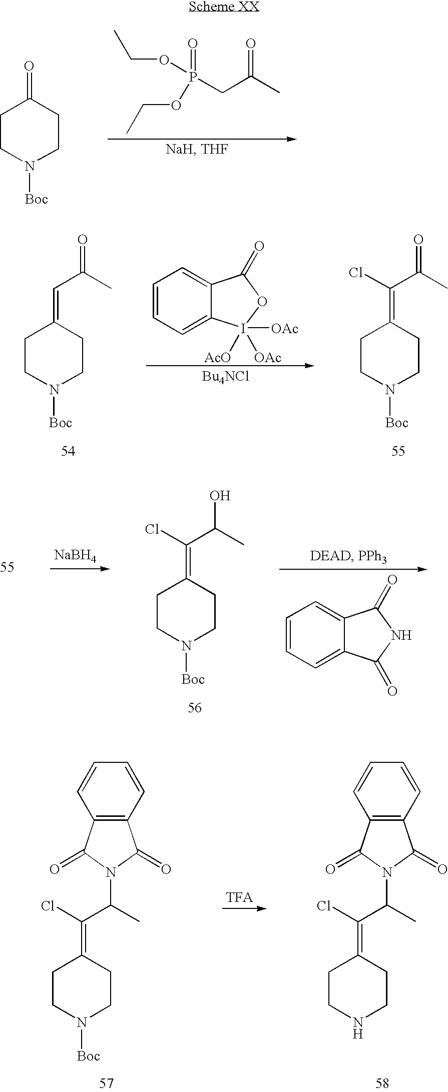

### 4-(2-Oxo-propylidene)piperidine-1 -carboxylic acid tert- buty ester (54)

Prepared by the same procedure as described in International Patent Publication WO0285901.

### 4-(1-Chloro-2-oxo-propylidene)piperidine-1-carboxylic Acid tert-butyl Ester (55)

A slurry of tetrabutylammonium chloride (11.1 g, 40.1 mmol) in CH₂Cl₂ (50 mL) at 25° C. was treated with 1,1,1-tris(acetoxy)-1,1-dihydro-1,2-benzodioxol-3(1H)-one (17.0 g, 40.1 mmol) and the resulting light yellow solution was allowed to stir for 10 min. The reaction mixture was treated with a solution of 54 in CH₂Cl₂ (50 mL) and the resulting solution was allowed to stir for 3 h. The light yellow solution was carefully poured into a 10% aqueous solution of sodium bicarbonate (100 mL), diluted with CH₂Cl₂(50 mL), to induce precipitation, filtered, and the precipitate was discarded. The resulting clear solution was washed with a 10% aqueous solution of sodium bicarbonate (1×100 mL), brine (1×100 mL), dried (MgSO₄), and concentrated in vacuo. Purification by MPLC (0-40% ethyl acetate/hexanes) afforded 55 (1.24 g, 34%) as a colorless oil. MS 274 (M+H).

### 4-(1-Chloro-2-hydroxypropylidene)piperidine-1-carboxylic Acid tert-butyl Ester (56)

A solution of 55 (1.24 g, 4.53 mmol) in ethanol (25 mL) was treated with sodium borohydride (102 mg, 2.72 mmol) at 25° C. After 1 h, the reaction mixture was concentrated in vacuo, diluted with ethyl acetate (40 mL), carefully treated with 5% aqueous hydrochloric acid (1×25 mL), the layers separated, and dried (MgSO₄). The resulting solution was concentrated in vacuo to afford 56 (902.1 mg, 72%) as a colorless residue that was used without further purification. MS 298 (M+Na).

### 4-[1-Chloro-2-(1,3-dioxo-1,3-dihydro-isoindol-2-yl)-propylidene]piperidine-1-carboxylic Acid tert-butyl Ester (57)

Prepared by the same procedure as in the synthesis of 26 except that 56 was used in place of 25. MS 427 (M+Na).

### 2-[(2-Chloro-1-methyl-2-(4-piperidinylidene)ethyl]-1H-isoindole-1,3(2H)-dione (58)

Prepared by the same procedure as in the synthesis of 27 except that 56 was used in place of 26. MS 305 (M+H).

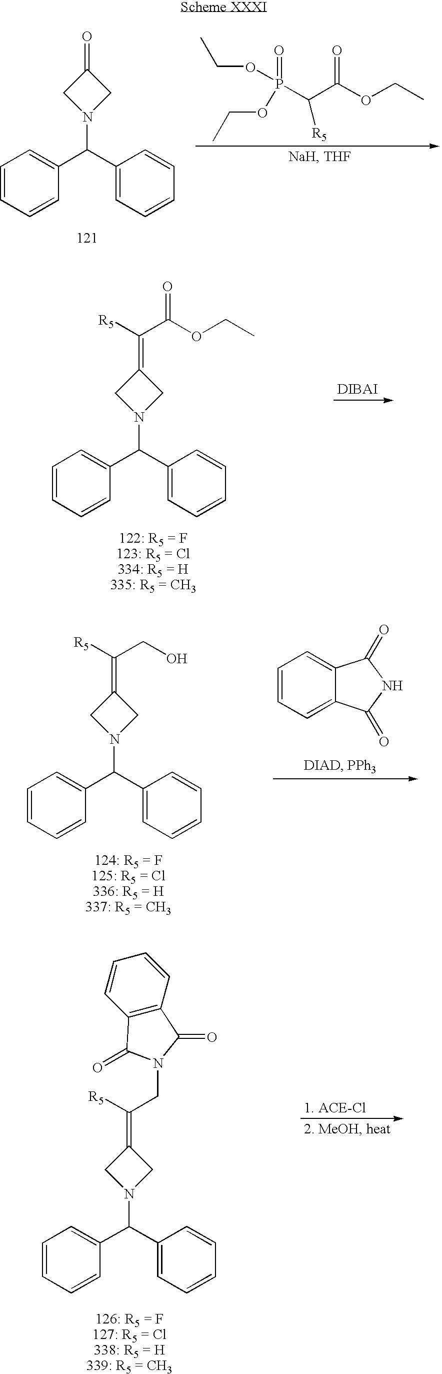

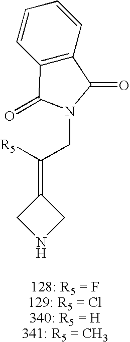

1-Benzhydrylazetidin-3-one (121) was prepared according to the procedure described in Claiborne, et al in International Publication WO 01/01988.

### Ethyl 1-[1-diphenylmethylazetidin-3-ylidene]-1-fluoroacetate (122; R₅═F)

Triethyl 2-fluoro-2-phosphonoacetate (0.63 mL, 3.10 mmol) was added to NaH (60% in oil, 115 mg, 2.87 mmol) in anhydrous THF (6 mL) at 0° C. After stirring for 15 minutes, a solution of ketone 121 (562 mg, 2.37 mmol) in anhydrous THF (6 mL) was added. The reaction was warmed to room temperature and stirred overnight. The reaction was diluted with ethyl acetate (100 mL), washed with saturated NaHCO₃ (2×100 mL), dried (MgSO₄), filtered and concentrated in vacuo. The crude material was chromatographed (100% CH₂Cl₂) to afford ester 122 (R₅═F) as a yellow oil (392 mg, 66%). MS 326 (M+H).

Ethyl 1-[1-diphenylmethylazetidin-3-ylidene]-1-chloroacetate (123; R₅═Cl)

This was prepared in a similar manner to the procedure described above except that triethyl 2-chloro-2-phosphonoacetate was utilized in place of triethyl 2-fluoro-2-phosphonoacetate in the reaction. Ester 123 (R₅═Cl) was isolated as a white solid (77%). MS 342, 344 (M+H).

Ethyl 1-[1-diphenylmethylazetidin-3-ylidene]acetate (334; R₅═H)

This was prepared in a similar manner as example 122 except that triethyl 2-phosphonoacetate was utilized in place of triethyl 2-fluoro-2-phosphonoacetate. Ester 334 (R₅═H) was isolated by chromatography (10-20% ethyl acetate/hexanes) as light yellow oil (59%). MS 308 (M+H).

### Ethyl 1-[1-diphenylmethylazetidin-3-ylidene]-2-methylacetate (335; R₅═CH₃)

This was prepared in a similar manner as for the synthesis of example 122 except that triethyl 2-methyl-2-phosphonoacetate was utilized in place of triethyl 2-fluoro-2-phosphonoacetate. Ester 335 (R₅═CH₃) was isolated by chromatography (10-20% ethyl acetate/hexanes) as a pale yellow solid (65%). MS 322 (M+H).

### 2-(1-Diphenylmethylazetidin-3-ylidene)-2-fluoroethanol (124; R₅═F)

DIBAL (1M in toluene, 4.2 mL, 4.2 mmol) was added to a solution of ester 122 (R₅═F) (510 mg, 1.56 mmol) in toluene (8 mL) at −78° C. over several minutes. The reaction was stirred for 5 hours and then quenched by the slow addition of a solution of methanol in toluene. The reaction was diluted with ethyl acetate (100 mL), washed with NaOH (1N, 2×50 mL), water (50 mL), dried (MgSO₄) and concentrated in vacuo to afford alcohol 124 (R₅═F, 289 mg, 65%) as a pale yellow solid after trituration with ether/hexanes. MS 284 (M+H).

### 2-(1-Diphenylmethylazetidin-3-ylidene)-2-chloroethanol (125; R₅═Cl)

This was prepared in a similar manner to the procedure described above except that ester 123 (R₅═Cl) was used in place of ester 122 (R₅═F). Alcohol 125 (R₅═Cl) was isolated by chromatography (20% ethyl acetate/hexanes) as a white solid (53%). MS 300, 302 (M+H).

### 2-(1-Diphenylmethylazetidin-3-ylidene)ethanol (336; R₅═H)

This was prepared in a similar manner as example 124 except that ester 334 (R₅═H) and DIBAL (1M in hexane) were used in place of ester 122 (R₅═F) and DIBAL (1M in toluene). The crude alcohol 336 (R₅═H) was used in the next reaction without further purification. MS 266 (M+H).

### 2-(1-Diphenylmethylazetidin-3-ylidene)-2-methyl Ethanol (337; R₅═CH₃)

This was prepared in a similar manner as for the synthesis of example 124 except that ester 335 (R₅═CH₃) and DIBAL (1M in hexane) were used in place of ester 122 (R₅═F) and DIBAL (1M in toluene). The crude alcohol 337 (R₅═CH₃) was used in the next reaction without further purification. MS 280 (M+H).

### 2-[2-(1-Diphenylmethylazetidin-3-ylidene)-2-fluoroethyl]isoindole-1,3-dione (126; R₅═F)

DIAD (0.89 mL, 4.489 mmol) was added to a solution of alcohol 124 (R₅═F) (1.00 g, 3.533 mmol), triphenyl phosphine (1.14 g, 4.34 mmol) and phthalimide (0.648 g, 4.527 mmol) in anhydrous THF (30 mL) at 0° C. The reaction was warmed to room temperature and stirred for 36 hours. The volatiles were evaporated and the residue chromatographed on silica gel (5% ethyl acetate/hexanes) to afford phthalimide 126 (R₅═F) (952 mg, 65%) as a white solid. MS 413 (M+H).

### 2-[2-(1-Diphenylmethylazetidin-3-ylidene)-2-chloroethyl]isoindole-1,3-dione (127; R₅═Cl)

This was prepared in a similar manner to the procedure described above except that alcohol 125 (R₅═Cl) was used in place of alcohol 124 (R₅═F) in the Mitsunobu reaction. Phthalimide 127 (R₅═Cl) was isolated by chromatography (15% ethyl acetate/hexanes) as a white solid (68%). MS 429, 431 (M+H).

### 2-[2-(1-Diphenylmethylazetidin-3-ylidene)-ethyl]isoindole-1,3-dione (338; R₅═H)

This was prepared in a similar manner as for the synthesis of example 126 except that alcohol 336 (R₅═H) was used in place of alcohol 124 (R₅═F). Phthalimide 338 (R₅═H) was isolated by chromatography (25% ethyl acetate/hexanes) as a pale yellow solid (87%). MS 395 (M+H).

### 2-[2-(1-Diphenylmethylazetidin-3-ylidene)-2-methylethyl]isoindole-1,3-dione (339; R₅═CH₃)

This was prepared in a similar manner as example 126 except that alcohol 337 (R₅═CH₃) was used in place of alcohol 124 (R₅═F). Phthalimide 339 (R₅═CH₃) was isolated by chromatography (25% ethyl acetate/hexanes) as a white solid (81%). MS 409 (M+H).

### 2-(2-Azetidin-3-ylidene-2-fluoroethyl)isoindole-1,3-dione Hydrochloride (128; R₅═F)

Phthalimide 126 (R₅═F) (350 mg, 0.8491 mmol) and ACE-Cl (0.50 mL, 4.65 mmol) in 1,2-dichloroethane (20 mL) were heated at reflux temperature under a nitrogen atmosphere for 24 hours. After cooling, the volatiles were evaporated and methanol (25 mL) was added to the resulting residue. This was heated at reflux temperature for 3 hours after which the methanol was evaporated to afford 128 (R₅═F) as a beige powder (230 mg, 96%). MS 247 (M+H).

### 2-(2-Azetidin-3-ylidene-2-chloroethyl)isoindole-1,3-dione Hydrochloride (129; R₅═Cl)

This was prepared in a similar manner to the procedure described above except that phthalimide 127 (R₅═Cl) was used in place of phthalimide 126 (R₅═F) in the reaction. The compound was isolated as a white powder (86%). MS 263, 265 (M+H).

### 2-(2-Azetidin-3-ylidene-ethyl)isoindole-1,3-dione Hydrochloride (340; R₅═H)

This was prepared in a similar manner as for the synthesis of example 128 except that phthalimide 338 (R₅═H) was used in place of phthalimide 126 (R₅═F). The crude material was purified by HPLC (reverse phase C-18 column, 10-50% acetonitrile/water containing 0.1% trifluoroacetic acid) to provide the trifluoroacetic acid salt of the title compound as a light brown oil (25%). MS 229 (M+H).

### 2-(2-Azetidin-3-ylidene-2-methylethyl)isoindole-1,3-dione Hydrochloride (341; R₅═CH₃)

This was prepared in a similar manner as example 128 except that phthalimide 339 (R₅═CH₃) was used in place of phthalimide 126 (R₅═F). The crude material was dissolved in a small amount of methanol, and then triturated with diethyl ether. The light brown solid was collected by filtration and dried to afford the title compound (85%). MS 243 (M+H).

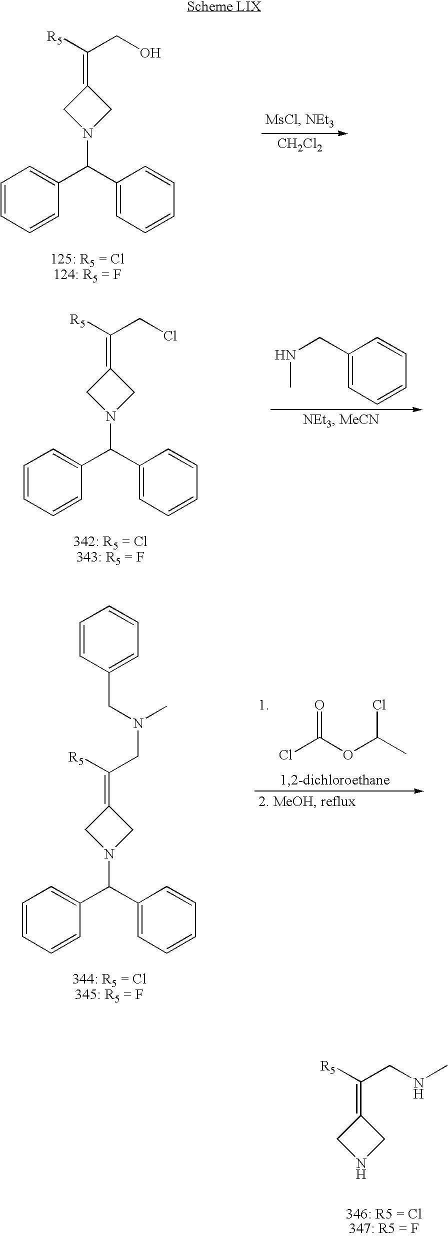

2-(1-Diphenylmethylazetidin-3-ylidene)-1,2-dichloroethane (342; R5=Cl) was prepared in an analogous manner to Z-236 except that compound 125 was used in place of compound Z-235. MS 319 (M+H).

### 2-(1-Diphenylmethylazetidin-3-ylidene)-2-chloro-1-(N-methyl-N-benzyl)ethylamine (344; R5=Cl)

To a solution of compound 342 (697 mg; 2.190 mmol) in acetonitrile (20 mL) was added N-methyl benzylamine (0.45 mL; 3.524 mmol). The resulting mixture was treated with triethylamine (1.53 mL; 10.951 mmol) and stirred at room temperature under a nitrogen atmosphere for 2 days. The reaction mixture was concentrated, the residue was diluted with ethyl acetate (50 mL) and washed with H₂O (50 mL). The water layer was extracted with ethyl acetate (50 mL×2). The organic layers were combined and dried over Na₂SO₄. Concentration of the filtrate in vacuo afforded the title compound 344 (882 mg; quantitative). MS 403 (M+H).

2-(Azetidin-3-ylidene)-2-chloro-1-(N-methyl)ethylamine hydrochloride salt (346; R5=Cl) was prepared in an analogous manner to 128 except that compound 344 was used in place of compound 126. The title compound (346) was obtained as white needles by dissolving the crude material in a small amount of methanol, then triturating with diethyl ether. MS 147 (M+H).

2-(Azetidin-3-ylidene)-2-fluoro-1-(N-methyl)ethylamine trifluoroacetic acid salt (347; R5=F) was prepared in an analogous manner as described for 346 except that the synthetic sequence began with compound 124 instead of compound 125 and proceeded through the analogous intermediates, 343 and 345. In addition, the crude amine was purified by HPLC (reverse phase C-18 column, 10-50% acetonitrile/water containing 0.1% trifluoroacetic acid) to afford a light brown oil as the trifluoroacetic acid salt. MS 131 (M+H).

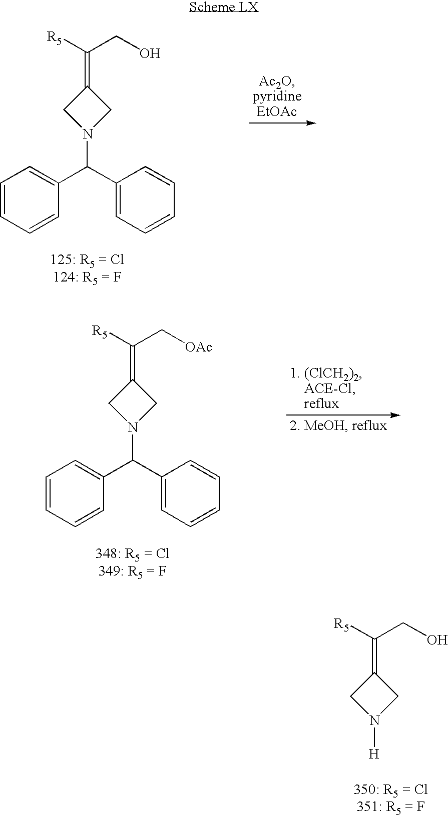

### 7-[3-(2-Hydroxy-1-chloro-ethylidene)azetidin-1 -yl]-1- cyclopropyl-8-methoxy-6-fluoro-4-oxo- 1,4- dihydroquinoline-3-carboxylic acid (350; R5=Cl).

### Step 1

Alcohol 125 (491 mg; 1.64 mmoles), pyridine (0.25 mL; 3.31 mmoles) and acetic anhydride (0.16 mL; 1.69 mmoles) in ethyl acetate (10 mL) were stirred at room temperature overnight. The reaction was poured into water (25 mL) and the organic layer was separated. The organic layer was washed with water, dried (MgSO₄), filtered and evaporated to afford acetate 348 as a viscous oil.

### Step 2

The above acetate (1.64 mmoles) and ACE-Cl (0.4 mL; 3.66 mmoles) in dichloroethane (10 mL) were heated at reflux temperature for 24 hours. The volatiles were evaporated and methanol (10 mL) was added to the residue. This was heated at reflux temperature for 4 hours after which the volatiles were evaporated. The crude material was triturated with ether to afford amino alcohol 350 as a solid (267 mg; 96%). MS 134 (M+H).

2-(Azetidin-3-ylidene)-2-fluoro-1-ethanol trifluoroacetic acid salt (351; R₅═F) was prepared in an analogous manner to 350 except that the synthetic sequence began with compound 124 instead of compound 125 and proceeded through the analogous intermediate, 349. In addition, the crude product was purified by HPLC (reverse phase C-18 column, 10-50% acetonitrile/water containing 0.1% trifluoroacetic acid) to afford a light brown oil. MS 118 (M+H).

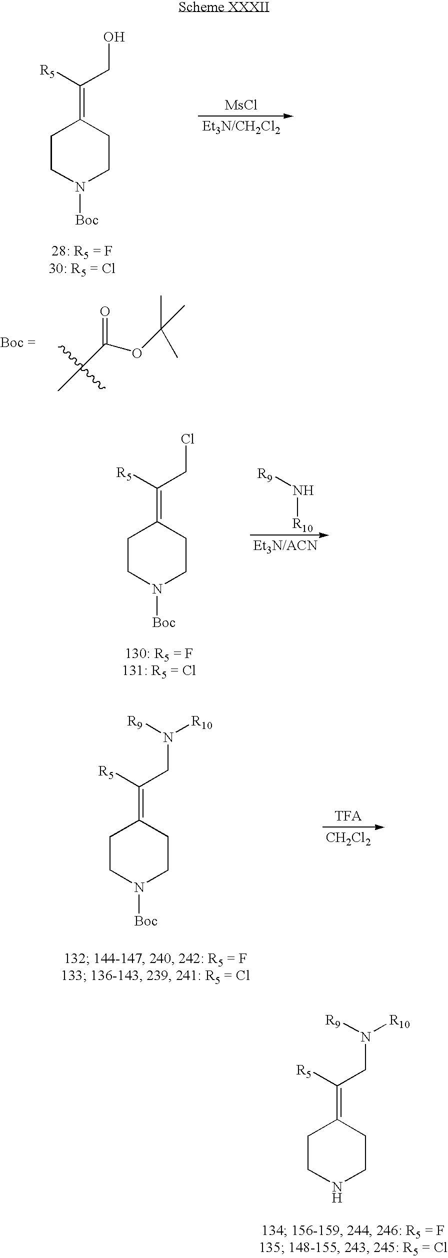

### t-Butyl 4-(1,2-dichloroethylidene)piperidinyl-1- carboxylate (131; R5=Cl)

A solution of 30 (R₅═Cl) (4.24 g, 16.20 mmol)) and triethylamine (6.8 mL, 48.60 mmol) in CH₂Cl₂ (120 mL) was treated with methanesulfonyl chloride (1.9 mL, 24.30 mmol) at 0° C., then warmed to rt and stirred over night. The resulting mixture was quenched by addition of saturated aq. NaHCO₃ (100 mL) and the product was extracted into CH₂Cl₂. Purification by flash chromatography (0-20% ethyl acetate/hexanes) afforded the title compound (3.1 g, 68%) as a white solid.

### t-Butyl 4-(2-chloro-1-fluoroethylidene)piperidinyl-1-carboxylate (130; R₅═F)

This was prepared in a similar manner to the procedure described above except that alcohol 28 (R₅═F) was used in place of alcohol 30 (R₅═Cl).

### t-Butyl 4-[2-(N-benzyl-N-methylamino)-1-chloroethylidene]piperidinyl-1-carboxylate (133; R₅═Cl; R₉=methyl; R₁₀=benzyl)

A solution of 131 (R₅═Cl) (600 mg, 2.14 mmol)) and triethylamine (1.5 mL, 10.71 mmol) in acetonitrile (18 mL) was treated with N-benzylmethylamine (0.45 mL, 3.43 mmol) at rt and stirred overnight. The resulting mixture was concentrated in vacuo, and the residue was diluted with ethyl acetate (20 mL), washed with water (2×10 mL), and dried (MgSO₄). Purification by flash chromatography (0-15% ethyl acetate/hexanes) afforded the title compound (690 mg, 88%) as a white solid. MS 365 (M+H).

### t-Butyl 4-[2-(N-benzyl-N-methylamino)-1-fluoroethylidene]piperidinyl-1-carboxylate (132; R₅═F; R₉=methyl; R₁₀=benzyl)

This was prepared in a similar manner to the procedure described above except that chloride 130 (R₅═F) was used in place of chloride 131 (R₅═Cl). MS 349 (M+H).

N-Benzyl-N-methyl-(2-chloro-2-piperidin-4-ylidene)ethylamine (135; R₅═Cl; R₉=methyl; R₁₀=benzyl)

A solution of 133 (R₅═Cl) (690 mg, 1.89 mmol) was dissolved in CH₂Cl₂ (15 mL) and was treated with trifluoroacetic acid (1.5 mL) at rt. After 5 h, the reaction mixture was concentrated in vacuo to afford the title compound (quant.) as an oil, which was used in the next step without further purification. MS 265 (M+H).

### Benzyl-(2-fluoro-2-piperidin-4-ylidene-ethyl)-methyl-amine (134; R₅═F; R₉=methyl; R₁₀=benzyl)

This was prepared in a similar manner to the procedure described above except that amine 132 (R₅═F) was used in place of amine 133 (R₅═Cl). MS 249 (M+H).

Table 9 lists the Boc-protected amines (136-147) and the derived amines (148-159) prepared by analogous procedures to those detailed above. The 2-(2-aminoethyl)-1H-isoindole-1,3(2H)-dione used in the preparation of 241 was synthesized by a similar procedure as that described in Tetrahedron: Asymmetry 2000, 11, 1907.

TABLE XX

| | | Boc-protected | | | | |
|---|---|---|---|---|---|---|
| NHR₉R₁₀ | Cpd | Amine | (M + H) | Cpd | Amine | (M + H) |
| 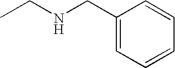 | 136 | 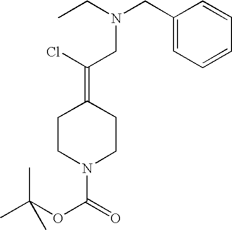 | 379 | 148 | 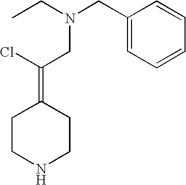 | 279 |
| 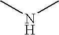 | 137 | 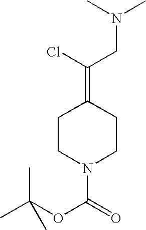 | 289 | 149 | 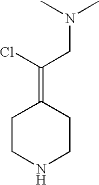 | 189 |
| 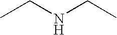 | 138 | 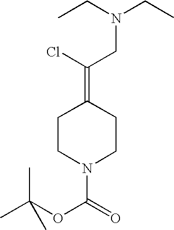 | 317 | 150 | 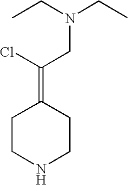 | 217 |
| 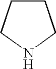 | 139 | 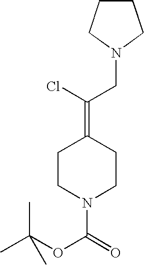 | 315 | 151 | 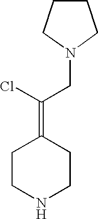 | 215 |
| 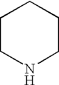 | 140 | 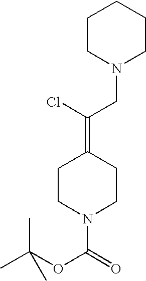 | 329 | 152 | 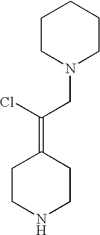 | 229 |
| 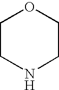 | 141 | 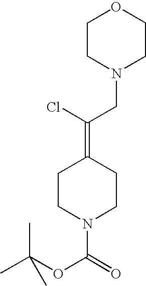 | 331 | 153 | 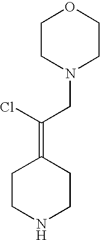 | 231 |
| 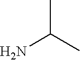 | 142 | 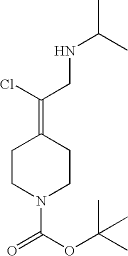 | 303 | 154 | 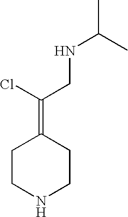 | 203 |
| 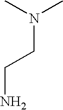 | 143 | 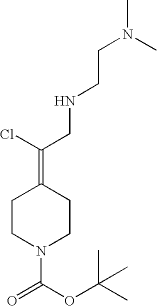 | 332 | 155 | 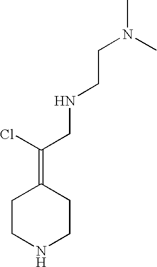 | 232 |
| 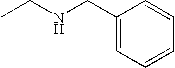 | 144 | 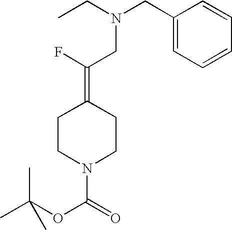 | 363 | 156 | 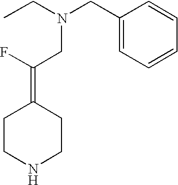 | 263 |
| 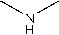 | 145 | 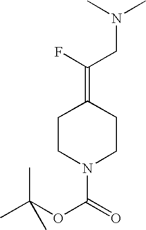 | 373 | 157 | 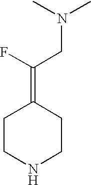 | 273 |
| 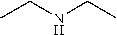 | 146 | 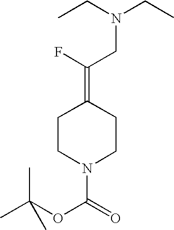 | 301 | 158 | 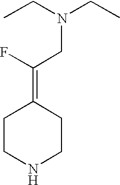 | 201 |
| 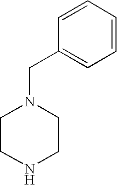 | 147 | 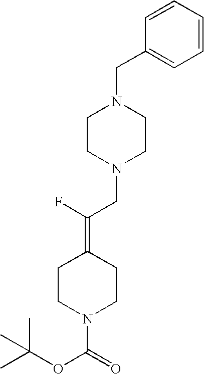 | 404 | 159 | 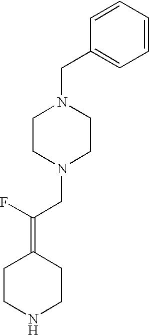 | 304 |
| 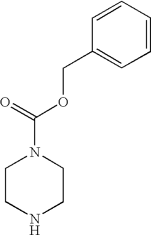 | 239 | 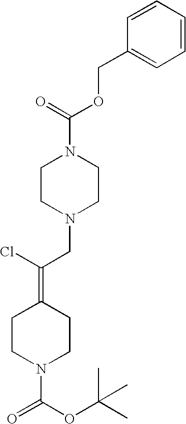 | 464 | 243 | 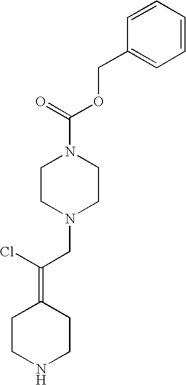 | 364 |
| 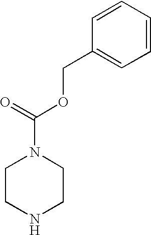 | 240 | 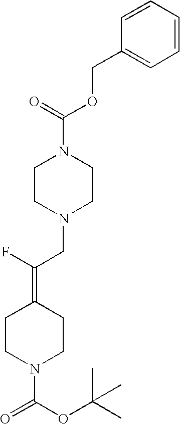 | 448 | 244 | 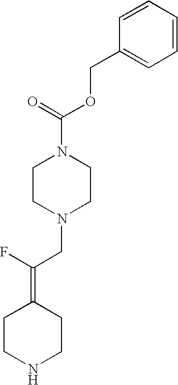 | 348 |
| 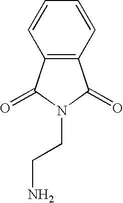 | 241 | 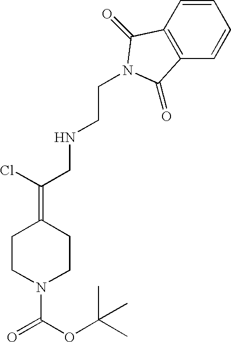 | 434 | 245 | 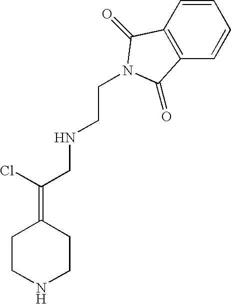 | 334 |
| 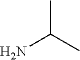 | 242 | 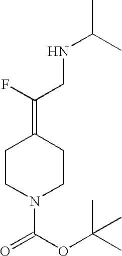 | 287 | 246 | 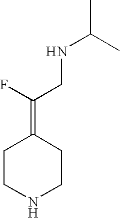 | 187 |

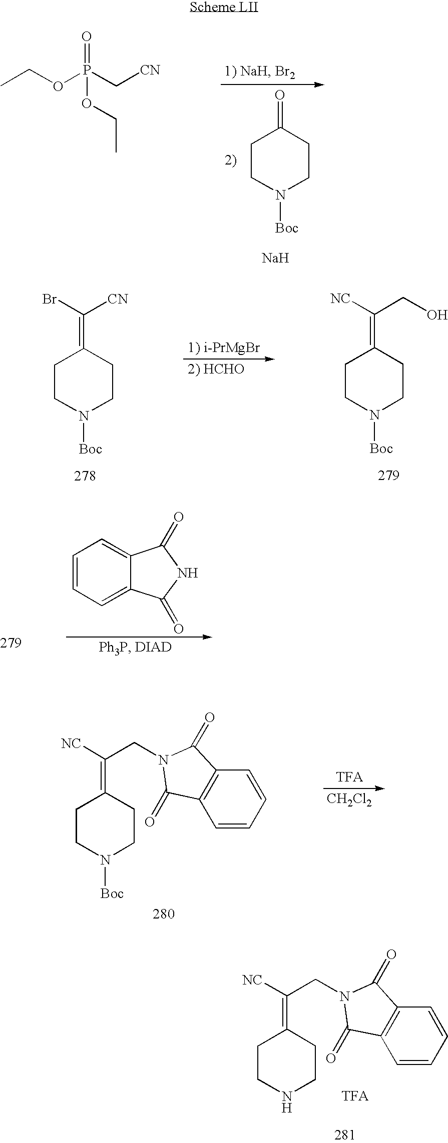

### 4-(Bromo-cyano-methylidene)-piperidine-1-carboxylic acid tert-butyl ester (278)

A slurry of sodium hydride (0.76 g, 19.0 mmol) in THF (75 mL) at 0° C. under nitrogen was carefully treated with diethyl cyanomethylphosphonate (3.4 g, 19.0 mmol) via a syringe. After gas evolution ceased, the reaction mixture was treated with bromine (3.04 g, 19.0 mmol) via a dropping funnel over 10 min., and the resulting mixture was allowed to stir for 2 h. The reaction mixture was treated with sodium hydride (0.76 g, 19.0 mmol) and the resulting slurry was allowed to stir for 30 min. at 0° C. A solution of 1-(tert-butoxycarbonyl)-4-piperidinone (2.52 g, 12.7 mmol) in THF (10 mL) was added dropwise over 10 min. and the resulting solution was allowed to stir at room temperature overnight. The reaction was quenched by the addition of water (50 mL) and the resulting mixture was diluted with ethyl acetate (100 mL), washed with sat. NH₄Cl (100 mL), brine (100 mL), and concentrated in vacuo. Purification by chromatography (EtOAc/hexanes=1:4) yielded the title compound (2.8 g, 74%) as a white solid. MS 301 (M+H).

### 4-(1-cyano-2-hydroxy-ethylidene)-piperidine-1-carboxylic Acid tert-butyl Ester (279)

To a solution of 2,6-diphenyl phenol (10.5 g, 42 mmol) in 60 mL CH₂Cl₂ at room temperature was added AlMe₃ (2.0 M in hexane, 10.5 mL, 21 mmol). Gas evolution was observed and the mixture was stirred at room temperature for 30 min. The solution was cooled to 0° C. and 1,3,5-trioxane (630 mg, 7 mmol) in 6 mL CH₂Cl₂ was added dropwise and the solution was stirred for 1 h.

In a separate flask 4-(bromo-cyano-methylidene)-piperidine-1-carboxylic acid tert-butyl ester (2.1 g, 7 mmol) was dissolved in 20 mL THF at −40° C. and to this solution was added isopropylmagnesium bromide (1M in THF, 8.4 mL, 8.4 mmol) dropwise. The mixture was stirred at −40° C. to −30° C. for 1.5 h.

The above freshly prepared Grignard reagent was added dropwise to the formaldehyde solution and the mixture was stirred at 0° C. for 1 h. Water was carefully added and the mixture was extracted with ethyl acetate. Purification by chromatography (EtOAc/hexanes=1:1) yielded the title compound (800 mg, 45%) as a white solid. MS 253 (M+H).

### 4-[1-cyano-2-(1,3-dioxo-1,3-dihydro-isoindol-2-yl)-ethylidene]-piperidine-1-carboxylic Acid tert-butyl Ester (280)

This compound was prepared in a similar manner as in the synthesis of 26 except that 279 was used in place of 25. MS 382 (M+H).

### 3-(1,3-dioxo-1,3-dihydro-isoindol-2-yl)-2-piperidin-4-ylidene-propionitrile (281)

This compound was prepared in a similar manner as in the synthesis of 27 except that 280 was used in place of 26. MS 282 (M+H).

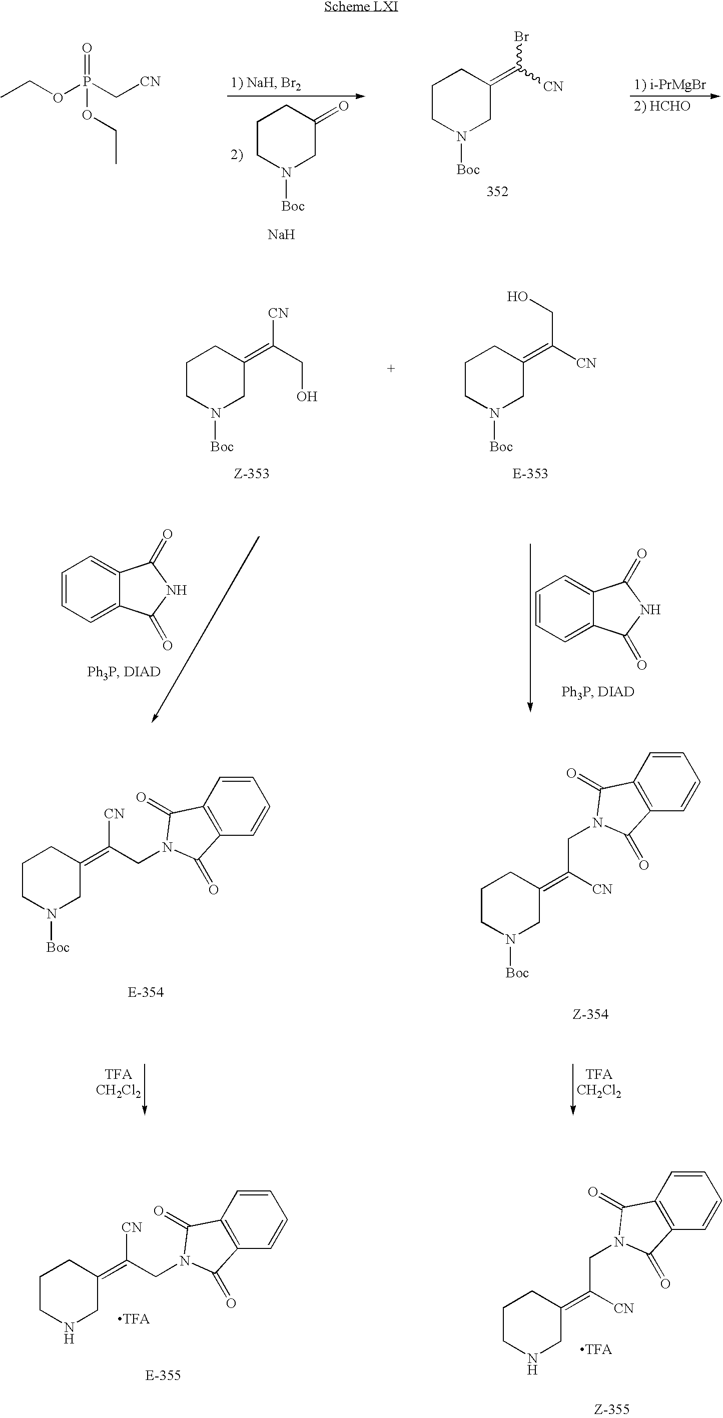

### 3-(Bromo-cyano-methylidene)-piperidine-1 -carboxylic acid tert-butyl ester (352)

352 was synthesized by an analogous procedure to that used to prepare compound 278 except that 1-(tert-butoxycarbonyl)-3-piperidinone was used in place of 1-(tert-butoxycarbonyl)-4-piperidinone. Purification by column chromatography (EtOAc/hexanes=1:5) yielded the title compound as a mixture of geometric isomers. MS 301 (M+H).

### (E) and (Z)-3-(1-cyano-2-hydroxy-ethylidene)-piperidine-1-carboxylic Acid tert-butyl Ester (Z-353 and E-353)

Z-353 and E-353 were prepared in a similar fashion as compound 279 above except that 352 was used in place of 278. Purification by column chromatography (EtOAc/hexanes=1:2->1:1->2:1) yielded a 3:2 ratio of the faster eluting isomer, (Z)-3-(1-cyano-2-hydroxy-ethylidene)-piperidine-1-carboxylic acid tert-butyl ester (Z-353), as an oil (MS 253 (M+H)) and the slower eluting isomer, (E)-3-(1-cyano-2-hydroxy-ethylidene)-piperidine-1-carboxylic acid tert-butyl ester (E-353), as a solid (MS 253 (M+H)).

### (E)-3-[1-cyano-2-(1,3-dioxo-1,3-dihydro-isoindol-2-yl)-ethylidene]-piperidine-1-carboxylic Acid tert-butyl Ester (E-354)

This compound was prepared in a similar manner as in the synthesis of 26 except that Z-353 was used in place of 25. MS 382 (M+H).

### (Z)-3-[1-cyano-2-(1,3-dioxo-1,3-dihydro-isoindol-2-yl)-ethylidene]-piperidine-1-carboxylic Acid tert-butyl Ester (Z-354)

This compound was prepared in a similar manner as in the synthesis of 26 except that E-353 was used in place of 25. MS 382 (M+H).

### (E)-3-(1,3-dioxo-1,3-dihydro-isoindol-2-yl)-2-piperidin-3-ylidene-propionitrile (E-355)

This compound was prepared in a similar manner as in the synthesis of 27 except that E-354 was used in place of 26. MS 282 (M+H).

### (Z)-3-(1,3-dioxo-1,3-dihydro-isoindol-2-yl)-2-piperidin-3-ylidene-propionitrile (Z-355)

This compound was prepared in a similar manner as in the synthesis of 27 except that Z-354 was used in place of 26. MS 282 (M+H).

### Final Product Preparation

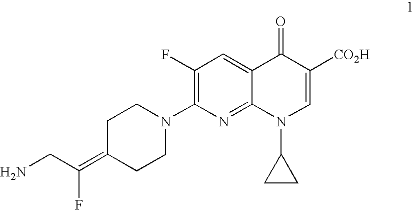

### 7-[4-(2-Amino-1-fluoro-ethylidene)-piperidin-1-yl]-1- cyclopropyl-6-fluoro-4-oxo- 1,4-dihydro- [1,8]naphthyridine-3-carboxylic acid (1)

A solution of amine 31 (612 mg, 1.57 mmol) and triethylamine (0.7 mL, 5.0 mmol) in acetonitrile (4 mL) was treated with 7-chloro-1-cyclopropyl-6-fluoro-4-oxo-1,4-dihydro-naphthpyridine-3-carboxylic acid (222 mg, 0.787 mmol) under nitrogen and the reaction mixture was allowed to stir for 12 h. The resulting mixture was concentrated in vacuo, and the residue was washed with water (3×10 mL). The residue was allowed to dry for 15 min. The solid was collected, resuspended in methanol (5 mL) and the reaction mixture was treated with hydrazine (1 mL). After 5 min, the reaction mixture was warmed to reflux and the resulting mixture was allowed to stir for 1 h. The reaction mixture was concentrated in vacuo, diluted with water and the solids were collected by filtration. The off white product was washed with water (3×20 mL), allowed to dry overnight to afford the title compound 1 (40.4 mg, 13%). MS 391 (M+H).

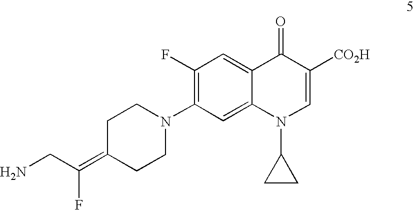

### 7-[4-(2-Amino-1-fluoro-ethylidene)-piperidin-1-yl]-l - cyclopropyl-6-fluoro-4-oxo-1,4-dihydro-quinoline-3- carboxylic acid trifluoroacetic acid salt (5)

A solution of amine 31 (311 mg, 0.80 mmol) and triethylamine (0.55 mL, 4.0 mmol) in acetonitrile (4 mL) was treated with diacetyl quinolinyl borate 17 (300 mg, 0.60 mmol) under nitrogen. After 5 min, the reaction mixture was warmed to reflux and the reaction mixture was allowed to stir for 12 h. The resulting mixture was allowed to cool to room temperature, concentrated in vacuo, and the residue was washed with water (3×10 mL). The residue was dissolved in tetrahydrofuran (3 mL) and treated with 10% aqueous hydrochloric acid (5 mL) at room temperature. After 30 min, the reaction mixture was concentrated in vacuo, diluted with water (10 min) and the solid collected by filtration. The solid residue was washed with water (3×5 mL) and allowed to dry for 15 min. The solid was collected and resuspended in methanol (5 mL) and the reaction mixture was treated with hydrazine (1 mL). After 5 min, the reaction mixture was warmed to reflux temperature and the resulting mixture was allowed to stir for 1 h. The reaction mixture was concentrated in vacuo and the residue purified by HPLC (reverse phase C-18 column, 0-55% acetonitrile/water containing 0.1% trifluoroacetic acid) to afford the trifluoroacetic acid salt of 5 (61.3 mg, 20%) as a light yellow solid. MS 390 (M+H).

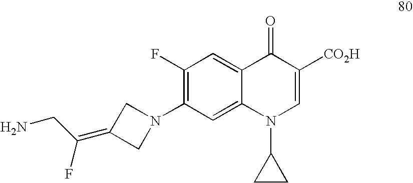

### 7-[3-(2-Amino-1-fluoro-ethylidene)-azetidin-1-yl]-1- cyclopropyl-6-fluoro-4-oxo-1,4-dihydro- [1,8]naphthyridine-3-carboxylic acid (80)

7-Chloro-1-cyclopropyl-6-fluoro-4-oxo-1,4-dihydronaphthyridine-3-carboxylic acid (57 mg, 0.2016 mmol), amine 128 (R₅═F) (67 mg, 0.2389 mmol) and triethylamine (0.5 mL) in acetonitrile (10 mL) were heated at reflux temperature overnight. After cooling, the volatiles were evaporated and the residue suspended in water (25 mL). The resulting solid was collected by filtration and dried. Ethanol (5 mL) was added to the solid followed by hydrazine (0.01 mL, 0.3138 mmol). The reaction mixture was heated at reflux temperature for 1 hour after which the volatiles were evaporated. Water (15 mL) was added to the residue and the resulting solid collected by filtration, washed with additional water and dried to afford 87 (49.1 mg, 69%) as an off-white powder. MS 363 (M+H).

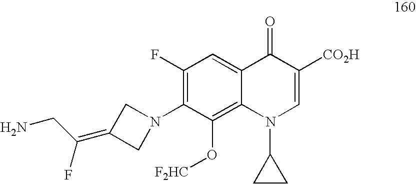

### 7-[3-(2-Amino-1-fluoro-ethylidene)-azetidin-1-yl]-1- cyclopropyl-8-difluoromethoxy-6-fluoro-4-oxo-1,4- dihydro-quinoline-3-carboxylic acid (160)

A solution of amine 128 (R₅═F) (83 mg, 0.2923 mmol), diacetyl quinolinyl borate 83 (111 mg, 0.2413 mmol) and triethylamine (0.5 mL) in acetonitrile (10 mL) were heated at reflux temperature overnight. The volatiles were evaporated and then THF (5 mL) and 10% aqueous HCl (4 mL) were added to the residue. This mixture was stirred for approximately 1 hour. The resulting solid was collected by filtration, washed with water and dried. Ethanol (4 mL) and hydrazine (0.01 mL) were added to the solid and the reaction heated at reflux temperature for 1.5 hours. The ethanol was evaporated in vacuo and water (20 mL) added to the remaining material. The solid was collected and dried to afford 160 as a yellow solid (20%). MS 428 (M+H).

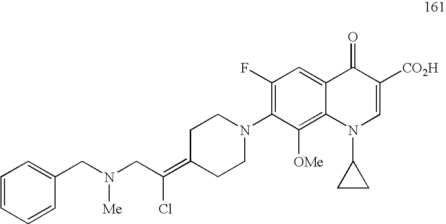

### 7-{4-[2-(Benzyl-methyl-amino)-1-chloro-ethylidene]- piperidin-1-yl}-1-cyclopropyl-6-fluoro-8-methoxy-4- oxo-1,4-dihydro-quinoline-3-carboxylic acid trifluoroacetic acid salt (161)

A solution of amine 135 (1.89 mmol) and triethylamine (1.2 mL, 8.59 mmol) in acetonitrile (15 mL) was treated with diacetyl quinolinyl borate 19 (727 mg, 1.72 mmol) under nitrogen. After 5 min, the reaction mixture was warmed to reflux temperature and the reaction mixture was allowed to stir for 24 h. The resulting mixture was allowed to cool to room temperature, and then concentrated in vacuo. The residue was dissolved in tetrahydrofuran (5 mL), treated with 10% aqueous hydrochloric acid (5 mL) at room temperature and stirred overnight. The resulting mixture was concentrated in vacuo and the residue purified by HPLC (reverse phase C-18 column, 30-90% acetonitrile/water containing 0.1% trifluoroacetic acid) to afford the trifluoroacetic acid salt of 161 (632 mg, 56%) as a yellow solid. MS 540 (M+H).

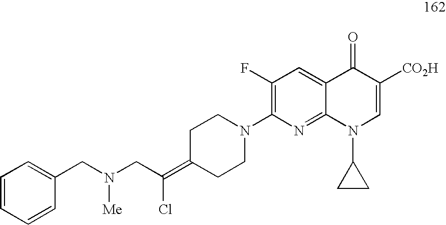

### 7-{4-[2-(Benzyl-methyl-amino)-1-chioro-ethylidene]- piperidin-1-yl}-1-cyclopropyl-6-fluoro-4-oxo-1,4- dihydro-[1,8]naphthyridine-3-carboxylic acid (162)

A solution of amine 135 (0.48 mmol) and triethylamine (0.28 mL, 2.0 mmol) in acetonitrile (7 mL) was treated with 7-chloro-1-cyclopropyl-6-fluoro-4-oxo-1,4-dihydro-[1,8]naphthyridine-3-carboxylic acid (113 mg, 0.40 mmol) under nitrogen. After 5 min, the reaction mixture was warmed to reflux temperature and the reaction mixture was allowed to stir for 24 h. The resulting mixture was allowed to cool to room temperature, concentrated in vacuo and the residue was diluted with water. The product was collected by filtration, and then washed with water and a small amount of methanol to afford the title compound (178 mg, 87%) as a white solid. MS 511 (M+H).

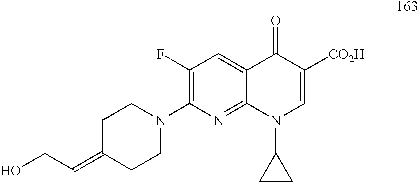

### 1-Cyclopropyl-6-fluoro-7-[4-(2-hydroxy-ethylidene)- piperidin-1-yl]-4-oxo-1,4-dihydro-[1,8]naphthyridine-3- carboxylic acid (163)

A solution of amine 103 (256 mg, 1.06 mmol) and triethylamine (0.5 mL, 3.55 mmol) in acetonitrile (4 mL) was treated with 7-chloro-1-cyclopropyl-6-fluoro-4-oxo-1,4-dihydro-naphthyridine-3-carboxylic acid (200 mg, 0.71 mmol) under nitrogen and the reaction mixture was allowed to stir for 16 h. The resulting mixture was concentrated in vacuo, and the residue was washed with water (3×10 mL) and allowed to dry overnight to afford the title compound 163 (105 mg, 40%). MS 374 (M+H).

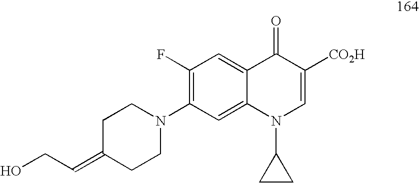

### 1-Cyclopropyl-6-fluoro-7-[4-(2-hydroxy-ethylidene)- piperid in-1-yl]-4-oxo-1,4-dihydro-quinoline-3- carboxylic acid (164)

A solution of amine 103 (146 mg, 0.61 mmol) and triethylamine (0.55 mL, 4.0 mmol) in acetonitrile (4 mL) was treated with diacetyl quinolinyl borate 17 (125 mg, 0.60 mmol) under nitrogen. After 5 min, the reaction mixture was warmed to reflux temperature and the reaction mixture was allowed to stir for 12 h. The resulting mixture was allowed to cool to room temperature, concentrated in vacuo, and the residue was washed with water (3×10 mL). The residue was dissolved in tetrahydrofuran (3 mL) and treated with 10% aqueous hydrochloric acid (5 mL) at room temperature. After 30 min, the reaction mixture was concentrated in vacuo, diluted with water (10 min) and the solid collected by filtration. The solid residue was washed with water (3×5 mL) and allowed to dry for 15 min. The solid was collected to afford 157 (5.1 mg, 2.2%) as a light yellow solid. MS 373 (M+H).

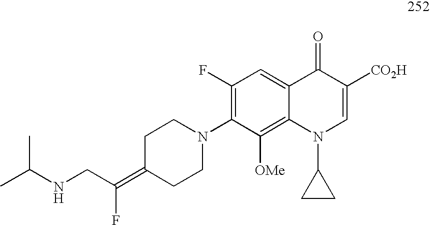

### 1-Cyclopropyl-6-fluoro-7-[4-(i -fluoro-2- isopropylamino-ethylidene)-piperidin-1-yl]-8-methoxy- 4-oxo-1,4-dihydro-quinoline-3-carboxylic Acid trifluoroacetic acid salt (252)

Amine 246 (0.12 mmol), difluoroborate ester 223 (34 mg, 0.10 mmol), and triethylamine (0.07 mL) in anhydrous acetonitrile (2 mL) were heated at reflux temperature under a nitrogen atmosphere for 24 hours. After cooling, the volatiles were evaporated in vacuo. Ethanol (5 mL) and triethylamine (0.5 mL) were added to the residue. This was heated at reflux temperature for 19 hours. The volatiles were evaporated and the residue was purified by HPLC (reverse phase C-18 column, 36-50% acetonitrile/water containing 0.1% trifluoroacetic acid) to afford the trifluoroacetic acid salt of 252 (11.4 mg, 20%) as a light yellow solid. MS 462 (M+H).

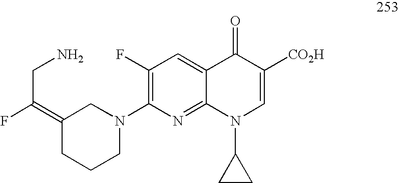

### (E)-7-[3-(2-Amino-1-fluoro-ethylidene)-piperidin-1-yl]- 1-cyclopropyl-6-fluoro-4-oxo- 1,4-dihydro- [1,8]naphthyridine-3-carboxylic acid trifluoroacetic acid salt (253)

A solution of amine E-232 (0.276 mmol) and triethylamine (0.16 mL, 1.152 mmol) in acetonitrile (5 mL) was treated with 7-chloro-1-cyclopropyl-6-fluoro-4-oxo-1,4-dihydro-[1,8]naphthyridine-3-carboxylic acid (65 mg, 0.230 mmol) under nitrogen. After 5 min, the reaction mixture was heated to reflux temperature and the reaction mixture was allowed to stir for 24 h. The resulting mixture was allowed to cool to room temperature and concentrated in vacuo. The residue was resuspended in methanol (5 mL) and the reaction mixture was treated with hydrazine (1 mL). The resulting mixture was stirred at room temperature overnight and concentrated in vacuo. The residue was purified by HPLC (reverse phase C-18 column, 25-40% acetonitrile/water containing 0.1% trifluoroacetic acid) to afford the trifluoroacetic acid salt of 253 (95.6 mg, 82%) as a white solid. MS 391 (M+H).

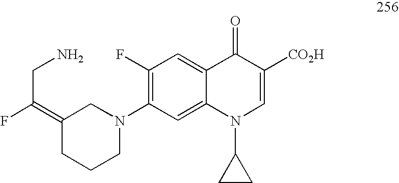

### (E)-7-[3-(2-Amino-1-fluoro-ethylidene)-piperidin-1-yl]-1-cyclopropyl-6-fluoro-4-oxo-1,4-dihydro-quinoline-3-carboxylic Acid Trifluoroacetic Acid Salt (256)

A solution of amine E-232 (0.260 mmol) and triethylamine (0.15 mL, 1.085 mmol) in acetonitrile (5 mL) was treated with diacetyl quinolinyl borate 17 (85 mg, 0.217 mmol) under nitrogen. After 5 min, the reaction mixture was heated to reflux temperature and the reaction mixture was allowed to stir for 24 h. The resulting mixture was allowed to cool to room temperature and concentrated in vacuo. The residue was dissolved in tetrahydrofuran (2 mL), treated with 10% aqueous hydrochloric acid (2 mL) at room temperature, and stirred overnight. The resulting mixture was concentrated in vacuo, the residue was resuspended in methanol (2 mL) and the reaction mixture was treated with hydrazine (0.5 mL). The resulting mixture was stirred at room temperature overnight and concentrated in vacuo. The residue was purified by HPLC (reverse phase C-18 column, 25-50% acetonitrile/water containing 0.1% trifluoroacetic acid) to afford the trifluoroacetic acid salt of 256 (27 mg, 25%) as a yellow solid. MS 390 (M+H).

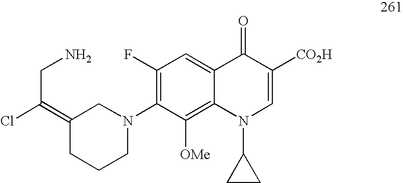

### (E)-7-[3-(2-Amino-1-chloro-ethylidene)-piperidin-1-yl]-1-cyclopropyl-6-fluoro-8-methoxy-4-oxo-1,4-dihydro-quinoline-3-carboxylic Acid Trifluoroacetic Acid Salt (261)

Amine E-233 (0.256 mmol), difluoroborate ester 223 (73 mg, 0.213 mmol), and triethylamine (0.15 mL) in anhydrous acetonitrile (5 mL) were heated at reflux temperature under a nitrogen atmosphere for 24 hours. After cooling, the volatiles were evaporated in vacuo. Ethanol (5 mL) and triethylamine (0.5 mL) were added to the residue. This was heated at reflux temperature for 19 hours. The volatiles were evaporated, the residue was resuspended in methanol (2 mL), and the reaction mixture was treated with hydrazine (0.5 mL). The resulting mixture was stirred at room temperature overnight and concentrated in vacuo. The residue was purified by HPLC (reverse phase C-18 column, 35-50% acetonitrile/water containing 0.1% trifluoroacetic acid) to afford the trifluoroacetic acid salt of 261 (26.6 mg, 23%) as a yellow solid. MS 436 (M+H).

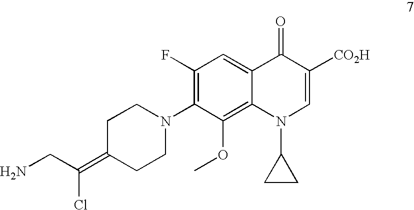

### 7-[4-(2-Amino-1-chloro-ethylidene)-piperidin-1-yl]-1-cyclopropyl-6-fluoro-8-methoxy-4-oxo-1,4-dihydro-quinoline-3-carboxylic Acid (7)

Amine 33 (34.95 mmol), difluoroborate ester 223 (7.29 g; 21.25 mmol) and triethylamine (35 mL) in anhydrous acetonitrile (250 mL) were heated at reflux temperature under a nitrogen atmosphere for 36 hours. After cooling, the volatiles were evaporated in vacuo. Ethanol (350 mL) and triethylamine (30 mL) were added to the residue. This was heated at reflux temperature for 38 hours. The volatiles were evaporated and water (250 mL) was added to the residue. The resulting solid was collected by filtration, washed with additional water and dried. This solid was suspended in ethanol (100 mL) and hydrazine (2.5 mL; 80 mmol) was added. The reaction was heated to 60° C. under a nitrogen atmosphere for 2 hours. The volatiles were evaporated. After the addition of water to the residue, the solid was collected, washed with water and dried to afford 7. MS 436 (M+H).

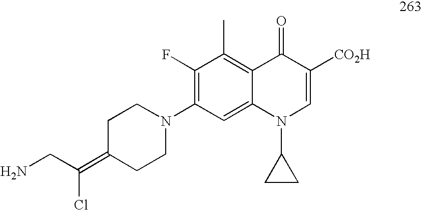

### 7-[4-(2-Amino-1-chloro-ethylidene)-piperidin-1-yl]-1-cyclopropyl-6-fluoro-5-methyl-4-oxo-1,4-dihydro-quinoline-3-carboxylic Acid (263)

Amine 33 (1.15 mmol), difluoroborate ester 224 (145 mg; 0.4433 mmol) and triethylamine (2.5 mL) in anhydrous acetonitrile (10 mL) were heated at reflux temperature under a nitrogen atmosphere for 48 hours. The volatiles were evaporated. 1,2-dichloroethane (10 mL), ethanol (20 mL) and triethylamine (2 mL) were added and the suspension heated at reflux temperature for 36 hours. The volatiles were evaporated. Water (30 mL) was added to the residue. The resulting solid was collected by filtration, washed with water and dried to afford the phthalimide-protected amine (226 mg, 93%). This solid (220 mg; 0.40 mmol) and hydrazine (0.05 mL; 1.59 mmol) suspended in methanol (30 mL) were heated at 60° C. for 5.5 hours. The volatiles were evaporated and water (25 mL) added to the gummy residue. After several minutes, a solid formed. This was filtered, washed with water and dried. 263 was isolated as a pale beige powder (146 mg; 87%). MS 420 (M+H).

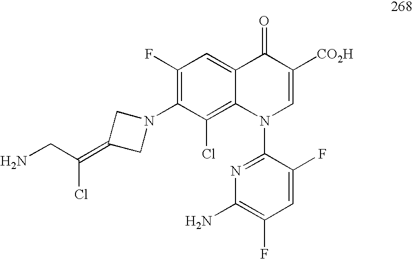

### 7-[3-(2-Amino-1-chloro-ethylidene)-azetidin-1-yl]-1-(6-amino-3,5-difluoro-pyridin-2-yl)-8-chloro-6-fluoro-4-oxo-1,4-dihydro-quinoline-3-carboxylic Acid (268)

1-(6-amino-3,5-difluoro-2-pyridinyl)-8-chloro-6,7-difluoro-1,4-dihydro-4-oxo-quinoline-3-carboxylic acid (0.412 mmol) (prepared by the methods described in WO97/11068 and U.S. Pat. No. 4,885,386), amine 129 (0.455 mmol), and triethylamine (0.3 mL) in anhydrous acetonitrile (10 mL) were heated at 55° C. under a nitrogen atmosphere for 1 hour. After cooling, the volatiles were evaporated in vacuo and water was added to the residue. The resulting solid was collected by filtration, washed with additional water and dried. This solid was suspended in methanol (10 mL) and treated with hydrazine (0.1 mL, 2.233 mmol). The reaction was stirred under a nitrogen atmosphere for 6 hours. The volatiles were evaporated. After the addition of water to the residue, the solid was collected, washed with methanol and dried to afford the title compound (37%). MS 501 (M+H).

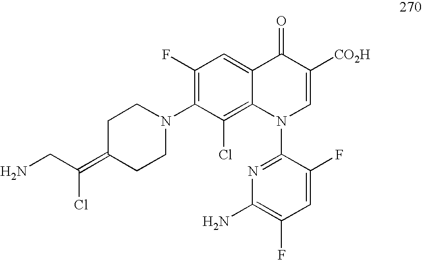

### 7-[4-(2-Amino-1-chloro-ethylidene)-piperidin-1-yl}-1-(6-amino-3,5-difluoropyridin-2-yl)-8-chloro-6-fluoro-4-oxo-1,4-dihydro-quinoline-3-carboxylic Acid (270)

Amine 33 (1.0 mmol), 1-(6-amino-3,5-difluoro-2-pyridinyl)-8-chloro-6,7-difluoro-1,4-dihydro-4-oxo-quinoline-3-carboxylic acid (0.5 mmol) (prepared by the methods described in WO 97/11068 and U.S. Pat. No. 4,885,386), and triethylamine (1.0 mmol) in anhydrous dimethylsulfoxide (1.5 mL) were heated at 70° C. under a nitrogen atmosphere for 1 hour. Then, ethanol (25 mL) was added to the reaction and it was heated at 90° C. for 10 minutes. After cooling, the resulting solid was collected by filtration, washed with additional ethanol and dried. This solid was suspended in ethanol (10 mL) and methylamine (33% in ethanol, 0.15 mL) was added. The reaction was stirred under a nitrogen atmosphere for 70 hours. The volatiles were evaporated. After the addition of water to the residue, the solid was collected, washed with water and dried to afford the title compound (57%). MS 529 (M+H).

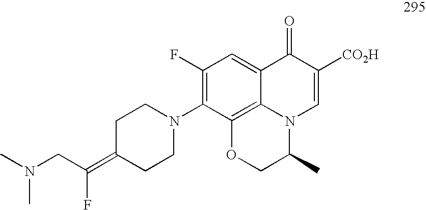

### 9-[4-(2-Dimethylamino-1-fluoro-ethylidene)-piperidin-1-yl]-8-fluoro-(3S)-3-methyl-6-oxo-2,3-dihydro-6H-1-oxa-3a-aza-phenalene-5-carboxylic Acid (295)

To a solution 157 (0.553 mmol) in acetonitrile (7 mL) was added S-(−)-9,10-difluoro-2,3-dihydro-3-methyl-7-oxo-7H-pyrido[1,2,3-de]-1,4-benzoxazine-6-carboxylic acid (0.0762 g, 0.271 mmol) and then triethylamine (0.79 mL). This mixture was refluxed for 5 days and then solvent was removed in vacuo. The resulting solid was dissolved in dimethylsulfoxide and purified by HPLC (reverse phase C-18 column, 10-90% acetonitrile/water containing 0.1% trifluoroacetic acid). The collected fractions were lyophilized to provide 0.0052 g of a yellow solid (3.5% yield for TFA salt). MS 434 (M+H).

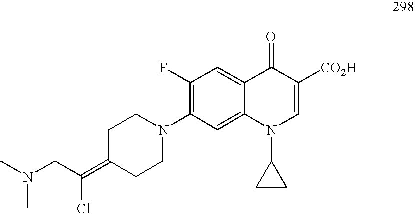

### 7-[4-(1-Chloro-2-dimethylamino-ethylidene)-piperidin-1-yl]-1-cyclopropyl-6-fluoro-4-oxo-1,4-dihydro-quinoline-3-carboxylic Acid Trifluoroacetic Acid Salt (298)

To 149 (0.506 mmol) in acetonitrile (7 mL) was added 1-cyclopropyl-1,4-dihydro-6,7-difluoro-4-oxo-quinoline-3-carboxylic acid (0.0699 g, 0.264 mmol) and then triethylamine (0.76 mL). This mixture was refluxed for 5 days and then solvent was removed in vacuo. The resulting solid was dissolved in dimethylsulfoxide and purified by HPLC (reverse phase C-18 column, 10-90% acetonitrile/water containing 0.1% trifluoroacetic acid). The collected fractions were lyophilized to provide 0.1024 g of a tan solid (71% yield for TFA salt). MS 434 (M+H).

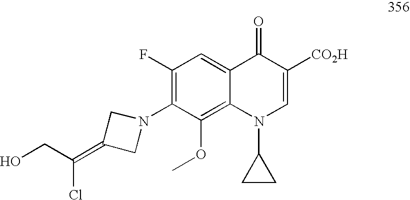

### 7-[3-(1-Chloro-2-hydroxy-ethylidene)-azetidin-1-yl]-1-cyclopropyl-6-fluoro-8-methoxy-4-oxo-1,4-dihydro-quinoline-3-carboxylic Acid (356)

Difluoroborate ester 223 (270 mg; 0.7870 mmoles), amino alcohol 350 (160 mg; 0.9411 mmoles) and triethylamine (0.5 mL) in acetonitrile (5 mL) were heated at reflux temperature for 48 hours. The volatiles were evaporated and ethanol (10 mL) and triethylamine (0.5 mL) were added to the residue. This was heated at reflux temperature overnight. After cooling, the volatiles were evaporated. Water (25 mL) was added to the residue and the resulting solid collected by filtration and dried to afford 356 as a beige solid (163 mg; 53%). MS 409, 411 (M+H).

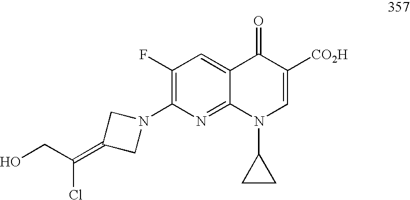

### 7-[3-(1-Chloro-2-hydroxy-ethylidene)-azetidin-1-yl]-1-cyclopropyl-6-fluoro-4-oxo-1,4-dihydro-[1,8]naphthyridine-3-carboxylic Acid (357)

7-Chloro-1-cyclopropyl-6-fluoro-4-oxo-1,4-dihydronaphthyridine-3-carboxylic acid (136 mg; 0.4811 mmoles), amino alcohol 350 (97 mg; 0.570 mmoles) and triethylamine (0.5 mL) in acetonitrile (10 mL) were heated at reflux temperature overnight. The volatiles were evaporated and water (25 mL) was added to the residue. The resulting solid was collected by filtration, washed with water and dried to afford 357 as a beige powder (168 mg; 92%). MS 380, 382 (M+H).

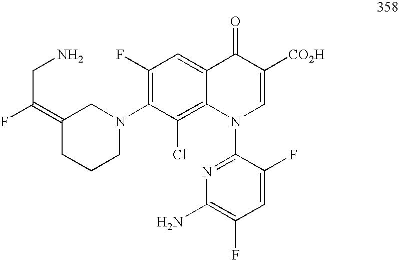

### (E)-1-(6-Amino-3,5-difluoro-pyridin-2-yl)-7-[3-(2-amino-1-fluoro-ethylidene)-piperidin-1-yl]-8-chloro-6-fluoro-4-oxo-1,4-dihydro-quinoline-3-carboxylic Acid Trifluoroacetate (358)

A solution of amine E-232 (0.267 mmol) and triethylamine (0.19 mL) in acetonitrile (1 mL) was treated with 1-(6-amino-3,5-difluoro-2-pyridinyl)-8-chloro-6,7-difluoro-1,4-dihydro-4-oxo-quinoline-3-carboxylic acid (94 mg, 0.243 mmol) (prepared by the methods described in WO 97/11068 and U.S. Pat. No. 4,885,386) under nitrogen. After 5 min, the reaction mixture was heated to reflux temperature and the reaction mixture was allowed to stir for 20 h. The resulting mixture was allowed to cool to room temperature and concentrated in vacuo. The residue was suspended in methanol (4 mL) and methylamine (33% in ethanol, 4 mL) was added. The reaction was stirred under a nitrogen atmosphere for 3.5 hours. The volatiles were evaporated. The residue was purified by HPLC (reverse phase C-18 column, 25-50% acetonitrile/water containing 0.1% trifluoroacetic acid) to afford the trifluoroacetic acid salt of 358 (50 mg, 33%) as a yellow solid. MS 512 (M+H).

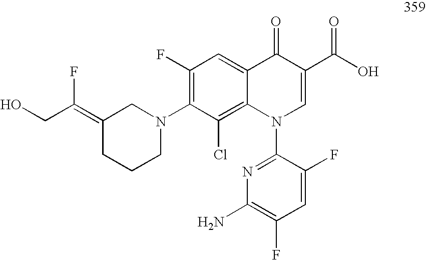

### (Z)-1-(6-Amino-3,5-difluoro-pyridin-2-yl)-8-chloro-6-fluoro-7-[3-(1-fluoro-2-hydroxy-ethylidene)-piperidin-1-yl]-4-oxo-1,4-dihydroquinoline-3-carboxylic Acid Trifluoroacetate (359)

A solution of amine Z-332 (0.4078 mmol) and triethylamine (0.3 mL) in acetonitrile (1.5 mL) was treated with 1-(6-amino-3,5-difluoro-2-pyridinyl)-8-chloro-6,7-difluoro-1,4-dihydro-4-oxo-quinoline-3-carboxylic acid (144 mg, 0.3706 mmol) (prepared by the methods described in WO 97/11068 and U.S. Pat. No. 4,885,386) under nitrogen. After 5 min, the reaction mixture was heated to reflux temperature and the reaction mixture was allowed to stir for 24 h. The resulting mixture was allowed to cool to room temperature and concentrated in vacuo. The residue was purified by HPLC (reverse phase C-18 column, 35-55% acetonitrile/water containing 0.1% trifluoroacetic acid) to afford the trifluoroacetic acid salt of 359 (63 mg, 27%) as a yellow solid. MS 513 (M+H).

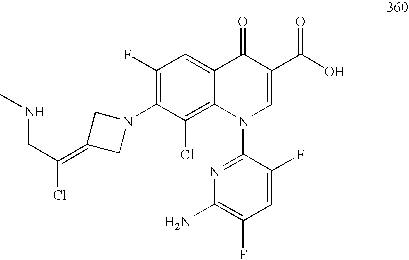

### 1-(6-Amino-3,5-difluoro-pyridin-2-yl)-8-chloro-7-[3-(1-chloro-2-methylamino-ethylidene)-azetidin-1-yl]-6-fluoro-4-oxo-1,4-dihydro-quinoline-3-carboxylic Acid (360)

Amine 346 (28 mg; 0.128 mmol), 6,7-difluoro-8-chloro-1-(6-amino-3,5-difluoropyridi-2-yl)-4-oxo-1,4-dihydronaphthyridine-3-carboxylic acid (45 mg; 0.116 mmol) and triethylamine (0.1 mL; 0.717 mmol) in anhydrous acetonitrile (4 mL) were heated at 55° C. under a nitrogen atmosphere for 18 hours. After cooling, the volatiles were evaporated in vacuo and water was added to the residue. The resulting solid was collected by filtration, washed with additional water and dried to give the crude material. Purification of the crude material by HPLC (reverse phase C-18 column, 10-50% acetonitrile/water containing 0.1% trifluoroacetic acid) provided the trifluoroacetic acid salt of the title compound (20 mg, 28%) as a pale yellow powder. MS 515 (M+H).

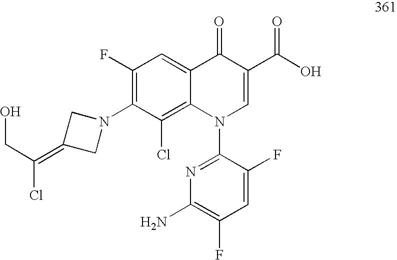

1-(6-Amino-3,5-difluoro-pyridin-2-yl)-8-chloro-7-[3-(1-chloro-2-hydroxy-ethylidene)-azetidin-1-yl]-6-fluoro-4-oxo-1,4-dihydro-quinoline-3-carboxylic acid (361) 361 was prepared in a similar manner as 360 except that amine 350 was used in place of amine 125, and the crude reaction product was purified by washing with a small amount of methanol. The resulting pale brown powder was collected by filtration. MS 502 (M+H).

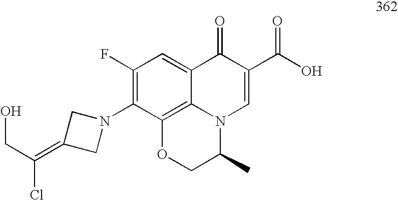

### (S)-9-[3-(1-Chloro-2-hydroxy-ethylidene)-azetidin-1-yl]-8-fluoro-3-methyl-6-oxo-2,3-dihydro-6H-1-oxa-3a-aza-phenalene-5-carboxylic Acid (362)

362 was prepared in an analogous manner to 161 except that amine 350 and diacetyl quinolinyl borate 319 were used in place of amine 135 and diacetyl quinolinyl borate 19. The trifluoroacetic acid salt of the title compound was isolated as yellow powder. MS 395 (M+H).

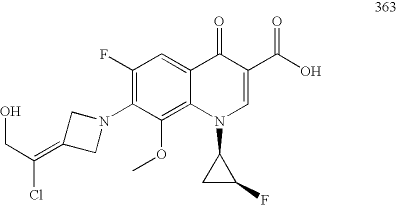

### 7-[3-(1-Chloro-2-hydroxy-ethylidene)-azetidin-1-yl]-6-fluoro-(1R,2S)-1-(2-fluoro-cyclopropyl)-8-methoxy-4-oxo-1,4-dihydro-quinoline-3-carboxylic Acid (363)

363 was prepared in a similar manner to 356 except difluoroborate ester 225 was used in place of difluoroborate ester 223. In addition, the crude material was purified by washing with a small amount of methanol. The resulting light brown powder was collected by filtration. MS 427 (M+H).

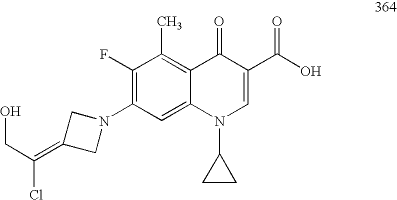

### 7-[3-(1-Chloro-2-hydroxy-ethylidene)-azetidin-1-yl]-1-cyclopropyl-6-fluoro-5-methyl-4-oxo-1,4-dihydro-quinoline-3-carboxylic Acid (364)

364 was prepared in a similar manner to 356 except that difluoroborate ester 224 was used in place of difluoroborate ester 223. The crude material was purified by HPLC (reverse phase C-18 column, 10-50% acetonitrile/water containing 0.1% trifluoroacetic acid) to afford the trifluoroacetic acid salt of the title compound as pale brown powder. MS 393 (M+H).

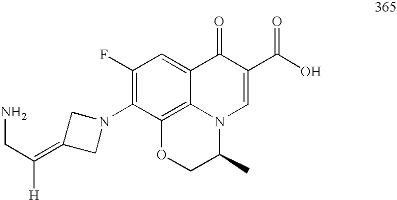

### (S)-9-[3-(2-Amino-ethylidene)-azetidin-1-yl]-8-fluoro-3-methyl-6-oxo-2,3-dihydro-6H-1-oxa-3a-aza-phenalene-5-carboxylic Acid (365)

365 was prepared in a similar manner as example 160 except that amine 340 and diacetyl quinolinyl borate 319 were used in place of amine 128 and diacetyl quinolinyl borate 83. MS 360 (M+H).

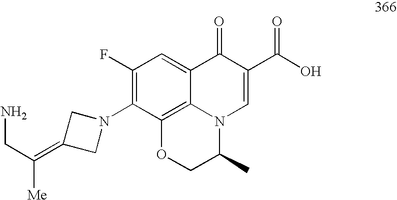

### (S)-9-[3-(2-Amino-1-methyl-ethylidene)-azetidin-1-yl]-8-fluoro-3-methyl-6-oxo-2,3-dihydro-6H-1-oxa-3a-aza-phenalene-5-carboxylic Acid (366)

366 was prepared in a similar manner as example 160 except that amine 341 and diacetyl quinolinyl borate 319 were used in place of amine 128 and diacetyl quinolinyl borate 83. MS 374 (M+H).

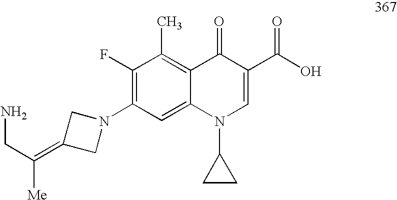

### 7-[3-(2-Amino-1-methyl-ethylidene)-azetidin-1-yl]-1-cyclopropyl-6-fluoro-5-methyl-4-oxo-1,4-dihydro-quinoline-3-carboxylic Acid (367)

367 was prepared in a similar manner as for the synthesis of example 7 except that amine 341 and difluoroborate ester 224 were used in place of amine 33 and difluoroborate ester 223. MS 372 (M+H).

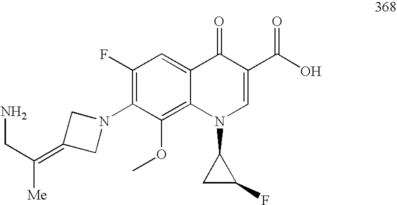

### 7-[3-(2-Amino-1-methyl-ethylidene)-azetidin-1-yl]-6-fluoro-(1R,2S)-1-(2-fluoro-cyclopropyl)-8-methoxy-4-oxo-1,4-dihydro-quinoline-3-carboxylic Acid (368)

368 was prepared in a similar fashion as example 7 except that amine 341 and difluoroborate ester 225 were used in place of amine 33 and difluoroborate ester 223. MS 406 (M+H).

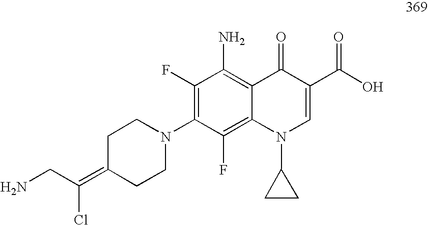

### 5-Amino-7-[4-(2-amino-1-chloro-ethylidene)-piperidin-1-yl]-1-cyclopropyl-6,8-difluoro-4-oxo-1,4-dihydro-quinoline-3-carboxylic Acid (369)

A solution of amine 33 (0.77 mmol) and triethylamine (0.21 mL, 1.5 mmol) in N-methyl-2-pyrrolidinone (NMP) (3 mL) was treated with 5-amino-1-cyclopropyl-6,7,8-trifluoro-4-oxo-1,4-dihydroquinoline-3-carboxylic acid (75 mg, 0.25 mmol) (prepared as described in EP 172651) and the reaction mixture was heated at 110° C. under nitrogen for 24 h. The resulting mixture was allowed to cool to room temperature, diluted with EtOH (15 mL), and filtered. The resulting solid was suspended in methanol (15 mL) and treated with hydrazine (0.2 mL). The reaction was allowed to stir for 24 h at room temperature. The reaction mixture was concentrated in vacuo, diluted with water and filtered. The resulting yellow solid was washed with water (3×20 mL) and ether (20 mL), and allowed to dry overnight to afford the title compound 369 (60 mg, 55%). MS 439 (M+H).

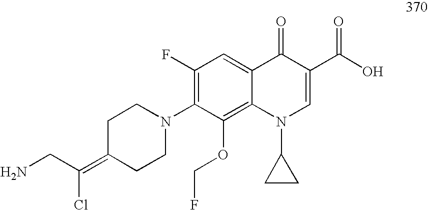

### 7-[4-(2-Amino-1-chloro-ethylidene)-piperidin-1-yl]-1-cyclopropyl-6-fluoro-8-fluoromethoxy-4-oxo-1,4-dihydro-quinoline-3-carboxylic Acid (370)

A solution of amine 33 (0.67 mmol) and triethylamine (0.3 mL, 2.2 mmol) in DMF (1 mL) was treated with 1-cyclopropyl-6,7-difluoro-8-fluoromethoxy-4-oxo-1,4-dihydroquinoline-3-carboxylic acid difluoroborate ester 321 (80 mg, 0.22 mmol) under nitrogen and the reaction mixture was heated at 40° C. for 24 h. The mixture was allowed to cool to room temperature and concentrated. The residue was dissolved in EtOH (4 mL), and Et₃N (0.3 mL, 2.2 mmol) was added to the solution. This solution was heated to reflux for 6 hours. After being cooled to room temperature, the reaction mixture was filtered. The resulting solid was suspended in methanol (10 mL) and treated with hydrazine (0.4 mL). The reaction mixture was allowed to stir for 24 hours at room temperature before it was concentrated in vacuo. The residue was washed with water (3×10 mL) and ether (10 mL), and allowed to dry overnight to afford the title compound 370. (54 mg, 54%). MS 454 (M+H).

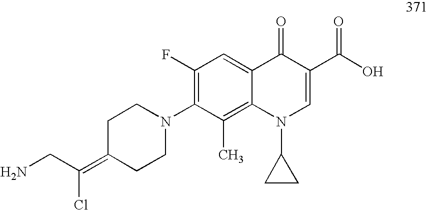

### 7-[4-(2-Amino-1-chloro-ethylidene)-piperidin-1-yl]-1-cyclopropyl-6-fluoro-8-methyl-4-oxo-1,4-dihydro-quinoline-3-carboxylic Acid (371)

A solution of amine 33 (1.8 mmol) and triethylamine (0.5 mL, 3.6 mmol) in DMF (2 mL) was treated with 1-cyclopropyl-6,7-difluoro-8-methyl-4-oxo-1,4-dihydroquinoline-3-carboxylic acid difluoroborate ester 322 (0.3 g, 0.92 mmol) and the reaction mixture was heated at 40° C. under nitrogen for 4 days. The mixture was allowed to cool to room temperature and was concentrated. The residue was dissolved in EtOH (5 mL), and Et₃N (0.5 mL, 3.6 mmol) was added to the solution. This solution was heated to reflux temperature for 16 hours, cooled to room temperature and concentrated. The residue was diluted with CH₂Cl₂ (100 mL), and washed with H₂O and brine. The organic solution was dried over MgSO₄ and concentrated. The residue was purified by silica gel chromatography (4% MeOH in CH₂Cl₂). The resulting solid product was suspended in methanol (10 mL) and treated with hydrazine (0.4 mL). The reaction mixture was allowed to stir for 24 hours at room temperature before it was concentrated in vacuo. The residue was purified by silica gel chromatography (7% MeOH in CH₂Cl₂) to afford the title compound 371 (30 mg, 8%). MS 420 (M+H).

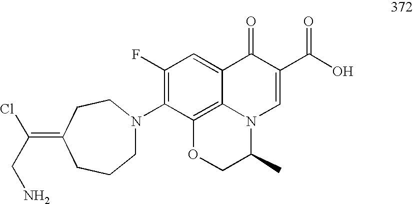

### (3S)-9-[(Z)-4-(2-Amino-1-chloro-ethylidene)-azepan-1-yl]-8-fluoro-3-methyl-6-oxo-2,3-dihydro-6H-1-oxa-3a-aza-phenalene-5-carboxylic Acid (372)

372 was prepared in a similar manner as example 160 except that amine Z-330 and diacetyl quinolinyl borate 319 were used in place of amine 128 and diacetyl quinolinyl borate 83. MS 436 (M+H).

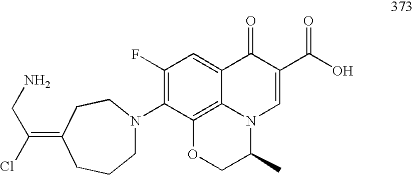

### (3S)-9-[(E)-4-(2-Amino-1-chloro-ethylidene)-azepan-1-yl]-8-fluoro-3-methyl-6-oxo-2,3-dihydro-6H-1-oxa-3a-aza-phenalene-5-carboxylic Acid (373) 373 was prepared in a similar manner as example 160 except that amine

E-330 and diacetyl quinolinyl borate 319 were used in place of amine 128 and diacetyl quinolinyl borate 83. MS 436 (M+H).

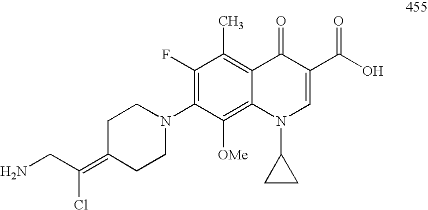

### 7-[4-(2-Amino-1-chloro-ethylidene)-piperidin-1-yl]-1-cyclopropyl-6-fluoro-8-methoxy-5-methyl-4-oxo-1,4-dihydro-quinoline-3-carboxylic Acid (455)

455 was prepared in a similar manner to 7-[4-(2-amino-1-chloro-ethylidene)piperidin-1-yl]-1-cyclopropyl-6-fluoro-8-fluoromethoxy-4-oxo-1,4-dihydroquinoline-3-carboxylic acid (370) except that difluoroquinolinyl borate ester 454 was used in place of 321. MS 450 (M+H).

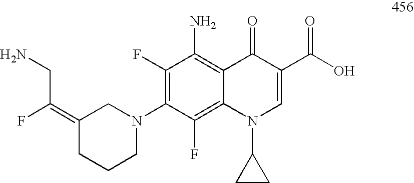

### 5-Amino-7-[(E)-3-(2-amino-1-fluoro-ethylidene)-piperidin-1-yl]-1-cyclopropyl-6,8-difluoro-4-oxo-1,4-dihydro-quinoline-3-carboxylic Acid (456)

456 was prepared in a similar fashion as 7-[4-(2-amino-1-chloro-ethylidene)piperidin-1-yl]-5-amino-1-cyclopropyl-6,8-difluoro-4-oxo-1,4-dihydroquinoline-3-carboxylic acid (369) except that amine E-232 was used in place of amine 33. MS 423 (M+H).

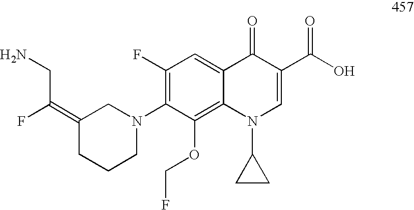

### 7-[(E)-3-(2-Amino-1-fluoro-ethylidene)-piperidin-1-yl]-1-cyclopropyl-6-fluoro-8-fluoromethoxy-4-oxo-1,4-dihydro-quinoline-3-carboxylic Acid (457)

457 was prepared in a similar fashion as 7-[4-(2-amino-1-chloro-ethylidene)piperidin-1-yl]-1-cyclopropyl-6-fluoro-8-fluoromethoxy-4-oxo-1,4-dihydroquinoline-3-carboxylic acid (370) except that amine E-232 was used in place of amine 33. MS 438 (M+H).

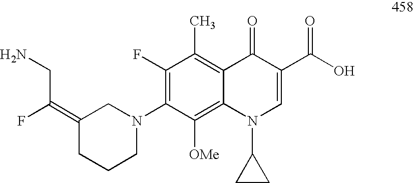

### 7-[(E)-3-(2-Amino-1-fluoro-ethylidene)-piperidin-1-yl]-1-cyclopropyl-6-fluoro-8-methoxy-5-methyl-4-oxo-1,4-dihydro-quinoline-3-carboxylic Acid (458)

458 was prepared in a similar manner as 7-[4-(2-amino-1-chloro-ethylidene)piperidin-1-yl]-1-cyclopropyl-6-fluoro-8-methoxy-5-methyl-4-oxo-1,4-dihydroquinoline-3-carboxylic acid (455) except that amine E-232 was used in place of amine 33. MS 434 (M+H).

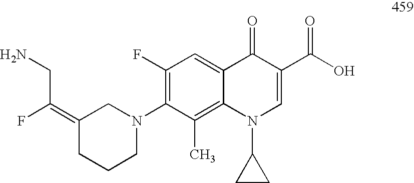

### 7-[(E)-3-(2-Amino-1-fluoro-ethylidene)-piperidin-1-yl]-1-cyclopropyl-6-fluoro-8-methyl-4-oxo-1,4-dihydro-quinoline-3-carboxylic Acid (459)

459 was prepared in a similar manner as 7-[4-(2-amino-1-chloro-ethylidene)piperidin-1-yl]-1-cyclopropyl-6-fluoro-8-methyl-4-oxo-1,4-dihydroquinoline-3-carboxylic acid (371) except that amine E-232 was used in place of amine 33. MS 404 (M+H).

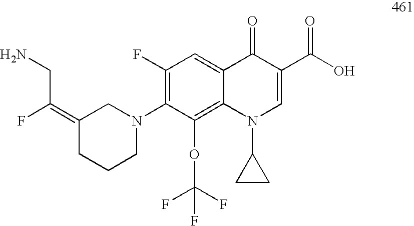

### 7-[(E)-3-(2-Amino-1-fluoro-ethylidene)-piperidin-1-yl]-1-cyclopropyl-6-fluoro-4-oxo-8-trifluoromethoxy-1,4-dihydro-quinoline-3-carboxylic Acid (461)

461 was prepared in a similar manner as 7-[4-(2-amino-1-chloro-ethylidene)piperidin-1-yl]-1-cyclopropyl-6-fluoro-8-fluoromethoxy-4-oxo-1,4-dihydroquinoline-3-carboxylic acid (370) except that difluoroborate ester 460 was used instead of 421 and amine E-232 was used instead of amine 33. MS 474 (M+H).

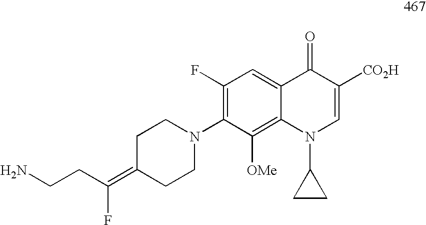

### 7-{4-[3-Amino-1-fluoro-propylidene]-piperidin-1-yl}-1-cyclopropyl-6-fluoro-8-methoxy-4-oxo-1,4-dihydro-quinoline-3-carboxylic Acid (467)

Compound 467 was prepared in a manner similar as 7-[4-(2-amino-1-chloro-ethylidene)piperidin-1-yl]-1-cyclopropyl-6-fluoro-8-fluoromethoxy-4-oxo-1,4-dihydroquinoline-3-carboxylic acid (370) except that 1-cyclopropyl-1,4-dihydro-6,7-difluoro-methoxy-4-oxo-quinoline-3-carboxylic acid difluoroborate ester (223) was used instead of 321 and amine 466 was used instead of amine 33. Compound 467 was obtained as a yellow solid (85% yield). MS 434 (M+H).

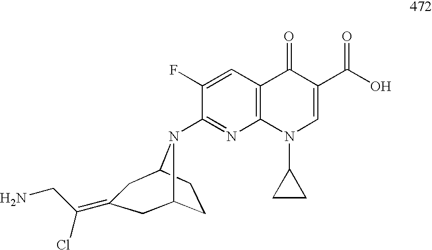

### 7-[3-(2-Amino-1-chloro-ethylidene)-8-aza-bicyclo[3.2.1]oct-8-yl]-1-cyclopropyl-6-fluoro-4-oxo-1,4-dihydro-[1,8]naphthyridine-3-carboxylic Acid Trifluoroacetic Acid Salt (472)

Compound 472 was prepared in a similar manner as compound 253 except that amine 471 was used instead of amine E-232. MS 433, 435 (M+H).

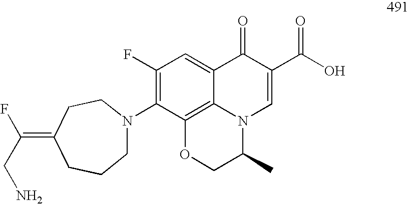

### (3S)-9-[(Z)-4-(2-Amino-1-fluoro-ethylidene)-azepan-1-yl]-8-fluoro-3-methyl-6-oxo-2,3-dihydro-6H-1-oxa-3a-aza-phenalene-5-carboxylic Acid (491)

491 was prepared in a similar manner as example 160 except that amine Z-476 and diacetyl quinolinyl borate 319 were used in place of amine 128 and diacetyl quinolinyl borate 83. MS 420 (M+H).

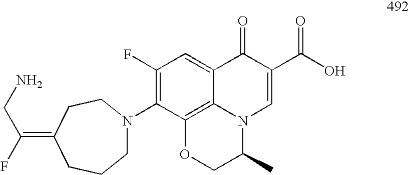

### (3S)-9-[(E)-4-(2-Amino-1-fluoro-ethylidene)-azepan-1-yl]-8-fluoro-3-methyl-6-oxo-2,3-dihydro-6H-1-oxa-3a-aza-phenalene-5-carboxylic Acid (492)

492 was prepared in a similar manner as example 160 except that amine E-476 and diacetyl quinolinyl borate 319 were used in place of amine 128 and diacetyl quinolinyl borate 83. MS 420 (M+H).

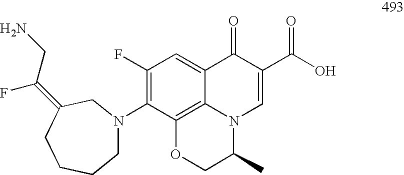

### 9-[(Z)-6-(2-Amino-1-fluoro-ethylidene)-[1,4]oxazepan-4-yl]-8-fluoro-(3S)-3-methyl-6-oxo-2,3-dihydro-6H-1-oxa-3a-aza-phenalene-5-carboxylic Acid (493)

493 was prepared in a similar manner as example 160 except that amine Z-489 and diacetyl quinolinyl borate 319 were used in place of amine 128 and diacetyl quinolinyl borate 83. MS 422 (M+H).

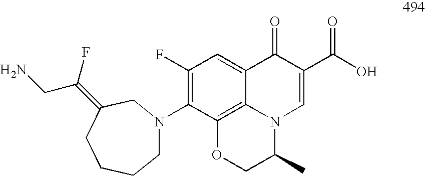

### 9-[(E)-6-(2-Amino-1-fluoro-ethylidene)-[1,4]oxazepan-4-yl]-8-fluoro-(3S)-3-methyl-6-oxo-2,3-dihydro-6H-1-oxa-3a-aza-phenalene-5-carboxylic Acid (494)

493 was prepared in a similar manner as example 160 except that amine E-489 and diacetyl quinolinyl borate 319 were used in place of amine 128 and diacetyl quinolinyl borate 83. MS 422 (M+H).

Table 4 lists the additional compounds of the instant invention prepared by the experimental procedures detailed above. In each case, the final product was obtained by condensing the amine listed in Table 4 with the appropriate heterocyclic nucleus or activated heterocyclic nucleus using analogous procedures to those above.

In the case of the naphthyridines 2-4, 59-63, 69, and 173-176 an analogous experimental procedure to that for compound 1 was used in their preparation. For the naphthyridines 171, 172, 185 and 271, an analogous procedure to that for 163 was used. For the naphthyridines 81, 183, 184, 377, 380, and 381 an analogous experimental procedure to that for 80 was used. For the naphthyridines 165-170, 177-182, 186, and 247 a similar procedure to that for 162 was used in their preparation. In the case of naphthyridines 254, 255, 374, 375, 376, 378, 379, and 495-500 an analogous experimental procedure to that for compound 253 was used in their preparation.

Quinolones 262, 273, 346, 393, 394, and 505-508 were prepared in an analogous manner to the preparation of 7 above. For the quinolones 6, 8-15, 64-66, 70, 71, 73, 78, 187, 188, 201-204, 206-208, 210, 272, and 274-277, an analogous experimental procedure to that for compound 5 was used in their preparation. In addition to the method described above, quinolone 7 could also be prepared by an analogous method to that used for 5. For the quinolones 76, 77, 189, 205, and 209, an analogous procedure to that for 160 was used in their preparation. For the quinolones 190-200 and 248-251 an analogous procedure to that for 161 was used. For the quinolone 211 a similar procedure to that for 163 was used in its preparation. In the case of the quinolones 257-260, 382-384, 386, 387, 395-402, and 501-504 an analogous experimental procedure to that for compound 256 was used in their preparation. Quinolones 269, 420, 422, and 423 were prepared in an analogous manner to quinolone 268. Quinolones 299, 300, and 385 were prepared in an analogous manner to quinolone 298. Quinolones 301, 447, and 448 were prepared in an analogous manner to quinolone 256. Quinolones 302, 303, 388-390, 392, 404-410, 424-427, 446, 449, 451, and 452 were prepared in an analogous fashion to quinolone 261. For the quinolones 411-414 and 450, an analogous experimental procedure to that for compound 358 was used in their preparation. Quinolones 415-417 were prepared in an analogous fashion to quinolone 359. Quinolone 418 was prepared in a similar fashion to quinolone 360. Quinolone 419 was prepared in an analogous fashion to 361.

TABLE 4

| Structure | Amine | Compound Number |
|---|---|---|
| 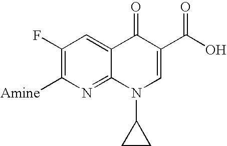 | 27 32 33 39 41 43 48 129 148 154 134 156 158 159 105 104 85 118 119 117 120 155 110 107 149 152 157 246 E-233 Z-233 Z-232 Z-326 E-326 E-238 Z-330 E-330 Z-476 E-476 Z-489 E-489 (S/Z- 490 (R/Z- 490 | 59 (M + H) = 373 2 (M + H) = 387 3 (M + H) = 407 60 (M + H) = 389 61 (M + H) = 416 62 (M + H) = 403 63 (M + H) = 391 81 (M + H) = 379, 381 165 (M + H) = 525 166 (M + H) = 449 167 (M + H) = 495 168 (M + H) = 509 169 (M + H) = 447 170 (M + H) = 550 171 (M + H) = 388 172 (M + H) = 392 69 (M + H) = 401 173 (M + H) = 413 174 (M + H) = 427 175 (M + H) = 419 176 (M + H) = 452 177 (M + H) = 478 178 (M + H) = 404 179 (M + H) = 464 180 (M + H) = 435 181 (M + H) = 475 182 (M + H) = 419 247 (M + H) = 433 254 (M + H) = 407 255 (M + H) = 407 374 (M + H) = 391 375 (M + H) = 373 376 (M + H) = 373 377 (M + H) = 387 378 (M + H) = 421 379 (M + H) = 421 495 (M + H) = 405 496 (M + H) = 405 497 (M + H) = 407 498 (M + H) = 407 499 (M + H) = 421 500 (M + H) = 421 |
| 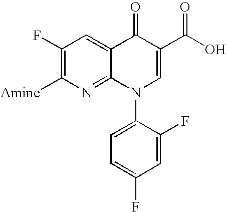 | 31 128 129 103 157 33 Z-238 E-238 | 4 (M + H) = 463 183 (M + H) = 435 184 (M + H) = 451, 453 185 (M + H) = 446 186 (M + H) = 491 271 (M + H) = 479 380 (M + H) = 459 381 (M + H) = 459 |
| 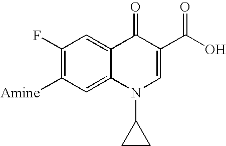 | 32 33 129 135 134 154 246 E-233 Z-233 128 157 152 Z-238 Z-232 Z-326 E-326 E-238 Z-330 E-330 Z-476 E-476 Z-489 E-489 | 64 (M + H) = 386 65 (M + H) = 406 76 (M + H) = 378, 380 187 (M + H) = 510 188 (M + H) = 494 248 (M + H) = 448 249 (M + H) = 432 257 (M + H) = 406 258 (M + H) = 406 272 (M + H) = 362 299 (M + H) = 418 300 (M + H) = 474 301 (M + H) = 386 382 (M + H) = 390 383 (M + H) = 372 384 (M + H) = 372 385 (M + H) = 386 386 (M + H) = 420 387 (M + H) = 420 501 (M + H) = 404 502 (M + H) = 404 503 (M + H) = 406 504 (M + H) = 406 |
| 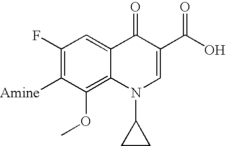 | 31 Z-53 E-53 Z-37 E-37 58 129 148 149 150 151 152 153 154 134 156 157 158 32 85 116 119 118 117 120 27 128 Z-238 E-232 Z-232 Z-326 E-326 341 E-238 Z-330 E-330 Z-233 Z-476 E-476 Z-489 E-489 | 6 (M + H) = 420 11 (M + H) = 434 12 (M + H) = 434 13 (M + H) = 422 14 (M + H) = 422 15 (M + H) = 450 189 (M + H) = 408, 410 190 (M + H) = 554 191 (M + H) = 464 192 (M + H) = 492 193 (M + H) = 490 194 (M + H) = 504 195 (M + H) = 506 196 (M + H) = 478 197 (M + H) = 524 198 (M + H) = 538 199 (M + H) = 448 200 (M + H) = 476 70 (M + H) = 402 71 (M + H) = 430 201 (M + H) = 446 202 (M + H) = 456 203 (M + H) = 442 78 (M + H) = 448 204 (M + H) = 481 212 (M + H) = 402 273 (M + H) = 392 302 (M + H) = 416 303 (M + H) = 420 388 (M + H) = 420 389 (M + H) = 402 390 (M + H) = 402 391 (M + H) = 388 392 (M + H) = 416 393 (M + H) = 450 394 (M + H) = 450 446 (M + H) = 436 505 (M + H) = 434 506 (M + H) = 434 507 (M + H) = 436 508 (M + H) = 436 |
| 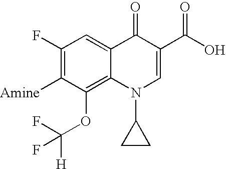 | 129 135 134 31 33 154 246 E-232 E-233 Z-232 Z-326 E-326 Z-233 | 205 (M + H) = 444, 446 206 (M + H) = 576 207 (M + H) = 560 208 (M + H) = 456 73 (M + H) = 472 250 (M + H) = 514 251 (M + H) = 498 259 (M + H) = 456 260 (M + H) = 472 395 (M + H) = 456 396 (M + H) = 438 397 (M + H) = 438 447 (M + H) = 472 |
| 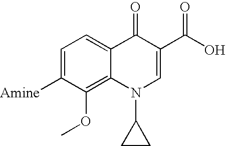 | 31 129 128 33 | 10 (M + H) = 402 209 (M + H) = 390, 392 274 (M + H) = 374 275 (M + H) = 418 |
| 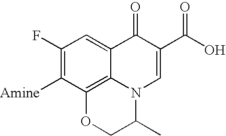 | 31 32 43 39 105 | 8 (M + H) = 406 9 (M + H) = 402 66 (M + H) = 418 210 (M + H) = 404 211 (M + H) = 427 |
| 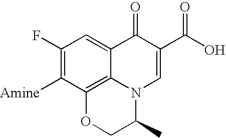 | 33 128 129 E-232 Z-232 E-233 E-326 Z-326 350 Z-238 Z-233 | 277 (M + H) = 422 276 (M + H) = 378 77 (M + H) = 394, 396 398 (M + H) = 406 399 (M + H) = 406 400 (M + H) = 422 401 (M + H) = 388 402 (M + H) = 388 403 (M + H) = 395 404 (M + H) = 402 448 (M + H) = 422 |
| 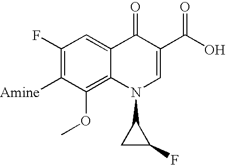 | 33 E-232 Z-232 E-233 Z-326 E-326 Z-238 Z-233 | 262 (M + H) = 454 405 (M + H) = 438 406 (M + H) = 438 407 (M + H) = 454 408 (M + H) = 420 409 (M + H) = 420 410 (M + H) = 434 449 (M + H) = 454 |
| 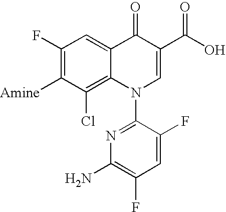 | 128 Z-232 E-233 Z-326 E-326 E-332 Z-333 104 347 351 340 341 Z-238 E-238 Z-233 | 269 (M + H) = 484 411 (M + H) = 512 412 (M + H) = 528 413 (M + H) = 494 414 (M + H) = 494 415 (M + H) = 513 416 (M + H) = 529 417 (M + H) = 513 418 (M + H) = 498 419 (M + H) = 485 420 (M + H) = 466 421 (M + H) = 480 422 (M + H) = 508 423 (M + H) = 508 450 (M + H) = 528 |
| 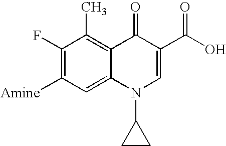 | E-232 Z-232 E-233 Z-326 E-326 Z-233 | 424 (M + H) = 404 425 (M + H) = 404 426 (M + H) = 420 427 (M + H) = 386 451 (M + H) = 386 452 (M + H) = 420 |

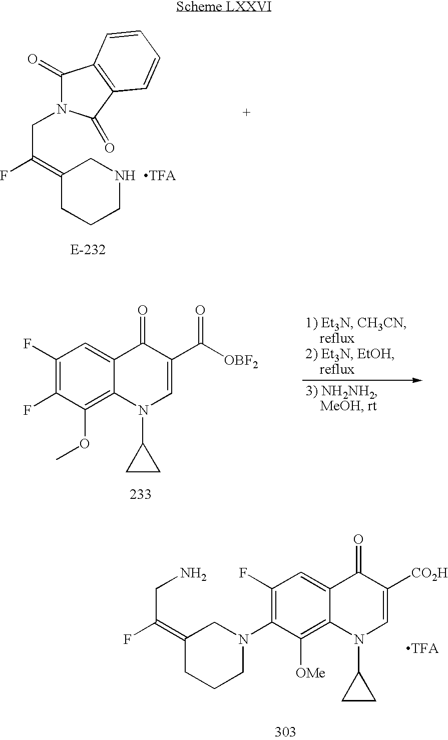

### (E)-7-[3-(2-Amino-1-fluoro-ethylidene)-piperidin-1-yl]-1-cyclopropyl-6-fluoro-8-methoxy-4-oxo-1,4-dihydro-quinoline-3-carboxylic Acid Trifluoroacetic Acid Salt (303)

Quinolone 303 was also synthesized according to the following procedure. Amine E-232 (104 mg, 0.267 mmol), difluoroborate ester 223 (76 mg, 0.222 mmol), and triethylamine (0.155 mL, 112 mg, 1.11 mmol) in anhydrous acetonitrile (5 mL) were heated at reflux temperature under a nitrogen atmosphere for 24 hours. After cooling, the volatiles were evaporated in vacuo. Ethanol (5 mL) and triethylamine (0.5 mL) were added to the residue. This was heated at reflux temperature for 21 hours. The volatiles were evaporated, the residue was resuspended in methanol (2 mL), and the reaction mixture was treated with hydrazine (0.5 mL). The resulting mixture was stirred at room temperature overnight and concentrated in vacuo. The residue was purified by HPLC (reverse phase C-18 column, 25-50% acetonitrile/water containing 0.1% trifluoroacetic acid) to afford the trifluoroacetic acid salt of 303 (36 mg, 30%) as a yellow solid. MS 420 (M+H). ¹H-NMR (d6-DMSO): δ 1.05-1.23 (m, 4H), 1.75 (m, 2H), 2.48 (m, 2H), 3.67-3.82 (m, 6H), 3.72 (s, 3H), 4.18 (m, 1H), 7.78 (d, 1H, J=10 Hz), 8.19 (brs, 3H), 8.70 (s, 1H), 14.9 (brs, 1H).

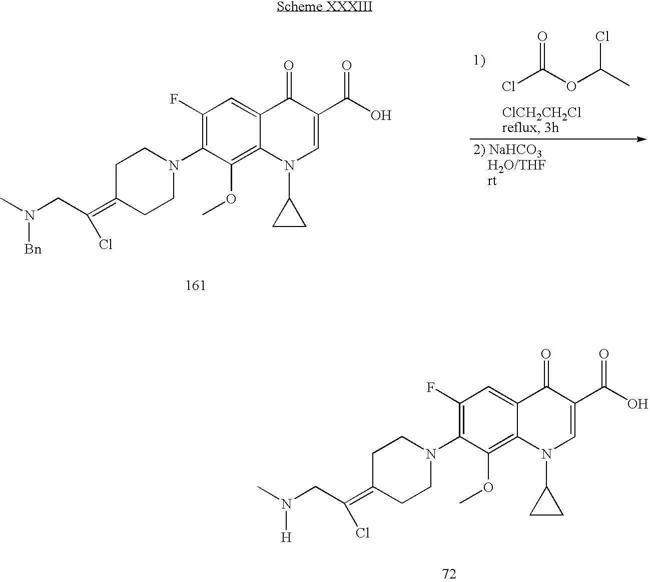

### 7-[4-(1-Chloro-2-methylamino-ethylidene)-piperidin-1-yl]-1-cyclopropyl-6-fluoro-8-methoxy-4-oxo-1,4-dihydroquinoline-3-carboxylic Acid Trifluoroacetic Acid Salt (72)

A solution of 161 (160 mg, 0.24 mmol) was dissolved in 1,2-dichloroethane (4 mL) and was treated with 1-chloroethyl chloroformate (0.8 mL, 7.3 mmol) under nitrogen. After 5 min, the reaction mixture was warmed to reflux temperature and the reaction mixture was allowed to stir for 3 h. The resulting mixture was allowed to cool to room temperature, and then it was concentrated in vacuo. The residue was dissolved in tetrahydrofuran (5 mL), adjusted to pH>7 by the addition of NaHCO₃ and water at room temperature and stirred overnight. The resulting mixture was concentrated in vacuo and the residue purified by HPLC (reverse phase C-18 column, 35-90% acetonitrile/water containing 0.1% trifluoroacetic acid) to afford the trifluoroacetic acid salt of 72 (37 mg, 27%) as a yellow solid. MS 450 (M+H).

Table 10 lists the final products (74, 75, 79, 213-220) prepared by an analogous procedure to that above.

TABLE 10

| | | | Compound | |
|---|---|---|---|---|
| Structure | R₅ | R₉ | Number | (M + H) |
| 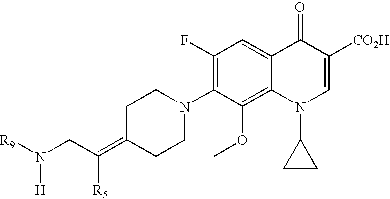 | Cl F | Et Me Et | 75 213 214 | 464 434 448 |
| 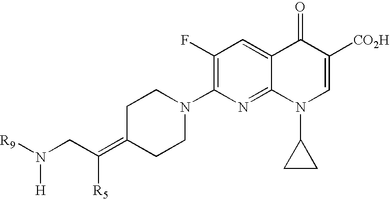 | Cl F | Me Et Me Et | 215 216 79 217 | 421 435 405 419 |
| 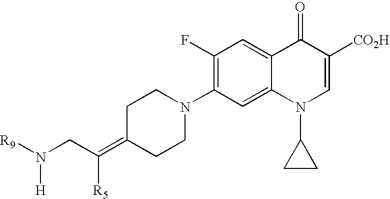 | Cl F | Me Me | 218 219 | 420 404 |
| 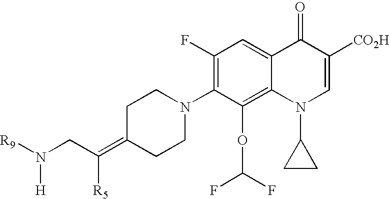 | Cl F | Me Me | 74 220 | 486 470 |

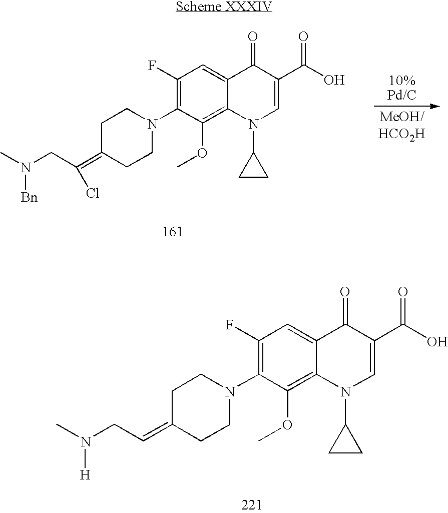

### 1-Cyclopropyl-6-fluoro-8-methoxy-7-[4-(2-methylamino-ethylidene)-piperidin-1-yl]-4-oxo-1,4-dihydro-quinoline-3-carboxylic Acid Trifluoroacetic Acid Salt (221)

A solution of 161 (70 mg, 0.11 mmol) in methanol/formic acid (v/v=20/1) (14 mL) was treated with 10% Pd/C (35 mg, 7.3 mmol) under nitrogen at rt and stirred for 3 h. The resulting mixture was filtered and concentrated in vacuo. The residue was purified by HPLC (reverse phase C-18 column, 35-90% acetonitrile/water containing 0.1% trifluoroacetic acid) to afford the trifluoroacetic acid salt of 221 (8.3 mg, 15%) as a yellow solid. MS 416 (M+H).

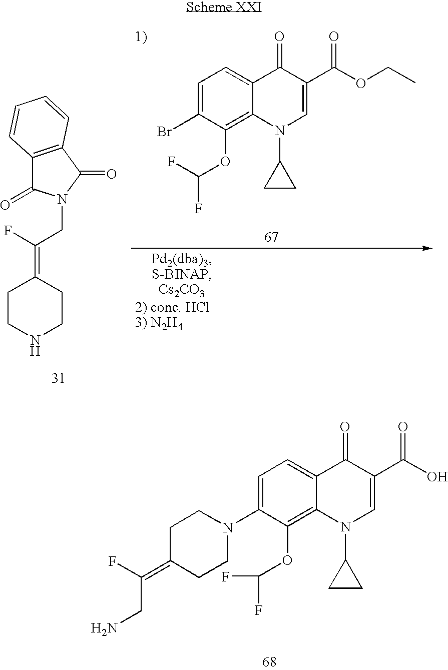

### 7-[4-(2-Amino-1-fluoro-ethylidene)-piperidin-1-yl]-1-cyclopropyl-8-difluoromethoxy-4-oxo-1,4-dihydro-quinoline-3-carboxylic Acid (68)

A solution of amine 31 (534 mg, 1.94 mmol) quinolone 67 (587 mg, 1.46 mmol) (prepared as described in EP1031569), cesium carbonate (717 mg, 2.2 mmol), (1S)-[1,1′-binaphthalene]-2,2′-diylbis[diphenylphosphine] (137 mg, 0.22 mmol) in toluene (75 mL) was treated with Pd₂(dba)₃ (66 mg, 0.072 mmol) and the reaction mixture was warmed to reflux. After 12 h, the resulting mixture was allowed to cool to room temperature, concentrated in vacuo, and the residue was washed with water (3×10 mL). Purification by MPLC (0-100% ethyl acetate/hexanes) afforded a yellow residue. The residue was dissolved in concentrated hydrochloric acid (5 mL) and warmed to reflux. After 3 h, the reaction mixture was concentrated in vacuo, diluted with water (10 min) and the solid collected by filtration. The solid residue was washed with water (3×5 mL) and allowed to dry for 15 min. The solid was collected and resuspended in methanol (5 mL) and the reaction mixture was treated with hydrazine (1 mL). After 5 min, the reaction mixture was warmed to reflux and the resulting mixture was allowed to stir for 1 h. The reaction mixture was concentrated in vacuo and purified by HPLC (reverse phase C-18 column, 0-55% acetonitrile/water containing 0.1% trifluoroacetic acid) to afford the trifluoroacetic acid salt of the title compound 68 (75 mg, 12%) as a light yellow solid. MS 438 (M+H).

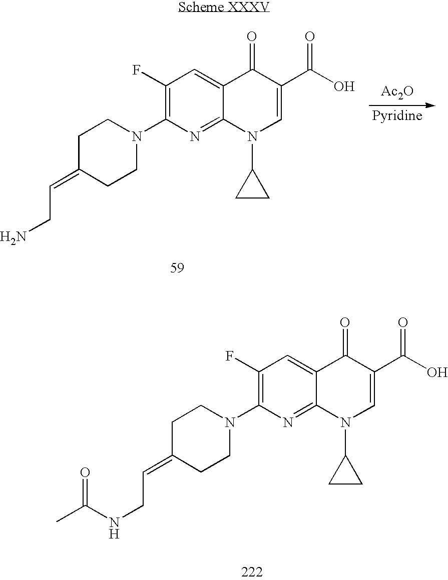

### 7-[4-(2-Acetylamino-ethylidene)-piperidin-1-yl]-1-cyclopropyl-6-fluoro-4-oxo-1,4-dihydro-[1,8]naphthyridine-3-carboxylic Acid (222)

A mixture of 59 (25 mg, 0.067 mmol) and acetic anhydride (94 μL, 0.100 mmol) in pyridine (1 mL) was allowed to stir for 12 h at 25° C. The resulting mixture was concentrated in vacuo, and the residue was washed with water (3×10 mL) and allowed to dry overnight to afford the title compound 222 (15 mg, 54%). MS 415 (M+H).

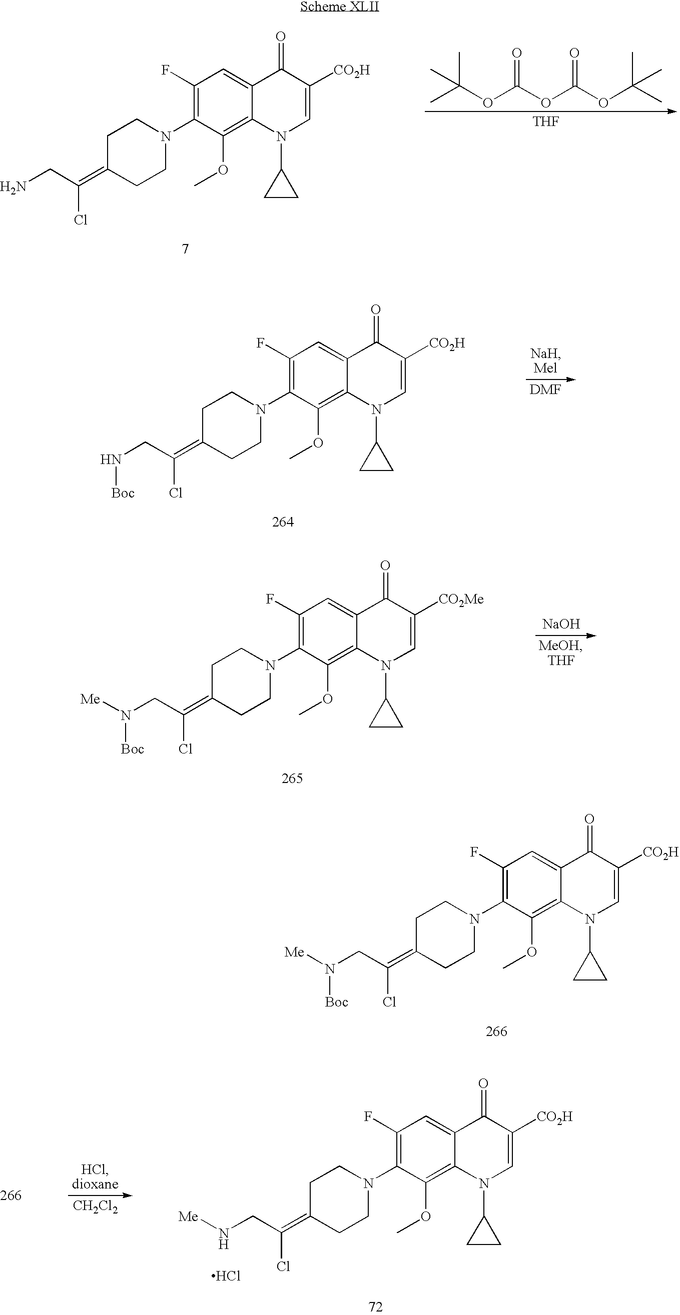

### 7-[4-(1-Chloro-2-methylamino-ethylidene)-piperidin-1-yl]-1-cyclopropyl-6-fluoro-8-methoxy-4-oxo-1,4-dihydro-quinoline-3-carboxylic Acid Hydrochloride Salt (72)

### Step 1

To 7-[4-(2-amino-1-chloroethylidene)piperidin-1-yl]-1-cyclopropyl-6-fluoro-8-methoxy-4-oxo-1,4-dihydroquinoline-3-carboxylic acid (7) (1.96 g; 4.496 mmol) in anhydrous THF (100 mL) at 0° C., was added di-t-butyldicarbonate (1.09 g; 4/994 mmol). The ice bath was removed and the reaction stirred at room temperature for 3 hours. The volatiles were evaporated to afford 7-[4-(2-N-t-butoxycarbonylamino-1-chloroethylidene)piperidin-1-yl]-1-cyclopropyl-6-fluoro-8-methoxy-4-oxo-1,4-dihydroquinoline-3-carboxylic acid (264) as a yellow foam (2.33 g; 97%). MS 536 (M+H).

### Step 2

To NaH (60% in oil, 191 mg; 4.98 mmol) in anhydrous DMF (4 mL) at 0° C., was added dropwise 264 from Step 1 (920 mg: 1.719 mmol) in anhydrous DMF (5 mL). The reaction was stirred for 15 minutes, then methyl iodide (0.23 mL; 3.69 mmol) was added. The reaction was warmed to room temperature and stirred for 24 hours. The reaction was carefully added dropwise to water (150 mL) with stirring. The solid was collected by filtration and dried to give methyl 7-[4-(2-N-t-butoxy-N-methylamino-1-chloroethylidene)piperidin-1-yl]-1-cyclopropyl-6-fluoro-8-methoxy-4-oxo-1,4-dihydroquinoline-3-carboxylate (265) as a cream-colored powder (886 mg; 92%). MS 564 (M+H).

### Step 3:

Ester 265 from Step 2 (255 mg; 0.4529 mmol) and 1N NaOH (1.5 mL) in 1:1 methanol:THF (10 mL total) were heated at 50° C. for 30 minutes under a nitrogen atmosphere. After cooling, the pH was adjusted to pH=4 with 1N HCl. The reaction mixture was extracted with ethyl acetate (50 mL). The organic layer was washed with water (2×25 mL), dried (MgSO₄), filtered and evaporated. The resulting waxy semi-solid was triturated with cold ether to afford 7-[4-(2-N-t-butoxycarbonyl-N-methylamino-1-chloroethylidene)piperidin-1-yl]-1-cyclopropyl-6-fluoro-8-methoxy-4-oxo-1,4-dihydroquinoline-3-carboxylic acid (266) as a pale yellow solid (212 mg; 86%). MS 550 (M+H).

### Step 4:

7-[4-(2-N-t-butoxycarbonyl-N-methylamino-1-chloroethylidene)piperidin-1-yl]-1-cyclopropyl-6-fluoro-8-methoxy-4-oxo-1,4-dihydroquinoline-3-carboxylic acid (266) from Step 3 (207 mg; 0.3770 mmol) and hydrochloric acid (4M in dioxane; 1.5 mL) in methylene chloride were stirred at room temperature overnight. Ether (5 mL) was added and the solid was collected by filtration, washed with additional ether, and dried to afford the title compound (72) as a yellow solid (183 mg; 93%). MS 450 (M+H).

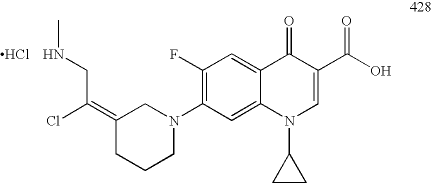

### (E)-7-[3-(1-Chloro-2-methylamino-ethylidene)-piperidin-1-yl]-1-cyclopropyl-6-fluoro-4-oxo-1,4-dihydroquinoline-3-carboxylic Acid Hydrochloride Salt (428)

This was prepared by the same procedure as in the synthesis of 72 above except that 257 was used in place of 7. MS 420 (M+H).

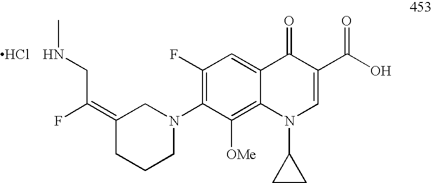

### (E)-1-Cyclopropyl-6-fluoro-7-[3-(1-fluoro-2-methylamino-ethylidene)-piperidin-1-yl]-8-methoxy-4-oxo-1,4-dihydro-quinoline-3-carboxylic Acid Hydrochloride Salt (453)

This was prepared by the same procedure as in the synthesis of 72 above except that 303 was used in place of 7. MS 434 (M+H).

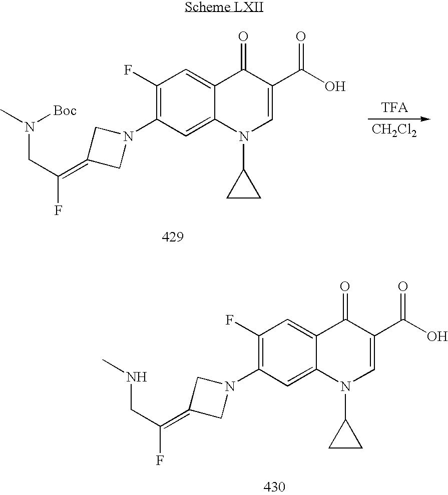

1-Cyclopropyl-6-fluoro-7-[3-(1-fluoro-2-methylamino-ethylidene)-azetidin-1-yl]-4-oxo-1,4-dihydro-quinoline-3-carboxylic acid trifluoroacetic acid salt (430)

The Boc-protected compound (429) was prepared in an analogous manner to compound 266 except that quinolone 272 was used in place of quinolone 7. To a solution of 429 (50 mg; 0.105 mmol) in CH₂Cl₂ (3.5 mL) was added trifluoroacetic acid (0.04 mL; 0.525 mmol) and the resulting solution stirred at room temperature under a nitrogen atmosphere for 2 hours. The reaction mixture was concentrated in vacuo. Methylene chloride was added (1 mL), and the residue was triturated with diethyl ether (5 mL). The solid was collected by filtration, washed with additional diethyl ether, and dried to afford the title compound (430) as a yellow-brown powder (38.6 mg, 75%). MS 376 (M+H).

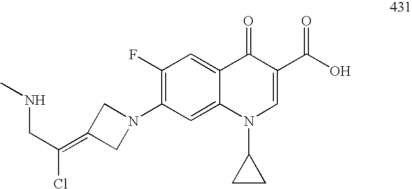

7-[3-(1-Chloro-2-methylamino-ethylidene)-azetidin-1-yl]-1-cyclopropyl-6-fluoro-4-oxo-1,4-dihydro-quinoline-3-carboxylic acid trifluoroacetic acid salt (431) was prepared in a similar manner as for the synthesis of 430 except that quinolone 76 was used in place of quinolone 272. The title compound (431) was isolated as off-white powder. MS 392 (M+H).

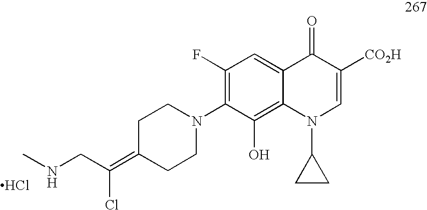

### 7-[4-(1-Chloro-2-methylamino-ethylidene)-piperidin-1-yl]-1-cyclopropyl-6-fluoro-8-hydroxy-4-oxo-1,4-dihydro-quinoline-3-carboxylic Acid Hydrochloride Salt (267)

Methyl 7-[4-(2-N-t-butoxycarbonyl-N-methylamino-1-chloroethylidene)piperidin-1-yl]-1-cyclopropyl-6-fluoro-8-methoxy-4-oxo-1,4-dihydroquinoline-3-carboxylate (265) (242 mg; 0.4298 mmol) in concentrated HCl (2 mL) was heated at 100° C. for 1 hour. The reaction was evaporated to dryness. Methanol (5 mL) was added to the residue followed by the dropwise addition of ether until the solution became cloudy. This was stirred for 30 minutes and the solid was collected, washed with ether and dried to afford title compound 266 as a pale yellow solid (125 mg; 62%). MS 436 (M+H).

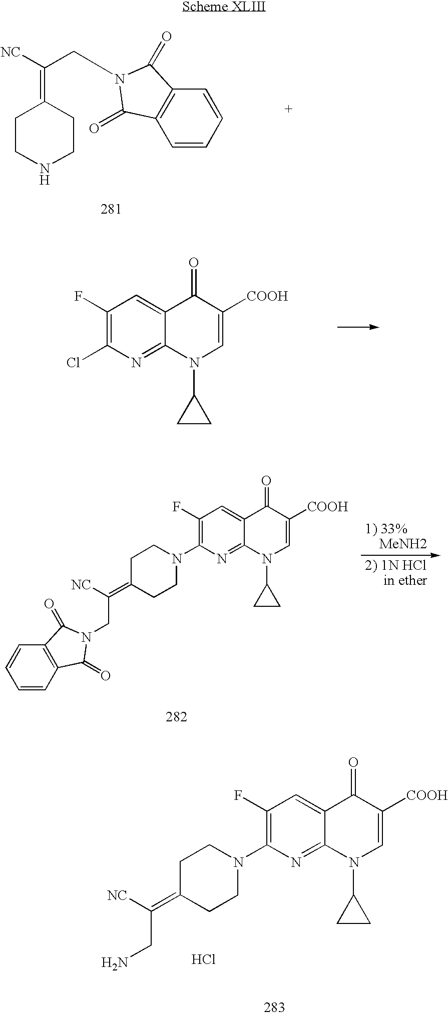

### 7-[4-(2-Amino-1-cyano-ethylidene)-piperidin-1-yl]-1-cyclopropyl-6-fluoro-4-oxo-1,4-dihydro-[1,8]naphthyridine-3-carboxylic Acid (283)

### Step 1

A solution of 3-(1,3-dioxo-1,3-dihydro-isoindol-2-yl)-2-piperidin-4-ylidene-propionitrile (281; 180 mg, 0.64 mmol) and triethylamine (0.6 mL) in acetonitrile (4 mL) was treated with 7-chloro-1-cyclopropyl-6-fluoro-4-oxo-1,4-dihydro-naphthpyridine-3-carboxylic acid (150 mg, 0.53 mmol) under nitrogen and the reaction mixture was allowed to stir at 60 deg. for 12 h. The resulting mixture was cooled down and the precipitate was filtered, washed with acetonitrile and dried to give the α,β-unsaturated nitrile (282) as a yellow solid (170 mg, 61%). MS 528 (M+H).

### Step 2

The α,β-unsaturated nitrile 282 (60 mg, 0.11 mmol) was dissolved in 3 mL methanol. To this solution was added 6 mL 33% methylamine in ethanol and the mixture was stirred at room temperature for 12 h. The solvent was concentrated and the residue was dissolved in CH₂Cl₂ (5 mL). To this solution was added 1N HCl in ethyl ether (0.15 mL) and the resulting precipitate was collected, washed with ether and dried to give 7-[4-(2-amino-1-cyano-ethylidene)-piperidin-1-yl]-1-cyclopropyl-6-fluoro-4-oxo-1,4-dihydro-[1,8]naphthyridine-3-carboxylic acid (283) (40 mg, 80%). MS 398 (M+H).

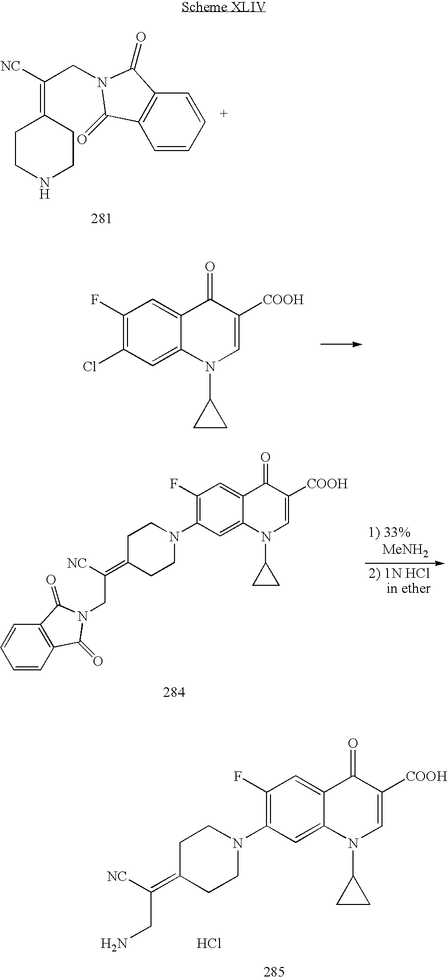

### 7-[4-(2-Amino-1-cyano-ethylidene)-piperidin-1-yl]-1-cyclopropyl-6-fluoro-4-oxo-1,4-dihydro-quinoline-3-carboxylic Acid (285)

### Step 1

The α,β-unsaturated nitrile (284) was prepared in a similar manner as for the synthesis of 282. MS 527 (M+H).

### Step 2

The title compound (285) compound was prepared in a similar manner as for the synthesis of 283. MS 397 (M+H).

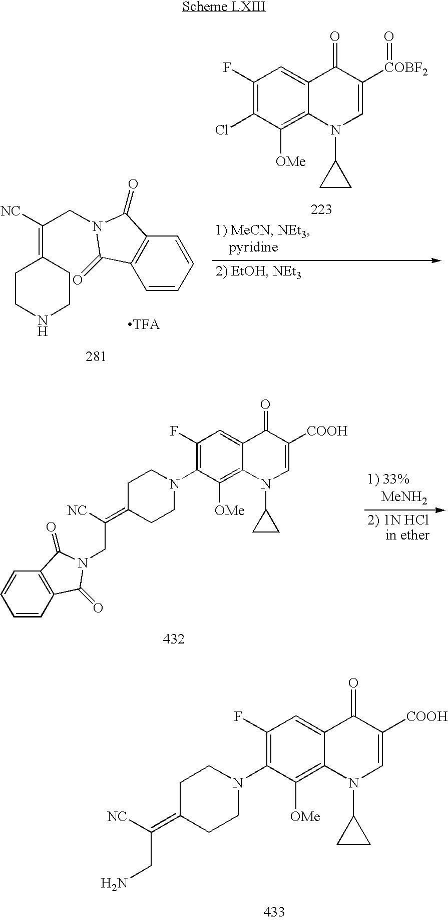

### 7-[4-(2-Amino-1-cyano-ethylidene)-piperidin-1-yl]-1-cyclopropyl-6-fluoro-8-methoxy-4-oxo-1,4-dihydro-quinoline-3-carboxylic Acid (433)

### Step 1

A solution of 3-(1,3-dioxo-1,3-dihydro-isoindol-2-yl)-2-piperidin-4-ylidene-propionitrile trifluoroacetate (281; 101 mg, 0.26 mmol) and triethylamine (0.15 mL) in acetonitrile (5 mL) was treated with 1-cyclopropyl-1,4-dihydro-6,7-difluoro-8-methoxy-4-oxo-quinoline-3-carboxylic acid difluoroborate ester (223; 69 mg, 0.2 mmol) under nitrogen and the reaction mixture was heated at reflux temperature for 48 h. Pyridine (1 mL) was added and the reaction mixture was heated at reflux temperature for another 5 h. The reaction mixture was evaporated, the residue was taken up in EtOH (5 mL), and triethylamine (0.5 mL) was added. The mixture was heated at reflux temperature for 12 h, cooled to room temperature and the precipitate was collected by filtration (30 mg, 27%). MS 557 (M+H).

### Step 2

The α,β-unsaturated nitrile 432 (28 mg, 0.05 mmol) was dissolved in 2 mL methanol. To this solution was added 4 mL 33% methylamine in ethanol and the mixture was stirred at room temperature for 8 h. The solvent was evaporated and the residue was dissolved in EtOH (3 mL). To this solution was added 1 N HCl in ethyl ether (0.1 mL) and the resulting precipitate was collected by filtration. Purification by HPLC (reverse phase C-18 column, 10-90% acetonitrile/water containing 0.1% trifluoroacetic acid) afforded the trifluoroacetic acid salt of 433 (15 mg, 56%). MS 427 (M+H).

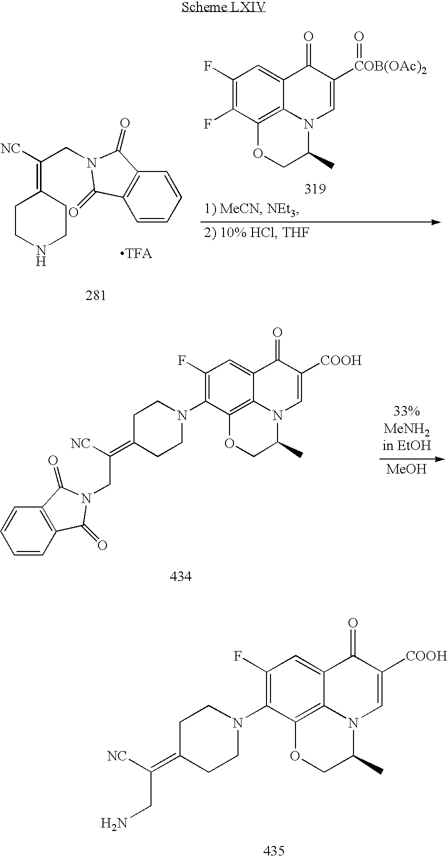

### 9-[4-(2-Amino-1-cyano-ethylidene)-piperidin-1-yl]-8-fluoro-(3S)-3-methyl-6-oxo-2,3-dihydro-6H-1-oxa-3a-aza-phenalene-5-carboxylic Acid (435)

### Step 1

A solution of 3-(1,3-dioxo-1,3-dihydro-isoindol-2-yl)-2-piperidin-4-ylidene-propionitrile trifluoroacetate (281; 103 mg, 0.26 mmol) and triethylamine (0.15 mL) in acetonitrile (5 mL) was treated with (S)-8,9-difluoro-2,3-dihydro-3-methyl-7-oxo-7H-pyrido[1,2,3-de]-1,4-benzoxazine-6-carboxylic acid diacetoxyborate ester (319; 69 mg, 0.2 mmol) under nitrogen and the reaction mixture was heated at reflux temperature for 24 h. The reaction mixture was evaporated, the residue was taken up in THF (5 mL), and 10% aqueous HCl (4 mL) was added. The mixture was stirred at room temperature for 2 h. The solvent was evaporated in vacuo until a solid precipitated, at which point it was collected by filtration. The solid was used without further purification in the next step.

### Step 2

The α,β-unsaturated nitrile 434 from the previous step was dissolved in 3 mL methanol. To this solution was added 6 mL 33% methylamine in ethanol and the mixture was stirred at room temperature for 8 h. The solvent was evaporated and the resulting solid residue was purified by HPLC (reverse phase C-18 column, 10-90% acetonitrile/water containing 0.1% trifluoroacetic acid) to provide the trifluoroacetate salt of 435 (25 mg, 24%). MS 413 (M+H).

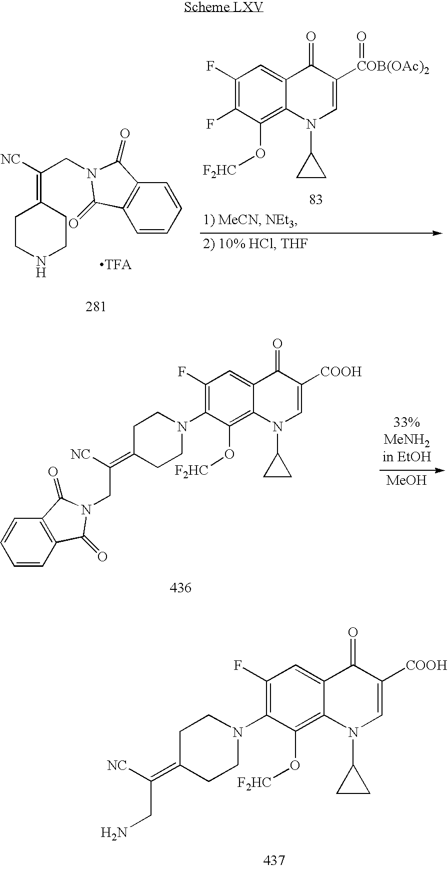

### 7-[4-(2-Amino-1-cyano-ethylidene)-piperidin-1-yl]-1-cyclopropyl-6-fluoro-8-difluoromethoxy-4-oxo-1,4-dihydro-quinoline-3-carboxylic Acid (437)

437 was synthesized by a similar procedure as 435 above except that diacetoxyborate ester 83 was used in place of diacetoxyborate ester 319. MS 463 (M+H).

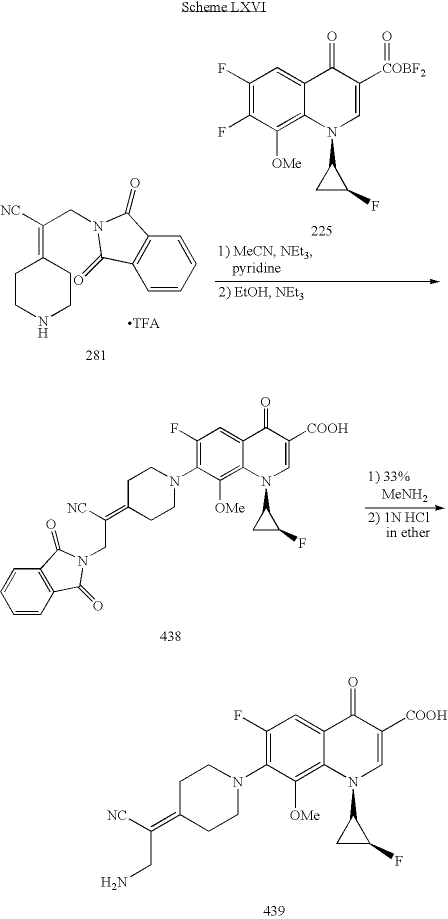

### 7-[4-(2-Amino-1-cyano-ethylidene)-piperidin-1-yl]-6-fluoro-(1R,2S)-1-(2-fluoro-cyclopropyl)-8-methoxy-4-oxo-1,4-dihydro-quinoline-3-carboxylic Acid (439)

439 was synthesized by a similar procedure as 433 above except that difluoroborate ester 225 was used in place of difluoroborate ester 223. MS 445 (M+H).

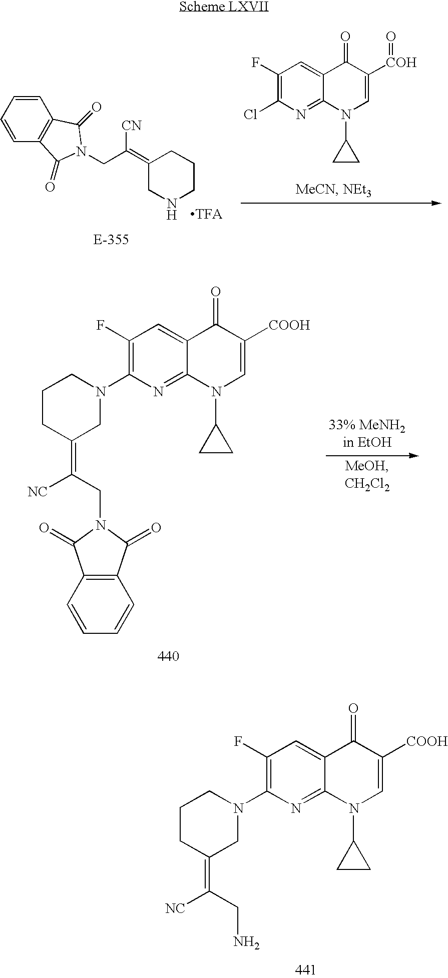

### (E)-7-[3-(2-Amino-1-cyano-ethylidene)-piperidin-1-yl]-1-cyclopropyl-6-fluoro-4-oxo-1,4-dihydro-[1,8]naphthyridine-3-carboxylic Acid (441)

### Step 1

A solution of (E)-3-(1,3-dioxo-1,3-dihydro-isoindol-2-yl)-2-piperidin-3-ylidene-propionitrile trifluoroacetate (E-355; 160 mg, 0.4 mmol) and triethylamine (0.2 mL) in acetonitrile (3 mL) was treated with 7-chloro-1-cyclopropyl-6-fluoro-4-oxo-1,4-dihydronaphthyridine-3-carboxylic acid (56 mg, 0.2 mmol) under nitrogen and the reaction mixture was heated at reflux temperature for 8 h. The reaction mixture was cooled to room temperature, the resulting precipitate was collected by filtration, and the solid (440; 50 mg, 48%) was used as such in the next step.

### Step 2

The α,β-unsaturated nitrile 440 from the previous step was dissolved in 3 mL methanol. To this solution was added 6 mL 33% methylamine in ethanol and the mixture was stirred at room temperature for 24 h. A solid precipitated out of solution. To the reaction mixture was added CH₂Cl₂ (3 mL) and the solution was stirred for an additional 12 h at room temperature. The solvent was evaporated and the resulting solid residue was purified by HPLC (reverse phase C-18 column, 10-100% acetonitrile/water containing 0.1% trifluoroacetic acid) to provide the trifluoroacetate salt of 441 (24 mg, 45%). MS 398 (M+H).

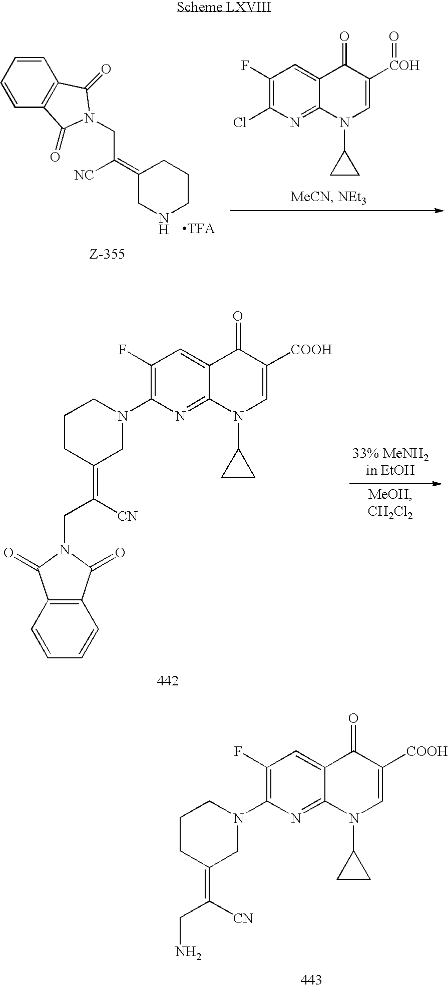

### (Z)-7-[3-(2-Amino-1-cyano-ethylidene)-piperidin-1-yl]-1-cyclopropyl-6-fluoro-4-oxo-1,4-dihydro-[1,8]naphthyridine-3-carboxylic Acid (443)

443 was synthesized by a similar procedure as 441 above except that (Z)-3-(1,3-dioxo-1,3-dihydro-isoindol-2-yl)-2-piperidin-3-ylidene-propionitrile trifluoroacetate (Z-355) was used in place of (E)-3-(1,3-dioxo-1,3-dihydro-isoindol-2-yl)-2-piperidin-3-ylidene-propionitrile trifluoroacetate (E-355). MS 398 (M+H).

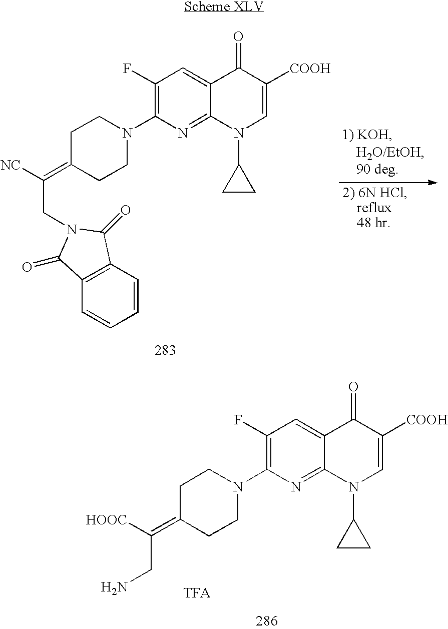

### 7-[4-(2-Amino-1-carboxy-ethylidene)-piperidin-1-yl]-1-cyclopropyl-6-fluoro-4-oxo-1,4-dihydro-[1,8]naphthyridine-3-carboxylic Acid (286)

Nitrile 283 (26 mg, 0.05 mmol) and potassium hydroxide (17 mg) were dissolved in ethanol (1 mL) and water (1 mL). The mixture was heated at 90 deg. for 1 h. The solution was cooled down and concentrated. The residue was acidified with 1N HCl and extracted with methylene chloride. The organic layer was concentrated to give a light yellow solid (30 mg). The above solid was dissolved in 10 mL 6N HCl and heated at reflux temperature for 48 h. The mixture was cooled down and purified by HPLC (reverse phase C-18 column, 10-50% acetonitrile/water containing 0.1% trifluoroacetic acid) to afford the trifluoroacetic acid salt of the title compound (4.0 mg, 15%). MS 417 (M+H).

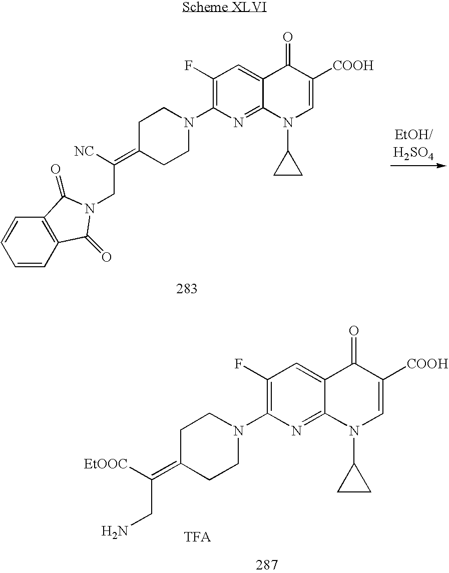

### 7-[4-(2-Amino-1-ethoxycarbonyl-ethylidene)-piperidin-1-yl]-1-cyclopropyl-6-fluoro-4-oxo-1,4-dihydro-[1,8]naphthyridine-3-carboxylic Acid (287)

The nitrile 283 (40 mg, 0.08 mmol) was dissolved in 10 mL ethanol and to this mixture was added 0.5 mL concentrated sulfuric acid. The reaction was heated at 160° C. for 1 h. under microwave irradiation. The mixture was cooled down and purified by HPLC (reverse phase C-18 column, 10-90% acetonitrile/water containing 0.1% trifluoroacetic acid) to afford the trifluoroacetic acid salt of the title compound (3.0 mg, 7%). MS 445 (M+H).

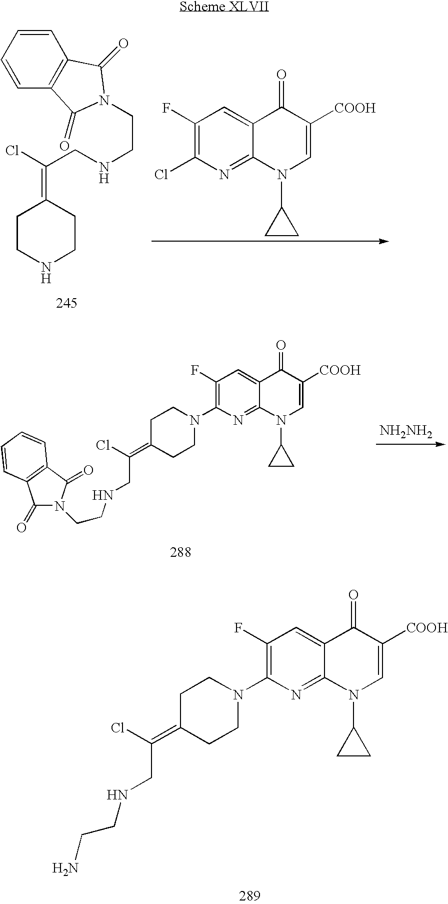

### 7-{4-[2-(2-Amino-ethylamino)-1-chloro-ethylidene]-piperidin-1-yl}-1-cyclopropyl-6-fluoro-4-oxo-1,4-dihydro-[1,8]naphthyridine-3-carboxylic Acid Trifluoroacetic Acid Salt (289)

### Step 1

Coupling of amine 245 with 7-chloro-1-cyclopropyl-6-fluoro-4-oxo-1,4-dihydro-naphthyridine-3-carboxylic acid was performed in a similar manner as for the preparation of 162 to give phthalimide 288. MS 581 (M+H).

### Step 2

Hydrazinolysis of phthalimide 288 to provide the title compound 289 was performed in a similar manner as for the preparation of 68. MS 450 (M+H).

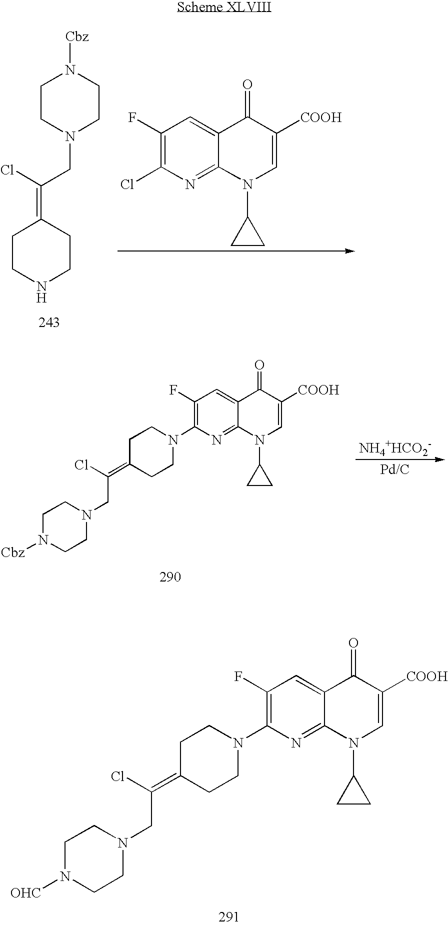

### 7-{4-[1-Chloro-2-(4-formyl)-piperazin-1-yl-ethylidene]-piperidin-1-yl}-1-cyclopropyl-6-fluoro-4-oxo-1,4-dihydro-[1,8]naphthyridine-3-carboxylic Acid Trifluoroacetic Acid Salt (291)

### Step 1

Coupling of amine 243 with 7-chloro-1-cyclopropyl-6-fluoro-4-oxo-1,4-dihydro-naphthyridine-3-carboxylic acid was performed in a similar manner as for the preparation of 162. MS 611 (M+H).

### Step 2

Cbz-protected piperazine derivative 290 (0.0505 g, 0.070 mmol) in tetrahydrofuran (3 mL), methanol (6 mL), and dimethylformamide (4 mL) was treated with ammonium formate (0.16 g, 2.6 mmol) and 10% palladium on carbon (0.0195 g, 0.018 mmol) added in three portions over 11 days. The mixture was filtered through celite and eluted with methanol. The mixture was concentrated, taken up in dimethylsulfoxide and purified by HPLC (reverse phase C-18 column, 10-90% acetonitrile/water containing 0.1% trifluoroacetic acid). The collected fractions were lyophilized to provide 0.0081 g of the title compound (291) as a white solid (19% yield). MS 504 (M+H).

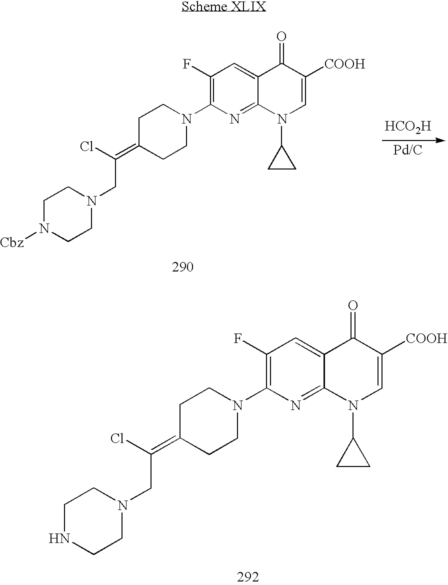

### 7-[4-(1-chloro-2-piperazin-1-yl-ethylidene)-piperidin-1-yl]-1-cyclopropyl-6-fluoro-4-oxo-1,4-dihydro-[1,8]naphthyridine-3-carboxylic Acid Trifluoroacetic Acid Salt (292)

To a solution of compound 290 (0.0475 g, 0.066 mmol) in methanol (9 mL) was added formic acid (0.5 mL, 13.2 mmol) and 10% palladium on carbon (0.0149 g, 0.014 mmol), and the mixture was stirred overnight. The mixture was filtered through celite and eluted with methanol. The mixture was concentrated, taken up in dimethylsulfoxide and purified by HPLC (reverse phase C-18 column, 10-90% acetonitrile/water containing 0.1% trifluoroacetic acid). The collected fractions were lyophilized to provide 0.0173 g of the title compound (292) as an orange solid (45%). MS 476 (M+H).

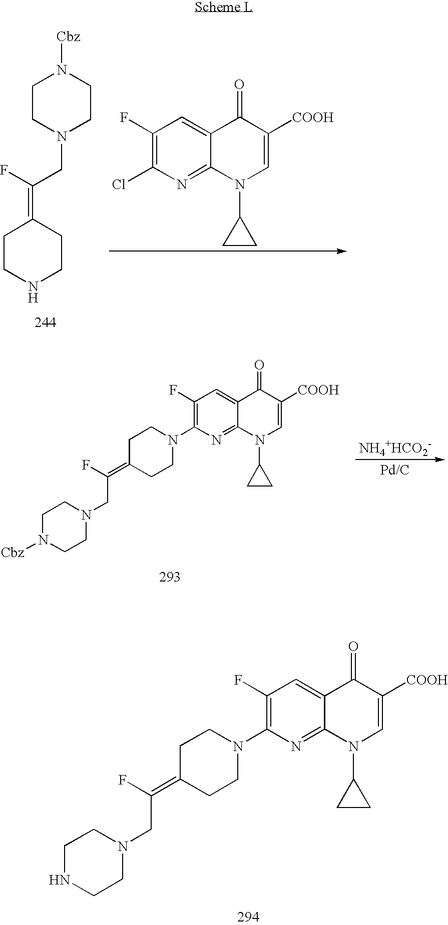

### 1-Cyclopropyl-6-fluoro-7-[4-(1-fluoro-2-piperazin-1-yl-ethylidene)-piperidin-1-yl]-4-oxo-1,4-dihydro-[1,8]naphthyridine-3-carboxylic Acid Trifluoroacetic Acid Salt (294)

### Step 1

Coupling of amine 244 with 7-chloro-1-cyclopropyl-6-fluoro-4-oxo-1,4-dihydro-naphthpyridine-3-carboxylic acid was performed in a similar manner as for the preparation of 162. MS 594 (M+H).

### Step 2

Cbz-protected piperazine derivative 293 (0.0733 g, 0.104 mmol) in tetrahydrofuran (1 mL) and methanol (3 mL) was treated with ammonium formate (0.0377 g, 0.60 mmol) and 10% palladium on carbon (0.005 g, 0.005 mmol) and stirred overnight. The mixture was filtered through celite and eluted with methanol. The mixture was concentrated, taken up in dimethylsulfoxide and purified by HPLC (reverse phase C-18 column, 10-90% acetonitrile/water containing 0.1% trifluoroacetic acid). The collected fractions were lyophilized to provide 0.0321 g of the title compound (294) as a white solid (54%). MS 460 (M+H).

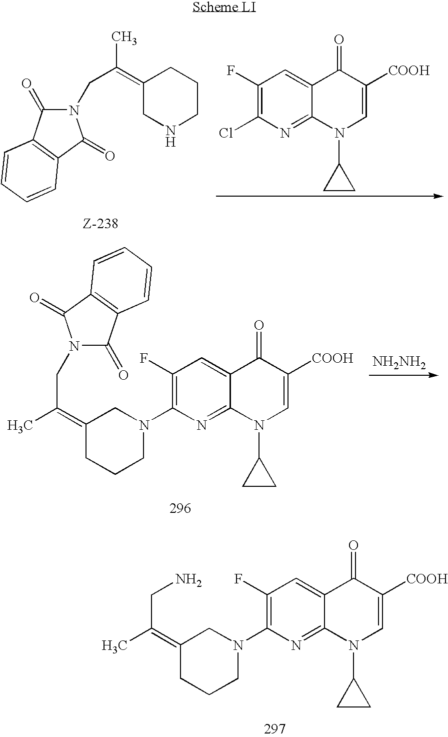

### (Z)-7-[3-(1-Amino-2-propyl idene)-piperidin-1-yl]-1-cyclopropyl-6-fluoro-4-oxo-1,4-dihydro-[1,8]naphthyridine-3-carboxylic Acid Trifluoroacetic Acid Salt (297)

### Step 1

Coupling of amine Z-238 with 7-chloro-1-cyclopropyl-6-fluoro-4-oxo-1,4-dihydro-naphthpyridine-3-carboxylic acid was performed in a similar manner as for the preparation of 162. MS 517 (M+H).

### Step 2

Hydrazinolysis of phthalimide 296 to provide the title compound (297) was performed in a similar manner as for the preparation of 68. MS 387 (M+H).

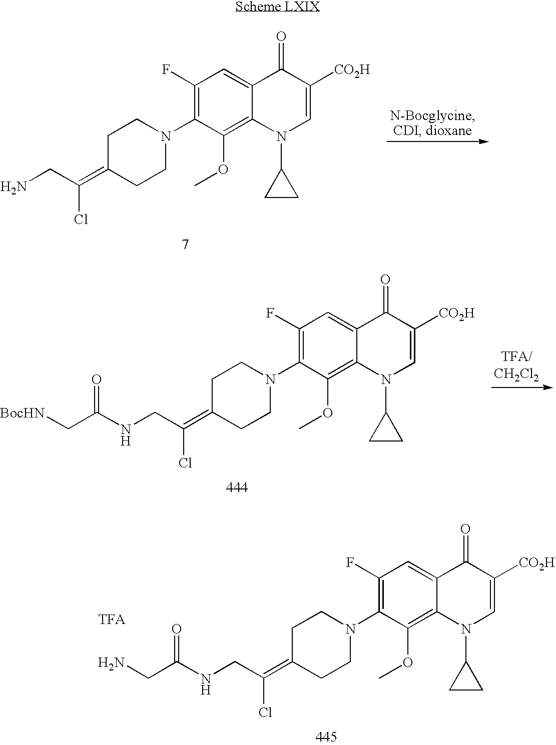

### 7-{4-[2-(2-Amino-acetylamino)-1-chloro-ethylidene]-piperidin-1-yl}-1-cyclopropyl-6-fluoro-8-methoxy-4-oxo-1,4-dihydro-quinoline-3-carboxylic Acid Trifluoroacetate Salt (445)

### Step 1

To N-Boc-glycine (115 mg; 0.6565 mmoles) in anhydrous dioxane (5.0 mL), was added carbonyl diimidazole (115 mg; 0.7092 mmoles). The resulting solution was stirred at room temperature for 2 hours and then at 45° C. for 30 minutes. Compound 7 (260 mg; 0.5963 mmoles) was added, the reaction cooled to room temperature and stirred overnight. The reaction mixture was poured into water (50 mL) and extracted with ethyl acetate (2×50 mL). The combined organic layers were washed with several portions of water, dried (MgSO₄), filtered and evaporated to afford amide 444 as a pale yellow powder (190 mg; 54% yield). MS 593, 595 (M+H).

### Step 2

Trifluoroacetic acid (1.0 mL) was added to 444 (190 mg; 0.3204 mmoles) in methylene chloride (5 mL). The reaction was stirred overnight at room temperature and the volatiles evaporated to afford crude material as the trifluoroacetate salt. This was triturated with ether to yield 445 as a pale yellow powder (192 mg; 100%). MS 493, 495 (M+H).

### Biological Activity

The compounds described in the present invention possess antibacterial activity due to their novel structure, and are useful as antibacterial agents for the treatment of bacterial infections in humans and animals.

Minimal inhibitory concentration (MIC) has been an indicator of in vitro antibacterial activity widely used in the art. The in vitro antimicrobial activity of the compounds was determined by the microdilution broth method following the test method from the National Committee for Clinical Laboratory Standards (NCCLS). This method is described in the NCCLS Document M7-A4, Vol. 17, No. 2, “Methods for Dilution Antimicrobial Susceptibility Test for Bacteria that Grow Aerobically—Fourth Edition”, which is incorporated herein by reference.

In this method two-fold serial dilutions of drug in cation adjusted Mueller-Hinton broth are added to wells in microdilution trays. The test organisms are prepared by adjusting the turbidity of actively growing broth cultures so that the final concentration of test organism after it is added to the wells is approximately 5×10⁴ CFU/well.

Following inoculation of the microdilution trays, the trays are incubated at 35° C. for 16-20 hours and then read. The MIC is the lowest concentration of test compound that completely inhibits growth of the test organism. The amount of growth in the wells containing the test compound is compared with the amount of growth in the growth-control wells (no test compound) used in each tray. As set forth in Table 5, compounds of the present invention were tested against a variety of pathogenic bacteria resulting in a range of activities depending on the organism tested.

TABLE 5
MIC Values (μg/mL) of Some Compounds of the Present Invention

MIC (μg/mL)
Organism

| Compound | A | B | C | D | E |
|---|---|---|---|---|---|
| 1 | 0.03 | 2 | 0.5 | 1 | 0.12 |
| 2 | 0.06 | 0.25 | 1 | 1 | 0.12 |
| 3 | 0.06 | 1 | 0.25 | 1 | 0.12 |
| 4 | 0.06 | ND* | 1 | 2 | 0.25 |
| 5 | 0.03 | ND | 1 | ND | 0.06 |
| 6 | 0.03 | ND | 0.5 | ND | 0.06 |
| 7 | 0.015 | 0.06 | 0.12 | 0.12 | 0.06 |
| 8 | 0.25 | 2 | 8 | 8 | 0.5 |
| 9 | 0.5 | 4 | >16 | >16 | 1 |
| 10 | 0.03 | ND | 0.5 | 1 | 0.12 |
| 11 | 0.03 | ND | 0.5 | 1 | 0.12 |
| 12 | 0.12 | 2 | 4 | 4 | 0.5 |
| 189 | <0.12 | 4 | 2 | 4 | 2 |
| 76 | 0.03 | 2 | 1 | 2 | 0.25 |
| 77 | 0.12 | 8 | 16 | 16 | 1 |
| 80 | 0.06 | 0.5 | 2 | 1 | 0.12 |
| 205 | 0.25 | 2 | 8 | 4 | 0.5 |
| 81 | 0.06 | 1 | 2 | 2 | 0.25 |
| 160 | 0.06 | 2 | 2 | 1 | 0.12 |
| 183 | 0.12 | ND | 2 | 2 | 0.25 |
| 182 | 0.06 | ND | 1 | 2 | 0.25 |
| 209 | 0.12 | ND | 8 | 16 | 2 |
| 166 | 0.5 | ND | 4 | 4 | 1 |
| 191 | 0.25 | 8 | 8 | 2 | 0.5 |
| 193 | 0.12 | 8 | 8 | 2 | 0.5 |
| 194 | 0.12 | 2 | 4 | 1 | 0.5 |
| 195 | 0.12 | 8 | 8 | 2 | 1 |
| 196 | 0.12 | ND | 4 | 2 | 0.5 |
| 199 | 0.06 | 1 | 2 | 1 | 0.25 |
| 206 | 1 | ND | 4 | 4 | 4 |
| 207 | 1 | 8 | 8 | 2 | 4 |
| 72 | 0.03 | 0.25 | 0.5 | 0.25 | 0.12 |
| 75 | 0.12 | 1 | 1 | 1 | 0.25 |
| 213 | 0.12 | ND | 2 | 2 | 0.5 |
| 214 | 0.06 | ND | 1 | 1 | 0.25 |
| 215 | 0.25 | 2 | 2 | 2 | 0.25 |
| 216 | 0.06 | 0.12 | 2 | 1 | 0.25 |
| 79 | 0.06 | ND | 2 | 1 | 0.25 |
| 217 | 0.25 | ND | 2 | 2 | 0.25 |
| 218 | 0.12 | ND | 2 | 2 | 0.25 |
| 219 | 0.5 | 8 | 8 | 4 | 0.5 |
| 74 | 0.06 | ND | 1 | 0.25 | 0.12 |
| 220 | 0.12 | 2 | 4 | 0.5 | 0.5 |
| 221 | 0.12 | 2 | 4 | 4 | 0.5 |
| 201 | 0.12 | ND | 2 | 8 | 0.5 |
| 208 | 0.06 | 0.25 | 2 | 0.25 | 0.25 |
| 73 | 0.06 | 0.5 | 1 | 0.25 | 0.12 |
| 202 | 0.5 | 16 | 16 | 8 | 2 |
| 173 | 0.5 | 8 | 16 | 8 | 1 |
| 70 | 0.03 | ND | 0.25 | 0.25 | 0.12 |
| 69 | 0.06 | ND | 1 | 2 | 0.25 |
| 71 | 0.03 | ND | 0.25 | 0.5 | 0.12 |
| 65 | 0.03 | ND | 0.5 | 1 | 0.12 |
| 14 | 0.03 | 2 | 1 | 1 | 0.25 |
| 64 | 0.12 | 8 | 2 | 8 | 1 |
| 163 | 0.12 | 4 | 4 | ND | 1 |
| 185 | 0.12 | 16 | 16 | ND | 1 |
| 59 | 0.06 | 8 | 8 | ND | 0.5 |
| 164 | 0.12 | 32 | >32 | ND | 2 |
| 172 | 0.06 | 32 | 16 | ND | 1 |
| 211 | 0.03 | 4 | 4 | ND | 0.5 |
| 60 | 2 | ND | 16 | ND | 16 |
| 61 | 0.12 | ND | 16 | ND | 1 |
| 210 | 8 | 16 | 16 | ND | >16 |
| 62 | 2 | >16 | >16 | ND | 8 |
| 204 | 0.25 | 0.5 | 0.5 | 0.5 | 0.12 |
| 176 | 0.25 | 0.5 | 1 | 1 | 0.12 |
| 175 | 0.5 | 2 | 2 | 1 | 0.25 |
| 78 | 1 | 2 | 1 | ND | 0.25 |
| 173 | 0.06 | 1 | 2 | 2 | 0.25 |
| 203 | 0.06 | 0.06 | 1 | 0.5 | 0.12 |
| 212 | 0.06 | 1 | 2 | 2 | 0.12 |
| 177 | 1 | 8 | >16 | >16 | 4 |
| 178 | 0.5 | 1 | 8 | 4 | 4 |
| 179 | 0.5 | >16 | >16 | >16 | 2 |
| 180 | 0.5 | ND | 8 | 2 | 0.5 |
| 181 | 1 | ND | >16 | 8 | 2 |
| 247 | 0.5 | ND | 8 | 2 | 0.5 |
| 248 | 0.5 | ND | 16 | 4 | 2 |
| 249 | 1 | ND | 16 | 4 | 2 |
| 252 | 0.5 | ND | 8 | 4 | 1 |
| 250 | 0.25 | ND | 8 | 0.5 | 0.5 |
| 251 | 0.5 | ND | 16 | >2 | 1 |
| 253 | 0.06 | 4 | >16 | 4 | 0.5 |
| 254 | 0.06 | ND | 4 | 4 | 0.25 |
| 255 | 0.5 | 2 | 4 | 16 | 4 |
| 256 | 0.03 | 1 | 1 | 1 | 0.06 |
| 257 | 0.03 | 0.5 | 0.5 | 0.5 | 0.06 |
| 258 | ≦4 | 8 | >16 | >16 | 8 |
| 261 | ≦0.015 | 0.06 | 0.06 | 0.06 | ≦0.015 |
| 259 | 0.03 | 0.5 | 1 | 0.12 | 0.06 |
| 260 | 0.03 | ND | 0.5 | 0.06 | 0.06 |
| 262 | ≦0.015 | ND | 1 | 1 | 0.25 |
| 263 | 0.03 | 0.5 | 1 | 0.5 | 0.12 |
| 267 | 0.12 | 2 | 2 | 2 | 0.5 |
| 268 | ≦0.015 | 0.06 | 0.12 | 0.12 | 0.03 |
| 269 | ≦0.015 | 0.25 | 0.06 | ND | 0.03 |
| 270 | 0.06 | ND | 4 | 2 | 0.5 |
| 271 | ≦0.015 | 1 | 1 | 1 | 0.12 |
| 272 | ≦0.015 | 4 | 0.5 | ND | 0.5 |
| 273 | 0.03 | 4 | 0.5 | ND | 4 |
| 274 | 0.06 | 4 | 1 | ND | 1 |
| 275 | 0.03 | 1 | 1 | 1 | 0.12 |
| 276 | ≦0.015 | 0.5 | 2 | 4 | 0.25 |
| 277 | ≦0.015 | 0.25 | 0.25 | 0.5 | 0.03 |
| 283 | 0.25 | 2 | 4 | 8 | 0.5 |
| 285 | 0.25 | ND | 8 | >16 | 1 |
| 286 | 4 | ND | >16 | >16 | 16 |
| 289 | 0.5 | ND | 1 | 2 | 0.25 |
| 291 | 2 | ND | >16 | 8 | 8 |
| 292 | 1 | ND | >16 | 16 | 4 |
| 294 | 2 | >16 | >16 | >16 | 8 |
| 295 | 0.5 | 16 | 16 | 8 | 2 |
| 297 | 0.25 | 16 | 16 | 16 | 1 |
| 298 | 0.25 | 16 | 16 | 8 | 1 |
| 299 | 0.5 | 16 | >16 | 2 | 1 |
| 300 | 0.5 | >16 | >16 | 2 | 2 |
| 301 | 0.12 | 8 | 8 | 8 | 0.25 |
| 302 | ≦0.03 | 1 | ≦0.5 | 1 | ≦0.12 |
| 303 | ≦0.015 | 0.25 | 0.12 | 0.25 | 0.03 |
| 445 | 0.12 | 16 | >16 | >16 | 1 |
| 356 | 0.06 | ≦4 | >16 | 8 | 0.5 |
| 357 | 0.25 | 8 | 16 | 4 | 1 |
| 374 | 0.06 | 2 | 16 | 4 | 0.5 |
| 375 | 0.12 | >16 | >16 | >16 | 8 |
| 382 | 0.5 | 8 | >16 | 16 | 2 |
| 383 | 0.12 | 8 | >16 | >16 | 1 |
| 384 | 1 | ND | >16 | >16 | 8 |
| 388 | ≦0.25 | 2 | 8 | 2 | 0.5 |
| 389 | ≦0.015 | 0.5 | 1 | 0.5 | 0.06 |
| 390 | 0.06 | ND | 2 | 4 | 0.5 |
| 395 | 0.06 | 4 | 8 | 0.5 | 0.5 |
| 396 | 0.03 | 2 | 4 | 0.5 | 0.25 |
| 397 | 0.06 | ND | 8 | 2 | 0.5 |
| 358 | 0.12 | 16 | 8 | 16 | 0.5 |
| 359 | 0.12 | ND | 8 | 16 | 1 |
| 411 | 0.06 | >16 | >16 | 16 | 1 |
| 412 | 0.12 | 16 | >16 | 8 | 1 |
| 413 | 0.25 | 16 | >16 | >16 | 2 |
| 414 | 0.25 | ND | >16 | >16 | 2 |
| 415 | 0.12 | ND | >16 | >16 | 4 |
| 398 | ≦0.015 | 0.25 | 0.5 | 1 | 0.03 |
| 399 | 0.5 | 4 | >16 | >16 | 2 |
| 400 | ≦0.015 | 0.25 | 0.5 | 0.5 | 0.06 |
| 401 | 1 | ND | >16 | >16 | 8 |
| 402 | 0.25 | 16 | >16 | >16 | 2 |
| 405 | ≦0.015 | ≦0.12 | 0.25 | 0.25 | ≦0.25 |
| 406 | 0.06 | 8 | 8 | 2 | 0.5 |
| 407 | ≦0.015 | 0.03 | 0.25 | 0.12 | 0.03 |
| 408 | 0.03 | 1 | 2 | 2 | 0.25 |
| 409 | 0.12 | ND | >16 | >16 | 1 |
| 424 | ≦0.015 | 0.06 | 0.5 | ≦1 | ≦1 |
| 425 | 0.06 | 4 | 16 | 8 | 1 |
| 426 | ≦0.015 | ≦0.5 | 1 | 0.25 | 0.06 |
| 427 | ≦0.015 | 2 | 4 | 1 | 0.12 |
| 428 | 0.25 | >16 | >16 | >16 | 1 |
| 416 | 0.06 | 16 | >16 | 16 | 1 |
| 417 | 0.03 | 8 | >16 | 4 | 0.5 |
| 430 | 0.12 | 8 | 2 | 4 | 0.5 |
| 431 | 0.06 | 4 | 1 | 2 | 0.25 |
| 360 | 0.06 | 16 | 8 | 8 | 0.5 |
| 418 | 0.06 | 16 | >16 | 16 | 1 |
| 361 | ≦0.015 | 0.12 | 0.12 | 0.06 | ≦0.015 |
| 419 | ≦0.015 | 0.03 | 0.25 | 0.06 | ≦0.015 |
| 362 | 0.03 | 8 | >16 | 8 | 0.5 |
| 363 | 0.03 | 4 | >16 | 16 | 0.5 |
| 364 | 0.06 | >16 | >16 | >16 | 2 |
| 420 | ≦0.015 | ND | 1 | 0.5 | 0.12 |
| 421 | 0.06 | ND | 2 | 1 | 0.25 |
| 365 | 0.12 | ND | >16 | >16 | 2 |
| 366 | 4 | ND | >16 | 8 | 4 |
| 391 | 0.06 | 8 | >16 | 8 | 2 |
| 367 | ≦0.015 | 0.25 | 1 | 1 | 0.12 |
| 368 | 0.25 | 8 | >16 | 8 | 1 |
| 369 | 0.03 | 0.06 | 0.5 | 0.12 | 0.03 |
| 380 | 0.12 | 8 | 8 | 4 | 0.5 |
| 376 | 0.25 | ND | >16 | >16 | 1 |
| 377 | 0.5 | 16 | 8 | >16 | 4 |
| 392 | 0.25 | 16 | >16 | 8 | 1 |
| 385 | 1 | >16 | >16 | >16 | 8 |
| 381 | 0.5 | 4 | >16 | >16 | 4 |
| 404 | 0.12 | 4 | 16 | >16 | 0.5 |
| 422 | 0.12 | >16 | >16 | >16 | 1 |
| 423 | 0.25 | >16 | >16 | >16 | 4 |
| 410 | ≦0.015 | ND | 0.5 | 1 | 0.06 |
| 393 | 0.25 | ND | 8 | 2 | 0.5 |
| 394 | 0.03 | 1 | 4 | 1 | 0.5 |
| 370 | 2 | >16 | >16 | 16 | 8 |
| 371 | ≦0.015 | 0.06 | 0.12 | 0.12 | 0.03 |
| 372 | 0.5 | ND | 16 | 8 | 2 |
| 373 | 0.06 | 0.5 | 2 | 1 | 0.12 |
| 386 | 0.25 | ND | 8 | 16 | 2 |
| 387 | 0.5 | >16 | 16 | 16 | 2 |
| 378 | 0.25 | ND | 4 | 16 | 1 |
| 379 | 0.25 | 1 | 8 | 16 | 2 |
| 433 | ≦0.015 | ND | 0.5 | 0.5 | 0.12 |
| 435 | 0.12 | ND | 4 | 8 | 0.5 |
| 437 | 0.06 | ND | 1 | 0.25 | 0.12 |
| 439 | 0.06 | ND | 2 | 2 | 0.25 |
| 441 | 0.5 | 8 | >16 | >16 | 8 |
| 443 | 0.5 | 8 | >16 | >16 | 8 |
| 446 | 0.25 | ND | 4 | 4 | 1 |
| 447 | 0.25 | ND | 16 | 1 | 1 |
| 448 | 0.25 | ND | 8 | 16 | 2 |
| 449 | 0.25 | ND | >16 | 16 | 2 |
| 450 | 0.12 | ND | 8 | 8 | 0.5 |
| 451 | 0.25 | ND | 16 | 1 | 1 |
| 452 | 0.5 | ND | 4 | >16 | 4 |
| 453 | 0.06 | ND | 4 | 2 | 0.25 |
| 455 | ≦0.015 | 0.12 | 2 | 1 | 0.25 |
| 456 | ≦0.015 | 0.06 | 0.12 | 0.12 | 0.03 |
| 457 | 0.25 | 2 | 8 | 4 | 1 |
| 458 | ≦0.015 | 0.03 | 0.25 | 0.25 | 0.06 |
| 459 | ≦0.015 | ND | 0.25 | 0.12 | 0.06 |
| 467 | 0.12 | 0.5 | 4 | 1 | 0.5 |
| 491 | 0.12 | ND | 8 | 16 | 1 |
| 492 | 0.06 | ND | 4 | 8 | 0.25 |
| 493 | 0.06 | ND | >16 | 1 | 0.5 |
| 494 | 1 | ND | >16 | >16 | 8 |
| 495 | 0.25 | ND | 2 | ND | 1 |
| 496 | 0.25 | ND | 0.25 | 16 | 1 |
| 497 | 1 | >1 | >16 | >16 | 4 |
| 498 | 1 | 4 | >16 | >16 | 16 |
| 499 | 8 | >16 | >16 | >16 | >16 |
| 500 | 2 | >16 | >16 | >16 | 16 |
| 501 | 0.25 | ND | 8 | 16 | 2 |
| 502 | 0.5 | 16 | 16 | >16 | 4 |
| 503 | 4 | ND | >16 | >16 | 16 |
| 504 | 2 | ND | 8 | 4 | 16 |
| 505 | 0.12 | ND | 4 | 1 | 0.25 |
| 506 | ≦0.015 | ND | 2 | ND | 0.25 |
| 507 | 0.06 | 0.12 | 2 | 2 | 0.25 |
| 508 | 0.12 | ≦4 | >16 | 8 | 1 |

*ND = not determined
(A: Staphylococcus aureus OC4172; strains B, C, and D are fluoroquinolone-resistant clinical isolates of Streptococcus pneumoniae that contain different constellations of amino acid substitutions in the QRDR region; E: Streptococcus pneumoniae ATCC 49619)

Throughout this application, various publications are cited. The disclosure of these publications is hereby incorporated by reference into this application to describe more fully the state of the art to which this invention pertains.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound having a structure according to Formula (I): 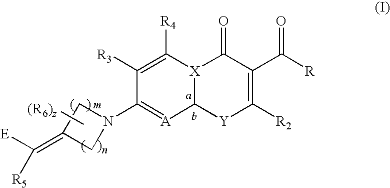
wherein:
n is an integer from 1 to 3;
m is an integer from 1 to 3;
z is an integer from 0 to 3;
R is selected from hydrogen, hydroxy, and alkoxy;
R₂ is hydrogen;
R₃ and R₄ are independently selected from hydrogen, halogen, amino, hydroxy, alkoxy, alkylthio, alkyl, alkenyl and alkynyl;
R₅ is selected from hydrogen, hydroxy, halogen, alkyl, aryl, alkoxy, cyano, CO₂R₁₃, and alkylthio;
R₆ is independently selected from alkyl, hydroxy, alkoxy, alkylthio, alkenyl, alkynyl, aryl, alkoxyimino, and halogen; or R₅ and R₆ join to form a 4- to 7-membered carbocyclic ring wherein each carbon atom of the ring can be optionally substituted with R₁₂, wherein R₁₂ is selected from the group consisting of halogen, amino, hydroxy, alkoxy, alkylthio, alkyl, alkenyl, alkynyl, oxo, alkoxyimino and hydroxyimino;
R₁₃ is hydrogen, alkyl, aryl, or a carboxylic acid protecting group;
E is selected from the group consisting of: 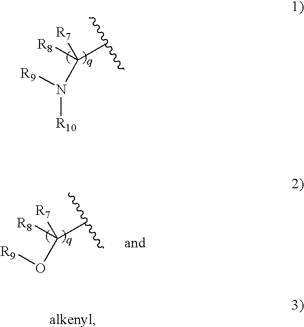
wherein,
q is an integer from 1 to 3;
In the case of 1), R₇ and R₈ are each independently selected from hydrogen and alkyl, or R₇ and R₈ join to form a 3 to 6 membered carbocyclic ring, or either of R₇ or R₈ can be joined independently to either of R₉ or R₁₀ to form a heterocyclic ring containing the nitrogen atom to which R₉ or R₁₀ are bonded, wherein
R₉ and R₁₀ are each independently selected from hydrogen, alkyl, aryl, arylalkyl, acyl, alkoxycarbonyl, or sulfonyl, or alternatively R₉ and R₁₀ join to form a heterocyclic ring containing the nitrogen atom to which they are bonded;
In the case of 2), R₇ and R₈ are each independently selected from hydrogen and alkyl, or R₇ and R₈ join to form a 3- to 6-membered carbocyclic ring, and R₉ is selected from hydrogen, alkyl, acyl, alkoxycarbonyl, or sulfonyl;
A is selected from N and C(R₁₁), wherein R₁₁ is selected from hydrogen, alkyl, halogen, hydroxy, alkoxy, alkylthio or cyano;
X is selected from C and N, where if X is C, then a is a double bond and b is a single bond, and if X is N, then a is a single bond and b is a double bond; and
Y is selected from N(R₁) and C(R₁), with the proviso that when Y is N(R₁), then X is C and when Y is C(R₁), then X is N; wherein
R₁ is selected from C3 to C6 cycloalkyl, C4 to C6 heterocycloalkyl, alkyl, alkene, a 6-membered aryl and a 6-membered heteroaryl; provided that
if A is C(R₁₁), X is C and Y is N(R₁), then R₁₁ and R₁ can join to form a 6-membered heterocyclic ring optionally substituted by one or more alkyl groups for a corresponding hydrogen atom; or
if A is C(R₁₁), X is C and Y is N(R₁), then R₂ and R₁ can join to form a monocyclic or bicyclic heterocyclic ring optionally substituted by one or more alkyl groups for a corresponding hydrogen atom; or
if A is C(R₁₁), X is C and Y is N(R₁), then R₂ and R can join to form a 5-membered heterocyclic ring optionally substituted by one or more alkyl groups for a corresponding hydrogen atom;
or an optical isomer, diastereomer or enantiomer thereof or a pharmaceutically acceptable salt, hydrate, or prodrug thereof.

2. The compound of claim 1, wherein A is C(OCH₃), C(OCHF₂), CH, CCl, or N.

3. The compound of claim 1, wherein A is C(R₁₁), X is C, Y is N(R₁) and R₁₁ and R₁ can join to form a 6-membered heterocyclic ring optionally substituted by one or more alkyl groups for a corresponding hydrogen atom.

4. The compound of claim 1, wherein Y is N(R₁) and R₁ is selected from C3 to C6 cycloalkyl or 6-membered heterocyclic ring.

5. The compound of claim 1, wherein E is:

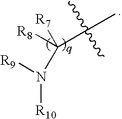

6. The compound of claim 1, wherein m is 1 and n is 1, m is 2 and n is 2, or m is 1 and n is 3.

7. The compound of claim 1, wherein z is 0 or R₆ is methyl and z is 1.

8. The compound of claim 4, wherein R₇ and R₈ are hydrogen.

9. The compound of claim 7, wherein q is 1.

10. The compound of claim 8, wherein R₉ is hydrogen, methyl, or ethyl and R₁₀ is hydrogen.

11. A compound having a structure according to Formula (Ia): 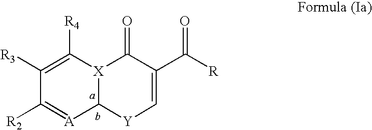
wherein:
R is selected from hydrogen, hydroxy or alkoxy;
R₂ is selected from the group consisting of: 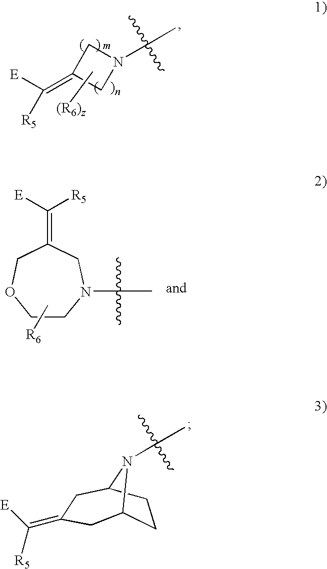
n is an integer selected from 1, 2 or 3;
m is an integer selected from 1, 2 or 3;
z is an integer selected from 0, 1, 2 or 3;
R₃ and R₄ are independently selected from hydrogen, halogen, amino, hydroxy, alkoxy, alkylthio, alkyl, alkenyl or alkynyl;
R₅ is selected from hydrogen, halogen, alkyl, aryl, alkoxy, cyano, CO₂R₁₃ or alkylthio;
R₆ is independently selected from alkyl, hydroxy, alkoxy, alkylthio, alkenyl, alkynyl, aryl, alkoxyimino or halogen, wherein alkyl is optionally mono- or disubstituted on an R₂ carbon atom;
alternatively, R₅ and R₆ join to form a 4- to 7-membered carbocyclic ring wherein each carbon atom of the ring can be optionally substituted with R₁₂;
R₁₂ is selected from the group consisting of halogen, amino, hydroxy, alkoxy, alkylthio, alkyl, alkenyl, alkynyl, oxo, alkoxyimino and hydroxyimino;
R₁₃ is hydrogen, alkyl, aryl, or a protecting group;
E is selected from the group consisting of: 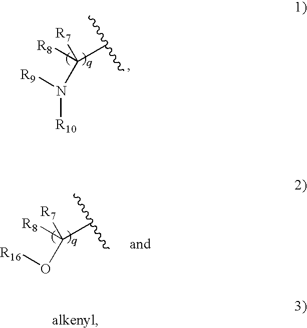
wherein,
q is an integer selected from 1, 2 or 3;
R₇ and R₈ are each independently selected from hydrogen or alkyl;
alternatively, R₇ and R₈ join to form a 3- to 6-membered carbocyclic ring;
alternatively, either of R₇ or R₈ can be joined independently to either of R₉ or R₁₀ to form a heterocyclic ring containing the nitrogen atom to which R₉ or R₁₀ are bonded;
R₉ and R₁₀ are each independently selected from hydrogen, alkyl, acyl, aminoacetyl, arylalkyl, alkoxycarbonyl or sulfonyl;
alternatively, R₉ and R₁₀ can be joined to form a heterocyclic ring containing the nitrogen atom to which they are bonded, wherein the heterocyclic ring is optionally substituted on one or two carbon atoms with oxo;
R₁₆ is selected from hydrogen, alkyl, acyl, alkoxycarbonyl or sulfonyl;
A is selected from N and C(R₁₁);
R₁₁ is selected from hydrogen, alkyl, haloalkyl, halogen, hydroxy, alkoxy, haloalkoxy, alkylthio or cyano;
X is selected from C and N, wherein if X is C, then a is a double bond and b is a single bond, and if X is N, then a is a single bond and b is a double bond;
Y is selected from N(R₁) and C(R₁), with the proviso that when Y is N(R₁), then X is C and when Y is C(R₁), then X is N;
R₁ is selected from an optionally substituted C3 to C6 cycloalkyl, C4 to C6 heterocycloalkyl, alkyl, alkene, 6-membered aryl or 6-membered heteroaryl;
wherein C₃ to C₆ cycloalkyl, aryl and heteroaryl are each optionally substituted with one, two or three substituents each selected from halogen, alkyl or amino;
provided that if A is C(R₁₁), X is C and Y is N(R₁), then R₁₁ and R₁ can join to form a 6-membered heterocyclic ring substituted with R₁₇;
R₁₇ is selected from hydrogen or alkyl;
or an optical isomer, diastereomer or enantiomer thereof; a pharmaceutically acceptable salt, hydrate, or prodrug thereof.

12. The compound of claim 11, wherein
R is hydroxy;
R₂ is selected from the group consisting of: 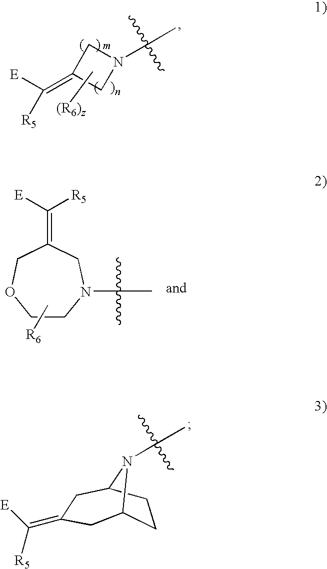
n is an integer selected from 1, 2 or 3;
m is an integer selected from 1 or 2;
z is an integer selected from 0, 1 or 2;
R₃ and R₄ are independently selected from hydrogen, halogen, amino or alkyl;
R₅ is selected from hydrogen, halogen, alkyl or cyano;
R₆ is independently selected from alkyl;
E is selected from the group consisting of: 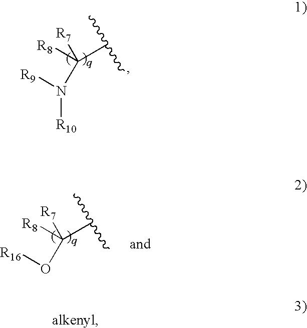
wherein,
q is 1;
R₇ and R₈ are each independently selected from hydrogen or alkyl;
R₉ and R₁₀ are each independently selected from hydrogen or alkyl;
alternatively, R₉ and R₁₀ can be joined to form a heterocyclic ring containing the nitrogen atom to which they are bonded, wherein the heterocyclic ring is optionally substituted on two carbon atoms with oxo;
R₁₆ is hydrogen;
A is selected from N and C(R₁₁);
R₁₁ is selected from hydrogen, alkyl, halogen, alkoxy or haloalkoxy;
X is C, wherein a is a double bond and b is a single bond;
Y is N(R₁);
R₁ is selected from an optionally substituted C3 to C6 cycloalkyl, 6-membered aryl or 6-membered heteroaryl;
wherein C₃ to C₆ cycloalkyl, aryl and heteroaryl are each optionally substituted with one, two or three substituents each selected from halogen or amino;
provided that if A is C(R₁₁), X is C and Y is N(R₁), then R₁₁ and R₁ can join to form a 6-membered heterocyclic ring substituted with R₁₇;
R₁₇ is alkyl;
or an optical isomer, diastereomer or enantiomer thereof; a pharmaceutically acceptable salt, hydrate, or prodrug thereof.

13. The compound of claim 11, wherein the compound is selected from the group consisting of Formula (Ib), Formula (Ic) and Formula (Id): 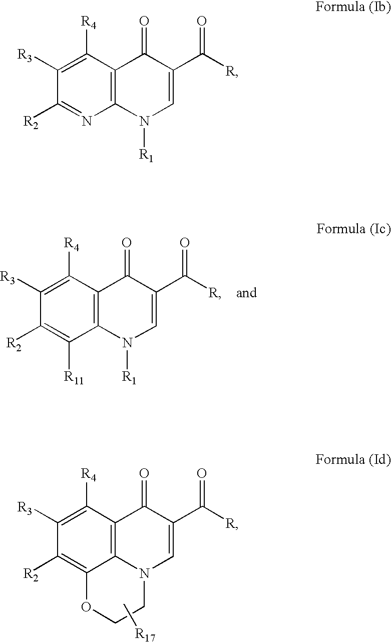
wherein all variables are as previously defined.

14. The compound of claim 13, wherein R₂ is selected from the group consisting of: 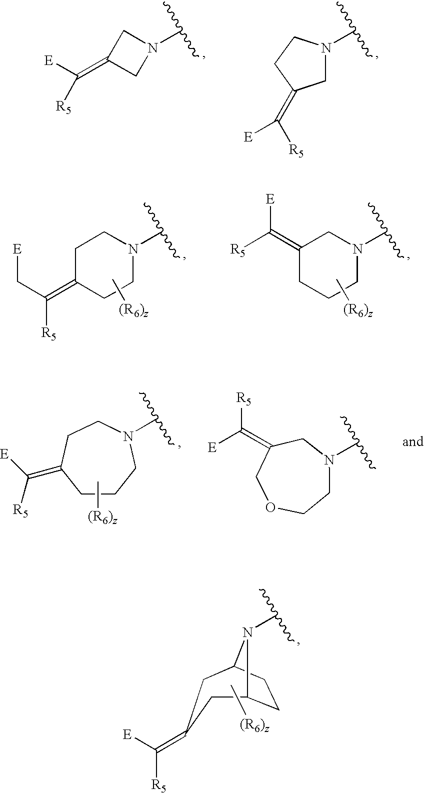
wherein all variables are as previously defined.

15. A compound selected from the group consisting of:
7-[4-(2-Amino-1-fluoro-ethylidene)-piperidin-1-yl]-1-cyclopropyl-6-fluoro-4-oxo-1,4-dihydro-[1,8]naphthyridine-3-carboxylic acid,
7-[4-(2-Amino-1-methyl-ethylidene)-piperidin-1-yl]-1-cyclopropyl-6-fluoro-4-oxo-1,4-dihydro-[1,8]naphthyridine-3-carboxylic acid,
7-[4-(2-Amino-1-chloro-ethylidene)-piperidin-1-yl]-1-cyclopropyl-6-fluoro-4-oxo-1,4-dihydro-[1,8]naphthyridine-3-carboxylic acid,
7-[4-(2-Amino-1-fluoro-ethylidene)-piperidin-1-yl]-1-(2,4-difluoro-phenyl)-6-fluoro-4-oxo-1,4-dihydro-[1,8]naphthyridine-3-carboxylic acid,
7-[4-(2-Amino-1-fluoro-ethylidene)-piperidin-1-yl]-1-cyclopropyl-6-fluoro-4-oxo-1,4-dihydro-quinoline-3-carboxylic acid,
7-[4-(2-Amino-1-methyl-ethylidene)-piperidin-1-yl]-1-cyclopropyl-6-fluoro-8-methoxy-4-oxo-1,4-dihydro-quinoline-3-carboxylic acid,
7-[4-(2-Amino-1-chloro-ethylidene)-piperidin-1-yl]-1-cyclopropyl-6-fluoro-8-methoxy-4-oxo-1,4-dihydro-quinoline-3-carboxylic acid,
9-[4-(2-Amino-1-fluoro-ethylidene)-piperidin-1-yl]-8-fluoro-3-methyl-6-oxo-2,3-dihydro-6H-1-oxa-3a-aza-phenalene-5-carboxylic acid,
9-[4-(2-Amino-1-methyl-ethylidene)-piperidin-1-yl]-8-fluoro-3-methyl-6-oxo-2,3-dihydro-6H-1-oxa-3a-aza-phenalene-5-carboxylic acid,
7-[4-(2-Amino-1-fluoro-ethylidene)-piperidin-1-yl]-1-cyclopropyl-8-methoxy-4-oxo-1,4-dihydro-quinoline-3-carboxylic acid,
(Z)-7-[4-(2-Amino-1-fluoro-ethylidene)-3-methyl-piperidin-1-yl]-1-cyclopropyl-6-fluoro-8-methoxy-4-oxo-1,4-dihydro-quinoline-3-carboxylic acid,
(E)-7-[4-(2-Amino-1-fluoro-ethylidene)-piperidin-1-yl]-1-cyclopropyl-7-6-fluoro-8-methoxy-4-oxo-1,4-dihydro-quinoline-3-carboxylic acid,
(E)-7-[3-(2-Amino-1-chloro-ethylidene)-pyrrolidin-1-yl]-1-cyclopropyl-6-fluoro-8-methoxy-4-oxo-1,4-dihydro-quinoline-3-carboxylic acid,
7-[4-(2-Amino-1-chloro-propylidene)-piperidin-1-yl]-1-cyclopropyl-6-fluoro-8-methoxy-4-oxo-1,4-dihydro-quinoline-3-carboxylic acid,
7-[4-(2-Amino-ethylidene)-piperidin-1-yl]-1-cyclopropyl-6-fluoro-4-oxo-1,4-dihydro-[1,8]naphthyridine-3-carboxylic acid,
7-[4-(2-Amino-ethylidene)-3-hydroxy-piperidin-1-yl]-1-cyclopropyl-6-fluoro-4-oxo-1,4-dihydro-[1,8]naphthyridine-3-carboxylic acid,
7-[4-(2-Amino-ethylidene)-3-methoxyimino-piperidin-1-yl]-1-cyclopropyl-6-fluoro-4-oxo-1,4-dihydro-[1,8]naphthyridine-3-carboxylic acid,
7-[4-(2-Amino-1-methyl-ethylidene)-3-hydroxy-piperidin-1-yl]-1-cyclopropyl-6-fluoro-4-oxo-1,4-dihydro-[1,8]naphthyridine-3-carboxylic acid,
7-[4-(2-Amino-1-methyl-ethylidene)-piperidin-1-yl]-1-cyclopropyl-6-fluoro-4-oxo-1,4-dihydro-quinoline-3-carboxylic acid,
7-[4-(2-Amino-1-chloro-ethylidene)-piperidin-1-yl]-1-1-cyclopropyl-6-fluoro-4-oxo-1,4-dihydro-quinoline-3-carboxylic acid,
9-[4-(2-Amino-1-methyl-ethylidene)-3-hydroxy-piperidin-1-yl]-1-8-fluoro-3-methyl-6-oxo-2,3-dihydro-6H-1-oxa-3a-aza-phenalene-5-carboxylic acid,
7-[4-(2-Amino-1-fluoro-ethylidene)-piperidin-1-yl]-1-cyclopropyl-8-difluoromethoxy-4-oxo-1,4-dihydro-quinoline-3-carboxylic acid,
7-[4-(1-Aminomethyl-propylidene)-piperidin-1-yl]-1-cyclopropyl-6-fluoro-4-oxo-1,4-dihydro-[1,8]naphthyridine-3-carboxylic acid,
7-[4-(2-Amino-1-methyl-ethylidene)-piperidin-1-yl]-1-cyclopropyl-6-fluoro-8-methoxy-4-oxo-1,4-dihydro-quinoline-3-carboxylic acid,
7-[4-(1-Aminomethyl-propylidene)-piperidin-1-yl]-1-cyclopropyl-6-fluoro-8-methoxy-4-oxo-1,4-dihydro-quinoline-3-carboxylic acid,
7-[4-(1-Chloro-2-methylamino-ethylidene)-piperidin-1-yl]-1-cyclopropyl-6-fluoro-8-methoxy-4-oxo-1,4-dihydroquinoline-3-carboxylic acid,
7-[4-(2-Amino-1-chloro-ethylidene)-piperidin-1-yl]-1-cyclopropyl-8-difluoromethoxy-6-fluoro-4-oxo-1,4-dihydro-quinoline-3-carboxylic acid,
1-Cyclopropyl-8-difluoromethoxy-6-fluoro-7-[4-(1-methyl-2-methylamino-ethylidene)-piperidin-1-yl]-4-oxo-1,4-dihydro-quinoline-3-carboxylic acid,
7-[4-(1-Chloro-2-ethylamino-ethylidene)-piperidin-1-yl]-1-cyclopropyl-6-fluoro-8-methoxy-4-oxo-1,4-dihydro-quinoline-3-carboxylic acid,
7-[4-(2-Amino-1-chloro-ethylidene)-azetidin-1-yl}-1-cyclopropyl-6-fluoro-4-oxo-1,4-dihydro-quinoline-3-carboxylic acid,
9-[3-(2-Amino-1-chloro-ethylidene)-azetidin-1-yl}-8-fluoro-(3S)-3-methyl-6-oxo-2,3-dihydro-6H-1-oxa-3a-aza-phenalene-5-carboxylic acid,
7-[4-(2-Amino-1-fluoro-ethylidene)-3,3-dimethyl-piperidin-1-yl]-1-cyclopropyl-6-fluoro-8-methoxy-4-oxo-1,4-dihydro-quinoline-3-carboxylic acid,
1-Cyclopropyl-6-fluoro-7-[4-(1-methyl-2-methylamino-ethylidene)-piperidin-1-yl]-4-oxo-1,4-dihydro-[1,8]naphthyridine-3-carboxylic acid,
7-[3-(2-Amino-1-fluoro-ethylidene)-azetidin-1-yl]-1-cyclopropyl-6-fluoro-4-oxo-1,4-dihydro-[1,8]naphthyridine-3-carboxylic acid,
7-[3-(2-Amino-1-chloro-ethylidene)-azetidin-1-yl}-1-cyclopropyl-6-fluoro-4-oxo-1,4-dihydro-[1,8]naphthyridine-3-carboxylic acid,
7-[3-(2-Amino-1-fluoro-ethylidene)-azetidin-1-yl]-1-cyclopropyl-8-difluoromethoxy-6-fluoro-4-oxo-1,4-dihydro-quinoline-3-carboxylic acid,
7-{4-[2-(Benzyl-methyl-amino)-1-chloro-ethylidene]-piperidin-1-yl}-1-cyclopropyl-6-fluoro-8-methoxy-4-oxo-1,4-dihydro-quinoline-3-carboxylic acid,
1-Cyclopropyl-6-fluoro-7-[4-(2-hydroxy-ethylidene)-piperidin-1-yl]-4-oxo-1,4-dihydro-[1,8]naphthyridine-3-carboxylic acid,
1-Cyclopropyl-6-fluoro-7-[4-(2-hydroxy-ethylidene)-piperidin-1-yl]-4-oxo-1,4-dihydro-quinoline-3-carboxylic acid,
7-[4-(1-Chloro-2-isopropylamino-ethylidene)-piperidin-1-yl]-1-cyclopropyl-6-fluoro-4-oxo-1,4-dihydro-[1,8]naphthyridine-3-carboxylic acid,
1-Cyclopropyl-6-fluoro-7-[4-(1-fluoro-2-hydroxy-ethylidene)-piperidin-1-yl]-4-oxo-1,4-dihydro-[1,8]naphthyridine-3-carboxylic acid,
7-[4-(1-Aminomethyl-but-3-enylidene)-piperidin-1-yl]-1-cyclopropyl-6-fluoro-4-oxo-1,4-dihydro-[1,8]naphthyridine-3-carboxylic acid,
7-[4-(2-Amino-1-fluoro-ethylidene)-3,3-dimethyl-piperidin-1-yl]-1-cyclopropyl-6-fluoro-4-oxo-1,4-dihydro-[1,8]naphthyridine-3-carboxylic acid,
7-[4-(2-Amino-1-bromo-ethylidene)-3,3-dimethyl-piperidin-1-yl]-1-cyclopropyl-6-fluoro-4-oxo-1,4-dihydro-[1,8]naphthyridine-3-carboxylic acid,
7-{4-[1-Chloro-2-(2-dimethylamino-ethylamino)-ethylidene]-piperidin-1-yl}-1-cyclopropyl-6-fluoro-4-oxo-1,4-dihydro-[1,8]naphthyridine-3-carboxylic acid,
7-[4-(1-Chloro-allylidene)-piperidin-1-yl]-1-cyclopropyl-6-fluoro-4-oxo-1,4-dihydro-[1,8]naphthyridine-3-carboxylic acid,
1-Cyclopropyl-7-[4-(2-ethoxycarbonyloxy-1-fluoro-ethylidene)-piperidin-1-yl]-6-fluoro-4-oxo-1,4-dihydro-[1,8]naphthyridine-3-carboxylic acid,
7-[4-(1-Chloro-2-dimethylamino-ethylidene)-piperidin-1-yl]-1-cyclopropyl-6-fluoro-4-oxo-1,4-dihydro-[1,8]naphthyridine-3-carboxylic acid,
7-[4-(1-Chloro-2-piperidin-1-yl-ethylidene)-piperidin-1-yl]-1-cyclopropyl-6-fluoro-4-oxo-1,4-dihydro-[1,8]naphthyridine-3-carboxylic acid,
1-Cyclopropyl-7-[4-(2-dimethylamino-1-fluoro-ethylidene)-piperidin-1-yl]-6-fluoro-4-oxo-1,4-dihydro-[1,8]naphthyridine-3-carboxylic acid,
7-[4-(1-Aminomethyl-but-3-enylidene)-piperidin-1-yl]-1-cyclopropyl-6-fluoro-4-oxo-1,4-dihydro-[1,8]naphthyridine-3-carboxylic acid,
1-(2,4-Difluoro-phenyl)-6-fluoro-7-[4-(2-hydroxy-ethylidene)-piperidin-1-yl]-4-oxo-1,4-dihydro-[1,8]naphthyridine-3-carboxylic acid,
7-[3-(2-Amino-1-chloro-ethylidene)-azetidin-1-yl]-1-cyclopropyl-6-fluoro-8-methoxy-4-oxo-1,4-dihydro-quinoline-3-carboxylic acid,
7-[4-(1-Chloro-2-dimethylamino-ethylidene)-piperidin-1-yl]-1-cyclopropyl-6-fluoro-8-methoxy-4-oxo-1,4-dihydro-quinoline-3-carboxylic acid,
7-[4-(1-Chloro-2-pyrrolidin-1-yl-ethylidene)-piperidin-1-yl]-1-cyclopropyl-6-fluoro-8-methoxy-4-oxo-1,4-dihydro-quinoline-3-carboxylic acid,
7-[4-(1-Chloro-2-piperidin-1-yl-ethylidene)-piperidin-1-yl]-1-cyclopropyl-6-fluoro-8-methoxy-4-oxo-1,4-dihydro-quinoline-3-carboxylic acid,
7-[4-(1-Chloro-2-morpholin-4-yl-ethylidene)-piperidin-1-yl]-1-cyclopropyl-6-fluoro-8-methoxy-4-oxo-1,4-dihydro-quinoline-3-carboxylic acid,
7-[4-(1-Chloro-2-isopropylamino-ethylidene)-piperidin-1-yl]-1-cyclopropyl-6-fluoro-8-methoxy-4-oxo-1,4-dihydro-quinoline-3-carboxylic acid,
1-Cyclopropyl-7-[4-(2-dimethylamino-1-fluoro-ethylidene)-piperidin-1-yl]-6-fluoro-8-methoxy-4-oxo-1,4-dihydro-quinoline-3-carboxylic acid,
7-[4-(1-Aminomethyl-3-hydroxy-propylidene)-piperidin-1-yl]-1-cyclopropyl-6-fluoro-8-methoxy-4-oxo-1,4-dihydro-quinoline-3-carboxylic acid,
7-[4-(1-Aminomethyl-3-methyl-but-3-enylidene)-piperidin-1-yl]-1-cyclopropyl-6-fluoro-8-methoxy-4-oxo-1,4-dihydro-quinoline-3-carboxylic acid,
7-[4-(1-Aminomethyl-but-3-enylidene)-piperidin-1-yl]-1-cyclopropyl-6-fluoro-8-methoxy-4-oxo-1,4-dihydro-quinoline-3-carboxylic acid,
7-[4-(2-Amino-1-bromo-ethylidene)-piperidin-1-yl]-1-cyclopropyl-6-fluoro-8-methoxy-4-oxo-1,4-dihydro-quinoline-3-carboxylic acid,
7-[3-(2-Amino-1-chloro-ethylidene)-azetidin-1-yl]-1-cyclopropyl-8-difluoromethoxy-6-fluoro-4-oxo-1,4-dihydro-quinoline-3-carboxylic acid,
7-{4-[2-(Benzyl-methyl-amino)-1-chloro-ethylidene]-piperidin-1-yl}-1-cyclopropyl-8-difluoromethoxy-6-fluoro-4-oxo-1,4-dihydro-quinoline-3-carboxylic acid
7-{4-[2-(Benzyl-methyl-amino)-1-fluoro-ethylidene]-piperidin-1-yl}-1-cyclopropyl-8-difluoromethoxy-6-fluoro-4-oxo-1,4-dihydro-quinoline-3-carboxylic acid,
7-[4-(2-Amino-1-fluoro-ethylidene)-piperidin-1-yl]-1-cyclopropyl-6-fluoro-4-oxo-1,4-dihydro-quinoline-3-carboxylic acid,
7-[3-(2-Amino-1-chloro-ethylidene)-azetidin-1-yl]-1-cyclopropyl-8-methoxy-4-oxo-1,4-dihydro-quinoline-3-carboxylic acid,
9-[4-(2-Amino-ethylidene)-3-hydroxy-piperidin-1-yl]-8-fluoro-3-methyl-6-oxo-2,3-dihydro-6H-1-oxa-3a-aza-phenalene-5-carboxylic acid,
8-Fluoro-9-[4-(2-hydroxy-1-methyl-ethylidene)-piperidin-1-yl]-3-methyl-6-oxo-2,3-dihydro-6H-1-oxa-3a-aza-phenalene-5-carboxylic acid,
7-[4-(2-Amino-ethylidene)-piperidin-1-yl]-1-cyclopropyl-6-fluoro-8-methoxy-4-oxo-1,4-dihydro-quinoline-3-carboxylic acid,
1-Cyclopropyl-6-fluoro-7-[4-(1-fluoro-2-methylamino-ethylidene)-piperidin-1-yl]-8-methoxy-4-oxo-1,4-dihydro-quinoline-3-carboxylic acid,
1-Cyclopropyl-7-[4-(2-ethylamino-1-fluoro-ethylidene)-piperidin-1-yl]-6-fluoro-8-methoxy-4-oxo-1,4-dihydro-quinoline-3-carboxylic acid,
7-[4-(1-Chloro-2-methylamino-ethylidene)-piperidin-1-yl]-1-cyclopropyl-6-fluoro-4-oxo-1,4-dihydro-[1,8]naphthyridine-3-carboxylic acid,
7-[4-(1-Chloro-2-methylamino-ethylidene)-piperidin-1-yl]-1-cyclopropyl-6-fluoro-4-oxo-1,4-dihydro-[1,8]naphthyridine-3-carboxylic acid,
1-Cyclopropyl-6-fluoro-7-[4-(1-methyl-2-methylamino-ethylidene)-piperidin-1-yl]-4-oxo-1,4-dihydro-[1,8]naphthyridine-3-carboxylic acid,
1-Cyclopropyl-6-fluoro-7-[4-(1-methyl-2-methylamino-ethylidene)-piperidin-1-yl]-4-oxo-1,4-dihydro-quinoline-3-carboxylic acid,
1-Cyclopropyl-6-fluoro-7-[4-(1-methyl-2-methylamino-ethylidene)-piperidin-1-yl]-4-oxo-1,4-dihydro-quinoline-3-carboxylic acid,
1-Cyclopropyl-8-difluoromethoxy-6-fluoro-7-[4-(1-methyl-2-methylamino-ethylidene)-piperidin-1-yl]-4-oxo-1,4-dihydro-quinoline-3-carboxylic acid,
1-Cyclopropyl-6-fluoro-8-methoxy-7-[4-(2-methylamino-ethylidene)-piperidin-1-yl]-4-oxo-1,4-dihydro-quinoline-3-carboxylic acid,
7-[4-(2-Acetylamino-ethylidene)-piperidin-1-yl]-1-cyclopropyl-6-fluoro-4-oxo-1,4-dihydro-[1,8]naphthyridine-3-carboxylic acid,
1-Cyclopropyl-6-fluoro-7-[4-(1-fluoro-2-isopropylamino-ethylidene)-piperidin-1-yl]-4-oxo-1,4-dihydro-[1,8]naphthyridine-3-carboxylic acid,
7-[4-(1-Chloro-2-isopropylamino-ethylidene)-piperidin-1-yl]-1-cyclopropyl-6-fluoro-4-oxo-1,4-dihydro-quinoline-3-carboxylic acid,
1-Cyclopropyl-6-fluoro-7-[4-(1-fluoro-2-isopropylamino-ethylidene)-piperidin-1-yl]-4-oxo-1,4-dihydro-quinoline-3-carboxylic acid,
7-[4-(1-Chloro-2-isopropylamino-ethylidene)-piperidin-1-yl]-1-cyclopropyl-8-difluoromethoxy-6-fluoro-4-oxo-1,4-dihydro-quinoline-3-carboxylic acid,
1-Cyclopropyl-8-difluoromethoxy-6-fluoro-7-[4-(1-fluoro-2-isopropylamino-ethylidene)-piperidin-1-yl]-4-oxo-1,4-dihydro-quinoline-3-carboxylic acid,
1-Cyclopropyl-6-fluoro-7-[4-(1-fluoro-2-isopropylamino-ethylidene)-piperidin-1-yl]-8-methoxy-4-oxo-1,4-dihydro-quinoline-3-carboxylic acid,
7-[3-(2-Amino-1-fluoro-ethylidene)-piperidin-1-yl]-1-cyclopropyl-6-fluoro-4-oxo-1,4-dihydro-[1,8]naphthyridine-3-carboxylic acid,
(E)-7-[3-(2-Amino-1-chloro-ethylidene)-piperidin-1-yl]-1-cyclopropyl-6-fluoro-4-oxo-1,4-dihydro-[1,8]naphthyridine-3-carboxylic acid,
(Z)-7-[3-(2-Amino-1-chloro-ethylidene)-piperidin-1-yl]-1-cyclopropyl-6-fluoro-4-oxo-1,4-dihydro-[1,8]naphthyridine-3-carboxylic acid,
7-[3-(2-Amino-1-fluoro-ethylidene)-piperidin-1-yl]-1-cyclopropyl-6-fluoro-4-oxo-1,4-dihydro-quinoline-3-carboxylic acid,
(E)-7-[3-(2-Amino-1-chloro-ethylidene)-piperidin-1-yl]-1-cyclopropyl-6-fluoro-4-oxo-1,4-dihydro-quinoline-3-carboxylic acid,
(Z)-7-[3-(2-Amino-1-chloro-ethylidene)-piperidin-1-yl]-1-cyclopropyl-6-fluoro-4-oxo-1,4-dihydro-quinoline-3-carboxylic acid,
(E)-7-[3-(2-Amino-1-fluoro-ethylidene)-piperidin-1-yl]-1-cyclopropyl-8-difluoromethoxy-6-fluoro-4-oxo-1,4-dihydro-quinoline-3-carboxylic acid,
(E)-7-[3-(2-Amino-1-chloro-ethylidene)-piperidin-1-yl]-1-cyclopropyl-8-difluoromethoxy-6-fluoro-4-oxo-1,4-dihydro-quinoline-3-carboxylic acid,
7-[3-(2-Amino-1-chloro-ethylidene)-piperidin-1-yl]-1-cyclopropyl-6-fluoro-8-methoxy-4-oxo-1,4-dihydro-quinoline-3-carboxylic acid,
7-[4-(2-Amino-1-chloro-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
7-[4-(2-Amino-1-chloro-ethylidene)-piperidin-1-yl]-1-cyclopropyl-6-fluoro-5-methyl-4-oxo-1,4-dihydro-quinoline-3-carboxylic acid,
7-[4-(1-Chloro-2-methylamino-ethylidene)-piperidin-1-yl]-1-cyclopropyl-6-fluoro-8-hydroxy-4-oxo-1,4-dihydro-quinoline-3-carboxylic acid,
7-[3-(2-Amino-1-chloro-ethylidene)-azetidin-1-yl]-1-(6-amino-3,5-difluoro-pyridin-2-yl)-8-chloro-6-fluoro-4-oxo-1,4-dihydro-quinoline-3-carboxylic acid,
1-(6-Amino-3,5-difluoro-pyridin-2-yl)-7-[3-(2-amino-1-fluoro-ethylidene)-azetidin-1-yl]-8-chloro-6-fluoro-4-oxo-1,4-dihydro-quinoline-3-carboxylic acid,
7-[4-(2-Amino-1-chloro-ethylidene)-piperidin-1-yl]-1-(6-amino-3,5-difluoro-pyridin-2-yl)-8-chloro-6-fluoro-4-oxo-1,4-dihydro-quinoline-3-carboxylic acid,
7-[4-(2-Amino-1-chloro-ethylidene)-piperidin-1-yl]-1-(2,4-difluoro-phenyl)-6-fluoro-4-oxo-1,4-dihydro-[1,8]naphthyridine-3-carboxylic acid,
7-[3-(2-amino-1-fluoro-ethylidene)-azetidin-1-yl]-1-cyclopropyl-6-fluoro-4-oxo-1,4-dihydro-quinoline-3-carboxylic acid,
7-[3-(2-amino-1-fluoro-ethylidene)-azetidin-1-yl]-1-cyclopropyl-6-fluoro-8-methoxy-4-oxo-1,4-dihydro-quinoline-3-carboxylic acid,
7-[3-(2-amino-1-fluoro-ethylidene)-azetidin-1-yl]-1-cyclopropyl-8-methoxy-4-oxo-1,4-dihydro-quinoline-3-carboxylic acid,
7-[4-(2-Amino-1-chloro-ethylidene)-piperidin-1-yl]-1-cyclopropyl-8-methoxy-4-oxo-1,4-dihydro-quinoline-3-carboxylic acid,
9-[3-(2-Amino-1-fluoro-ethylidene)-azetidin-1-yl]-8-fluoro-(3S)-3-methyl-6-oxo-2,3-dihydro-6H-1-oxa-3a-aza-phenalene-5-carboxylic acid,
9-[4-(2-Amino-1-chloro-ethylidene)-piperidin-1-yl]-8-fluoro-(3S)-3-methyl-6-oxo-2,3-dihydro-6H-1-oxa-3a-aza-phenalene-5-carboxylic acid,
7-[4-(2-Amino-1-cyano-ethylidene)-piperidin-1-yl]-1-cyclopropyl-6-fluoro-4-oxo-1,4-dihydro-[1,8]naphthyridine-3-carboxylic acid,
7-[4-(2-Amino-1-cyano-ethylidene)-piperidin-1-yl]-1-cyclopropyl-6-fluoro-4-oxo-1,4-dihydro-quinoline-3-carboxylic acid,
7-[4-(2-Amino-1-carboxy-ethylidene)-piperidin-1-yl]-1-cyclopropyl-6-fluoro-4-oxo-1,4-dihydro-[1,8]naphthyridine-3-carboxylic acid,
7-{4-[2-(2-Amino-ethylamino)-1-chloro-ethylidene]-piperidin-1-yl}-1-cyclopropyl-6-fluoro-4-oxo-1,4-dihydro-[1,8]naphthyridine-3-carboxylic acid,
7-{4-[1-Chloro-2-(4-formyl-piperazin-1-yl)-ethylidene]-piperidin-1-yl}-1-cyclopropyl-6-fluoro-4-oxo-1,4-dihydro-[1,8]naphthyridine-3-carboxylic acid,
7-[4-(1-chloro-2-piperazin-1-yl-ethylidene)-piperidin-1-yl]-1-cyclopropyl-6-fluoro-4-oxo-1,4-dihydro-[1,8]naphthyridine-3-carboxylic acid,
1-Cyclopropyl-6-fluoro-7-[4-(1-fluoro-2-piperazin-1-yl-ethylidene)-piperidin-1-yl]-4-oxo-1,4-dihydro-[1,8]naphthyridine-3-carboxylic acid,
9-[4-(2-Dimethylamino-1-fluoro-ethylidene)-piperidin-1-yl]-8-fluoro-(3S)-3-methyl-6-oxo-2,3-dihydro-6H-1-oxa-3a-aza-phenalene-5-carboxylic acid,
(Z)-7-[3-(1-Amino-2-propylidene)-piperidin-1-yl]-1-cyclopropyl-6-fluoro-4-oxo-1,4-dihydro-[1,8]naphthyridine-3-carboxylic acid,
7-[4-(1-Chloro-2-dimethylamino-ethylidene)-piperidin-1-yl]-1-cyclopropyl-6-fluoro-4-oxo-1,4-dihydro-quinoline-3-carboxylic acid,
1-Cyclopropyl-7-[4-(2-dimethylamino-1-fluoro-ethylidene)-piperidin-1-yl]-6-fluoro-4-oxo-1,4-dihydro-quinoline-3-carboxylic acid,
7-[4-(1-Chloro-2-piperidin-1-yl-ethylidene)-piperidin-1-yl]-1-cyclopropyl-6-fluoro-4-oxo-1,4-dihydro-quinoline-3-carboxylic acid,
(Z)-7-[3-(2-Amino-1-methyl-ethylidene)-piperidin-1-yl]-1-cyclopropyl-6-fluoro-4-oxo-1,4-dihydro-quinoline-3-carboxylic acid,
(Z)-7-[3-(2-Amino-1-methyl-ethylidene)-piperidin-1-yl]-1-cyclopropyl-6-fluoro-8-methoxy-4-oxo-1,4-dihydro-quinoline-3-carboxylic acid,
(E)-7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid,
7-[3-(1-Chloro-2-hydroxy-ethylidene)-azetidin-1-yl]-1-cyclopropyl-6-fluoro-8-methoxy-4-oxo-1,4-dihydro-quinoline-3-carboxylic acid,
7-[3-(1-Chloro-2-hydroxy-ethylidene)-azetidin-1-yl]-1-cyclopropyl-6-fluoro-4-oxo-1,4-dihydro-[1,8]naphthyridine-3-carboxylic acid,
(E)-1-(6-Amino-3,5-difluoro-pyridin-2-yl)-7-[3-(2-amino-1-fluoro-ethylidene)-piperidin-1-yl]-8-chloro-6-fluoro-4-oxo-1,4-dihydro-quinoline-3-carboxylic acid trifluoroacetate,
(Z)-1-(6-Amino-3,5-difluoro-pyridin-2-yl)-8-chloro-6-fluoro-7-[3-(1-fluoro-2-hydroxy-ethylidene)-piperidin-1-yl]-4-oxo-1,4-dihydroquinoline-3-carboxylic acid trifluoroacetate,
1-(6-Amino-3,5-difluoro-pyridin-2-yl)-8-chloro-7-[3-(1-chloro-2-methylamino-ethylidene)-azetidin-1-yl]-6-fluoro-4-oxo-1,4-dihydro-quinoline-3-carboxylic acid,
1-(6-Amino-3,5-difluoro-pyridin-2-yl)-8-chloro-7-[3-(1-chloro-2-hydroxy-ethylidene)-azetidin-1-yl]-6-fluoro-4-oxo-1,4-dihydro-quinoline-3-carboxylic acid,
(S)-9-[3-(1-Chloro-2-hydroxy-ethylidene)-azetidin-1-yl]-8-fluoro-3-methyl-6-oxo-2,3-dihydro-6H-1-oxa-3a-aza-phenalene-5-carboxylic acid,
7-[3-(1-Chloro-2-hydroxy-ethylidene)-azetidin-1-yl]-6-fluoro-(1R,2S)-1-(2-fluoro-cyclopropyl)-8-methoxy-4-oxo-1,4-dihydro-quinoline-3-carboxylic acid,
7-[3-(1-Chloro-2-hydroxy-ethylidene)-azetidin-1-yl]-1-cyclopropyl-6-fluoro-5-methyl-4-oxo-1,4-dihydro-quinoline-3-carboxylic acid,
9-[3-(2-Amino-ethylidene)-azetidin-1-yl]-8-fluoro-(3S)-3-methyl-6-oxo-2,3-dihydro-6H-1-oxa-3a-aza-phenalene-5-carboxylic acid,
9-[3-(2-Amino-1-methyl-ethylidene)-azetidin-1-yl]-8-fluoro-(3S)-3-methyl-6-oxo-2,3-dihydro-6H-1-oxa-3a-aza-phenalene-5-carboxylic acid,
7-[3-(2-Amino-1-methyl-ethylidene)-azetidin-1-yl]-1-cyclopropyl-6-fluoro-5-methyl-4-oxo-1,4-dihydro-quinoline-3-carboxylic acid,
7-[3-(2-Amino-1-methyl-ethylidene)-azetidin-1-yl]-6-fluoro-(1R,2S)-1-(2-fluoro-cyclopropyl)-8-methoxy-4-oxo-1,4-dihydro-quinoline-3-carboxylic acid,
5-Amino-7-[4-(2-amino-1-chloro-ethylidene)-piperidin-1-yl]-1-cyclopropyl-6,8-difluoro-4-oxo-1,4-dihydro-quinoline-3-carboxylic acid,
7-[4-(2-Amino-1-chloro-ethylidene)-piperidin-1-yl]-1-cyclopropyl-6-fluoro-8-fluoromethoxy-4-oxo-1,4-dihydro-quinoline-3-carboxylic acid,
7-[4-(2-Amino-1-chloro-ethylidene)-piperidin-1-yl]-1-cyclopropyl-6-fluoro-8-methyl-4-oxo-1,4-dihydro-quinoline-3-carboxylic acid,
(Z)-9-[4-(2-Amino-1-chloro-ethylidene)-azepan-1-yl]-8-fluoro-(3S)-3-methyl-6-oxo-2,3-dihydro-6H-1-oxa-3a-aza-phenalene-5-carboxylic acid,
9-[(E)-4-(2-Amino-1-chloro-ethylidene)-azepan-1-yl]-8-fluoro-(3S)-3-methyl-6-oxo-2,3-dihydro-6H-1-oxa-3a-aza-phenalene-5-carboxylic acid,
(Z)-7-[3-(2-Amino-1-fluoro-ethylidene)-piperidin-1-yl]-1-cyclopropyl-6-fluoro-4-oxo-1,4-dihydro-[1,8]naphthyridine-3-carboxylic acid,
(Z)-7-[3-(2-Amino-ethylidene)-piperidin-1-yl]-1-cyclopropyl-6-fluoro-4-oxo-1,4-dihydro-[1,8]naphthyridine-3-carboxylic acid,
(E)-7-[3-(2-Amino-ethylidene)-piperidin-1-yl]-1-cyclopropyl-6-fluoro-4-oxo-1,4-dihydro-[1,8]naphthyridine-3-carboxylic acid,
(E)-7-[3-(2-Amino-1-methyl-ethylidene)-piperidin-1-yl]-1-cyclopropyl-6-fluoro-4-oxo-1,4-dihydro-[1,8]naphthyridine-3-carboxylic acid,
(Z)-7-[4-(2-Amino-1-chloro-ethylidene)-azepan-1-yl]-1-cyclopropyl-6-fluoro-4-oxo-1,4-dihydro-[1,8]naphthyridine-3-carboxylic acid,
(E)-7-[4-(2-Amino-1-chloro-ethylidene)-azepan-1-yl]-1-cyclopropyl-6-fluoro-4-oxo-1,4-dihydro-[1,8]naphthyridine-3-carboxylic acid,
(Z)-7-[3-(2-Amino-1-methyl-ethylidene)-piperidin-1-yl]-1-(2,4-difluoro-phenyl)-6-fluoro-4-oxo-1,4-dihydro-[1,8]naphthyridine-3-carboxylic acid,
(E)-7-[3-(2-Amino-1-methyl-ethylidene)-piperidin-1-yl]-1-(2,4-difluoro-phenyl)-6-fluoro-4-oxo-1,4-dihydro-[1,8]naphthyridine-3-carboxylic acid,
(Z)-7-[3-(2-Amino-1-fluoro-ethylidene)-piperidin-1-yl]-1-cyclopropyl-6-fluoro-4-oxo-1,4-dihydro-quinoline-3-carboxylic acid,
(Z)-7-[3-(2-Amino-ethylidene)-piperidin-1-yl]-1-cyclopropyl-6-fluoro-4-oxo-1,4-dihydro-quinoline-3-carboxylic acid,
(E)-7-[3-(2-Amino-ethylidene)-piperidin-1-yl]-1-cyclopropyl-6-fluoro-4-oxo-1,4-dihydro-quinoline-3-carboxylic acid,
(E)-7-[3-(2-Amino-1-methyl-ethylidene)-piperidin-1-yl]-1-cyclopropyl-6-fluoro-4-oxo-1,4-dihydro-quinoline-3-carboxylic acid,
(Z)-7-[4-(2-Amino-1-chloro-ethylidene)-azepan-1-yl]-1-cyclopropyl-6-fluoro-4-oxo-1,4-dihydro-quinoline-3-carboxylic acid,
(E)-7-[4-(2-Amino-1-chloro-ethylidene)-azepan-1-yl]-1-cyclopropyl-6-fluoro-4-oxo-1,4-dihydro-quinoline-3-carboxylic acid,
(Z)-7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid,
(Z)-7-[3-(2-Amino-ethylidene)-piperidin-1-yl]-1-cyclopropyl—6-fluoro-8-methoxy-4-oxo-1,4-dihydro-quinoline-3-carboxylic acid,
(E)-7-[3-(2-Amino-ethylidene)-piperidin-1-yl]-1-cyclopropyl-6-fluoro-8-methoxy-4-oxo-1,4-dihydro-quinoline-3-carboxylic acid,
7-[3-(2-Amino-1-methyl-ethylidene)-azetidin-1-yl]-1-cyclopropyl-6-fluoro-8-methoxy-4-oxo-1,4-dihydro-quinoline-3-carboxylic acid,
(E)-7-[3-(2-Amino-1-methyl-ethylidene)-piperidin-1-yl]-1-cyclopropyl-6-fluoro-8-methoxy-4-oxo-1,4-dihydro-quinoline-3-carboxylic acid,
(Z)-7-[4-(2-Amino-1-chloro-ethylidene)-azepan-1-yl]-1-cyclopropyl-6-fluoro-8-methoxy-4-oxo-1,4-dihydro-quinoline-3-carboxylic acid,
(E)-7-[4-(2-Amino-1-chloro-ethylidene)-azepan-1-yl]-1-cyclopropyl-6-fluoro-8-methoxy-4-oxo-1,4-dihydro-quinoline-3-carboxylic acid,
(Z)-7-[3-(2-Amino-1-fluoro-ethylidene)-piperidin-1-yl]-1-cyclopropyl-8-difluoromethoxy-6-fluoro-4-oxo-1,4-dihydro-quinoline-3-carboxylic acid,
(Z)-7-[3-(2-Amino-ethylidene)-piperidin-1-yl]-1-cyclopropyl-8-difluoromethoxy-6-fluoro-4-oxo-1,4-dihydro-quinoline-3-carboxylic acid,
(E)-7-[3-(2-Amino-ethylidene)-piperidin-1-yl]-1-cyclopropyl-8-difluoromethoxy-6-fluoro-4-oxo-1,4-dihydro-quinoline-3-carboxylic acid,
(E)-9-[3-(2-Amino-1-fluoro-ethylidene)-piperidin-1-yl]-8-fluoro-(3S)-3-methyl-6-oxo-2,3-dihydro-6H-1-oxa-3a-aza-phenalene-5-carboxylic acid,
(Z)-9-[3-(2-Amino-1-fluoro-ethylidene)-piperidin-1-yl]-8-fluoro-(3S)-3-methyl-6-oxo-2,3-dihydro-6H-1-oxa-3a-aza-phenalene-5-carboxylic acid,
(E)-9-[3-(2-Amino-1-chloro-ethylidene)-piperidin-1-yl]-8-fluoro-(3S)-3-methyl-6-oxo-2,3-dihydro-6H-1-oxa-3a-aza-phenalene-5-carboxylic acid,
(E)-9-[3-(2-Amino-ethylidene)-piperidin-1-yl]-8-fluoro-(3S)-3-methyl-6-oxo-2,3-dihydro-6H-1-oxa-3a-aza-phenalene-5-carboxylic acid,
(Z)-9-[3-(2-Amino-ethylidene)-piperidin-1-yl]-8-fluoro-(3S)-3-methyl-6-oxo-2,3-dihydro-6H-1-oxa-3a-aza-phenalene-5-carboxylic acid,
9-[3-(1-Chloro-2-hydroxy-ethylidene)-azetidin-1-yl]-8-fluoro-(3S)-3-methyl-6-oxo-2,3-dihydro-6H-1-oxa-3a-aza-phenalene-5-carboxylic acid,
(Z)-9-[3-(2-Amino-1-methyl-ethylidene)-piperidin-1-yl]-8-fluoro-(3S)-3-methyl-6-oxo-2,3-dihydro-6H-1-oxa-3a-aza-phenalene-5-carboxylic acid,
(E)-7-[3-(2-Amino-1-fluoro-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(Z)-7-[3-(2-Amino-1-fluoro-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(E)-7-[3-(2-Amino-1-chloro-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(Z)-7-[3-(2-Amino-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(E)-7-[3-(2-Amino-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(Z)-7-[3-(2-Amino-1-methyl-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(Z)-1-(6-Amino-3,5-difluoro-pyridin-2-yl)-7-[3-(2-amino-1-fluoro-ethylidene)-piperidin-1-yl]-8-chloro-6-fluoro-4-oxo-1,4-dihydro-quinoline-3-carboxylic acid,
(E)-1-(6-Amino-3,5-difluoro-pyridin-2-yl)-7-[3-(2-amino-1-chloro-ethylidene)-piperidin-1-yl]8-chloro-6-fluoro-4-oxo-1,4-dihydro-quinoline-3-carboxylic acid,
(Z)-1-(6-Amino-3,5-difluoro-pyridin-2-yl)-7-[3-(2-amino-ethylidene)-piperidin-1-yl]8-chloro-6-fluoro-4-oxo-1,4-dihydro-quinoline-3-carboxylic acid,
(E)-1-(6-Amino-3,5-difluoro-pyridin-2-yl)-7-[3-(2-amino-ethylidene)-piperidin-1-yl]8-chloro-6-fluoro-4-oxo-1,4-dihydro-quinoline-3-carboxylic acid,
(E)-1-(6-Amino-3,5-difluoro-pyridin-2-yl)-8-chloro-6-fluoro-7-[3-(1-fluoro-2-hydroxy-ethylidene)-piperidin-1-yl]-4-oxo-1,4-dihydro-quinoline-3-carboxylic acid,
(Z)-1-(6-Amino-3,5-difluoro-pyridin-2-yl)-8-chloro-7-[3-(1-chloro-2-hydroxy-ethylidene)-piperidin-1-yl]-6-fluoro-4-oxo-1,4-dihydro-quinoline-3-carboxylic acid,
1-(6-Amino-3,5-difluoro-pyridin-2-yl)-8-chloro-6-fluoro-7-[4-(1-fluoro-2-hydroxy-ethylidene)-piperidin-1-yl]-4-oxo-1,4-dihydro-quinoline-3-carboxylic acid,
1-(6-Amino-3,5-difluoro-pyridin-2-yl)-8-chloro-6-fluoro-7-[3-(1-fluoro-2-methylamino-ethylidene)-azetidin-1-yl]-4-oxo-1,4-dihydro-quinoline-3-carboxylic acid,
1-(6-Amino-3,5-difluoro-pyridin-2-yl)-8-chloro-6-fluoro-7-[3-(1-fluoro-2-hydroxy-ethylidene)-azetidin-1-yl]-4-oxo-1,4-dihydro-quinoline-3-carboxylic acid,
1-(6-Amino-3,5-difluoro-pyridin-2-yl)-7-[3-(2-amino-ethylidene)-azetidin-1-yl]-8-chloro-6-fluoro-4-oxo-1,4-dihydro-quinoline-3-carboxylic acid,
1-(6-Amino-3,5-difluoro-pyridin-2-yl)-7-[3-(2-amino-1-methyl-ethylidene)-azetidin-1-yl]-8-chloro-6-fluoro-4-oxo-1,4-dihydro-quinoline-3-carboxylic acid,
(Z)-1-(6-Amino-3,5-difluoro-pyridin-2-yl)-7-[3-(2-amino-1-methyl-ethylidene)-piperidin-1-yl]-8-chloro-6-fluoro-4-oxo-1,4-dihydro-quinoline-3-carboxylic acid,
(E)-1-(6-Amino-3,5-difluoro-pyridin-2-yl)-7-[3-(2-amino-1-methyl-ethylidene)-piperidin-1-yl]-8-chloro-6-fluoro-4-oxo-1,4-dihydro-quinoline-3-carboxylic acid,
(E)-7-[3-(2-Amino-1-fluoro-ethylidene)-piperidin-1-yl]-1-cyclopropyl-6-fluoro-5-methyl-4-oxo-1,4-dihydro-quinoline-3-carboxylic acid,
(Z)-7-[3-(2-Amino-1-fluoro-ethylidene)-piperidin-1-yl]-1-cyclopropyl-6-fluoro-5-methyl-4-oxo-1,4-dihydro-quinoline-3-carboxylic acid,
(E)-7-[3-(2-Amino-1-chloro-ethylidene)-piperidin-1-yl]-1-cyclopropyl-6-fluoro-5-methyl-4-oxo-1,4-dihydro-quinoline-3-carboxylic acid,
(Z)-7-[3-(2-Amino-ethylidene)-piperidin-1-yl]-1-cyclopropyl-6-fluoro-5-methyl-4-oxo-1,4-dihydro-quinoline-3-carboxylic acid,
(E)-7-[3-(1-Chloro-2-methylamino-ethylidene)-piperidin-1-yl]-1-cyclopropyl-6-fluoro-4-oxo-1,4-dihydroquinoline-3-carboxylic acid,
1-Cyclopropyl-6-fluoro-7-[3-(1-fluoro-2-methylamino-ethylidene)-azetidin-1-yl]-4-oxo-1,4-dihydro-quinoline-3-carboxylic acid,
7-[3-(1-Chloro-2-methylamino-ethylidene)-azetidin-1-yl]-1-cyclopropyl-6-fluoro-4-oxo-1,4-dihydro-quinoline-3-carboxylic acid,
7-[4-(2-Amino-1-cyano-ethylidene)-piperidin-1-yl]-1-cyclopropyl-6-fluoro-8-methoxy-4-oxo-1,4-dihydro-quinoline-3-carboxylic acid,
9-[4-(2-Amino-1-cyano-ethylidene)-piperidin-1-yl]-8-fluoro-(3S)-3-methyl-6-oxo-2,3-dihydro-6H-1-oxa-3a-aza-phenalene-5-carboxylic acid,
7-[4-(2-Amino-1-cyano-ethylidene)-piperidin-1-yl]-1-cyclopropyl-8-difluoromethoxy-6-fluoro-4-oxo-1,4-dihydro-quinoline-3-carboxylic acid,
7-[4-(2-Amino-1-cyano-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(E)-7-[3-(2-Amino-1-cyano-ethylidene)-piperidin-1-yl]-1-cyclopropyl-6-fluoro-4-oxo-1,4-dihydro-[1,8]naphthyridine-3-carboxylic acid,
(Z)-7-[3-(2-Amino-1-cyano-ethylidene)-piperidin-1-yl]-1-cyclopropyl-6-fluoro-4-oxo-1,4-dihydro-[1,8]naphthyridine-3-carboxylic acid,
7-{4-[2-(2-Amino-acetylamino)-1-chloro-ethylidene]-piperidin-1-yl}-1-cyclopropyl-6-fluoro-8-methoxy-4-oxo-1,4-dihydro-quinoline-3-carboxylic acid,
(Z)-7-[3-(2-Amino-1-chloro-ethylidene)-piperidin-1-yl]-1-cyclopropyl-6-fluoro-8-methoxy-4-oxo-1,4-dihydro-quinoline-3-carboxylic acid,
(Z)-7-[3-(2-Amino-1-chloro-ethylidene)-piperidin-1-yl]-1-cyclopropyl-8-difluoromethoxy-6-fluoro-4-oxo-1,4-dihydro-quinoline-3-carboxylic acid,
(Z)-9-[3-(2-Amino-1-chloro-ethylidene)-piperidin-1-yl]-8-fluoro-(3S)-3-methyl-6-oxo-2,3-dihydro-6H-1-oxa-3a-aza-phenalene-5-carboxylic acid,
(Z)-7-[3-(2-Amino-1-chloro-ethylidene)-piperidin-1-yl]-6-fluoro-(1R,2S)-1-(2-fluoro-cyclopropyl)-8-methoxy-4-oxo-1,4-dihydro-quinoline-3-carboxylic acid,
(Z)-7-[3-(2-Amino-1-chloro-ethylidene)-piperidin-1-yl]-1-(6-amino-3,5-difluoro-pyridin-2-yl)-8-chloro-6-fluoro-4-oxo-1,4-dihydro-quinoline-3-carboxylic acid,
(E)-7-[3-(2-Amino-ethylidene)-piperidin-1-yl]-1-cyclopropyl-6-fluoro-5-methyl-4-oxo-1,4-dihydro-quinoline-3-carboxylic acid,
(Z)-7-[3-(2-Amino-1-chloro-ethylidene)-piperidin-1-yl]-1-cyclopropyl-6-fluoro-5-methyl-4-oxo-1,4-dihydro-quinoline-3-carboxylic acid,
(E)-1-Cyclopropyl-6-fluoro-7-[3-(1-fluoro-2-methylamino-ethylidene)-piperidin-1-yl]-8-methoxy-4-oxo-1,4-dihydro-quinoline-3-carboxylic acid,
7-[4-(2-Amino-1-chloro-ethylidene)-piperidin-1-yl]-1-cyclopropyl-6-fluoro-8-methoxy-5-methyl-4-oxo-1,4-dihydro-quinoline-3-carboxylic acid,
5-Amino-7-[(E)-3-(2-amino-1-fluoro-ethylidene)-piperidin-1-yl]-1-cyclopropyl-6,8-difluoro-4-oxo-1,4-dihydro-quinoline-3-carboxylic acid,
7-[(E)-3-(2-Amino-1-fluoro-ethylidene)-piperidin-1-yl]-1-cyclopropyl-6-fluoro-8-fluoromethoxy-4-oxo-1,4-dihydro-quinoline-3-carboxylic acid,
7-[(E)-3-(2-Amino-1-fluoro-ethylidene)-piperidin-1-yl]-1-cyclopropyl-6-fluoro-8-methoxy-5-methyl-4-oxo-1,4-dihydro-quinoline-3-carboxylic acid,
7-[(E)-3-(2-Amino-1-fluoro-ethylidene)-piperidin-1-yl]-1-cyclopropyl-6-fluoro-8-methyl-4-oxo-1,4-dihydro-quinoline-3-carboxylic acid,
7-{4-[3-Amino-1-fluoro-propylidene]-piperidin-1-yl}-1-cyclopropyl-6-fluoro-8-methoxy-4-oxo-1,4-dihydro-quinoline-3-carboxylic acid,
(3S)-9-[(Z)-4-(2-Amino-1-fluoro-ethylidene)-azepan-1-yl]-8-fluoro-3-methyl-6-oxo-2,3-dihydro-6H-1-oxa-3a-aza-phenalene-5-carboxylic acid,
(3S)-9-[(E)-4-(2-Amino-1-fluoro-ethylidene)-azepan-1-yl]-8-fluoro-3-methyl-6-oxo-2,3-dihydro-6H-1-oxa-3a-aza-phenalene-5-carboxylic acid,
9-[(Z)-6-(2-Amino-1-fluoro-ethylidene)-[1,4]oxazepan-4-yl]-8-fluoro-(3S)-3-methyl-6-oxo-2,3-dihydro-6H-1-oxa-3a-aza-phenalene-5-carboxylic acid,
9-[(E)-6-(2-Amino-1-fluoro-ethylidene)-[1,4]oxazepan-4-yl]-8-fluoro-(3S)-3-methyl-6-oxo-2,3-dihydro-6H-1-oxa-3a-aza-phenalene-5-carboxylic acid,
(Z)-7-[4-(2-Amino-1-fluoro-ethylidene)-azepan-1-yl]-1-cyclopropyl-6-fluoro-4-oxo-1,4-dihydro-[1,8]naphthyridine-3-carboxylic acid,
(E)-7-[4-(2-Amino-1-fluoro-ethylidene)-azepan-1-yl]-1-cyclopropyl-6-fluoro-4-oxo-1,4-dihydro-[1,8]naphthyridine-3-carboxylic acid,
(Z)-7-[6-(2-Amino-1-fluoro-ethylidene)-[1,4]oxazepan-4-yl]-1-cyclopropyl-6-fluoro-4-oxo-1,4-dihydro-[1,8]naphthyridine-3-carboxylic acid,
(E)-7-[6-(2-Amino-1-fluoro-ethylidene)-[1,4]oxazepan-4-yl]-1-cyclopropyl-6-fluoro-4-oxo-1,4-dihydro-[1,8]naphthyridine-3-carboxylic acid,
(Z)-7-[6-(2-Amino-1-fluoro-ethylidene)-(2S)-2-methyl-[1,4]oxazepan-4-yl]-1-cyclopropyl-6-fluoro-4-oxo-1,4-dihydro-[1,8]naphthyridine-3-carboxylic acid,
(Z)-7-[6-(2-Amino-1-fluoro-ethylidene)-(2R)-2-methyl-[1,4]oxazepan-4-yl]-1-cyclopropyl-6-fluoro-4-oxo-1,4-dihydro-[1,8]naphthyridine-3-carboxylic acid,
(Z)-7-[4-(2-Amino-1-fluoro-ethylidene)-azepan-1-yl]-1-cyclopropyl-6-fluoro-4-oxo-1,4-dihydro-quinoline-3-carboxylic acid,
(E)-7-[4-(2-Amino-1-fluoro-ethylidene)-azepan-1-yl]-1-cyclopropyl-6-fluoro-4-oxo-1,4-dihydro-quinoline-3-carboxylic acid,
(Z)-7-[6-(2-Amino-1-fluoro-ethylidene)-[1,4]oxazepan-4-yl]-1-cyclopropyl-6-fluoro-4-oxo-1,4-dihydro-quinoline-3-carboxylic acid,
(E)-7-[6-(2-Amino-1-fluoro-ethylidene)-[1,4]oxazepan-4-yl]-1-cyclopropyl-6-fluoro-4-oxo-1,4-dihydro-quinoline-3-carboxylic acid,
(Z)-7-[4-(2-Amino-1-fluoro-ethylidene)-azepan-1-yl]-1-cyclopropyl-6-fluoro-8-methoxy-4-oxo-1,4-dihydro-quinoline-3-carboxylic acid,
(E)-7-[4-(2-Amino-1-fluoro-ethylidene)-azepan-1-yl]-1-cyclopropyl-6-fluoro-8-methoxy-4-oxo-1,4-dihydro-quinoline-3-carboxylic acid,
(Z)-7-[6-(2-Amino-1-fluoro-ethylidene)-[1,4]oxazepan-4-yl]-1-cyclopropyl-6-fluoro-8-methoxy-4-oxo-1,4-dihydro-quinoline-3-carboxylic acid, and
(E)-7-[6-(2-Amino-1-fluoro-ethylidene)-[1,4]oxazepan-4-yl]-1-cyclopropyl-6-fluoro-8-methoxy-4-oxo-1,4-dihydro-quinoline-3-carboxylic acid.
or an optical isomer, diastereomer or enantiomer thereof or a pharmaceutically acceptable salt, hydrate, or prodrug thereof.

16. The compound of claim 15 or a pharmaceutically acceptable salt thereof selected from the group consisting of a trifluoroacetate or hydrochloride salt.

17. A compound of claim 1 selected from the group consisting of:

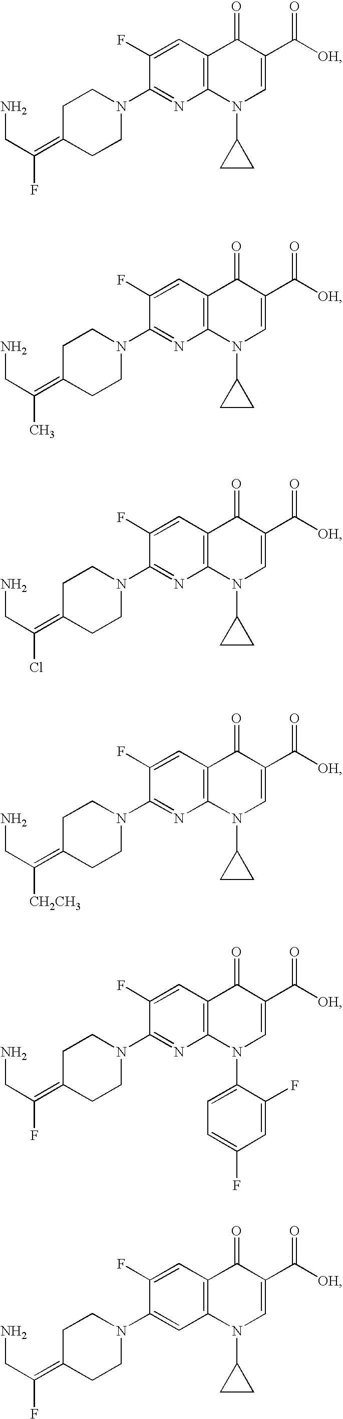

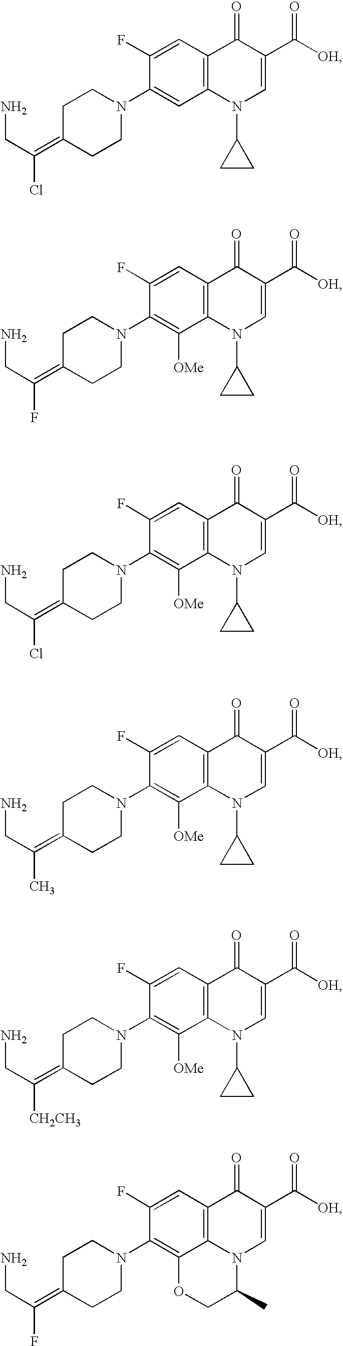

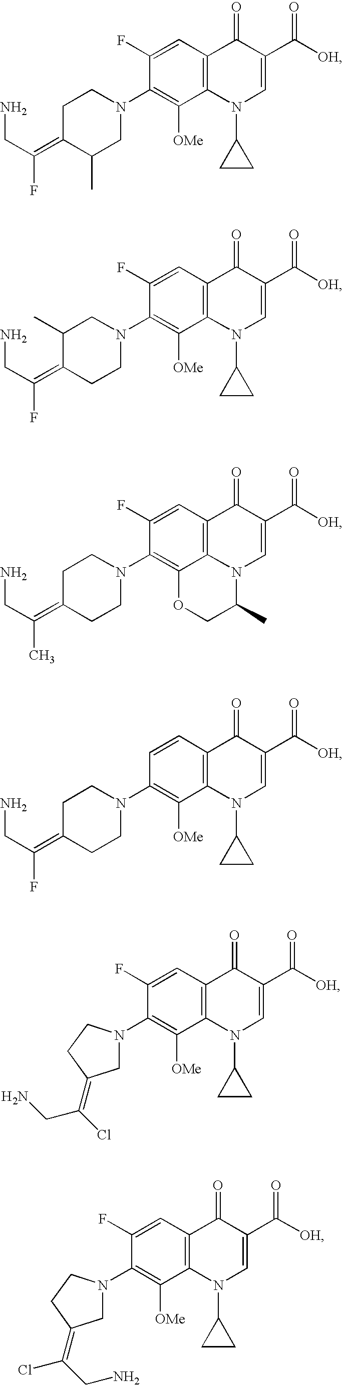

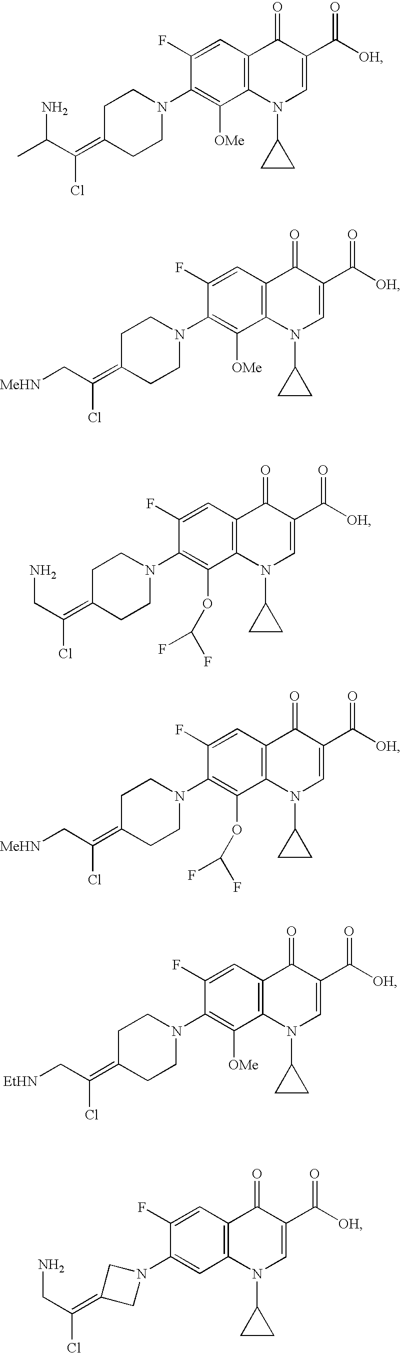

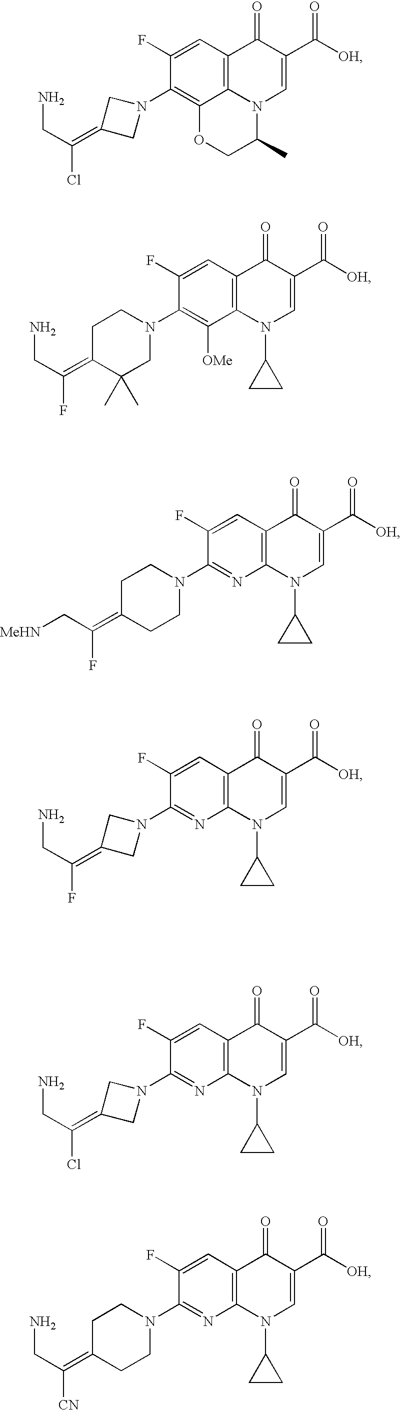

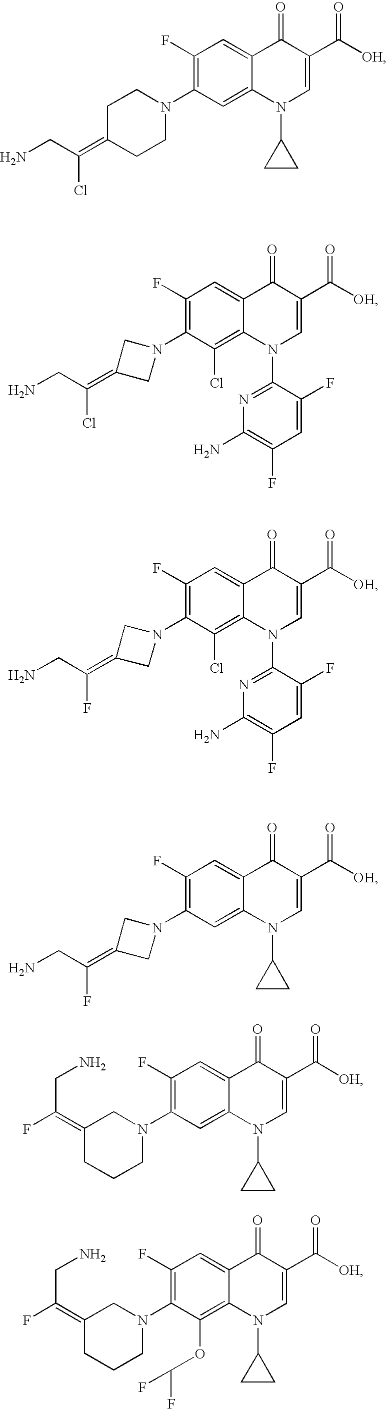

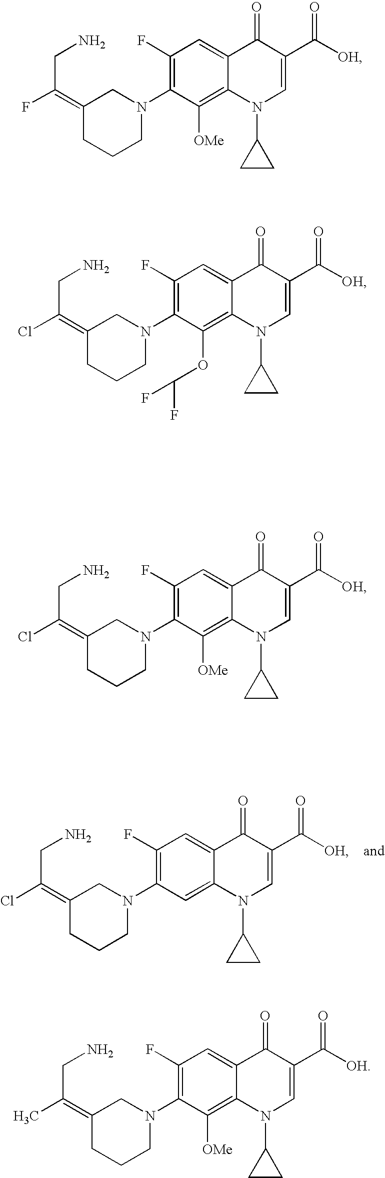

18. The compound of claim 1 having the formula:

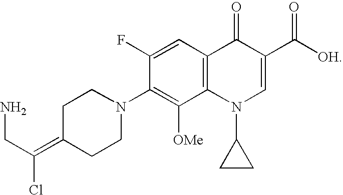

19. The compound of claim 1 having the formula:

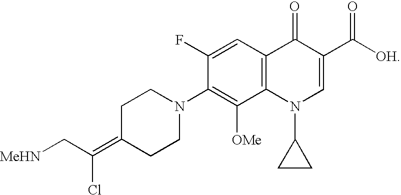

20. The compound of claim 1 having the formula:

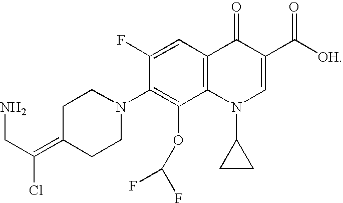

21. A method of treating a subject having a condition caused by or contributed to by bacterial infection, which comprises administering to said mammal a therapeutically effective amount of the compound according to claim 1.

22. A method of treating a subject having a condition caused by or contributed to by bacterial infection, which comprises administering to said mammal a therapeutically effective amount of the compound according to claim 11.

23. A method of preventing a subject from suffering from a condition caused by or contributed to by bacterial infection, which comprises administering to the subject a prophylactically effective dose of a compound according to claim 1.

24. A method of preventing a subject from suffering from a condition caused by or contributed to by bacterial infection, which comprises administering to the subject a prophylactically effective dose of a compound according to claim 11.
